SEQUENCE LISTING (137 sequences)

SEQ ID NO: 1 - protein, 181 aa, synthetic construct
NSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVS

SEQ ID NO: 2 - protein, 454 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 3 - protein, 15 aa, synthetic construct
EPKSCDKTHTCPPCP

SEQ ID NO: 4 - protein, 760 aa, Homo sp.
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 5 - protein, 630 aa, Homo sapiens
MELQPPEASIAVVSIPRQLPGSHSEAGVQGLSAGDDSELGSHCVAQTGLELLASGDPLPSASQNAEMIETGSDCVTQAGLQLLASSDPPALASKNAEVTGTMSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 6 - protein, 220 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 7 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 8 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 9 - protein, 220 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWVNYKTTPPVLDSDGSFFLYSKLTVTKEEWQQGFVFSCSVMHEALHNHYTQKSLSLSPGK

SEQ ID NO: 10 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWVNYKTTPPVLDSDGSFFLYSKLTVTKEEWQQGFVFSCSVMHEALHNHYTQKSLSLSPGK

SEQ ID NO: 11 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWVNYKTTPPVLDSDGSFFLYSKLTVTKEEWQQGFVFSCSVMHEALHNHYTQKSLSLSPGK

SEQ ID NO: 12 - protein, 220 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 13 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 14 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWSNYKTTPPVLDSDGSFFLYSKLTVSKEEWQQGFVFSCSVMHEALHNHYTQKSLSLSPGK

SEQ ID NO: 15 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSRFVLENNYKTTPPVLDSDGSFFLYSKLTVDKSRWQQGNIFACNVYHEALHNHYTFKNLALSPGK

SEQ ID NO: 16 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSRFVLENNYKTTPPVLDSDGSFFLYSKLTVDKSRWQQGEIFACNVYHEALHNHYTFKNLALSPGK

SEQ ID NO: 17 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSQYELENNYKTTPPVLDSDGSFFLYSKLTVDKSRWQQGNVFACNVYHEALHNHYTFKNLALSPGK

SEQ ID NO: 18 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSHYELENNYKTTPPVLDSDGSFFLYSKLTVDKSRWQQGNVFACNVYHEALHNHYTFKNLALSPGK

SEQ ID NO: 19 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSRFVLENNYKTTPPVLDSDGSFFLYSKLTVDKSRWQQGNVFACNVYHEALHNHYTFKNLALSPGK

SEQ ID NO: 20 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSRFVLENNYKTTPPVLDSDGSFFLYSKLTVDKSRWQQGEVFACNVYHEALHNHYTFKNLALSPGK

SEQ ID NO: 21 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSRFVLENNYKTTPPVLDSDGSFFLYSKLTVDKSRWQQGEIFACNVYHEALHNHYTFKNLSLSPGK

SEQ ID NO: 22 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSRFVLENNYKTTPPVLDSDGSFFLYSKLTVDKSRWQQGEIFACNVYHEALHNHYTFKNLRLSPGK

SEQ ID NO: 23 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSRFVLENNYKTTPPVLDSDGSFFLYSKLTVDKSRWQQGEIFACNVYHEALHNHYTFKNLHLSPGK

SEQ ID NO: 24 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSRFVLENNYKTTPPVLDSDGSFFLYSKLTVDKSRWQQGEIFACNVYHEALHNHRTFKNLRLSPGK

SEQ ID NO: 25 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSHYELENNYKTTPPVLDSDGSFFLYSKLTVDKSRWQQGEIFACNVYHEALHNHYTFKNLALSPGK

SEQ ID NO: 26 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSQFHLENNYKTTPPVLDSDGSFFLYSKLTVDKSRWQQGNIFACNVYHEALHNHYTFKNLLLSPGK

SEQ ID NO: 27 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSRFVLENNYKTTPPVLDSDGSFFLYSKLTVDKSRWQQGNTFACNVYHEALHNHYTFKNLALSPGK

SEQ ID NO: 28 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSRFVLENNYKTTPPVLDSDGSFFLYSKLTVDKSRWQQGEIFACNVYHEALHNHYTFKNLKLSPGK

SEQ ID NO: 29 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSRFVLENNYKTTPPVLDSDGSFFLYSKLTVDKSRWQQGEIFACNVYHEALHNHWTFKNLRLSPGK

SEQ ID NO: 30 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWNSQYLLENNYKTTPPVLDSDGSFFLYSKLTVDKSRWQQGEIFACNVYHEALHNHYTFKNLALSPGK

SEQ ID NO: 31-33 - skipped (3 entries)

SEQ ID NO: 34 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWRTYKPYETNYKTTPPVLDSDGSFFLYSKLTVDKSRWQQGDIFVCDVMHEALHNHFTIKKLSLSPGK

SEQ ID NO: 35 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWRTYKPYETNYKTTPPVLDSDGSFFLYSKLTVDKSRWQQGDIFVCDVLHEALHNHFTIKKLSLSPGK

SEQ ID NO: 36 - protein, 125 aa, synthetic construct
QVQLVESGGGLVQAGGSLRLSCAASGRAFSSNGMGWFRQAPGKEREFVATISCSGTSTYYADSVKGRFTISRDNAKNTVYLQMNSLKPEDTAVYFCAADTRHCMGYTKDEGYAYWGQGTQVTVSS

SEQ ID NO: 37 - protein, 8 aa, synthetic construct
GRAFSSNG

SEQ ID NO: 38 - protein, 8 aa, synthetic construct
ISCSGTST

SEQ ID NO: 39 - protein, 16 aa, synthetic construct
AADTRHCMGYTKDEGY

SEQ ID NO: 40 - protein, 247 aa, synthetic construct
QVQLVESGGGVVQPGRSLRLSCAASGFTFSSYGMHWVRQAPGKGLEWVAIISYDGSNKHYADSVKGRFTISRDNSKNTLYLQMNSLRAEDTAVYYCARAPSSFYFDYWGQGTLVTVSSGTEGKSSGSGSESKSTDIVMTQSPDSLAVSLGERATINCKSSQSVLFSSNNKNYLAWYQQKPGQPPKLLIYWASTRESGVPDRFSGSGSGTDFTLTISSLQAEDVAVYYCQQYYSTPPTFGQGTKVVIK

SEQ ID NO: 41 - protein, 8 aa, synthetic construct
GFTFSSYG

SEQ ID NO: 42 - protein, 8 aa, synthetic construct
ISYDGSNK

SEQ ID NO: 43 - protein, 11 aa, synthetic construct
ARAPSSFYFDY

SEQ ID NO: 44 - protein, 12 aa, synthetic construct
QSVLFSSNNKNY

SEQ ID NO: 45 - skipped

SEQ ID NO: 46 - protein, 9 aa, synthetic construct
QQYYSTPPT

SEQ ID NO: 47 - protein, 238 aa, synthetic construct
QVQLQESGPGLVKPSETLSLTCTVSGGSISSSYWSWIRQPPGKGLEWIGYIYNSGSTNYNPSLKSRVTISVDTSKNQFSLKLSSVTAADTAVYYCARETYYFDYWGQGTLVTVSSGTEGKSSGSGSESKSTDIVMTQSQKFMSTSVGDRVSVTCKASQNVGINVAWYQKKPGQPPKALIYSAAYRYSGVPDRFTGSGSGTDFTLTISNVQSEDLAEYFCQQYNSYPLTFGSGTKLEIK

SEQ ID NO: 48 - protein, 8 aa, synthetic construct
GGSISSSY

SEQ ID NO: 49 - protein, 7 aa, synthetic construct
IYNSGST

SEQ ID NO: 50 - protein, 9 aa, synthetic construct
ARETYYFDY

SEQ ID NO: 51 - protein, 6 aa, synthetic construct
QNVGIN

SEQ ID NO: 52 - skipped

SEQ ID NO: 53 - protein, 9 aa, synthetic construct
QQYNSYPLT

SEQ ID NO: 54 - protein, 8 aa, synthetic construct
  VAR_SEQ 1: note = X is S or T
  VAR_SEQ 2: note = X is E or R
  VAR_SEQ 7: note = X is any amino acid
XXYKPYXT

SEQ ID NO: 55-56 - skipped (2 entries)

SEQ ID NO: 57 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWFSGGAKETNYKTTPPVLDSDGSFFLYSKLTVDKSRWQQGNLFACEVMHEALHNHYTYKLLSLSPGK

SEQ ID NO: 58 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWFSDGSKETNYKTTPPVLDSDGSFFLYSKLTVDKSRWQQGNLFACEVMHEALHNHYTYKLLSLSPGK

SEQ ID NO: 59 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWFYDDSKLTNYKTTPPVLDSDGSFFLYSKLTVDKSRWQPRGLFACEVMHEALHNHYTYKLLGESPGK

SEQ ID NO: 60 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWFYGNAKETNYKTTPPVLDSDGSFFLYSKLTVDKSRWQQGNLFACEVMHEALHNHYTYKLLSLSPGK

SEQ ID NO: 61 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWFYEAQKLNNYKTTPPVLDSDGSFFLYSKLTVDKSRWQQGNLFACEVMHEALHNHYTYKLLSLSPGK

SEQ ID NO: 62 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWFSEGSKETNYKTTPPVLDSDGSFFLYSKLTVDKSRWQQGNLFACEVMHEALHNHYTYKLLSLSPGK

SEQ ID NO: 63 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWFSGAQKESNYKTTPPVLDSDGSFFLYSKLTVDKSRWQQGNLFACEVMHEALHNHYTYKLLSLSPGK

SEQ ID NO: 64 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWFSGAQKLTNYKTTPPVLDSDGSFFLYSKLTVDKSRWQQGNLFACEVMHEALHNHYTYKLLSLSPGK

SEQ ID NO: 65 - protein, 448 aa, synthetic construct
EVQLVQSGAEVKKPGESLKISCKGSGYSFTNYWLGWVRQMPGKGLEWMGDIYPGGDYPTYSEKFKVQVTISADKSISTAYLQWSSLKASDTAMYYCARSGNYDEVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 219 aa, synthetic construct
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQSTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 221 aa, synthetic construct
EVQLVQSGAEVKKPGESLKISCKGSGYSFTNYWLGWVRQMPGKGLEWMGDIYPGGDYPTYSEKFKVQVTISADKSISTAYLQWSSLKASDTAMYYCARSGNYDEVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 68 - protein, 219 aa, synthetic construct; identical to SEQ ID NO: 66

SEQ ID NO: 69 - protein, 118 aa, synthetic construct
EVQLVQSGAEVKKPGESLKISCKGSGYSFTNYWLGWVRQMPGKGLEWMGDIYPGGDYPTYSEKFKVQVTISADKSISTAYLQWSSLKASDTAMYYCARSGNYDEVAYWGQGTLVTVSS

SEQ ID NO: 70 - protein, 112 aa, synthetic construct
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQSTHVPWTFGQGTKVEIK

SEQ ID NO: 71 - protein, 10 aa, synthetic construct
GYSFTNYWLG

SEQ ID NO: 72 - protein, 17 aa, synthetic construct
DIYPGGDYPTYSEKFKV

SEQ ID NO: 73 - protein, 9 aa, synthetic construct
SGNYDEVAY

SEQ ID NO: 74 - protein, 16 aa, synthetic construct
RSSQSLVHSNGNTYLH

SEQ ID NO: 75 - protein, 7 aa, synthetic construct
KVSNRFS

SEQ ID NO: 76 - protein, 9 aa, synthetic construct
SQSTHVPWT

SEQ ID NO: 77 - protein, 448 aa, synthetic construct
EVQLVQSGAEVKKPGESLKISCKGSGYSFMNYWLGWVRQMPGKGLEWMGDIYPGGDYPTYSEKFKVQVTISADKSISTAYLQLSSLKASDTAMYYCARSGNYDEVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 219 aa, synthetic construct; identical to SEQ ID NO: 66

SEQ ID NO: 79 - protein, 221 aa, synthetic construct
EVQLVQSGAEVKKPGESLKISCKGSGYSFMNYWLGWVRQMPGKGLEWMGDIYPGGDYPTYSEKFKVQVTISADKSISTAYLQLSSLKASDTAMYYCARSGNYDEVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 80 - protein, 219 aa, synthetic construct; identical to SEQ ID NO: 66

SEQ ID NO: 81 - protein, 118 aa, synthetic construct
EVQLVQSGAEVKKPGESLKISCKGSGYSFMNYWLGWVRQMPGKGLEWMGDIYPGGDYPTYSEKFKVQVTISADKSISTAYLQLSSLKASDTAMYYCARSGNYDEVAYWGQGTLVTVSS

SEQ ID NO: 82 - protein, 112 aa, synthetic construct; identical to SEQ ID NO: 70

SEQ ID NO: 83 - protein, 10 aa, synthetic construct
GYSFMNYWLG

SEQ ID NO: 84 - protein, 17 aa, synthetic construct
DIYPGGDYPTYSEKFKV

SEQ ID NO: 85 - protein, 9 aa, synthetic construct
SGNYDEVAY

SEQ ID NO: 86 - protein, 16 aa, synthetic construct
RSSQSLVHSNGNTYLH

SEQ ID NO: 87 - protein, 7 aa, synthetic construct
KVSNRFS

SEQ ID NO: 88 - protein, 9 aa, synthetic construct
SQSTHVPWT

SEQ ID NO: 89 - protein, 229 aa, synthetic construct
QSMQESGPGLVKPSQTLSLTCTVSGFSLSSYAMSWIRQHPGKGLEWIGYIWSGGSTDYASWAKSRVTISKTSTTVSLKLSSVTAADTAVYYCARRYGTSYPDYGDASGFDPWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 215 aa, synthetic construct
AIQLTQSPSSLSASVGDRVTITCRASQSISSYLAWYQQKPGKAPKLLIYRASTLASGVPSRFSGSGSGTDFTLTISSLQPEDFATYYCQQNYASSNVDNTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 91 - protein, 10 aa, synthetic construct
GFSLSSYAMS

SEQ ID NO: 92 - protein, 16 aa, synthetic construct
YIWSGGSTDYASWAKS

SEQ ID NO: 93 - protein, 17 aa, synthetic construct
RYGTSYPDYGDASGFDP

SEQ ID NO: 94 - protein, 11 aa, synthetic construct
RASQSISSYLA

SEQ ID NO: 95 - protein, 7 aa, synthetic construct
RASTLAS

SEQ ID NO: 96 - protein, 12 aa, synthetic construct
QQNYASSNVDNT

SEQ ID NO: 97 - protein, 446 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 446 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPCVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 99 - protein, 214 aa, synthetic construct
DIQMTQSPSSLSASVGDRVTITCRTSENIYNNLAWYQQKPGKSPKLLIYAATNLADGVPSRFSGSGSGTDYTLTISSLQPEDFATYYCQHFWGTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 219 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 101 - protein, 214 aa, synthetic construct; identical to SEQ ID NO: 99

SEQ ID NO: 102 - protein, 116 aa, synthetic construct
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSS

SEQ ID NO: 103 - protein, 107 aa, synthetic construct
DIQMTQSPSSLSASVGDRVTITCRTSENIYNNLAWYQQKPGKSPKLLIYAATNLADGVPSRFSGSGSGTDYTLTISSLQPEDFATYYCQHFWGTPLTFGGGTKVEIK

SEQ ID NO: 104 - protein, 10 aa, synthetic construct
GYTFTNYWMH

SEQ ID NO: 105 - protein, 17 aa, synthetic construct
EINPINGRSNYAEKFQG

SEQ ID NO: 106 - protein, 9 aa, synthetic construct
ARGTRAMHY

SEQ ID NO: 107 - protein, 11 aa, synthetic construct
RTSENIYNNLA

SEQ ID NO: 108 - protein, 7 aa, synthetic construct
AATNLAD

SEQ ID NO: 109 - protein, 9 aa, synthetic construct
QHFWGTPLT

SEQ ID NO: 110 - protein, 243 aa, synthetic construct
QVQLQQWGAGLLKPSETLSLTCAVYGGSFSGYYWTWIRQPPGKGLEWIGEFIHSGSTNYNPSLKSRVTISVDTSKNQFSLKLSSVTAADTAVYFCARGSMDSSGFYSFDFWGQGTLVTVSSGTEGKSSGSGSESKSTSYEVTQPPSVSVSPGQTASITCSGHKLGDKFASWYQQKPGQSPVLVIYQDRKRPSGIPERFSGSNSGNTATLTISGTQAMDEADYYCQTWYSSTVIFGGGTKLTVL

SEQ ID NO: 111 - protein, 8 aa, synthetic construct
GGSFSGYY

SEQ ID NO: 112 - protein, 7 aa, synthetic construct
FIHSGST

SEQ ID NO: 113 - protein, 15 aa, synthetic construct
ARGSMDSSGFYSFDF

SEQ ID NO: 114 - protein, 6 aa, synthetic construct
KLGDKF

SEQ ID NO: 115 - protein, 6 aa, synthetic construct
QDRKRP

SEQ ID NO: 116 - protein, 9 aa, synthetic construct
QTWYSSTVI

SEQ ID NO: 117 - protein, 244 aa, synthetic construct
EVQLLESGGGLVQPGGSLRLSCADSGFTFSSYAMNWVRQAPGKGLEWVSGISGSGGHTYYADSVKGRFTVSRDNSKNTLYLQMNSLRAEDTAVYYCAREGYDSSGYNPFDYWGQGTQVTVSSGTEGKSSGSGSESKSTSYELTQPPSVSVSPGQTASITCSGDKLGDKYASWYQQKPGQSPVLVIYQDSKRPSGIPERFSGSNSGNTATLTISGTQAMDEADYYCQAWDSSTVVFGGGTKLTVL

SEQ ID NO: 118 - protein, 11 aa, synthetic construct
GFTFSSYAMNW

SEQ ID NO: 119 - protein, 8 aa, synthetic construct
ISGSGGHT

SEQ ID NO: 120 - protein, 15 aa, synthetic construct
AREGYDSSGYNPFDY

SEQ ID NO: 121 - protein, 7 aa, synthetic construct
KLGDKYA

SEQ ID NO: 122 - protein, 7 aa, synthetic construct
QDSKRPS

SEQ ID NO: 123 - protein, 9 aa, synthetic construct
QAWDSSTVV

SEQ ID NO: 124 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWTNYKTTPPVLDSDGSFFLYSKLTVTKSEWQQGFVFSCSVMHEALHNHYTQKSLSLSPGK

SEQ ID NO: 125 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWVNYKTTPPVLDSDGSFFLYSKLTVTKSEWQQGFVFSCSVMHEALHNHYTQKSLSLSPGK

SEQ ID NO: 126 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWFYGDSKLTNYKTTPPVLDSDGSFFLYSKLTVDKSRWQQGNLFACEVMHEALHNHYTYKLLSLSPGK

SEQ ID NO: 127 - protein, 220 aa, synthetic construct
EVQLVESGGGLVQPGGSLRLSCAVSGFSLTTYGVDWVRQAPGKGLEWVGIIWGDGTTHYHSALISRFTISKDNSKNTVYLQMNSLRAEDTAVYYCAKPGGASWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 128 - protein, 214 aa, synthetic construct
DIQMTQSPSSLSASVGDRVTITCKASQLVGTNVAWYQQKPGKSPKALFYSASYRNQGVPSRFSGSGSGTDFTLTISSLQPEDFATYFCQHYNSYPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 220 aa, synthetic construct; identical to SEQ ID NO: 127

SEQ ID NO: 130 - protein, 214 aa, synthetic construct
DIQMTQSPSSLSASVGDRVTITCKASQFVGTAVAWYQQKPGKSPKALFYSASYRNEGVPSRFSGSGSGTDFTLTISSLQPEDFATYFCQHYNSYPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 240 aa, synthetic construct
EVQLVESGGGLVQPGGSLRLSCAVSGFSLTTYGVDWVRQAPGKGLEWVGIIWGDGTTHYHSALISRFTISKDNSKNTVYLQMNSLRAEDTAVYYCAKPGGASWGQGTLVTVSSGGGGSGGGGSGGGGSDIQMTQSPSSLSASVGDRVTITCKASQLVGTNVAWYQQKPGKSPKALFYSASYRNQGVPSRFSGSGSGTDFTLTISSLQPEDFATYFCQHYNSYPFTFGQGTKVEIKEPKSS

SEQ ID NO: 132 - protein, 240 aa, synthetic construct
EVQLVESGGGLVQPGGSLRLSCAVSGFSLTTYGVDWVRQAPGKGLEWVGIIWGDGTTHYHSALISRFTISKDNSKNTVYLQMNSLRAEDTAVYYCAKPGGASWGQGTLVTVSSGGGGSGGGGSGGGGSDIQMTQSPSSLSASVGDRVTITCKASQFVGTAVAWYQQKPGKSPKALFYSASYRNEGVPSRFSGSGSGTDFTLTISSLQPEDFATYFCQHYNSYPFTFGQGTKVEIKEPKSS

SEQ ID NO: 133 - protein, 122 aa, synthetic construct
EVQLVESGGGLVQPGGSLRLSCVASGIIFSNHAVSWYRRAPGKQREVVAFLGSGTSTYYADSVKGRFTISRDNAKNTGYLQMNSLKPEDTGVYYCNVYRYEGPGTQVTVSSASEPKSSDKTH

SEQ ID NO: 134 - protein, 122 aa, synthetic construct
EVQLVESGGGLVQPGGSLRLSCVASGIIFSNHAVSWYRRAPGKQREVVAFLGSGSSTYYADSVKGRFTISRDNAKNTGYLQMNSLKPEDTGVYYCNVYRYEGPGTQVTVSSASEPKSSDKTH

SEQ ID NO: 135 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVLWSSRYVLENNYKTTPPVLDSDGSFFLYSKLTVDKSRWQQGNVFACNVYHEALHNHYTFKNLALSPGK

SEQ ID NO: 136 - protein, 111 aa, synthetic construct
EVQLVESGGGLVQPGGSLRLSCVASGIIFSNHAVSWYRRAPGKQREVVAFLGSGNSYYYADSVKGRFTISRDNAKNTGYLQMNSLKPEDTGVYYCNVYRYEGPGTQVTVSS

SEQ ID NO: 137 - protein, 111 aa, synthetic construct
EVQLVESGGGLVQPGGSLRLSCVASGIIFSNHAVSWYRRAPGKQREVVAFLGSGNSYYYADSVKGRFTISRDNAKNTGYLQMNSLKPEDTGVYYCNVARYEGPGTQVTVSS

(19) United States
(12) Patent Application Publication
(10) Pub. No.: US 2026/0308269 A1
(43) Pub. Date: Oct. 8, 2026

(54) DUAL TRANSPORTERS AND METHODS OF USE

(71) Applicant: Denali Therapeutics Inc., South San Francisco, CA (US)

(72) Inventors: Padma AKKAPEDDI, Millbrae, CA (US); Darren CHAN, South San Francisco, CA (US); Gerald Maxwell CHERF, Encinitas, CA (US); Kylie S. CHEW, Los Altos, CA (US); Mark S. DENNIS, San Carlos, CA (US); Mihalis S. KARIOLIS, San Mateo, CA (US); Arash MOSHKFOROUSH, San Francisco, CA (US); Robert G. THORNE, San Francisco, CA (US); Robert C. WELLS, Foster City, CA (US); Joy Yu ZUCHERO, San Mateo, CA (US)

(21) Appl. No.: 19/485,744

(22) PCT Filed: Jun. 18, 2024
(86) PCT No.: PCT/US2024/034457

§ 371 Date: Nov. 19, 2025

Related U.S. Application Data
(60) Provisional application No. 63/509,148, filed on Jun. 20, 2023.

(51) Int. Cl. C07K 16/28; A61K 39/00; A61K 47/68; A61P 25/28; C07K 14/78

(52) U.S. Cl. CPC C07K 16/2881; A61K 47/6879; A61P 25/28; C07K 14/78; C07K 16/2896; A61K 2039/505; C07K 2317/31; C07K 2317/526; C07K 2317/55; C07K 2317/565; C07K 2317/622; C07K 2317/92

(57) ABSTRACT

Described are dual transporters and methods of making and using the dual transporters. The dual transporters bind to a blood brain barrier transport protein and a second protein that is expressed on the luminal surface of the blood brain barrier or is a brain retention protein. The dual transporters improve delivery to the central nervous system.

### CROSS-REFERENCE TO RELATED APPLICATIONS

This application claims the benefit of U.S. Provisional Application No. 63/509,148, filed Jun. 20, 2023, which is incorporated herein by reference.

### SEQUENCE LISTING

The Sequence Listing written in file DNL-027-05_SeqListing.xml is 131 kilobytes in size, was created Jun. 14, 2024, and is hereby incorporated by reference.

### BACKGROUND

The blood-brain barrier (BBB) blocks the passage of most macromolecules from the periphery into the brain and thus limits the uses of large molecule therapeutics where brain exposure is required. The BBB thus presents a significant obstacle in delivering therapeutic molecules to the brain or central nervous system (CNS) to treat neurological disorders or conditions of the CNS. In addition to the need for improved delivery of therapeutic molecules across the BBB, improved peripheral delivery to specific tissues is also desired.

Moieties that bind individual receptors, such as the transferrin receptor (TfR) or CD98hc, have been used to deliver cargo across the BBB. Nevertheless, improved compositions and methods that enhance transport across the BBB or are directed to specific peripheral tissues beyond what these individual binding moieties alone provide is therefore desired.

### SUMMARY

Described are dual transporters comprising a first binding region that specifically binds to a BBB transport protein and a second binding region that specifically binds to a second protein, wherein the second protein is expressed on the luminal surface of the BBB or is a brain retention protein, and wherein the second protein is different from the BBB transport protein. In some embodiments, the first binding region specifically binds a BBB transport protein and increases transport of the dual transporter (or a molecule linked to the dual transporter) across the BBB, and the second binding protein specifically binds the second protein and increases retention (i.e., exposure time) of the dual transporter (or a molecule linker to the dual transported) in the CNS. In some embodiments, the BBB transport protein is the transferrin receptor 1 (TfR) or CD98hc (also termed 4F2 cell-surface antigen heavy chain). In some embodiments, the BBB transport protein is alkaline phosphatase (ALPL). The second protein can be a BBB surface protein (a protein expressed on the luminal surface of brain endothelial cells comprising the BBB) or a brain retention protein. In some embodiments, the BBB surface protein is a second BBB transport protein. In some embodiments, the second protein is selected from the group consisting of TfR, CD98hc, Large neutral amino acids transporter small subunit 1 (CD98 light chain or CD981c), glucose transporter 1 (GLUT1), major facilitator superfamily domain-containing protein 2A (MFSD2A), carbonic anhydrase IV (CA-IV), alkaline phosphatase (ALPL). Low density lipoprotein receptor. Insulin-like growth factor 1 receptor (IGFTR), Insulin-like growth factor 2 receptor, IgG receptor FcRn large subunit p51, Low density lipoprotein receptor-related protein 1, Low density lipoprotein receptor-related protein 2, Insulin receptor, Cell cycle control protein 50A, Transmembrane protein 50A, Basigin, Leptin Receptor, Claudin-5. P-selectin, Lactoferrin receptor, Folate receptor. Sodium-dependent lysophosphatidylcholine symporter 1. Solute carrier organic anion transporter family member 1C1, Sodium-coupled neutral amino acid transporter 5, LDL receptor-related protein 8, High affinity cationic amino acid transporter 1, Sodium- and chloride-dependent taurine transporter, Insulin-like growth factor-binding protein 7. Solute carrier family 40 member 1, Zinc transporter 6, heparin-binding epidermal growth factor-like growth factor, and Myelin-oligodendrocyte glycoprotein (MOG). Binding to a BBB transport protein and a second protein significantly increases uptake of the dual transporter, and optionally a therapeutic agent linked to the dual transporter, into the brain, significantly increases its retention in the brain (i.e., brain exposure time), or both, as compared to a molecule that binds the BBB transport protein but not the second protein. In some embodiments, binding to a BBB transport protein and a second protein significantly increases uptake of the dual transporter, and optionally a therapeutic agent linked to the dual transporter, into cells or tissue that express the BBB transport protein and the second protein, significantly increases retention of the dual transporter, and optionally a therapeutic agent linked to the dual transporter those cells or tissue that express the BBB transport protein and the second protein, or both, as compared to a molecule that binds the BBB transport protein but not the second protein.

In some embodiments, the BBB transport protein is TfR and the second protein is a brain retention protein. In some embodiments, the brain retention protein is CD98hc or MOG or a protein selected from the group consisting of GLUT1, MFSD2A, IGF1R, and CA-IV. In some embodiments, the BBB transport protein is TfR and the second protein is CD98hc. In some embodiments, the dual transporter comprises a TfR binding region and a CD98hc binding region. In some embodiments, the BBB transport protein is TfR and the second protein is MOG. In some embodiments, the dual transporter comprises a TfR binding region and a MOG binding region. In some embodiments, the BBB transport protein is CD98hc and the second protein is a brain retention protein. In some embodiments, the dual transporter comprises a CD98hc binding region and a brain retention protein binding region.

The described dual transporters can be used to improve or increase delivery of a therapeutic agent to the CNS. In some embodiments, the dual transporter is linked to the therapeutic agent. Linking of a dual transporter to a therapeutic agent increases delivery of the therapeutic agent to the CNS relative to the level of delivery of the therapeutic agent to the CNS in the absence of linkage to the dual transporter. In some embodiments, linking of a dual transporter to a therapeutic agent increases delivery of the therapeutic agent to the CNS relative to the level of delivery to the CNS of the therapeutic agent linked to a molecule that binds the BBB transport protein, but not the second protein. In some embodiments, a therapeutic agent, such as an antibody, is modified to contain a first binding region that specifically binds to a BBB transport protein and a second binding region that specifically binds to a second protein, wherein the second protein is expressed on the luminal surface of the BBB or is a brain retention protein, and wherein the second protein is different from the BBB transport protein (e.g., a therapeutic agent, such as an antibody, can be modified to contain dual transporter function). Modifying a therapeutic agent, such as an antibody, to contain dual transporter function, increases delivery of the therapeutic agent to the CNS relative to the level of delivery of the therapeutic agent to the CNS in the absence of modifying the agent to contain dual transporter function.

Described are methods of improving delivery of an agent, such as a therapeutic agent, to the CNS comprising linking a dual transporter to the agent or modifying the agent to contain a first binding region that specifically binds to a BBB transport protein and a second binding region that specifically binds to a second protein, wherein the second protein is expressed on the luminal surface of the BBB or is a brain retention protein, and wherein the second protein is different from the BBB transport protein (e.g., modifying the agent to contain dual transporter function). Improving delivery of the agent to the CNS includes, but is not limited to, (a) increasing the level of the agent delivered to the CNS relative to the level of agent delivered to the CNS in the absence of the dual transporter; (b) decreasing a dose (amount) of the agent required to be administered to the subject to achieve a therapeutic affect or level of the agent delivered to the CNS relative to a dose of the agent required to be administered to the subject to achieve a therapeutic affect or level of the agent the CNS in the absence of the dual transporter; (c) increasing potency of the agent relative to the potency of the agent in the absence of the dual transporter: (d) increasing the safety (e.g., reducing toxicity) of the agent relative to the safety of the agent in the absence of the dual transporter; (e) improving biodistribution and/or pharmacokinetics of the agent relative biodistribution and/or pharmacokinetics of the agent in the absence of the dual transporter: (f) increasing Cmax of brain uptake of an agent relative to the Cmax of brain uptake of the agent in the absence of the dual transporter; (g) increasing specificity of delivery of an agent to the brain relative to specificity of delivery of an agent to the brain in the absence of the dual transporter. Linking a dual transporter to the agent, or modifying the agent to have dual transporter function, also improves delivery of the agent to the CNS relative to linking the agent to a binding region that specifically binds to a BBB transport protein in the absence of the second binding region that specifically binds to the second protein.

In some embodiments, binding to the BBB transport protein and the second protein reduces the dose of a therapeutic agent required to be administered to a subject to achieve a desired or therapeutic level of the therapeutic agent in the CNS (e.g., brain) relative to the dose required for the therapeutic agent alone or the therapeutic agent linked to a molecule that binds the BBB transport protein, but not the second protein. Reducing dosage can provide for improved safety margins for a patient.

The described dual transporters can be used to improve delivery of a therapeutic agent to the peripheral cells or tissue (cells that are not part of the CNS) that express the BBB transport protein and the second protein. Peripheral cells or tissue that express the BBB transport protein and the second protein include, but are not limited to, proliferating cells, cancer cells and tumors. In some embodiments, the dual transporter is linked to the therapeutic agent. Linking of a dual transporter to a therapeutic agent improves delivery of the therapeutic agent to the peripheral cells or tissue relative to the level of delivery of the therapeutic agent to the peripheral cells or tissue in the absence of linkage to the dual transporter. In some embodiments, linking of a dual transporter to a therapeutic agent improves delivery of the therapeutic agent to the peripheral cells or tissue relative to the level of delivery to the peripheral cells or tissue relative of the therapeutic agent linked to a molecule that binds the BBB transport protein, but not the second protein. In some embodiments, a therapeutic agent, such as an antibody, is modified to contain a first binding region that specifically binds to a BBB transport protein and a second binding region that specifically binds to a second protein, wherein the second protein is expressed on the luminal surface of the BBB or is a brain retention protein, and wherein the second protein is different from the BBB transport protein (e.g., a therapeutic agent, such as an antibody, can be modified to contain dual transporter function). Modifying a therapeutic agent, such as an antibody, to contain dual transporter function, improves delivery of the therapeutic agent to the peripheral cells or tissue relative to the level of delivery of the therapeutic agent to the peripheral cells or tissue in the absence of modifying the agent to contain dual transporter function.

Described are methods of improving delivery of an agent, such as a therapeutic agent, to peripheral cells or tissue (cells that are not part of the CNS) that express the BBB transport protein and the second protein comprising linking a dual transporter to the agent or modifying the agent to contain a first binding region that specifically binds to a BBB transport protein and a second binding region that specifically binds to a second protein, wherein the second protein is expressed on the luminal surface of the BBB or is a brain retention protein, and wherein the second protein is different from the BBB transport protein (e.g., modifying the agent to contain dual transporter function). Peripheral cells or tissue that express the BBB transport protein and the second protein include, but are not limited to, proliferating cells, cancer cells and tumors. Improving delivery of the agent to the peripheral cells or tissue includes, but is not limited to, (a) increasing the level of the agent delivered to the peripheral cells or tissue relative to the level of agent delivered to the peripheral cells or tissue in the absence of the dual transporter: (b) decreasing a dose (amount) of the agent required to be administered to the subject to achieve a therapeutic affect or level of the agent delivered to the peripheral cells or tissue relative to a dose of the agent required to be administered to the subject to achieve a therapeutic affect or level of the agent the peripheral cells or tissue in the absence of the dual transporter: (c) increasing potency of the agent relative to the potency of the agent in the absence of the dual transporter; (d) increase uptake of the agent into the peripheral cells or tissue relative to the level of uptake of agent into the peripheral cells or tissue in the absence of the dual transporter: (e) increasing the safety of the agent relative to the safety of the agent in the absence of the dual transporter; and/or (f) improve biodistribution and/or pharmacokinetics of the agent relative biodistribution and/or pharmacokinetics of the agent in the absence of the dual transporter. Linking a dual transporter to the agent, or modifying the agent to have dual transporter function, also improves delivery of the agent to the peripheral cells or tissue relative to linking the agent to a binding region that specifically binds to a BBB transport protein in the absence of the second binding region that specifically binds to the second protein.

In some embodiments, binding to the BBB transport protein and the second protein reduces the dose of a therapeutic agent required to be administered to a subject to achieve a desired or therapeutic level of the therapeutic agent in the peripheral cells or tissue relative to the dose required for the therapeutic agent alone or the therapeutic agent linked to a molecule that binds the BBB transport protein, but not the second protein. Reducing dosage can provide for improved safety margins for a patient.

### BRIEF DESCRIPTION OF THE FIGURES

FIG. 1 are schematic diagrams of exemplary dual transporters having heterodimeric Fc polypeptides modified to specifically bind (A) a BBB transport protein (first binding region) and a second protein (second binding region) or (B) TfR (anti-TfR) and CD98hc (anti-CD98hc). The heterodimeric Fc polypeptide is fused to Fabs. The Fabs can bind to one or two therapeutic targets.

FIG. 2A-B are schematic diagrams of exemplary dual transporters having a VHH-Fc/Fab-Fc format. A shows a dual transporter having a Fab domain having a first binding region and a VHH domain having a second binding region. B shows a dual transporter having a Fab domain that specifically binds TfR (anti-TfR) and a VHH domain that specifically binds CD98hc (anti-CD98hc).

FIG. 2C-D are schematic diagrams of exemplary dual transporters having a scFv domain and VHH domain linked to a full-length antibody. The scFv is linked to the C-terminal end of one heavy chain of the antibody and the VHH is linked to the C-terminal end of the other heavy chain of the antibody. A shows a dual transporter having a scFv having a first binding region and a VHH having a second binding region. B shows a dual transporter having a scFv that specifically binds TfR (anti-TfR) and a VHH that specifically binds CD98hc (anti-CD98hc)

FIG. 3A-C are schematics of additional exemplary dual transporters. A shows a dual transporter comprising a full-length antibody having first and second binding regions (e.g., scFv's) each linked to a C-terminal end of a light chain of the antibody. The antibody Fabs can bind to one or two therapeutic targets. B shows a dual transporter comprising a full-length antibody having a first binding region (e.g., a scFab) linked to a heavy chain of the antibody and Fab arm of the antibody that specifically binds to the second protein. The second Fab arm of the antibody can bind to a therapeutic target. C shows a dual transporter molecule having a DVD-Ig format. The DVD-Ig comprises a full-length antibody having an additional Fv fragment linked to each Fab arm. The example in C shows an additional Fv fragment having a first binding region linked to a Fab arm having a second binding region. The other addition Fv fragment and the other Fab arm can bind to one or two therapeutic targets.

FIG. 3D-F are schematics of additional exemplary dual transporters. D shows a dual transporter comprising a includes a full-length antibody having an anti-TfR scFv linked to a C-terminal end of one light chain of the antibody and an anti-Cd98hc scFv linked to a C-terminal end of the other light chain of the antibody. E shows a dual transporter comprising a full-length antibody having one Fab arm that binds to CD98hc and having an anti-TfR scFab linked to a C-terminal end of one heavy chain of the antibody. The other Fab arm of the antibody can bind to a therapeutic target. F shows a DVD-Ig dual transporter molecule having an anti-TfR Fv fragment linked to a Fab arm that specifically binds CD98hc. The other Fv fragment and Fab arm of the DVD-Ig can bind to one or two therapeutic targets.

FIG. 4 are graphs showing binding of exemplary antibody transport vehicles (ATVs) that bind only TfR, ATVs that bind only CD98hc, dual transporters that bind both TfR and CD98hc and a negative control antibody that binds neither TfR nor CD98hc, to HEK293T cells that express both TfR and CD98hc.

FIG. 5 are graphs showing binding of certain ATVs that bind only TfR, ATVs that bind only CD98hc, dual ATVs that bind both TfR and CD98hc, and a negative control antibody that binds neither TfR nor CD98hc to HEK293T cells that express both TfR and CD98hc.

FIG. 6 are graphs of one experiment showing plasma and brain concentration of certain dual transporters and corresponding single-binding controls after a single 50 mg/kg intravenous administration in mice.

FIG. 7 are graphs showing plasma and brain concentration of certain dual transporters after single intravenous administration in mice as shown in Example 8, wherein molecule format match standards are used.

FIG. 8 are graphs showing plasma and brain concentration of certain dual transporters and corresponding single-binding controls after a single 25 mg/kg or 10 mg/kg intravenous administration in mice.

FIG. 9 are graphs showing plasma pharmacokinetics of certain dual transporters having varied TfR and CD98hc binding affinities. These data demonstrate that TfR affinity can be a stronger driver of plasma clearance than CD98hc for dual binding molecules.

FIG. 10 are graphs showing brain pharmacokinetics of certain dual transporters having varied TfR and CD98hc binding affinities. These data demonstrate that among the TfR and CD98hc affinities tested in a dual binding format, stronger TfR affinity drive faster, higher uptake, while stronger CD98hc affinity drives increased brain retention (i.e., brain exposure time).

FIG. 11 are graphs showing kidney pharmacokinetics of certain dual transporters having varied TfR and CD98hc binding affinities. These data demonstrate that biodistribution of dual binding molecules to kidney is strongly correlated with CD98hc affinity.

FIG. 12 are graphs showing bone marrow pharmacokinetics of certain dual transporters having varied TfR and CD98hc binding affinities. These data demonstrate that biodistribution of dual binding molecules to bone marrow is strongly correlated with TfR affinity.

FIG. 13 shows quantification of the brain regional biodistribution of certain dual transporters having varied TfR and CD98hc binding affinities. These data demonstrate that binding both TfR and CD98hc results in a relative increase in biodistribution to the medula and pons not observed with molecules that only bind CD98hc or TfR.

FIG. 14 shows quantification of CNS cellular biodistribution of certain dual transporters having varied TfR and CD98hc binding affinities. These data demonstrate that for dual binding molecules TfR mediated biodistribution to neurons can be reduced in CD98hc affinity dependent manner.

FIG. 15 are graphs showing peripheral and brain pharmacokinetics of dual transporters of varying formats (Example 7) and transporter affinities.

FIG. 16 are graphs showing peripheral and brain pharmacokinetics of ATV^{TfR}:MOG1 molecules demonstrating that MOG1 binding drives retention and reduces TfR mediated clearance from brain.

### DETAILED DESCRIPTION

### I. Definitions

As used herein, the singular forms “a,” “an,” and “the” include plural referents unless the content clearly dictates otherwise. Thus, for example, reference to “a polypeptide” may include two or more such molecules, and the like.

As used herein, the terms “about” and “approximately.” when used to modify an amount specified in a numeric value or range, indicates the numeric value as well as reasonable deviations from the value known to the skilled person in the art. In some embodiments, the term “about” means within the typical ranges of tolerances in the art. In some embodiments, the term “about” means within 1 or 2 standard deviations from the mean. In some embodiments, the term “about” means ±10%. In some embodiments, the term “about” means ±5%. When the term “about” is present before a series of numbers or a range, it is understood that “about” can modify each of the numbers in the series or range.

A “transferrin receptor” or “TfR” as used in the context of this invention refers to transferrin receptor protein 1 (encoded by the TfR1 (CD71) gene). The human transferrin receptor 1 polypeptide sequence is set forth in SEQ ID NO:15. Transferrin receptor protein 1 sequences from other species are also known (e.g., chimpanzee, accession number XP 003310238.1; rhesus monkey, NP_001244232.1; dog. NP_001003111.1: cattle, NP_001193506.1; mouse, NP 035768.1; rat, P_073203.1; and chicken, P_990587.1). The term “transferrin receptor” also encompasses allelic variants of exemplary reference sequences, e.g., human sequences, that are encoded by a gene at a transferrin receptor protein 1 chromosomal locus. Full-length transferrin receptor protein includes a short N-terminal intracellular region, a transmembrane region, and a large extracellular domain. The extracellular domain is characterized by three domains: a protease-like domain, a helical domain, and an apical domain. The apical domain sequence of human transferrin receptor 1 is set forth in SEQ ID NO:1.

“CD98hc” or “CD98 heavy chain” refers to 4F2 cell-surface antigen heavy chain and is encoded by the SLC3A2 gene. CD98hc is also known as 4F2 heavy chain. The human CD98hc sequence is set forth in SEQ ID NO:5 and UNIPROT Accession No. P08195. CD98hc sequences from other species are also known (e.g., mouse, UNIPROT Accession No. P10852 and cynomolgus monkey, UNIPROT Accession No. G8F3Z0).

“MOG” refers to Myelin Oligodendrocyte Glycoprotein and is encoded by the MOG gene (also termed: BTNL11, BTN6, MOG Alpha-G, MOG AluA, MOG AluB, MOGIG2, and NRCLP7). The human MOG sequence is set forth in: NCBI Gene: 4340 and UniProtKB/Swiss-Prot: Q16653.

“BBB transport protein” includes any membrane-bound receptor that is expressed at the BBB and is capable of transporting a molecule that binds to the receptor across the BBB. Examples of such receptors include TfR and CD98hc, as well as others described herein. In some embodiments, a BBB transport protein is TfR. In some embodiments, a BBB transport protein is CD98hc.

“BBB surface proteins” are proteins that are more highly expressed on endothelial cells of the BBB relative to peripheral endothelial cells. Although not required, BBB surface proteins can have limited or even no expression in the periphery (tissue or cells outside of the BBB or CNS) of the animal. BBB surface proteins can be either transporter proteins. e.g. proteins that use receptor-mediated transcytosis to transport another molecule, or proteins that are not transporters.

“Brain retention proteins” are proteins that are (1) expressed in the brain; (2) present on a cell surface or are extracellular; and (3) themselves not therapeutic targets with respect to binding by a dual transporter. Brain retention proteins include, but are not limited to, extracellular structural proteins such as MOG and receptors such as CD98hc. Although not required, Brain retention proteins can have limited or even no expression in the periphery of the animal. A brain retention protein can be, but is not limited to, CD98hc. MOG. GLUT1, MFSD2A, IGF1R, or CA-IV. In some embodiments, a brain retention protein is selected from the group consisting of CD98hc, MOG, GLUT1, MFSD2A, IGF1R, and CA-IV.

An “Fc polypeptide” refers to the C-terminal region of a naturally occurring immunoglobulin heavy chain polypeptide that is characterized by an Ig fold as a structural domain. An Fc polypeptide contains constant region sequences including at least the CH2 peptide and/or the CH3 peptide and may contain at least part of the hinge region. In general, an Fc polypeptide does not contain a variable region.

A “modified Fc polypeptide” is an Fc polypeptide that has at least one mutation, e.g., a substitution, deletion or insertion, as compared to a wild-type immunoglobulin heavy chain Fc polypeptide sequence, but retains the overall Ig fold or structure of the native Fc polypeptide.

An “Fc polypeptide dimer” refers to a dimer of two Fc polypeptides. In some embodiments, an Fc polypeptide dimer is capable of binding an Fc receptor (e.g., FcγR). In an Fc polypeptide dimer, the two Fc polypeptides dimerize by the interaction between the two CH3 antibody constant domains. In some embodiments, the two Fc polypeptides may also dimerize via one or more disulfide bonds that form between the hinge domains of the two dimerizing Fc domain monomers. An Fc polypeptide dimer can be a heterodimer or a homodimer. An Fc polypeptide dimer may comprise two wild-type Fc polypeptides, a wild-type Fc polypeptide and a modified Fc polypeptide, or two modified Fc polypeptides. For an Fc polypeptide dimer comprising two modified Fc polypeptide, the two modified Fc polypeptides may be the same or different.

“CH3 peptide” and “CH2 peptide” refer to immunoglobulin constant region domain polypeptides. In the context of IgG antibodies, a CH3 peptide refers to the segment of amino acids from about position 341 to about position 447 as numbered according to the EU numbering scheme, and a CH2 peptide polypeptide refers to the segment of amino acids from about position 231 to about position 340 as numbered according to the EU numbering scheme. CH2 and CH3 peptide polypeptides may also be numbered by the EVIGT (ImMunoGeneTics) numbering scheme in which the CH2 peptide numbering is 1-110 and the CH3 peptide numbering is 1-107, according to the IMGT Scientific chart numbering (IMGT website). CH2 and CH3 peptides are part of the Fc region of an immunoglobulin. In the context of IgG antibodies, an Fc region refers to the segment of amino acids from about position 231 to about position 447 as numbered according to the EU numbering scheme. As used herein, the term “Fc region” may also include at least a part of a hinge region of an antibody.

The term “antibody” refers to a protein with an immunoglobulin fold that specifically binds to an antigen via its variable regions (antibody antigen binding domains). The term encompasses intact polyclonal antibodies, intact monoclonal antibodies, single chain antibodies, multispecific antibodies such as bispecific antibodies, monospecific antibodies, monovalent antibodies, chimeric antibodies, humanized antibodies, and human antibodies. The term “antibody,” as used herein, includes both full-length antibodies (e.g, IgG1 antibody that has two heavy chains made up of variable heavy (V_{H}), CH1, CH2, and CH3 domains and two light chains made up of variable light (V_{L}) and CL domains) and also antibody fragments that retain antigen-binding specificity. Antibody fragments include but are not limited to. Fab, F(ab′)₂, scFab, Fv, scFv, bivalent scFv, VHH, vNAR, and nanobody. Antibodies can contain light chains that are classified as either kappa or lambda. Antibodies can contain heavy chains that are classified as gamma, mu, alpha, delta, or epsilon, which in turn define the immunoglobulin classes. IgG, IgM, IgA, IgD and IgE, respectively.

The terms “wild-type,” “native,” and “naturally occurring” with respect to a CH3 or CH2 peptide are used herein to refer to a domain that has a sequence that occurs in nature.

The term “Fcγ receptor” or “FcγR” refers to one type of Fc receptors, which are classified based on the type of antibody that they recognized. FcγRs include several members, FcγRI (CD64). FcγRIIA (CD32), FcγRIIB (CD32), FcγRIIIA (CD16a), and FcγRIIIB (CD16b), which differ in their antibody affinities due to different molecular structures. FcγRs bind to the Fc portion of IgG class of antibodies and are crucial for inducing phagocytosis of opsonized microbes. FcγRs are found on the cell surface of cells in the immune system. FcγRs are responsible for eliciting immune system effector functions and are activated upon binding of the Fc portion of an antibody to the receptor. FcγRs mediate immune functions, e.g., binding to antibodies that are attached to infected cells or invading pathogens, stimulating phagocytic or cytotoxic cells to destroy microbes or infected cells by antibody-mediated phagocytosis or ADCC.

The term “variable region” refers to a domain in an antibody heavy chain or light chain derived from a germline Variable (V) gene, Diversity (D) (heavy chain only) gene, and Joining (J) gene (and not derived from a Constant (Cμ and Cδ) gene segment), and that gives an antibody its specificity for binding to an antigen. Typically, an antibody variable region comprises four conserved “framework” regions interspersed with three hypervariable “complementarity determining regions” (CDRs).

A variant, with respect to a given sequence, is an alteration in a nucleic acid or amino acid sequence relative to a reference (e.g., wild-type or most common) sequence. A variant can be a naturally occurring sequence (e.g., an allelic variant) and non-naturally occurring sequence. A non-naturally occurring variant domain refers to a variant or mutant domain that is not present in a cell in nature and that is produced by genetic modification (e.g. using genetic engineering technology or mutagenesis techniques) of a native domain. An alteration in a nucleic acid or amino acid sequence (e.g., a mutation) can include one or more substitutions, one or more insertions, one or more deletions, or combinations thereof.

The term “conservative substitution,” “conservative mutation,” or “conservatively modified variant” refers to an alteration that results in the substitution of an amino acid with another amino acid that can be categorized as having a similar feature. Examples of categories of conservative amino acid groups defined in this manner can include: a “charged/polar group” including Glu (Glutamic acid or E), Asp (Aspartic acid or D), Asn (Asparagine or N), Gln (Glutamine or Q), Lys (Lysine or K), Arg (Arginine or R), and His (Histidine or H); an “aromatic group” including Phe (Phenylalanine or F), Tyr (Tyrosine or Y), Tip (Tryptophan or W), and (Histidine or H); and an “aliphatic group” including Gly (Glycine or G). Ala (Alanine or A), Val (Valine or V), Leu (Leucine or L), He (Isoleucine or I), Met (Methionine or M), Ser (Serine or S), Thr (Threonine or T), and Cys (Cysteine or C). Within each group, subgroups can also be identified. For example, the group of charged or polar amino acids can be sub-divided into sub-groups including: a “positively-charged subgroup” comprising Lys, Arg and His; a “negatively-charged sub-group” comprising Glu and Asp; and a “polar sub-group” comprising Asn and Gln. In another example, the aromatic or cyclic group can be sub-divided into sub-groups including: a “nitrogen ring sub-group” comprising Pro, His and Trp; and a “phenyl sub-group” comprising Phe and Tyr. In another further example, the aliphatic group can be sub-divided into sub-groups, e.g., an “aliphatic non-polar sub-group” comprising Val, Leu, Gly, and Ala; and an “aliphatic slightly-polar sub-group” comprising Met, Ser, Thr, and Cys. Examples of categories of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free —OH can be maintained; and Gln for Asn or vice versa, such that a free —NH₂ can be maintained. In some embodiments, hydrophobic amino acids are substituted for naturally occurring hydrophobic amino acids, e.g. in the active site, to preserve hydrophobicity.

“Binding affinity” refers to the strength of the non-covalent interaction between two molecules, e.g., between single binding region or site on a protein and a target. Binding affinity may be quantified by measuring an equilibrium dissociation constant (KD), which refers to the dissociation rate constant (k_{d}, time⁻¹) divided by the association rate constant (kₐ, time⁻¹M⁻¹). KD can be determined by measurement of the kinetics of complex formation and dissociation, e.g., using Surface Plasmon Resonance (SPR) methods, e.g., a Biacore™ system; kinetic exclusion assays such as KinExA®; and BioLayer interferometry (e.g., using the ForteBio® Octet® platform). The term “binding affinity” includes not only formal binding affinities, such as those reflecting 1:1 interactions between a polypeptide and its target, but also apparent affinities for which KD's are calculated that may reflect avid binding (avidity).

The term “specifically binds” or “selectively binds” to a target (e.g., a BBB protein, e.g., TfR or CD98hc) when referring to a binding region (e.g., BBB binding region) refers to a binding reaction whereby the binding region binds to the target with greater affinity, greater avidity, and/or greater duration than it binds to a structurally different target. A binding region may have at least 5-fold. 6-fold, 7-fold, 8-fold, 9-fold, 10-fold, 20-fold, 25-fold, 50-fold. 100-fold, 1.000-fold. 10.000-fold, or greater affinity to a specific target compared to an unrelated target when assayed under the same affinity assay conditions. The term “specific binding,” “specifically binds to,” or “is specific for” a particular target can be exhibited, for example, by a molecule having an equilibrium dissociation constant KD for the target to which it binds of, e.g., 10⁴ M or smaller (e.g., 10⁵ M, 10⁶ M (1000 nM), 10⁷ M (100 nM), 10⁸ M (10 nM), 10⁻⁹ M (1 nM), 10⁻¹⁰ M, 10⁻¹¹ M, or 10⁻¹² M). In some embodiments, a binding region specifically binds to a target (e.g., protein) that is conserved among species, (e.g., structurally conserved among species).

The term “amino acid” refers to naturally occurring and synthetic amino acids, as well as amino acid analogs and amino acid mimetics that function in a manner similar to the naturally occurring amino acids.

Naturally occurring amino acids are those encoded by the genetic code, as well as those amino acids that are later modified, e.g., hydroxyproline, γ-carboxyglutamate and O-phosphoserine. “Amino acid analogs” refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g. norleucine) or modified peptide backbones, but retain the same basic chemical structure as a naturally occurring amino acid. “Amino acid mimetics” refers to chemical compounds that have a structure that is different from the general chemical structure of an amino acid, but that function in a manner similar to a naturally occurring amino acid.

Naturally occurring α-amino acids include, without limitation, alanine (Ala, A), cysteine (Cys, C), aspartic acid (Asp, D), glutamic acid (Glu, E), phenylalanine (Phe, F), glycine (Gly, G), histidine (His, H), isoleucine (Ile, I), arginine (Arg, R), lysine (Lys, K), leucine (Leu, L), methionine (Met, M), asparagine (Asn, N), proline (Pro, P), glutamine (Gln, Q), serine (Ser, S), threonine (Thr, T), valine (Val, V), tryptophan (Trp, W), tyrosine (Tyr, Y), and combinations thereof. Stereoisomers of naturally occurring a-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lvs), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof, Amino acids may be referred to herein by either their commonly known three letter symbols or by the one-letter symbols recommended by the IUPAC-IUB Biochemical Nomenclature Commission.

As used herein, the term “mutant” with respect to a mutant polypeptide or mutant polynucleotide is used interchangeably with “variant.” A variant with respect to a given wild-type CH3 or CH2 peptide reference sequence can include naturally occurring allelic variants. A “non-naturally” occurring CH3 or CH2 peptide refers to a variant or mutant domain that is not present in a cell in nature and that is produced by genetic modification. e.g., using genetic engineering technology or mutagenesis techniques, of a native CH3 peptide or CH2 peptide polynucleotide or polypeptide. A “variant” includes any domain comprising at least one amino acid mutation with respect to wild-type. Mutations may include substitutions, insertions, and deletions. Substitutions of a single amino acid can be indicated by a single letter amino acid symbol indicating the amino acid prior to the substitution, followed by a number indicating the amino acid position, followed by a one letter amino acid symbol indicating the amino acid substitution (e.g., T366W indicates a threonine at position 366 is modified to be a tryptophan). Substitutions of a single amino acid can also be indicated by a number indicating the amino acid position at which this is a substitution followed by a one letter amino acid symbol indicating the amino acid substitution (e.g., 366W indicates a tryptophan at position).

A “polypeptide” is a polymer of two or more amino acid residues in a single chain. The term applies to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L and D amino acids.

The term “protein” as used herein refers to either a polypeptide, a polypeptide dimer, or polypeptide multimer. A polypeptide dimer can be a homodimer or a heterodimer. A polypeptide multimer can be a homomultimer or a heteromultimer. A heteromultimer may comprise two or more copies of any give single chain polypeptide. For example, an immunoglobulin is a heteromultimer comprises two heavy chains and two light chains. The tow heavy chains can be the same or different the two light chains can be the same or different. The single polypeptide chains of a dimer or multimer may be joined by one or more covalent bonds (e.g., a disulfide bonds), by non-covalent interactions, or by a combination thereof.

The terms “identical” or percent “identity,” in the context of two or more nucleic acid or polypeptide sequences, refer to two or more sequences or subsequences that are the same or have a specified percentage of nucleic acid or amino acid residues (e.g., at least 60%, at least 65%, at least 70%), at least 75%, at least 80%, at least 85%, at least 90%, or at least 95% or greater) that are identical over a specified region when compared and aligned for maximum correspondence over a comparison window or designated region, as measured using a sequence comparison algorithm or by manual alignment and visual inspection. For sequence comparison, typically one sequence acts as a reference sequence to which a candidate sequence is compared. Alignment can be performed using various methods available to one of skill in the art. e.g., visual alignment or using publicly available software using known algorithms to achieve maximal alignment. Such programs include the BLAST programs, ALIGN, ALIGN-2 (Genentech, South San Francisco, Calif.) or Megalign (DNASTAR). The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

The terms “corresponding to,” “determined with reference to,” or “numbered with reference to” when used in the context of the identification of a given nucleotide or amino acid residue in a nucleic acid or polypeptide sequence, refers to the position of the residue of a specified reference sequence when the given amino acid sequence is maximally aligned and compared to the reference sequence. The sequence that is aligned to the reference sequence need not be the same length as the reference sequence.

The “EU numbering scheme” is generally used in the art when referring to a residue in an antibody heavy chain constant region. The EU numbering scheme is shown below with respect to SEQ ID NO: 2 (Clone CH3C.35.23.2 with knob mutation):

```
      QVQLVES
      GGGVVQPGRSLRLSCAASGFAFSSYGMHWVRQAPGKGLEWVAVIWFDGTK
      KYYTDSVKGRFTISRDNSKNTLYLQMNTLRAEDTAVYYCARDRGIGARRG
EU 120 130 140 150
                                            | | | |
      PYYMDVWGKGTTVTVSSASTKGPSVFPLAPSSKSTSGGTAALGCLVKDYF
EU 160 170 180 190 200
                        | | | | |
      PEPVTVSWNSGALTSGVHTFPAVLQSSGLYSLSSVVTVPSSSLGTQTYIC
EU 210 220 230 240 250
                        | | | | |
      NVNHKPSNTKVDKKVEPKSCDKTHTCPPCPAPELLGGPSVFLFPPKPKDT
EU 260 270 280 290 300
                        | | | | |
      LMISRTPEVTCVVVDVSHEDPEVKENWYVDGVEVHNAKTKPREEQYNSTY
EU 310 320 330 340 350
                        | | | | |
      RVVSVLTVLHQDWLNGKEYKCKVSNKALPAPIEKTISKAKGQPREPQVYT
EU 360 370 380 390 400
                        | | | | |
      LPPSRDELTKNQVSLWCLVKGFYPSDIAVEWESYGTEWANYKTTPPVLDS
EU 410 420 430 440
                        | | | |
      DGSFFLYSKLTVTKEEWQQGFVFSCSVMHEALHNHYTQKSLSLSPGK.
```

The term “subject,” “individual,” and “patient.” as used interchangeably herein, refer to a mammal, including but not limited to humans, non-human primates, rodents (e.g., rats, mice, and guinea pigs), rabbits, cows, pigs, horses, and other mammalian species. In some embodiments, the subject is a human.

The terms “treat,” “treatment,” and the like, mean the methods or steps taken to provide relief from, or amelioration or alleviation of the number, severity, adverse effect, and/or frequency of one or more symptoms or pathological consequences of a disease, disorder, or condition in a subject. Treatment can be prophylactic in terms of preventing or partially preventing a disease, or a symptom or condition of the disease. Preventing includes providing prophylaxis with respect to the occurrence or recurrence of a disease in a subject that may be predisposed to the disease but has not yet been diagnosed with the disease. Preventing also includes providing prophylaxis with respect to the occurrence or recurrence of a symptom or pathological consequence of a disease in a subject that may be predisposed symptom or pathological consequence of the disease but has not yet been diagnosed with the symptom or pathological consequence the disease. Treatment can also be prophylactic in terms of delaying onset of a disease, or a symptom or condition of the disease. Delaying development of a disease or symptom or pathological consequence of the disease indicates deferring, hindering, slowing, retarding, stabilizing, suppressing, and/or postponing development of the disease or symptom or pathological consequence of the disease. The delay can be of varying lengths of time, depending on the history of the disease and/or individual being treated. Treating can include inhibiting the disease, disorder or condition, e.g., impeding its progress; and relieving the disease, disorder, or condition, e.g., causing regression of the disease, disorder and/or condition. Treatment can also mean prolonging survival as compared to expected survival in the absence of treatment. Treatment can be therapeutic in terms of a partial or complete cure of a disease, condition, symptom or adverse effect attributed to the disease, disorder, or condition. The term treatment can include: (a) preventing the disease from occurring in a subject who may be predisposed to the disease but has not yet been diagnosed as having it; (b) inhibiting the disease, i.e., arresting its development; and (c) relieving the disease, i.e., mitigating or ameliorating the disease and/or its symptoms or conditions. Treating can refer to both therapeutic treatment alone, prophylactic treatment alone, or both therapeutic and prophylactic treatment. Those in need of treatment (subjects in need thereof) can include those previously diagnosed with a disease, disorder, or condition, or those identified as being at risk of developing a disease, disorder, or condition. Treating the disease, disorder, or condition can include ameliorating at least one symptom of the particular disease, disorder, or condition, even if the underlying pathophysiology is not affected.

The term “pharmaceutically acceptable excipient” refers to a non-active pharmaceutical ingredient that is biologically or pharmacologically compatible for use in humans or animals, such as but not limited to a buffer, carrier, or preservative.

An “effective amount” of an agent, e.g., a dual transporter or a pharmaceutical formulation containing a dual transporter, in the context of administration, refers to an amount effective, at dosages/amounts and for periods of time necessary, to achieve a desired result, such as a therapeutic or prophylactic result.

A “therapeutically effective amount” of an agent. e.g., a dual transporter or a pharmaceutical formulation containing a dual transporter, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered.

A “dose,” “unit dose,” or “dosage” refers to physically discrete units suitable for use in a subject, each unit containing a predetermined quantity of active pharmaceutical ingredient and/or a pharmaceutical composition.

The term “administer” refers to a method of delivering agents, compounds, or compositions to the desired site of biological action. These methods include, but are not limited to, topical delivery, parenteral delivery, intravenous delivery, intradermal delivery, intramuscular delivery, intrathecal delivery, colonic delivery, rectal delivery, or intraperitoneal delivery. In one embodiment, the compositions described herein are administered intravenously. A molecule that is “administered peripherally” means that the molecule is not administered directly to CNS (e.g., not administered intrathecally or directly into the brain, such as intracerebroventricularly).

### II. Dual Transporters

Dual transporters, compositions containing the dual transporters, nucleic acids encoding the dual transporters, and methods of manufacturing dual transporters are described.

Methods of using the dual transporter deliver a therapeutic agent to the brain or CNS to treat diseases or conditions of the brain or CNS are also described.

The described dual transporters and compositions are capable of crossing the BBB. The dual transporters can be linked, directly or indirectly, to a therapeutic agent. The therapeutic agent can be, but is not limited to, a drug, a cytotoxic agent, a DNA or RNA molecule, or a chemical moiety.

The dual transporters comprise a first binding region that specifically binds a BBB transport protein (e.g., TfR or CD98hc) (means for binding a BBB transport protein), and a second binding region that specifically binds a second protein (means for binding the second protein), wherein the second protein is expressed on the luminal surface of the BBB or is a brain retention protein, and wherein the second protein is different from the BBB transport protein. The combination of binding the two different proteins can increase transport of the molecule across the BBB, decrease efflux from the brain or degradation of the dual transporter, or a combination thereof. Without wishing to be bound by a particular theory, the use of a dual transporter that binds to two targets that are expressed on the luminal surface of the BBB may increase the local concentration of the molecule at the BBB surface, thereby facilitating engagement with the BBB transport protein (e.g., TfR or CD98hc). In addition, binding to a CNS target that results in brain retention (brain retention protein), which can reduce efflux or degradation of the molecule, can also enhance brain concentrations and increase the persistence (retention) of the molecule in the brain.

The described dual transporters can have a first binding region having weaker affinity to the BBB transport protein relative to a molecule that binds the BBB transport protein but does bind not the second protein while maintaining brain exposure (e.g., concentration in brain) that is equal to or increased relative to the molecule that binds the BBB transport protein but not the second protein. The first binding region of a dual transporter can bind the BBB transport protein with about 1.1 to about 10-fold weaker affinity relative to a molecule that binds the BBB transport protein but not the second protein while maintaining brain exposure (e.g., concentration in brain) that is equal to or increased relative to the molecule that binds the BBB transport protein but not the second protein. In some embodiments, the first binding region of a dual transporter can bind the BBB transport protein with about 1.1 to about 5-fold weaker affinity relative to a molecule that binds the BBB transport protein but not the second protein. In some embodiments, the first binding region of a dual transporter can bind the BBB transport protein with about 2 to about 10-fold weaker affinity relative to a molecule that binds the BBB transport protein but not the second protein. In some embodiments, the first binding region of a dual transporter can bind the BBB transport protein with about 2 to about 5-fold weaker affinity relative to a molecule that binds the BBB transport protein but not the second protein. Having weaker affinity to the BBB transport protein may allow for a lower dose of a therapeutic and/or improved safety margin for a dual transporter relative to a therapeutic delivered solely by the BBB transport protein in the absence of binding to the second protein.

In some embodiments, the first binding region of a dual transporter specifically binds the BBB transport protein with an equal or weaker affinity relative to the specific binding of the second binding region to the second protein. In some embodiments, the first binding region of a dual transporter specifically binds the BBB transport protein with a weaker affinity relative to the specific binding of the second binding region to the second protein. In some embodiments, the first binding region of a dual transporter specifically binds TfR with a weaker affinity relative to the specific binding of the second binding protein to CD98hc.

Described are dual transporters comprising: a first binding region that specifically binds a BBB transport protein (e.g., TfR or CD98hc) and a second binding region that specifically binds a second, different BBB protein that is a BBB surface protein or a brain retention protein. A dual transporter can independently be monovalent or multivalent for binding to the BBB transport protein and/or the second protein. In some embodiments, the dual transporter is monovalent for binding both the BBB transport protein and the second protein. Without wishing to be bound by a particular theory, binding of the dual transporter to the second BBB transport protein may improve brain delivery by increasing efficacy or rate of transport across the BBB, increasing local concentration of the transporter at the BBB surface thereby enhancing transport mediated by binding of the dual transporter to the BBB transport protein, and/or increasing retention (i.e., brain exposure time) of the dual transporter in the brain.

A BBB surface protein is a protein that is present on the luminal or abluminal surface of endothelial cells of the BBB. In some embodiments, a second protein is enriched in cells of the BBB. Enriched indicates the protein is present in cells of the BBB (e.g., BBB endothelial cells) at a higher level than the protein is present in most other cells of the body. The BBB is a term used to describe the microvasculature of the CNS. The BBB includes the network of blood vessels and associated tissue comprising the neurovascular network that is made up of closely spaced cells and helps keep harmful substances from reaching the brain. CNS vessels are continuous nonfenestrated vessels having properties that allow them to tightly regulate the movement of molecules, ions, and cells between the blood and the CNS. Blood vessels of the BBB are made up of two main cell types: endothelial cells that form the walls of the blood vessels, and mural cells that sit on the ablumenal surface of the endothelial cell layer. In some embodiments, the BBB protein comprises a protein that is present on the surface of endothelial cells of the CNS.

In some embodiments, the BBB transport protein and/or the second protein is a protein that undergoes receptor-mediated transcytosis. In some embodiments, the BBB transport protein undergoes receptor-mediated transcytosis. In some embodiments, the second protein undergoes receptor-mediated transcytosis. In some embodiments, the BBB transport protein and the second protein undergo receptor-mediated transcytosis.

In some embodiments, the BBB transport protein is TfR and the second protein is selected from the group comprising: CD98hc. Large neutral amino acids transporter small subunit 1 (CD98 light chain), glucose transporter 1 (GLUT1), major facilitator superfamily domain-containing protein 2A (MFSD2A), carbonic anhydrase IV (CA-IV), Low density lipoprotein receptor, Insulin-like growth factor 1 receptor (IGF1R). Insulin-like growth factor 2 receptor. IgG receptor FcRn large subunit p51, Low density lipoprotein receptor-related protein 1. Low density lipoprotein receptor-related protein 2, Insulin receptor, Cell cycle control protein 50A, Transmembrane protein 50A. Basigin, Leptin Receptor, Claudin-5, P-selectin, Lactoferrin receptor. Folate receptor, Sodium-dependent lysophosphatidylcholine symporter 1, Solute carrier organic anion transporter family member 1C1, Sodium-coupled neutral amino acid transporter 5, LDL receptor-related protein 8, High affinity cationic amino acid transporter 1, Sodium- and chloride-dependent taurine transporter, Insulin-like growth factor-binding protein 7, Solute carrier family 40 member 1, Zinc transporter 6, heparin-binding epidermal growth factor-like growth factor, and Myelin-oligodendrocyte glycoprotein (MOG). In some embodiments, the BBB transport protein is TfR and the second protein is CD98hc.

In some embodiments, the BBB transport protein is TfR or CD98hc and the second protein is a brain retention protein.

In some embodiments, the BBB transport protein is ALPL and the second protein is a brain retention protein. See Moyer T. C. et al., BioRxiv. 2024 Mar. 14; doi:10.1101/2024.03.12.584703 for description of exemplary ALPL binding regions.

In some embodiments, the BBB transport protein is TfR or CD98hc and the second protein is MOG. See Nakano R. et al., PLoS One. 2019 Apr. 12:14(4):e0214404 for description of exemplary MOG binding regions.

In some embodiments, the BBB transport protein is TfR or CD98hc and the second protein is GLUT1 (also known as solute carrier family 2 member 1, or SLC2A1).

In some embodiments, the BBB transport protein is TfR or CD98hc and the second protein is IGF1R.

In some embodiments, the BBB transport protein is TfR or CD98hc and the second protein is MFSD2A (major facilitator superfamily domain-containing protein 2A).

In some embodiments, the BBB transport protein is TfR or CD98hc the second protein is CA-IV. See Shay T. et al., Sci. Adv. 9, eadg6618 (2023) 19 Apr. 2023).

In some embodiments, the BBB transport protein is TfR and the second protein is a brain retention protein.

In some embodiments, the BBB transport protein is TfR and the second protein is CD98hc.

In some embodiments, the BBB transport protein is TfR and the second protein is MOG.

In some embodiments, the BBB transport protein is TfR and the second protein is GLUT1.

In some embodiments, the BBB transport protein is TfR and the second protein is IGF1R.

In some embodiments, the BBB transport protein is TfR and the second protein is MFSD2A.

In some embodiments, the BBB transport protein is TfR and the second protein is CA-IV.

In some embodiments, the BBB transport protein is CD98hc and the second protein is selected from the group comprising: TfR, Large neutral amino acids transporter small subunit 1 (CD98 light chain), Low density lipoprotein receptor, Insulin-like growth factor 1 receptor, Insulin-like growth factor 2 receptor, IgG receptor FcRn large subunit p51, Low density lipoprotein receptor-related protein 1, Low density lipoprotein receptor-related protein 2, Insulin receptor, Cell cycle control protein 50A, Transmembrane protein 50A, Basigin, Leptin Receptor, Claudin-5, P-selectin, Lactoferrin receptor, Folate receptor, Sodium-dependent lysophosphatidylcholine symporter 1, Solute carrier organic anion transporter family member 1C1, Sodium-coupled neutral amino acid transporter 5. LDL receptor-related protein 8, High affinity cationic amino acid transporter 1, Sodium- and chloride-dependent taurine transporter, Insulin-like growth factor-binding protein 7, Solute carrier family 40 member 1, Zinc transporter 6, heparin-binding epidermal growth factor-like growth factor, and Myelin-oligodendrocyte glycoprotein (MOG). In some embodiment, the BBB transport protein is CD98hc and the second protein is TfR.

In some embodiments, the BBB transport protein is CD98hc and the second protein is MOG.

In some embodiments, the BBB transport protein is CD98hc and the second protein is GLUT1.

In some embodiments, the BBB transport protein is CD98hc and the second protein is IGF1R.

In some embodiments, the BBB transport protein is CD98hc and the second protein is MFSD2A.

In some embodiments, the BBB transport protein is CD98hc and the second protein is CA-IV.

Additional BBB transport proteins include those expressed by brain endothelial cell genes associated with either receptor-mediated transcytosis or small molecule transport (see Zhang et al. Fluids and Barriers or the CNS (2020) 17:47 and Yang A C et al. Nature (2022) 603:885-892).

The first binding region can be, but is not limited to,
(a) a first peptide that specifically binds the BBB transport protein,
(b) a first antibody antigen binding domain that specifically binds the BBB transport protein,
(c) a first Fc polypeptide modified to specifically bind the BBB transport protein,
(d) a first CH3 peptide modified to specifically bind the BBB transport protein,
(e) a first fibronectin type III domain peptide modified to specifically bind the BBB transport protein, or
(f) a first bicyclic peptide that specifically binds the BBB transport protein.
   An antibody antigen binding domain can be, but is not limited to, a Fab, a single chain Fab (scFab), a heavy chain only antibody variable domain (nanobody, e.g., a VHH or a vNAR), a Fv fragment, or a single chain variable fragment (scFv).

In some embodiments, the first binding region can be, but is not limited to,
(a) a first peptide that specifically binds TfR,
(b) a first antibody antigen binding domain that specifically binds TfR,
(c) a first Fc polypeptide modified to specifically bind TfR.
(d) a first CH3 peptide modified to specifically bind TfR,
(e) a first fibronectin type III domain peptide modified to specifically bind TfR, or
(f) a first bicyclic peptide that specifically binds TfR.
   An antibody antigen binding domain can be, but is not limited to, a Fab, a single chain Fab (scFab), a heavy chain only antibody variable domain (nanobody, e.g., a VHH or a vNAR), a Fv fragment, or a single chain variable fragment (scFv).

In some embodiments, the first binding region can be, but is not limited to,
(a) a first peptide that specifically binds CD98hc,
(b) a first antibody antigen binding domain that specifically binds CD98hc,
(c) a first Fc polypeptide modified to specifically bind CD98hc,
(d) a first CH3 peptide modified to specifically bind CD98hc,
(e) a first fibronectin type III domain peptide modified to specifically bind CD98hc, or
(f) a first bicyclic peptide that specifically binds CD98hc.
   An antibody antigen binding domain can be, but is not limited to, a Fab, a single chain Fab (scFab), a heavy chain only antibody variable domain (nanobody, e.g., a VHH or a vNAR), a Fv fragment, or a single chain variable fragment (scFv).

The second binding region can be, but is not limited to,
(a) a second peptide that specifically binds the second protein,
(b) a second antibody antigen binding domain that specifically binds the second protein,
(c) a second Fc polypeptide modified to specifically bind the second protein,
(d) a second CH3 peptide modified to specifically bind the second protein,
(e) a second fibronectin type III domain peptide modified to specifically bind the second protein, or
(f) a second bicyclic peptide that specifically binds the second protein.
   An antibody antigen binding domain can be, but is not limited to, a Fab, a scFab, a heavy chain only antibody variable domain (nanobody, e.g., a VHH or a vNAR), a Fv fragment, or a scFv.

In some embodiments, the second binding region can be, but is not limited to,
(a) a second peptide that specifically binds CD98hc,
(b) a second antibody antigen binding domain that specifically binds CD98hc,
(c) a second Fc polypeptide modified to specifically bind CD98hc,
(d) a second CH3 peptide modified to specifically bind CD98hc,
(e) a second fibronectin type III domain peptide modified to specifically bind CD98hc, or
(f) a second bicyclic peptide that specifically binds to CD98hc.
   An antibody antigen binding domain can be, but is not limited to, a Fab, a scFab, a heavy chain only antibody variable domain (nanobody, e.g., a VHH or a vNAR), a Fv fragment, or a scFv.

In some embodiments, the second binding region can be, but is not limited to,
(a) a second peptide that specifically binds MOG,
(b) a second antibody antigen binding domain that specifically binds MOG,
(c) a second Fc polypeptide modified to specifically bind MOG,
(d) a second CH3 peptide modified to specifically bind MOG,
(e) a second fibronectin type III domain peptide modified to specifically bind MOG, or
(f) a second bicyclic peptide that specifically binds the second protein.
   An antibody antigen binding domain can be, but is not limited to, a Fab, a scFab, a heavy chain only antibody variable domain (nanobody, e.g., a VHH or a vNAR), a Fv, fragment, or a scFv.

An antibody antigen binding domain comprises the antigen binding domain of an immunoglobulin or a peptide having a structure similar to the antigen binding domain of an immunoglobulin. The immunoglobulin can be, but is not limited to, an IgG, IgM, IgE. IgA, IgD, or a heavy chain antibody. An antibody antigen binding domain can be, but is not limited to, a Fab, a scFab, a Fv fragment, a scFv, or a heavy chain only antibody variable domain (nanobody. e.g., a VHH or a vNAR).

The term “Fab” refers to an antigen-binding fragment consisting of a light chain variable region (V_{L}) and a light chain constant region (together the antibody light chain), and a heavy chain variable region (V_{H}) and a heavy chain CH1 constant region (together an antibody Fd fragment).

The term “single-chain Fab” or “scFab” refers to an antigen-binding fragment consisting of a Fab wherein the Fd fragment and the light chain linked together via a peptide linker. The linker can connect the N-terminus of the Fd fragment with the C-terminus of the light chain or the N-terminus of the light chain with the C-terminus of the Fd fragment.

The term “Fv fragment” refers to an antigen-binding fragment consisting of a V_{H} and a V_{L} that together form a binding site for an antigen.

The term “single-chain variable fragment” or “scFv” refers to an antigen-binding fragment consisting of a heavy chain variable region and a light chain variable region linked together via a peptide linker. The linker can connect the N-terminus of the V_{H} with the C-terminus of the V_{L} or the N-terminus of the V_{L} with the C-terminus of the V_{H}. An scFv lacks constant regions. Modified scFv and methods of modifying a scFv to bind to a target protein are described in WO 2022/258841 (which is incorporated herein by reference). Exemplary scFv's that bind TfR as also described herein, e.g., in Table 15.

The term “nanobody” refers to an antibody fragment consisting of a single monomeric variable antibody domain. Nanobodies derived from camelid heavy chain antibodies can be referred to as “VHH” fragments. Nanobodies derived from cartilaginous fish heavy chain antibodies can be referred to as “vNARs.” Modified VHH fragments and methods of modifying a VHH fragment to bind to a target protein, including TfR and CD98hc, are described in WO 2019/246288, WO 2021/205361, WO 2020/056327, WO 2022/103769, and WO 2023/023166 (each of which is incorporated herein by reference). Exemplary nanobodies that bind TfR as also described herein, e.g., in Table 15.

In some embodiments, a modified CH3 peptide comprises a CH3 peptide that is modified to bind a target protein, such as a BBB transport protein (e.g., TfR or CD98hc), a BBB cell surface protein, or a brain retention protein. Modified CH3 peptide and methods of modifying a CH3 peptide to bind to a target protein are described in US20180237496, US20200223935, and PCT/US2022/053220 (each of which is incorporated herein by reference). Exemplary modified CH3 peptides that bind TfR and CD98 as also described herein, e.g., in Tables 2, 8, 17, and 18.

In some embodiments, a peptide that specifically binds a BBB transport protein, BBB cell surface protein or brain retention protein comprises a heavy chain complementary determining region 3 (CDRH3) peptide that specifically binds a BBB transport protein, BBB cell surface protein, or brain retention protein. The CDRH3 can be inserted into a constant domain of an antibody, such as a CH3 peptide, a CH2 peptide, a CH1 peptide, or a CL peptide as described in described in WO 2023/087017 (incorporated herein by reference).

An Fc polypeptide or CH3 peptide used herein can be derived from an IgG, e.g., a human IgG1, IgG2, IgG3, or IgG4.

A modified fibronectin type III (FN3) domain comprises a FN3 domain that is modified to bind a target protein, such as a BBB transport protein (e.g., TfR or CD98hc), a BBB cell surface protein, or a brain retention protein. A FN3 domain is a consensus sequence of multiple FN3 domains from human Tenascin-C. Modified FN3 domains and methods of modifying a FN3 domain to bind to a target protein are described in US20100216708 (which is incorporated herein by reference). Specific examples of FN3 domains that bind TfR (CD71) are described in WO2021/076546 and WO2022/221505, each incorporated herein by reference.

A bicyclic peptide comprises a synthetic short peptide constrained by a chemical linker to form two loops. The chemical linker can form linkages to 3 different amino acids in the peptide. Bicyclic peptides can be made that specifically bind to target proteins, such as a BBB transport protein (e.g., TfR or CD98hc), a BBB cell surface protein, or a brain retention protein. Bicyclic peptides and methods of making bicyclic peptides that bind to a target protein are described in U.S. Pat. Nos. 8,680,022, 8,685,890, 8,778,844 (each of which is incorporated herein by reference). Specific bicyclic peptides that bind TfR are described in WO2022/101633, hereby incorporated by reference.

In some embodiments, a dual transporter comprises a multi-specific antibody or multi-specific antibody-like molecule.

In some embodiments, a dual transporter comprises a bispecific antibody or bispecific antibody-like molecule.

In some embodiments, a dual transporter comprises a bispecific antibody comprising a first antibody antigen binding domain that specifically binds a BBB transport protein (e.g., TfR or CD98hc) and a second antibody antigen binding domain that specifically binds a second, different protein that is a BBB surface protein or a brain retention protein. Each antibody antigen binding domain can be, but is not limited to, a Fab, a scFab, a Fv fragment, a scFv, or a nanobody. A bispecific antibody dual transporter can have two antigen binding domains of the same type (e.g., two Fabs, two scFabs, two Fv fragments, two scFvs, or two nanobodies) or a bispecific antibody dual transporter can have two antigen binding domains of two different types (e.g., a Fab and an scFab. Fv fragment, a scFv, or a nanobody: a scFab and a Fab, a Fv fragment, a scFv or a nanobody; an Fv fragment and a Fab, a scFab, a scFv, or a nanobody; a scFv and a Fab, a scFab, a Fv fragment, or a nanobody; or a nanobody and a Fab, a scFab, a Fv fragment, or a scFv). In a particular embodiment, the bispecific antibody contains a Fab and scFv.

In some embodiments, a dual transporter comprises a bispecific antibody comprising a first antibody heavy chain and a first antibody light chain, wherein the first antibody heavy chain and the first antibody light chain form an antibody variable domain that specifically binds a BBB transport protein (e.g., TfR or CD98hc), and a second antibody heavy chain and a second antibody light chain, wherein the second antibody heavy chain and the second antibody light chain form an antibody variable domain that specifically binds a second, different protein that is a BBB surface protein or a brain retention protein.

In some embodiments, a dual transporter comprises a bispecific heavy chain antibody wherein a first heavy chain of the heavy chain antibody specifically binds a BBB transport protein (e.g., TfR or CD98hc) and a second heavy chain of the heavy chain antibody specifically binds a second, different protein that is a BBB surface protein or a brain retention protein.

In some embodiments, a dual transporter comprises a bispecific F(ab′)₂ comprising a first Fab that specifically binds a BBB transport protein (e.g., TfR or CD98hc) and a second Fab that specifically binds a second, different protein that is a BBB surface protein or a brain retention protein. The dual transporter bispecific F(ab′)2 may or may not be linked to one or more Fc polypeptides, one or more CH2 peptides, one or more CH3 peptides, an Fc dimer or a CH3 peptide dimer. In some embodiments, the first Fab is linked to a first Fc polypeptide, a first CH2 peptide, or a first CH3 peptide; and the second Fab is linked to a second Fc polypeptide, second CH2 peptide, or a second CH3 peptide.

In some embodiments a dual transporter comprises a bispecific sc(Fv)₂ comprising an a first scFv that specifically binds a BBB transport protein (e.g., TfR or CD98hc) and a second scFv that specifically binds a second, different protein that is a BBB surface protein or a brain retention protein. The dual transporter bispecific sc(Fv)₂ may or may not be linked to one or more Fc polypeptides, one or more CH2 peptides, one or more CH3 peptides, an Fc dimer or a CH3 peptide dimer. In some embodiments, the first scFv is linked to a first Fc polypeptide, a first CH2 peptide, or a first CH3 peptide; and the second scFv is linked to a second Fc polypeptide, second CH2 peptide, or a second CH3 peptide. In some embodiments, a dual transporter bispecific sc(Fv)₂ comprises a diabody. In some embodiments, a dual transporter comprises a bispecific minibody, comprising a first scFv linked to a first CH3 peptide and second scFv linked to a second CH3 peptide wherein the first scFv specifically binds a BBB transport protein (e.g., TfR or CD98hc), and wherein the second scFv specifically binds a second, different protein that is a BBB surface protein or a brain retention protein, and wherein the first and second CH3 peptides form a dimer.

In some embodiments, a dual transporter comprises a bispecific nanobody comprising an a first nanobody that specifically binds a BBB transport protein (e.g., TfR or CD98hc) and a second nanobody that specifically binds a second, different protein that is a BBB surface protein or a brain retention protein. The dual transporter bispecific nanobody may or may not be linked to one or more Fc polypeptides, one or more CH2 peptides, one or more CH3 peptides, an Fc dimer or a CH3 peptide dimer. In some embodiments, the first nanobody is linked to a first Fc polypeptide, a first CH2 peptide, or a first CH3 peptide; and the second nanobody is linked to a second Fc polypeptide, second CH2 peptide, or a second CH3 peptide. The first and second nanobodies can independently be, a vNAR or a VHH. In some embodiments, a dual transporter bispecific nanobody comprises a (vNAR)₂ peptide, a (VHH)₂ peptide, a vNAR-VHH peptide, or a VHH-vNAR peptide.

In some embodiments, a dual transporter comprises a first binding region that specifically binds a BBB transport protein (e.g., TfR or CD98hc) linked to a bispecific antibody that binds to the second protein and therapeutic target. In some embodiments, a dual transporter comprises a second binding region that specifically binds to a BBB surface protein or a brain retention protein linked to a bispecific antibody that binds to the BBB transport protein and therapeutic target.

In some embodiments, a dual transporter comprises a tri-specific antibody or tri-specific antibody-like molecule (dual transporter tri-specific antibody). In some embodiments, a dual transporter tri-specific antibody comprises a first antibody antigen binding domain the specifically binds a BBB transport protein (e.g., TfR or CD98hc), a second antibody antigen binding domain that specifically binds a second, different protein that is a BBB surface protein or a brain retention protein, and a third antibody antigen binding domain the specifically binds a target protein.

A dual transporter tri-specific antibody is not limited to any particular configuration of antigen binding domains. A tri-specific antibody-like molecule can be, but is not limited to, a trimeric Fab ((Fab)₃), a trimeric scFv (triabody or (scFv)₃), or a combination thereof. In some embodiments, a dual transporter tri-specific antibody comprises a bispecific immunoglobulin (e.g., IgG) linked to a Fab, scFab, a Fv fragment, a scFv, or a nanobody.

In some embodiments, a dual transporter comprises a tetra-specific antibody or tetra-specific antibody-like molecule (dual transporter tetra-specific antibody). In some embodiments, a dual transporter tetra-specific antibody comprises a first antibody antigen binding domain the specifically binds a BBB transport protein (e.g., TfR or CD98hc), a second antibody antigen binding domain that specifically binds a second, different protein that is a BBB surface protein or a brain retention protein, a third antibody antigen binding domain the specifically binds a first target protein, and a fourth antibody antigen binding domain that specifically binds a second target protein. The first target protein and the second target protein may be the same or different.

A dual transporter tetra-specific antibody is not limited to any particular configuration of antigen binding domains. A tetra-specific antibody-like molecule can be, but is not limited to, a tetrameric Fab ((Fab)₃), a tetrameric scFv (tetrabody or (scFv)₄), tetrameric nanobody, or a combination thereof. In some embodiments, a dual transporter tetra-specific antibody comprises an immunoglobulin (e.g., IgG) linked to a two antigen binding domains independently selected from the group consisting of: a Fab, a scFab, a Fv fragment, a scFv, or a nanobody. In some embodiments, a dual transporter comprises a dual-variable domain immunoglobulin (DVD-Ig). A DVD-Ig comprises a tetravalent immunoglobulin having two variable domains on each Fab arm.

In some embodiments, a dual transporter tetra-specific antibody comprises a therapeutic antibody linked to a first Fab, scFab, scFv, or nanobody that specifically binds to the BBB transport protein and a second Fab, scFab, scFv, or nanobody that specifically binds to the second protein. The first Fab, scFab, scFv or nanobody and the second Fab, scFab, scFv, or nanobody are independently linked to a heavy chain, alight chain, or a Fc of the therapeutic antibody. In some embodiments, the first Fab, scFab, scFv or nanobody and/or the second Fab, scFab, scFv, or nanobody are linked to a C-terminal end of a heavy chain of the therapeutic antibody. In some embodiments, the BBB transport protein is TfR. In some embodiments, the BBB transport protein is CD98hc. In some embodiments, the BBB transport protein is TfR and the second protein is CD98hc.

In some embodiments, a dual transporter comprises a first binding region that specifically binds a BBB transport protein (e.g., TfR or CD98hc) and a second binding region that specifically binds a second, different protein that is a BBB surface protein or a brain retention protein, wherein the first binding region and the second binding region are linked to each other.

In some embodiments, a dual transporter comprises a first binding region that specifically binds a BBB transport protein (e.g., TfR or CD98hc) and a second binding region that specifically binds a second, different protein that is a BBB surface protein or a brain retention protein, wherein the first binding region and the second binding region are linked to a scaffold.

The scaffold can be, but is not limited to, a polypeptide, a protein, an antibody, or an antibody fragment. The antibody scaffold be, but is not limited to, an IgG, an IgA, an IgD, an IgE, an IgM, or a heavy chain antibody. An antibody scaffold can be monospecific or multi-specific (e.g., bi-specific), antibody. The antibody fragment scaffold can be, but is not limited to, an Fc polypeptide, an Fc dimer, an CH2 peptide, a CH2 dimer, a CH3 peptide, a CH3 dimer, a Fab fragment, a multimeric Fab, an F(ab′)₂ fragment, a multimeric F(ab′)₂ fragment, an scFab, a multimeric scFab, an scFv, a multimeric scFv, a nanobody, or a multimeric nanobody. The antibody fragment scaffold can be monospecific or multi-specific (e.g., bi-specific). An antibody scaffold or antibody fragment scaffold can comprise a therapeutic antibody or therapeutic antibody fragment. An antibody scaffold or antibody fragment scaffold can be derived from a therapeutic antibody or therapeutic antibody fragment. An antibody scaffold or antibody fragment scaffold can contain an antibody antigen binding domain from a therapeutic antibody.

In some embodiments, a dual transporter comprises a first binding region that specifically binds a BBB transport protein (e.g., TfR or CD98hc) and a second binding region that specifically binds a second, different protein that is a BBB surface protein or a brain retention protein, wherein the first binding and the second binding region are linked to an antibody scaffold. The first binding region and the second binding region can be independently linked to a variable domain region, a light chain, a C-terminal end of a light chain, a N-terminal end of a light chain, a light chain variable region, a light chain constant region, a heavy chain, a C-terminal end of a heavy chain, a N-terminal end of a heavy chain, a heavy chain variable region, a heavy chain CH1 region, a heavy chain CH2 region, or a heavy chain CH3.

In some embodiments, the first binding region is linked to a C-terminal end of one light chain of an antibody scaffold and the second binding region is linked to a C-terminal end of the other light chain of the antibody scaffold.

In some embodiments, the first binding region is linked to a C-terminal end of one heavy chain of an antibody scaffold and the second binding region is linked to a C-terminal end of the other heavy chain of the antibody scaffold.

In some embodiments, the first binding region is linked to a C-terminal end of a light chain of an antibody scaffold and the second binding region is linked to a C-terminal end of a heavy chain of the antibody scaffold.

In some embodiments, the first binding region is linked to a N-terminal end of one light chain of an antibody scaffold and the second binding region is linked to a N-terminal end of the other light chain of the antibody scaffold.

In some embodiments, the first binding region is linked to a N-terminal end of one heavy chain of an antibody scaffold and the second binding region is linked to a N-terminal end of the other heavy chain of the antibody scaffold.

In some embodiments, the first binding region is linked to a N-terminal end of a light chain of an antibody scaffold and the second binding region is linked to a N-terminal end of a heavy chain of the antibody scaffold.

In some embodiments, the first binding region is linked to a C-terminal end of a light chain of an antibody scaffold and the second binding region is linked to a N-terminal end of a light chain or a heavy chain of the antibody scaffold.

In some embodiments, the first binding region is linked to a C-terminal end of a heavy chain of an antibody scaffold and the second binding region is linked to a N-terminal end of a light chain or a heavy chain of the antibody scaffold.

In some embodiments, the first binding region is linked to a N-terminal end of a light chain of an antibody scaffold and the second binding region is linked to a C-terminal end of a light chain or a heavy chain of the antibody scaffold.

In some embodiments, the first binding region is linked to a N-terminal end of a heavy chain of an antibody scaffold and the second binding region is linked to a C-terminal end of a light chain or a heavy chain of the antibody scaffold.

The first binding region and can be, but is not limited to, a peptide, a modified CH3 peptide, modified tenascin C peptide, a bicyclic peptide, a Fab, a scFab, scFv, or a nanobody. The second binding region and can be, but is not limited to, a peptide, a modified CH2 peptide, a modified CH3 peptide, modified tenascin C peptide, a bicyclic peptide, a Fab, a scFab, scFv, or a nanobody.

In some embodiments, the first binding region comprises an scFv linked to a C-terminal end of one light chain of an antibody scaffold and the second binding region comprises a scFv linked to a C-terminal end of the other light chain of the antibody scaffold. In some embodiments, the first binding region specifically binds to TfR. In some embodiments, the first binding region specifically binds to CD98hc. In some embodiments, the first binding region specifically binds to TfR and the second binding region specifically binds to CD98hc.

In some embodiments, the first binding region and/or the second comprises an scFv, wherein the N-terminal end of the scFv is linked to the C terminal end of one light chain of an antibody scaffold and the C-terminal end of the scFv is linked to the N-terminal end of the other light chain of the antibody scaffold.

In some embodiments, a dual transporter comprises a DVD-Ig, wherein the DVD-Ig comprises two Fv fragments (antibody variable regions) on each Fab arm of the DVD-Ig. A DVD-Ig thus contains four Fv fragments: a first Fv fragment, a second Fv fragment, a third Fv fragment and a fourth Fv fragment. At least one of the Fv fragments specifically binds to the BBB transport protein and at least one of the Fv fragments specifically binds to the second protein. In some embodiments, one of the Fv fragments binds to the BBB transport protein, one of the Fv fragments binds to the second protein, and the other two Fv fragments specifically bind to one or two target proteins (e.g., therapeutic targets). The DVD-Ig dual transporter is not limited to any particular arrangement of Fv fragments. The Fv fragment that specifically binds the BBB transport protein can be the first, second third, or fourth Fv fragment. Similarly, the Fv fragment that specifically binds the second protein can be the first, second third, or fourth Fv fragment, provided the Fv fragment that binds the second protein is different from the Fv fragment that specifically binds the BBB transport protein. In some embodiments, a first Fab arm of the DVD-Ig comprises a first Fv fragment that specifically binds to a target protein and a second Fv fragment that specifically binds to the BBB transport protein and a second Fab arm of the DVD-Ig comprises a third Fv fragment that specifically binds to a target protein and a fourth Fv fragment that specifically binds to the second protein. In some embodiments, a first Fab arm of the DVD-Ig comprises a first Fv fragment that specifically binds to the BBB transport protein and a second Fv fragment that specifically binds to the second protein and a second Fab arm of the DVD-Ig comprises third and fourth Fv fragments that specifically binds to one or two target protein.

In some embodiments, a dual transporter comprises a first binding region comprising a first Fc polypeptide or CH3 modified to specifically bind a BBB transport protein (e.g., TfR or CD98hc). The second binding region may be provided by a second peptide that specifically binds the second protein, a second antibody antigen binding domain that specifically binds the second protein, a second Fc polypeptide modified to specifically bind the second protein, a second CH3 peptide modified to specifically bind the second protein, a second fibronectin type III domain peptide modified to specifically bind the second protein, or a second bicyclic peptide that specifically binds the second protein. In some embodiments, the BBB transport protein is TfR. In some embodiments, the BBB transport protein is CD98hc. In some embodiments, the BBB transport protein is TfR and the second protein is CD98hc.

In some embodiments, a dual transporter comprises a second binding region comprising a second Fc polypeptide or CH3 modified to specifically bind a second, different protein that is a BBB surface protein or a brain retention protein. The first binding region may be provided by a first peptide that specifically binds a BBB transport protein (e.g., TfR or CD98hc), a first antibody antigen binding domain that specifically binds the BBB transport protein, a first Fc polypeptide modified to specifically bind the BBB transport protein, a first CH3 peptide modified to specifically bind the BBB transport protein, a first fibronectin type III domain peptide modified to specifically bind the BBB transport protein, or a first bicyclic peptide that specifically binds the BBB transport protein. In some embodiments, the second protein is CD98hc. In some embodiments, the BBB transport protein is TfR, and the second protein is CD98hc.

In some embodiments, a dual transporter comprises an Fc dimer, wherein the Fc dimer comprises a first Fc polypeptide comprising a CH3 peptide modified to specifically bind TfR and a second Fc polypeptide comprising a CH3 peptide modified to specifically bind CD98hc.

An Fc dimer or an Fc polypeptide as described herein may further comprise a partial or full hinge region. The hinge region can be from any immunoglobulin subclass or isotype. An illustrative immunoglobulin hinge is an IgG hinge region, such as an IgG1 hinge region, e.g., human IgG1 hinge amino acid sequence EPKSCDKTHTCPPCP (SEQ ID NO:3). The Fc polypeptide, which may comprise a hinge or partial hinge region, is further fused to a therapeutic molecule (e.g., a therapeutic peptide; e.g., a therapeutic antibody or fragment thereof).

In some embodiments, a dual transporter comprising an Fc dimer is linked to one or more Fabs, one or more F(ab′)2s, one or more scFabs, one or more scFvs, or one or more nanobodies. In some embodiments, a dual transporter comprising an Fc dimer is linked to one or more therapeutic molecules. The therapeutic molecule can be, but is not limited to, a therapeutic Fab, a therapeutic F(ab′)2, a therapeutic scFab, a scFv, or a therapeutic nanobodies.

In some embodiments, a dual transporter comprises a therapeutic antibody having a first Fc polypeptide or CH3 peptide modified to specifically bind a BBB transport protein (e.g., TfR or CD98hc) and a second Fc polypeptide or CH3 peptide modified to specifically bind a second, different protein that is a BBB surface protein or a brain retention protein. The therapeutic antibody may be monospecific of bispecific.

In some embodiments, a dual transporter comprises a therapeutic antibody having a first Fc polypeptide or CH3 peptide modified to specifically bind TfR and a second Fc polypeptide or CH3 peptide modified to specifically bind a second, different protein that is a BBB surface protein or a brain retention protein. The therapeutic antibody may be monospecific of bispecific.

In some embodiments, a dual transporter comprises a therapeutic antibody having a first Fc polypeptide or CH3 peptide modified to specifically bind CD98hc and a second Fc polypeptide or CH3 peptide modified to specifically bind a second, different protein that is a BBB surface protein or a brain retention protein. The therapeutic antibody may be monospecific of bispecific.

In some embodiments, a dual transporter comprises a therapeutic antibody having a first Fc polypeptide or CH3 peptide modified to specifically bind TfR and a second Fc polypeptide or CH3 peptide modified to specifically bind CD98hc. The therapeutic antibody may be monospecific of bispecific.

Any of the described dual transporters may further comprise one or more therapeutic molecules. The therapeutic molecule can be, but is not limited to, small molecule drug or a therapeutic peptide. A therapeutic peptide can be, but is not limited to, an antibody, a Fab, a F(ab′)2, a scFab, a Fv fragment, scFvs, or a nanobody.

In some embodiments, a dual transport comprises a TfR binding region and a second binding region that specifically binds to a second protein, wherein the second protein is expressed on the luminal surface of the BBB or is a brain retention protein, and wherein the second protein is different from the BBB transport protein. In some embodiments, a dual transporter comprises a TfR binding region and a second binding region that binds CD98hc, MOG, GLUT1, MFSD2A, IGFTR, or CA-IV. It has been demonstrated herein (see Examples) that dual transporters having both TfR- and CD98hc-binding unexpectedly provide both (i) higher brain Cmax (maximum concentration in the brain), and (ii) longer brain exposure times (i.e., AUC (area under the curve)) relative to a BBB transporter having either TfR or CD98hc binding alone. TfR binding alone provides lower brain Cmax levels and shorter brain exposure times relative to the dual transporters provided herein. CD98hc binding alone provides lower brain Cmax levels relative to the dual transporters provided herein. Improvement in brain Cmax and brain was also observed for a TfR biding region-MOG binding region dual transporter.

By combining the two different binding regions, the dual transporters provide increased brain Cmax and increased brain exposure times (i.e., retention). Thus, the described dual transporters can be used to developing therapeutics that cross the BBB or to improve brain delivery for therapeutic compounds linked to the dual transporters. Linking a therapeutic compound to a described dual transporter can also be used to improve efficacy or therapeutic index of therapeutic compounds whose target is in the brain or CNS.

Cmax and brain exposure time can be further optimized by adjusting the affinities of the first (e.g., TfR) binding region and the second (e.g., CD98hc, MOG, GLUT1. MFSD2A, or CA-IV) binding region. For example, it has been demonstrated (see Examples) that relatively strong TfR affinity paired with relatively strong CD98hc affinity results in the higher brain Cmax levels and moderate brain exposure times, relative to strong TfR affinity paired with weaker CD98hc affinity. Moderate to weak TfR affinity paired with strong CD98hc affinity results in moderate brain Cmax and longer brain exposure times, relative to relatively strong TfR affinity paired with relatively strong CD98hc affinity. Moderate to weak TfR paired with weak CD98hc affinity results in lower brain levels and moderate brain exposure times. Longest brain exposure times with observed with moderate to weak TfR affinity paired with strong CD98hc affinity. Thus, in some embodiments, a dual transporter comprises a first binding region that specifically binds to a blood brain barrier (BBB) transport protein (e.g., TfR) with moderate to week affinity and a second binding region that specifically binds to a second protein (e.g., CD98hc, MOG, GLUT1, MFSD2A, or CA-IV) wherein the second protein is expressed on the luminal surface of the BBB or is a brain retention protein (and is different from the BBB transport protein) with strong affinity.

It has also been demonstrated herein that, as the ratio of TfR binding affinity to CD98hc binding affinity increases (i.e., above 10-fold), brain Cmax increases while brain exposure time decreases. Conversely, as the ratio of CD98hc binding affinity to TfR binding affinity increase (i.e., above 10-fold), brain Cmax decreases and brain exposure time increases. Thus, adjusting the affinities of the first and second binding regions for the respective target proteins, can be used to provide dual transporters with a desired brain Cmax levels and brain exposure times. In some embodiments, brain Cmax and brain retention can be modulated by altering the affinities of the first and second binding regions for their respect target proteins. Such modulation can be used to alter brain Cmax and brain retention for different therapeutic, discovery, or research purposes.

In some embodiments, the first and second binding regions of a dual transporter can independently have weak, moderate, or strong affinities for the respective targets.

In some embodiments, the first binding region has weak affinity to its target protein and the second binding region has weak affinity to its target protein. In some embodiments, the first binding region has weak affinity to its target protein and the second binding region has moderate affinity to its target protein. In some embodiments, the first binding region has weak affinity to its target protein and the second binding region has strong affinity to its target protein. In some embodiments, the first binding region has weak affinity to TfR and the second binding region has weak affinity to CD98hc. In some embodiments, the first binding region has weak affinity to TfR and the second binding region has moderate affinity to CD98hc. In some embodiments, the first binding region has weak affinity to TfR and the second binding region has strong affinity to CD98hc. In some embodiments, the first binding region has weak affinity to TfR and the second binding region has weak affinity to MOG. In some embodiments, the first binding region has weak affinity to TfR and the second binding region has moderate affinity to MOG. In some embodiments, the first binding region has weak affinity to TfR and the second binding region has strong affinity to MOG.

In some embodiments, the first binding region has moderate affinity to its target protein and the second binding region has weak affinity to its target protein. In some embodiments, the first binding region has moderate affinity to its target protein and the second binding region has moderate affinity to its target protein. In some embodiments, the first binding region has moderate affinity to its target protein and the second binding region has strong affinity to its target protein. In some embodiments, the first binding region has moderate affinity to TfR and the second binding region has weak affinity to CD98hc. In some embodiments, the first binding region has moderate affinity TfR and the second binding region has moderate affinity to CD98hc. In some embodiments, the first binding region has moderate affinity to TfR and the second binding region has strong affinity to CD98hc. In some embodiments, the first binding region has moderate affinity to TfR and the second binding region has weak affinity to MOG. In some embodiments, the first binding region has moderate affinity TfR and the second binding region has moderate affinity to MOG. In some embodiments, the first binding region has moderate affinity to TfR and the second binding region has strong affinity to MOG.

In some embodiments, the first binding region has strong affinity to its target protein and the second binding region has weak affinity to its target protein. In some embodiments, the first binding region has strong affinity to its target protein and the second binding region has moderate affinity to its target protein. In some embodiments, the first binding region has strong affinity to its target protein and the second binding region has strong affinity to its target protein. In some embodiments, the first binding region has strong affinity to TfR and the second binding region has weak affinity to CD98hc. In some embodiments, the first binding region has strong affinity TfR and the second binding region has moderate affinity to CD98hc. In some embodiments, the first binding region has strong affinity to TfR and the second binding region has strong affinity to CD98hc. In some embodiments, the first binding region has strong affinity to TfR and the second binding region has weak affinity to MOG. In some embodiments, the first binding region has strong affinity to TfR and the second binding region has moderate affinity to MOG. In some embodiments, the first binding region has strong affinity to TfR and the second binding region has strong affinity to MOG.

Strong affinity indicates the affinity of the first or second binding region for its respective target protein is less than 250 nM. In some embodiments, a first or second binding region having strong affinity has an affinity to its target protein of about 50 nM to about 250 nM. In some embodiments, a first or second binding region having strong affinity has an affinity to its target protein of 150 nM±100 nM, 150 nM±75 nM, or 150 nM±50 nM. In some embodiments, a first or second binding region having strong affinity has an affinity to its target protein of about 50 nM, about 75 nM, about 100 nM, about 125 nM, about 150 nM, about 175 nM, about 200 nM, about 225 nM, or about 250 nM. Moderate affinity indicates the affinity of the first or second binding region for its respective target protein is about 300 nM to about 900 nM, about 400 nM to about 800 nm, about 500 to about 700 nM, or about 600 nM. In some embodiments, a first or second binding region having moderate affinity has an affinity to its target protein of 600 nM±300 nM, 600 nM±200 nM, or 600 nM±100 nM. In some embodiments, a first or second binding region having moderate affinity has an affinity to its target protein of about 400 nM, about 450 nM, about 500 nM, about 550 nM, about 600 nM, about 650 nM, about 700 nM, about 750 nM, or about 800 nM. Weak affinity indicates the affinity of the first or second binding region for its respective target protein is about 900 nM to about 6000 nM, about 1000 nM to about 6000 nM, about 900 nM to about 5000 nm, about 1000 nM to about 5000 nm, about 900 to about 4000 nM, about 1000 to about 4000 nM, about 900 to about 3000 nM, about 1000 to about 3000 nM, about 900 to about 2500 nM, about 1000 to about 2500 nM, about 1500 to about 6000 nM, about 1500 to about 5000 nM, about 1500 to about 4000 nM, about 1500 to about 3000 nM, about 1500 to about 2500 nM, about 2000 to about 6000 nM, about 2000 to about 5000 nM, about 2000 to about 4000 nM, or about 2000 to about 3000 nM. In some embodiments, affinity is measure using a Surface Plasmon Resonance (SPR) methods (e.g., using a Biacore™ system), kinetic exclusion assays (e.g., using a KinExA® system), and/or BioLayer interferometry (e.g., using the ForteBio® Octet® platform). It will be appreciated by those skilled the in the art that affinity measurements are subject to a degree of variability. Therefore, the above affinities may vary within a reasonable range depending on the assay or the protein.

The affinity of a binding region for its target protein can vary depending on the context of the binding region. For example, the affinity of an isolated TfR-binding polypeptide for TfR can be different when measured for the isolate TfR-binding polypeptide compared to its affinity to TfR when combined with a second binding region to form a dual transporter. Therefore, in some embodiments, the affinity values and ranges provided above correspond to the affinity of the binding region for its target protein when the binding region is part of a dual transporter. In other words, the affinities of the first and second binding regions for their target proteins correspond to the affinities of the dual transporter for the first and second binding region target proteins.

In some embodiments, a first binding region that binds TfR with strong affinity, binds TfR with an affinity of less than 250 nM. In some embodiments, a first binding region that binds TfR with strong affinity, binds TfR with an affinity 50 nM to about 250 nM. In some embodiments, a first binding region that binds TfR with strong affinity, binds TfR with an affinity 150 nM±100 nM, 150 nM±75 nM, or 150 nM±50 nM. In some embodiments, a first binding region that binds TfR with strong affinity, binds TfR with an affinity of about 50 nM, about 75 nM, about 100 nM, about 125 nM, about 150 nM, about 175 nM, about 200 nM, about 225 nM, or about 250 nM. In some embodiments, a first binding region that binds TfR with moderate affinity, binds TfR with an affinity of about 300 nM to about 900 nM, about 400 nM to about 800 nm, about 500 to about 700 nM, or about 600 nM. In some embodiments, a first binding region that binds TfR with moderate affinity, binds TfR with an affinity of 600 nM±300 nM, 600 nM±200 nM, or 600 nM±100 nM. In some embodiments, a first binding region that binds TfR with moderate affinity, binds TfR with an affinity of about 400 nM, about 450 nM, about 500 nM, about 550 nM, about 600 nM, about 650 nM, about 700 nM, about 750 nM, or about 800 nM. In some embodiments, a first binding region that binds TfR with weak affinity, binds TfR with an affinity of about 900 nM to about 6000 nM, about 1000 nM to about 6000 nM, about 900 nM to about 5000 nm, about 1000 nM to about 5000 nm, about 900 to about 4000 nM, about 1000 to about 4000 nM, about 900 to about 3000 nM, about 1000 to about 3000 nM, about 900 to about 2500 nM, about 1000 to about 2500 nM, about 1500 to about 6000 nM, about 1500 to about 5000 nM, about 1500 to about 4000 nM, about 1500 to about 3000 nM, about 1500 to about 2500 nM, about 2000 to about 6000 nM, about 2000 to about 5000 nM, about 2000 to about 4000 nM, or about 2000 to about 3000 nM.

In some embodiments, a first binding region that binds CD98hc with strong affinity, binds CD98hc with an affinity of less than 250 nM. In some embodiments, a first binding region that binds CD98hc with strong affinity, binds CD98hc with an affinity 50 nM to about 250 nM. In some embodiments, a first binding region that binds CD98hc with strong affinity, binds CD98hc with an affinity 150 nM f 100 nM, 150 nM±75 nM, or 150 nM±50 nM. In some embodiments, a first binding region that binds CD98hc with strong affinity, binds CD98hc with an affinity of about 50 nM, about 75 nM, about 100 nM, about 125 nM, about 150 nM, about 175 nM, about 200 nM, about 225 nM, or about 250 nM. In some embodiments, a first binding region that binds CD98hc with moderate affinity, binds CD98hc with an affinity of about 300 nM to about 900 nM, about 400 nM to about 800 nm, about 500 to about 700 nM, or about 600 nM. In some embodiments, a first binding region that binds CD98hc with moderate affinity, binds CD98hc with an affinity of 600 nM±300 nM, 600 nM±200 nM, or 600 nM f 100 nM. In some embodiments, a first binding region that binds CD98hc with moderate affinity, binds CD98hc with an affinity of about 400 nM, about 450 nM, about 500 nM, about 550 nM, about 600 nM, about 650 nM, about 700 nM, about 750 nM, or about 800 nM. In some embodiments, a first binding region that binds CD98hc with weak affinity, binds CD98hc with an affinity of about 900 nM to about 6000 nM, about 1000 nM to about 6000 nM, about 900 nM to about 5000 nM, about 1000 nM to about 5000 nm, about 900 nM to about 5000 nM, about 1000 to about 4000 nM, about 900 nM to about 3000 nM, about 1000 to about 3000 nM, about 900 nM to about 2500 nM, about 1000 to about 2500 nM, about 1500 to about 6000 nM, about 1500 to about 5000 nM, about 1500 to about 4000 nM, about 1500 to about 3000 nM, about 1500 to about 2500 nM, about 2000 to about 6000 nM, about 2000 to about 5000 nM, about 2000 to about 4000 nM, or about 2000 to about 3000 nM.

In some embodiments, a first binding region that binds MOG with strong affinity, binds MOG with an affinity of less than 250 nM. In some embodiments, a first binding region that binds MOG with strong affinity, binds MOG with an affinity 50 nM to about 250 nM. In some embodiments, a first binding region that binds MOG with strong affinity, binds MOG with an affinity 150 nM±100 nM, 150 nM f 75 nM, or 150 nM±50 nM. In some embodiments, a first binding region that binds MOG with strong affinity, binds MOG with an affinity of about 50 nM, about 75 nM, about 100 nM, about 125 nM, about 150 nM, about 175 nM, about 200 nM, about 225 nM, or about 250 nM. In some embodiments, a first binding region that binds MOG with moderate affinity, binds MOG with an affinity of about 300 nM to about 900 nM, about 400 nM to about 800 nm, about 500 to about 700 nM, or about 600 nM. In some embodiments, a first binding region that binds MOG with moderate affinity, binds MOG with an affinity of 600 nM±300 nM, 600 nM±200 nM, or 600 nM f 100 nM. In some embodiments, a first binding region that binds MOG with moderate affinity, binds MOG with an affinity of about 400 nM, about 450 nM, about 500 nM, about 550 nM, about 600 nM, about 650 nM, about 700 nM, about 750 nM, or about 800 nM. In some embodiments, a first binding region that binds MOG with weak affinity, binds MOG with an affinity of about 900 nM to about 6000 nM, about 1000 nM to about 6000 nM, about 900 nM to about 5000 nM, about 1000 nM to about 5000 nm, about 900 nM to about 4000 nM, about 1000 to about 4000 nM, about 900 nM to about 3000 nM, about 1000 to about 3000 nM, about 900 nM to about 2500 nM, about 1000 to about 2500 nM, about 1500 to about 6000 nM, about 1500 to about 5000 nM, about 1500 to about 4000 nM, about 1500 to about 3000 nM, about 1500 to about 2500 nM, about 2000 to about 6000 nM, about 2000 to about 5000 nM, about 2000 to about 4000 nM, or about 2000 to about 3000 nM.

In some embodiments, a dual transporter comprises:
(a) a first binding region that binds to TfR with an affinity of about 900 nM to about 6000 nM and a second binding region binds to CD98hc with an affinity of about 900 nM to about 6000 nM;
(b) a first binding region that binds to TfR with an affinity of about 900 nM to about 6000 nM and a second binding region binds to CD98hc with an affinity of about 300 nM to about 900 nM; or
(c) a first binding region that binds to TfR with an affinity of about 900 nM to about 6000 nM and a second binding region binds to CD98hc with an affinity of less than about 250 nM.

In some embodiments, a dual transporter comprises:
(a) a first binding region that binds to TfR with an affinity of about 1000 nM to about 6000 nM and a second binding region that binds to CD98hc with an affinity of about 1000 nM to about 6000 nM;
(b) a first binding region that binds to TfR with an affinity of about 1000 nM to about 6000 nM and a second binding region binds to CD98hc with an affinity of about 300 nM to about 900 nM; or
(c) a first binding region that binds to TfR with an affinity of about 1000 nM to about 6000 nM and a second binding region binds to CD98hc with an affinity of less than about 250 nM.

In some embodiments, a dual transporter comprises a first binding region that binds to TfR with an affinity of about 900 nM to about 6000 nM and a second binding region binds to CD98hc with an affinity of about 50 nM to about 250 nM.

In some embodiments, a dual transporter comprises a first binding region that binds to TfR with an affinity of about 900 nM to about 6000 nM and a second binding region binds to CD98hc with an affinity of about 300 nM to about 900 nM.

In some embodiments, a dual transporter comprises a first binding region that binds to TfR with an affinity of about 900 nM to about 6000 nM and a second binding region binds to CD98hc with an affinity of about 50 nM to about 900 nM.

In some embodiments, a dual transporter comprises:
(a) a first binding region that binds to TfR with an affinity of about 300 nM to about 900 nM and a second binding region that binds to CD98hc with an affinity of about 900 nM to about 6000 nM;
(b) a first binding region that binds to TfR with an affinity of about 300 nM to about 900 nM and a second binding region that binds to CD98hc with an affinity of about 1000 nM to about 6000 nM;
(c) a first binding region that binds to TfR with an affinity of about 300 nM to about 900 nM and a second binding region that binds to CD98hc with an affinity of about 300 nM to about 900 nM; or
(d) a first binding region that binds to TfR with an affinity of about 300 nM to about 900 nM and a second binding region that binds to CD98hc with an affinity of less than about 250 nM; or
In some embodiments, a dual transporter comprises a first binding region that binds to TfR with an affinity of about 300 nM to about 900 nM and a second binding region that binds to CD98hc with an affinity of about 50 nM to about 250 nM.

In some embodiments, a dual transporter comprises a first binding region that binds to TfR with an affinity of about 300 nM to about 900 nM and a second binding region that binds to CD98hc with an affinity of about 300 nM to about 900 nM.

In some embodiments, a dual transporter comprises a first binding region that binds to TfR with an affinity of about 300 nM to about 900 nM and a second binding region that binds to CD98hc with an affinity of about 50 nM to about 900 nM.

In some embodiments, a dual transporter comprises:
(a) a first binding region that binds to TfR with an affinity of less than about 250 nM and a second binding region that binds to CD98hc with an affinity of about 1000 nM to about 6000 nM;
(b) a first binding region that binds to TfR with an affinity of less than about 250 nM and a second binding region that binds to CD98hc with an affinity of about 300 nM to about 900 nM; or
(c) a first binding region that binds to TfR with an affinity of less than about 250 nM and a second binding region that binds to CD98hc with an affinity of less than about 250 nM.

In some embodiments, a dual transporter comprises a first binding region that binds to TfR with an affinity of about 50 nM to about 250 nM and a second binding region that binds to CD98hc with an affinity of about 300 nM to about 900 nM.

In some embodiments, a dual transporter comprises a first binding region that binds to TfR with an affinity of about 50 nM to about 250 nM and a second binding region that binds to CD98hc with an affinity of about 50 nM to about 250 nM.

In some embodiments, a dual transporter comprises a first binding region that binds to TfR with an affinity of about 50 nM to about 250 nM and a second binding region that binds to CD98hc with an affinity of about 50 nM to about 900 nM.

In some embodiments, a dual transporter comprises:
(a) a first binding region that binds to TfR with an affinity of about 300 nM to about 6000 nM and a second binding region that binds to CD98hc with an affinity of about 900 nM to about 6000 nM;
(c) a first binding region that binds to TfR with an affinity of about 300 nM to about 6000 nM and a second binding region that binds to CD98hc with an affinity of about 300 nM to about 900 nM;
(d) a first binding region that that binds to TfR with an affinity of about 300 nM to about 6000 nM and a second binding region that binds to CD98hc with an affinity of less than about 250 nM;
(e) a first binding region that binds to TfR with an affinity of about 300 nM to about 6000 nM and a second binding region that binds to CD98hc with an affinity of about 50 nM to 250 nM; or
(f) a first binding region that binds to TfR with an affinity of about 300 nM to about 6000 nM and a second binding region that binds to CD98hc with an affinity of about 50 nM to 900 nM.

In some embodiments, in order to increase both uptake and retention in the brain of a dual transporter, the affinity of the dual transporter to the BBB transport protein (e.g., TfR) is about 50 nM to about 1000 nM, and the affinity to the second protein is about 1 nM to about 500 nM. In some embodiments, the affinity of the dual transporter to the BBB transport protein (e.g., TfR) is about 100 to about 600 nM and the affinity to the second protein is about 50 nM to about 300 nM. In some embodiments, the affinity of the dual transporter to the BBB transport protein (e.g., TfR) is about 100 nM to about 400 nM, and the affinity to the second protein is about 50 nM to about 200 nM.

In some embodiments, in order to increase brain uptake while maximizing brain retention of a dual transporter, the affinity of the dual transporter to the BBB transport protein (e.g., TfR) is about 250 to about 6000 nM, and the affinity to the second protein is about 1 to about 250 nM. In some embodiments, the affinity of the dual transporter to the BBB transport protein (e.g., TfR) is about 250 to about 1000 nM, and the affinity to the second protein is about 1 to about 100 nM. In some embodiments, the affinity of the dual transporter to the BBB transport protein (e.g., TfR) is about 50 to about 250 nM, and the affinity to the second protein is about 1 to about 100 nM.

In some embodiments, if low affinity to the BBB transport protein is desired, the affinity of the dual transporter to the BBB transport protein (e.g., TfR) is about 1000 to about 6000 nM, and the affinity to the second protein is about 1 to about 500 nM. In some embodiments, the affinity of the dual transporter to the BBB transport protein (e.g., TfR) is about 1000 to about 6000 nM, and the affinity to the second protein is about 250 to about 1000 nM. In some embodiments, the affinity of the dual transporter to the BBB transport protein (e.g., TfR) is about 1000 to about 6000 nM, and the affinity to the second protein is about 500 to about 6000 nM.

In some embodiments, in order to increase both uptake and retention in the brain of a dual transporter, the affinity of the dual transporter to TfR is about 50 to about 1000 nM, and the affinity to CD98hc is about 1 to about 500 nM. In some embodiments, the affinity of the dual transporter for TfR is about 100 to about 600 nM and the affinity to CD98hc is about 50 nM to about 300 nM. In some embodiments, the affinity of the dual transporter for TfR is about 100 to about 400 nM, and the affinity to CD98hc is about 50 to about 200 nM.

In some embodiments, in order to increase brain uptake while maximizing brain retention of a dual transporter, the affinity of the dual transporter to TfR is about 250 to about 6000 nM, and the affinity to CD98hc is about 1 to about 250 nM. In some embodiments, the affinity of the dual transporter for TfR is about 250 to about 1000 nM, and the affinity to CD98hc is about 1 to about 100 nM. In some embodiments, the affinity of the dual transporter for TfR is about 50 to about 250 nM, and the affinity to CD98hc is about 1 to about 100 nM.

In some embodiments, if low TfR affinity is desired, the affinity of the dual transporter to TfR is about 1000 to about 6000 nM, and the affinity to CD98hc is about 1 to about 500 nM. In some embodiments, the affinity of the dual transporter to TfR is about 1000 to about 6000 nM, and the affinity to CD98hc is about 250 to about 1000 nM. In some embodiments, the affinity of the dual transporter to TfR is about 1000 to about 6000 nM, and the affinity to CD98hc is about 500 to about 6000 nM.

In some embodiments, in order to increase transport of a dual transporter across the BBB, the affinity of the dual transporter to the BBB transport protein is about 5- to about 10-fold greater than the affinity to the second brain retention protein. In some embodiments, the BBB transport protein is TfR, the affinity of the dual transporter to TfR is about 50 to about 1000 nM, and the affinity of the dual transporter the second protein is about 5- to about 10-fold less than the affinity to the TfR.

In some embodiments, in order to increase CNS retention, the affinity of the dual transporter to the second protein is 5- to 10-fold greater than the affinity to the BBB transport protein. In some embodiments, the BBB transport protein is TfR, the affinity of the dual transporter to TfR is about 50 to about 6000 nM, and the affinity of the dual transport to the second brain retention protein is about 5- to about 10-fold greater than the affinity to TfR.

As evidenced by dual transporters containing a TfR binding region and a MOG binding region having similar properties to dual transporters containing a TfR binding region and a CD98hc binding region, it is appreciated that the above properties of a dual transporter are likely apply to other BBB transport protein/second protein combinations (e.g., GLUT1, IGF1R. MFSD2A, and CA-IV).

A dual transporter, or a component thereof, as described herein, may be fused to a therapeutic peptide via a linker. The linker can be, but is not limited to, a peptide linker (e.g., a hinge region). The peptide linker may be configured such that it allows for the rotation of the therapeutic peptide and the Fc polypeptide or Fc dimer to each other; and/or is resistant to digestion by proteases. In some embodiments, the linker may be a flexible linker. e.g., containing amino acids such as Gly, Asn, Ser, Thr, Ala, and the like. Such linkers are designed using known parameters. For example, the linker may have repeats, such as Gly-Ser or (Gly)ₘ(Ser)ₙ repeats. Linkers can be any appropriate length. Peptide linkers can include at least 2 or 3 amino acids, for example, 5-50, 5-25, 5-15, 5-10, 10-50, 10-25, or 10-15 amino acids.

In some embodiments, the first binding region is linked a therapeutic molecule. In some embodiments, the second binding region is linked a therapeutic molecule. In some embodiments, both the first and second binding regions are linked to therapeutic molecule. In some embodiments, the first binding region is linked a first therapeutic molecule and the second binding region is linked a second therapeutic agent.

In some embodiments, the dual transporter, or a component of a dual transporter is linked or fused to a peptide or protein useful in protein purification (e.g., a polyhistidine, an epitope tags (e.g., FLAG, c-Myc, hemagglutinin tags and the like), a glutathione S transferase, a thioredoxin, a protein A, a protein G, or a maltose binding protein). The peptide or protein may be linked or fused to the dual transporter or the component of the dual transporter via a cleavable linker (e.g., a protease cleavage site, such as a cleavage site for Factor Xa or Thrombin).

### A. Transferrin Receptor (TfR)-Binding Region

The transferrin receptor (TfR) (also termed cluster of differentiation 71 (CD71)) binds transferrin (Tf) and performs a critical role in cellular iron uptake through interaction with iron-bound transferrin. TfR is highly expressed by brain capillary endothelial cells (BCECs) forming the BBB. TfR is a 90 kDa type II transmembrane glycoprotein consisting of 760 amino acids that is found as a dimer (180 kDa) linked by disulfide bonds on the cell surface. The TfR1 monomer is composed of a large extracellular, C-terminal domain of 671 amino acids containing the Tf binding site, a transmembrane domain (28 amino acids), and an intracellular N-terminal domain (61 amino acids). The C-terminal extracellular domain contains three N-linked glycosylation sites at asparagine residues 251, 317, and 727 and one O-linked glycosylation site at threonine 104, which are all required for adequate function of the receptor.

A TfR-binding region (means for binding TfR) is a molecule such as a polypeptide or a region or domain of a larger polypeptide or protein that specifically binds to a TfR, such as a human TfR.

In some embodiments, the TfR-binding region binds to an apical domain of TfR. The apical domain comprises residues 189-383 of human TfR. In some embodiments, the TfR-binding region binds at an epitope that comprises position 208 of the full-length human TfR sequence. In some embodiments, a TfR-binding region binds to the apical domain of the TfR at an epitope that comprises positions 158, 188, 199, 207, 208, 209, 210, 211, 212, 213, 214, 215, and/or 294 of the full-length human TfR sequence (SEQ ID NO:4).

In some embodiments, binding of the TfR-binding region to TfR does not inhibit binding of transferrin to TfR. In some embodiments, binding of the dual transporter to TfR does not inhibit binding of transferrin to TfR. In some embodiments, binding of transferrin to TfR is inhibited by less than about 50% (e.g., less than about 45%, 40%, 35%, 30%, 25%, 20%, 15%, 10%, or 5%). In some embodiments, binding of transferrin to TfR is inhibited by less than about 20% (e.g., less than about 19%. 18% 17%, 16%, 15%, 14%, 13%, 12%, 11%, 10%, 9%, 8%, 7%. 6%, 5%, 4%, 3%, 2%, or 1%).

### 1. Group I Modified CH3 Peptide TfR Binding Regions

### a) Group Ia

In some embodiments, a TfR-binding region comprising a modified Fc polypeptide (e.g., modified CH3 peptide) comprises four, five, six, seven, eight, or nine substitutions in a set of amino acid positions consisting of: 384, 386, 387, 388, 389, 413, 415, 416, and 421; wherein the positions are determined with reference to EU numbering. In some embodiments, the substitutions are selected from: a Y at position 384; a T at position 386; an E at position 387; a W at position 388; a V, S, or A at position 389; a T or S at position 413; an E at position 415; an E at position 416; and a F at position 421.

In some embodiments, a TfR-binding region comprising a modified Fc polypeptide (e.g., modified CH3 peptide) comprises four, five, six, seven, eight, or nine substitutions selected from: a Y at position 384; a T at position 386; an E at position 387; a W at position 388; a V, S, or A at position 389; a T or S at position 413; an E at position 415; an E at position 416; and a F at position 421.

In some embodiments, a TfR-binding region comprising a modified Fc polypeptide (e.g., modified CH3 peptide) comprises: a Y at position 384; a T at position 386; an E at position 387; a W at position 388; a V, S, or A at position 389; a T or S at position 413; an E at position 415; an E at position 416; and a F at position 421.

In some embodiments, the TfR-binding region comprising a modified antibody heavy chain constant domain or Fc polypeptide (e.g., modified CH3 peptide) comprises a Y at position 391, a K at position 392, a S at position 424; and S at position 426.

### b) Group Ib

In some embodiments, a TfR-binding region comprising a modified Fc polypeptide (e.g., modified CH3 peptide) comprises five, six, seven, eight, nine, ten, eleven, twelve, thirteen, fourteen, fifteen, or sixteen substitutions in a set of amino acid positions consisting of: 380, 384, 386, 387, 388, 389, 390, 391, 392, 413, 414, 415, 416, 421, 424, and 426; wherein the positions are determined with reference to EU numbering. In some embodiments, the substitutions are selected from: a L, Q, S, V, W, or Y at position 380; a F, L, M, P, V, W, Y, or I at position 384; a F, H, I, L, N, P, T, or V at position 386; a D, E, I, or V at position 387; a W, F, or Y at position 388; an A, G, I, S, T, or V at position 389; an A, D, E, F, G, H, K, L, Q, R, S, T, V, or Y at position 390; a F, Q, S, T, or V at position 391; a Q, F, or H at position 392; an A, E, H, I, L, P, S, or T at position 413; a R, G, or P at position 414; a D, E, G, T, P, Q, or R at position 415; a D, E, N, or T at position 416; a F, H, K, W, or Y at position 421; a T, W, E, or K at position 424; and a C, P, M, W, or G at position 426.

In some embodiments, a TfR-binding region comprising a modified antibody heavy chain constant domain or Fc polypeptide (e.g., modified CH3 peptide) comprises: a F, L, M, P, V, W, Y, or I at position 384; a F, H, I, L, N, P, T, or V at position 386; a W, F, or Y at position 388; an A, G, I, S, T, or V at position 389; and; a F, H, K, W, or Y at position 421. In some embodiments, the TfR-binding region further comprises a V at position 387.

In some embodiments, a TfR-binding region comprising a modified antibody heavy chain constant domain or Fc polypeptide (e.g., modified CH3 peptide) comprises a F, L, M, P, V, W, Y, or I at position 384; a F, H, I, L, N, P, T, or V at position 386; a W, F, or Y at position 388; an A, G, I, S, T, or V at position 389; and a F, H, K, W, or Y at position 421 and one, two, three, four, five, six, seven, eight, nine, ten, or eleven substitutions in a set of amino acid positions consisting of: 380, 387, 390, 391, 392, 413, 414, 415, 416, 424, and 426; wherein the positions are determined with reference to EU numbering. In some embodiments, the substitutions are selected from: a L, Q, S, V, W, or Y at position 380; a D, E, I, or V at position 387; an A, D, E, F, G, H, K, L, Q, R, S, T, V, or Y at position 390; a F, Q, S, T, or V at position 391; a Q, F, or H at position 392; an A, E, H, I, L, P, S, or T at position 413; a R, G, or P at position 414; a D, E, G, T, P, Q, or R at position 415; a D, E, N, or T at position 416; a T, W, E, or K at position 424; and a C, P, M, W, or G at position 426.

In some embodiments, a TfR-binding region comprising a modified antibody heavy chain constant domain or Fc polypeptide (e.g., modified CH3 peptide) comprises a F, L, M, P, V, W, Y, or I at position 384; a F, H, I, L, N, P, T, or V at position 386; a W, F, or Y at position 388; an A, G, I, S, T, or V at position 389; and a F, H, K, W, or Y at position 421 and one, two, three, four, five, six, seven, eight, nine, ten, or eleven substitutions selected from: a L, Q, S, V, W, or Y at position 380; a D, E, I, or V at position 387; an A, D, E, F, G, H, K, L, Q, R, S, T, V, or Y at position 390; a F, Q, S, T, or V at position 391; a Q, F, or H at position 392; an A, E, H, I, L, P, S, or T at position 413; a R, G, or P at position 414; a D, E, G, T, P, Q, or R at position 415; a D, E, N, or T at position 416; a T, W, E, or K at position 424; and a C, P, M, W, or G at position 426.

In some embodiments, a TfR-binding region comprising a modified antibody heavy chain constant domain or Fc polypeptide (e.g., modified CH3 peptide) comprises a F, L, M, P, V, W, Y, or I at position 384; a F, H, I, L, N, P, T, or V at position 386; a V at position 387, a W, F, or Y at position 388; an A, G, I, S, T, or V at position 389; and a F, H, K, W, or Y at position 421 and one, two, three, four, five, six, seven, eight, nine, or ten substitutions selected from: a L, Q, S, V, W, or Y at position 380; a F, H, I, L, N, P, T, or V at position 386; an A, D, E, F, G, H, K, L, Q, R, S, T, V, or Y at position 390; a F, Q, S, T, or V at position 391; a Q, F, or H at position 392; an A, E, H, I, L, P, S, or T at position 413; a R, G, or P at position 414; a D, E, G, T, P, Q, or R at position 415; a D, E, N, or T at position 416; a T, W, E, or K at position 424; and a C, P, M, W, or G at position 426.

In some embodiments, the TfR-binding region comprising a modified antibody heavy chain constant domain or Fc polypeptide (e.g., modified CH3 peptide) comprises a Y at position 391, a K at position 392, a S at position 424; and S at position 426.

In some embodiments, a TfR-binding region comprising a modified CH3 peptide comprises four, five, six, seven, eight, nine, ten, eleven, twelve, thirteen, fourteen, fifteen, or sixteen amino acid substitutions in a set of amino acid positions consisting of: 380, 384, 386, 387, 388, 389, 390, 391, 392, 413, 414, 415, 416, 421, 424, and 426; wherein the amino acid at each of positions 380, 384, 386, 387, 388, 389, 390, 391, 392, 413, 414, 415, 416, 421, 424, and 426 can be any of the amino acids provided in Table 1.

In some embodiments, a TfR-binding region comprises a modified CH3 peptide having the amino acid substitutions of any of the clones provided in Tables 3-5. Affinities (Kd) for human and TfR are shown in Table 20 for the indicated TfR-binding domains.

TABLE 1
Modified CH3 peptides: possible amino acids and the indicated positions.

| | Position (according to EU numbering) | | | | | | | | | | | | | | | |
|---|---|---|---|---|---|---|---|---|---|---|---|---|---|---|---|---|
| | 380 | 384 | 386 | 387 | 388 | 389 | 390 | 391 | 392 | 413 | 414 | 415 | 416 | 421 | 424 | 426 |
| WT Fc | E | N | Q | P | E | N | N | Y | K | D | K | S | R | N | S | S |
| Possible | E | F | F | D | W | A | A | F | K | A | K | D | D | F | S | S |
| amino | L | L | H | E | | G | D | Q | Q | D | R | E | E | H | T | C |
| acids at | Q | M | I | I | | I | E | S | F | E | G | G | N | K | W | P |
| each | S | P | L | P | | S | F | T | H | H | P | S | R | W | E | M |
| position | V | V | N | V | | T | G | V | | I | | T | T | Y | K | W |
| | W | W | P | | | V | H | Y | | L | | P | | | | G |
| | Y | Y | T | | | | K | | | P | | Q | | | | |
| | | I | V | | | | L | | | S | | R | | | | |
| | | | | | | | N | | | T | | | | | | |
| | | | | | | | Q | | | | | | | | | |
| | | | | | | | R | | | | | | | | | |
| | | | | | | | S | | | | | | | | | |
| | | | | | | | T | | | | | | | | | |
| | | | | | | | V | | | | | | | | | |
| | | | | | | | Y | | | | | | | | | |

In some embodiments, a TfR-binding region comprising a modified CH3 peptide comprises a polypeptide having at least 85%, at least 90%, at least 95%, or 100% identify to the amino acid sequence of any of SEQ ID NOs:6-14 and 124-125, clone CH3C.35.23.2 with knob mutation, clone CH3C.35.23.2 with knob and LALA mutations, clone CH3C.35.23.2 with knob and LALAPG mutations, clone CH3C.35.23.3 with knob mutation, clone CH3C.35.23.3 with knob and LALA mutations, clone CH3C.35.23.3 with knob and LALAPG mutations, clone CH3C.35.23.4 with knob mutation, clone CH3C.35.23.4 with knob and LALA mutations, clone CH3C.35.23.4 with knob and LALAPG mutations, clone TV35.d1.9 with knob mutation, clone TV35.d1.9 with knob and LALA mutations, clone TV35.d1.9 with knob and LALAPG mutations, clone TV35.d1.10 with knob mutation, clone TV35.d1.10 with knob and LALA mutations, clone TV35.d1.10 with knob and LALAPG mutations. Any of the above can contain hole mutations instead of knob mutations.

In some embodiments, a TfR-binding region comprising a modified CH3 peptide comprises a polypeptide having at least 85%, at least 90%, at least 95%, or 100% identify to amino acids 111-217 of any of SEQ ID NOs:6-14 and 124-125.

TABLE 2
Fc peptides having modified CH3 domains that specifically bind TfR.

| SEQ | | |
|---|---|---|
| ID | | |
| NO. | Amino acid sequence | Description |
| 6 | PCPAPELLGGPSVFLFPPKPKDTLMISRTPEVTCVVVDVSHEDPEVKFN | Clone CH3C.35.23.2 |
| | WYVDGVEVIINAKTKPREEQYNSTYRVVSVLTVLIIQDWLNGKEYKC | with knob mutation |
| | KVSNKALPAPIEKTISKAKGQPREPQVYTLPPSRDELTKNQVSLWCLV | |
| | KGFYPSDIAVEWESYGTEWANYKTTPPVLDSDGSFFLYSKLTVTKEE | |
| | WQQGFVFSCSVMHEALHNHYTQKSLSLSPGK | |
| 7 | PCPAPEAAGGPSVFLFPPKPKDTLMISRTPEVTCVVVDVSHEDPEVKFN | Clone CH3C.35.23.2 |
| | WYVDGVEVHNAKTKPREEQYNSTYRVVSVLTVLHQDWLNGKEYKC | with knob and LALA |
| | KVSNKALPAPIEKTISKAKGQPREPQVYTLPPSRDELTKNQVSLWCLV | mutations |
| | KGFYPSDIAVEWESYGTEWANYKTTPPVLDSDGSFFLYSKLTVTKEE | |
| | WQQGFVFSCSVMHEALHNHYTQKSLSLSPGK | |
| 8 | PCPAPEAAGGPSVFLFPPKPKDTLMISRTPEVTCVVVDVSHEDPEVKFN | Clone CH3C.35.23.2 |
| | WYVDGVEVHNAKTKPREEQYNSTYRVVSVLTVLHQDWLNGKEYKC | with knob and LALAPG |
| | KVSNKALGAPIEKTISKAKGQPREPQVYTLPPSRDELTKNQVSLWCLV | mutations |
| | KGFYPSDIAVEWESYGTEWANYKTTPPVLDSDGSFFLYSKLTVTKEE | |
| | WQQGFVFSCSVMHEALHNHYTQKSLSLSPGK | |
| 9 | PCPAPELLGGPSVFLFPPKPKDTLMISRTPEVTCVVVDVSHEDPEVKFN | Clone CH3C.35.23.3 |
| | WYVDGVEVHNAKTKPREEQYNSTYRVVSVLTVLHQDWLNGKEYKC | with knob mutation |
| | KVSNKALPAPIEKTISKAKGQPREPQVYTLPPSRDELTKNQVSLWCLV | |
| | KGFYPSDIAVEWESYGTEWVNYKTTPPVLDSDGSFFLYSKLTVTKEE | |
| | WQQGFVFSCSVMHEALHNHYTQKSLSLSPGK | |
| 10 | PCPAPEAAGGPSVFLFPPKPKDTLMISRTPEVTCVVVDVSHEDPEVKFN | Clone CH3C.35.23.3 |
| | WYVDGVEVHNAKTKPREEQYNSTYRVVSVLTVLHQDWLNGKEYKC | with knob and LALA |
| | KVSNKALPAPIEKTISKAKGQPREPQVYTLPPSRDELTKNQVSLWCLV | mutations |
| | KGFYPSDIAVEWESYGTEWVNYKTTPPVLDSDGSFFLYSKLTVTKEE | |
| | WQQGFVFSCSVMHEALHNHYTQKSLSLSPGK | |
| 11 | PCPAPEAAGGPSVFLFPPKPKDTLMISRTPEVTCVVVDVSHEDPEVKFN | Clone CH3C.35.23.3 |
| | WYVDGVEVHNAKTKPREEQYNSTYRVVSVLTVLHQDWLNGKEYKC | with knob and LALAPG |
| | KVSNKALGAPIEKTISKAKGQPREPQVYTLPPSRDELTKNQVSLWCLV | mutations |
| | KGFYPSDIAVEWESYGTEWVNYKTTPPVLDSDGSFFLYSKLTVTKEE | |
| | WQQGFVFSCSVMHEALHNHYTQKSLSLSPGK | |
| 12 | PCPAPELLGGPSVFLFPPKPKDTLMISRTPEVTCVVVDVSHEDPEVKFN | Clone CH3C.35.23.4 |
| | WYVDGVEVHNAKTKPREEQYNSTYRVVSVLTVLHQDWLNGKEYKC | with knob mutation |
| | KVSNKALPAPIEKTISKAKGQPREPQVYTLPPSRDELTKNQVSLWCLV | |
| | KGFYPSDIAVEWESYGTEWSNYKTTPPVLDSDGSFFLYSKLTVSKEEW | |
| | QQGFVFSCSVMIIEALINIIYTQKSLSLSPGK | |
| 13 | PCPAPEAAGGPSVFLFPPKPKDTLMISRTPEVTCVVVDVSHEDPEVKFN | Clone CH3C.35.23.4 |
| | WYVDGVEVHNAKTKPREEQYNSTYRVVSVLTVLHQDWLNGKEYKC | with knob and LALA |
| | KVSNKALPAPIEKTISKAKGQPREPQVYTLPPSRDELTKNQVSLWCLV | mutations |
| | KGFYPSDIAVEWESYGTEWSNYKTTPPVLDSDGSFFLYSKLTVSKEEW | |
| | QQGFVFSCSVMHEALHNHYTQKSLSLSPGK | |
| 14 | PCPAPEAAGGPSVFLFPPKPKDTLMISRTPEVTCVVVDVSHEDPEVKFN | Clone CH3C.35.23.4 |
| | WYVDGVEVHNAKTKPREEQYNSTYRVVSVLTVLHQDWLNGKEYKC | with knob and LALAPG |
| | KVSNKALGAPIEKTISKAKGQPREPQVYTLPPSRDELTKNQVSLWCLV | mutations |
| | KGFYPSDIAVEWESYGTEWSNYKTTPPVLDSDGSFFLYSKLTVSKEEW | |
| | QQGFVFSCSVMHEALHNHYTQKSLSLSPGK | |
| 124 | PCPAPEAAGGPSVFLFPPKPKDTLMISRTPEVTCVVVDVSHEDPEVKFN | Clone TV35.d1.9 |
| | WYVDGVEVHNAKTKPREEQYNSTYRVVSVLTVLHQDWLNGKEYKC | LALAPG knob |
| | KVSNKALGAPIEKTISKAKGQPREPQVYTLPPSRDELTKNQVSLWCLV | |
| | KGFYPSDIAVEWESYGTEWTNYKTTPPVLDSDGSFFLYSKLTVTKSEW | |
| | QQGFVFSCSVMHEALHNHYTQKSLSLSPGK | |
| 125 | PCPAPEAAGGPSVFLFPPKPKDTLMISRTPEVTCVVVDVSHEDPEVKFN | Clone TV35.d1.10 |
| | WYVDGVEVHNAKTKPREEQYNSTYRVVSVLTVLHQDWLNGKEYKC | LALAPG knob |
| | KVSNKALGAPIEKTISKAKGQPREPQVYTLPPSRDELTKNQVSLWCLV | |
| | KGFYPSDIAVEWESYGTEWVNYKTTPPVLDSDGSFFLYSKLTVTKSE | |
| | WQQGFVFSCSVMHEALHNHYTQKSLSLSPGK | |

### c) Group Ic

In some embodiments, a TfR-binding region comprising a modified antibody heavy chain constant domain or Fc polypeptide (e.g., modified CH3 peptide) comprises six, seven, eight, nine, and ten substitutions in a set of amino acid positions consisting of: 384, 386, 387, 388, 389, 390, 391, 413, 416, and 421; wherein the positions are determined with reference to EU numbering. In some embodiments, the substitutions are selected from: a L, M, V, W, or Y at position 384; a F, H, L, P, or T at position 386; an E or V at position 387; a W at position 388; an A, G, S, or V at position 389; an A, D, E, F, G, H, K, L, Q, S, or T at position 390; a F, Q, S, T, or V at position 391; an A, E, L, P, S, or T at position 413; a D, E, N, or T at position 416; and a F, H, W, or Y at position 421.

In some embodiments, a TfR-binding region comprising a modified antibody heavy chain constant domain or Fc polypeptide (e.g., modified CH3 peptide) comprises: a L, M, V, W, or Y at position 384; a F, H, L, P, or T at position 386; a W at position 388; an A, G, S, or V at position 389; a D, E, N, or T at position 416; and a F, H, W, or Y at position 421. In some embodiments, the TfR-binding region further comprises a V at position 387.

In some embodiments, a TfR-binding region comprising a modified antibody heavy chain constant domain or Fc polypeptide (e.g., modified CH3 peptide) comprises a L, M, V, W, or Y at position 384; a F, H, L, P, or T at position 386; a W at position 388; an A, G, S, or V at position 389; a D, E, N, or T at position 416; and a F, H, W, or Y at position 421; and one, two, three, or four substitutions in a set of amino acid positions consisting of: 387, 390, 391, and 413; wherein the positions are determined with reference to EU numbering. In some embodiments, the substitutions are selected from: an E or V at position 387; an A, D, E, F, G, H, K, L, Q, S, or T at position 390; a F, Q, S, T, or V at position 391; and an A, E, L, P, S, or T at position 413.

In some embodiments, a TfR-binding region comprising a modified antibody heavy chain constant domain or Fc polypeptide (e.g., modified CH3 peptide) comprises a L, M, V, W, or Y at position 384; a F, H, L, P, or T at position 386; a W at position 388; an A, G, S, or V at position 389; a D, E, N, or T at position 416; and a F, H, W, or Y at position 421; and one, two, three, or four substitutions selected from: an E or V at position 387; an A, D, E, F, G, H, K, L, Q, S, or T at position 390; a F, Q, S, T, or V at position 391; and an A, E, L, P, S, or T at position 413.

In some embodiments, a TfR-binding region comprising a modified antibody heavy chain constant domain or Fc polypeptide (e.g., modified CH3 peptide) comprises a L, M, V, W, or Y at position 384; a F, H, L, P, or T at position 386; a V at position 387, a W at position 388; an A, G, S, or V at position 389; a D, E, N, or T at position 416; and a F, H, W, or Y at position 421; and one, two, or three substitutions selected from: an A, D, E, F, G, H, K, L, Q, S, or T at position 390; a F, Q, S, T, or V at position 391; and an A, E, L, P, S, or T at position 413.

In some embodiments, a TfR-binding region comprises a modified CH3 peptide having the amino acid substitutions of any of the clones provided in Table 3.

TABLE 3
Modified CH3 peptides: possible amino acids and the indicated positions.

| | clone | | | | | | | | | |
|---|---|---|---|---|---|---|---|---|---|---|
| | Position (according to EU numbering) | | | | | | | | | |
| | 384 | 386 | 387 | 388 | 389 | 390 | 391 | 413 | 416 | 421 |
| Wild-type | N | Q | P | E | N | N | Y | D | R | N |
| CH3C.1 | L | L | V | W | V | G | | A | T | W |
| CH3C.2 | Y | T | V | W | S | H | | S | E | Y |
| CH3C.3 | Y | T | E | W | S | Q | | E | D | H |
| CH3C.4 | V | T | P | W | A | L | | L | E | W |
| CH3C.17 | Y | T | V | W | S | K | | S | E | F |
| CH3C.18 | L | H | V | W | A | V | | P | T | W |
| CH3C.21 | L | L | V | W | V | G | | P | T | W |
| CH3C.25 | M | H | V | W | V | G | | D | T | W |
| CH3C.34 | L | L | V | W | V | F | S | P | T | W |
| CH3C.35 | Y | T | E | W | S | S | | T | E | F |
| CH3C.44 | Y | T | E | W | S | | | S | E | F |
| CH3C.51 | L | H | V | W | V | G | | S | E | W |
| CH3C.3.1-3 | L | H | V | W | V | A | T | P | T | W |
| CH3C.3.1-9 | L | P | V | W | V | H | T | P | T | W |
| CH3C.3.2-5 | L | H | V | W | V | D | Q | P | T | W |
| CH3C.3.2-19 | L | H | V | W | V | | Q | P | T | W |
| CH3C.3.2-1 | L | H | V | W | V | | F | P | T | W |
| CH3C.3.4-1 | W | F | V | W | S | T | | P | N | F |
| CH3C.3.4-19 | W | H | V | W | S | T | | P | N | Y |
| CH3C.3.2-3 | L | H | V | W | V | E | Q | P | T | W |
| CH3C.3.2-14 | L | H | V | W | V | G | V | P | T | W |
| CH3C.3.2-24 | L | H | V | W | V | H | T | P | T | W |
| CH3C.3.4-26 | W | T | V | W | G | T | | P | N | Y |
| CH3C.3.2-17 | L | H | V | W | V | G | T | P | T | W |

### d) Group Id

In some embodiments, a TfR-binding region comprising a modified antibody heavy chain constant domain or Fc polypeptide (e.g., modified CH3 peptide) comprises eight, nine, ten, or eleven substitutions in a set of amino acid positions consisting of: 380, 384, 386, 387, 388, 389, 390, 413, 415, 416, and 421; wherein the positions are determined with reference to EU numbering. In some embodiments, the substitutions are selected from: a W or L at position 380; a F or Y at position 384; a T at position 386; an E at position 387; a W at position 388; a S, A, or V at position 389; a S at position 390; a S or T at position 413; an E at position 415; an E at position 416; and a F at position 421.

In some embodiments, a TfR-binding region comprising a modified antibody heavy chain constant domain or Fc polypeptide (e.g., modified CH3 peptide) comprises: a F or Y at position 384; a T at position 386; an E at position 387; a W at position 388; a S, A, or V at position 389; a S or T at position 413; an E at position 416; and a F at position 421.

In some embodiments, a TfR-binding region comprising a modified antibody heavy chain constant domain or Fc polypeptide (e.g., modified CH3 peptide) comprises a F or Y at position 384; a T at position 386; an E at position 387; a W at position 388; a S, A, or V at position 389; a S or T at position 413; an E at position 416; and a F at position 421; and one or two substitutions in a set of amino acid positions consisting of: 390 and 415; wherein the positions are determined with reference to EU numbering. In some embodiments, the substitutions are selected from: a S at position 390 and an E at position 415.

In some embodiments, a TfR-binding region comprising a modified antibody heavy chain constant domain or Fc polypeptide (e.g., modified CH3 peptide) comprises a F or Y at position 384; a T at position 386; an E at position 387; a W at position 388; a S, A, or V at position 389; a S or T at position 413; an E at position 416; and a F at position 421; and one or two substitutions selected from: a S at position 390 and an E at position 415.

In some embodiments, a TfR-binding region comprises a modified CH3 peptide having the amino acid substitutions of any of the clones provided in Table 4.

TABLE 4
Modified CH3 peptides: possible amino acids and the indicated positions.

| | clone | | | | | | | | | | |
|---|---|---|---|---|---|---|---|---|---|---|---|
| | Position (according to EU numbering) | | | | | | | | | | |
| | 380 | 384 | 386 | 387 | 388 | 389 | 390 | 413 | 415 | 416 | 421 |
| Wild-type | E | N | Q | P | E | N | N | D | S | R | N |
| CH3C.35.20.1 | . | F | T | E | W | S | S | T | E | E | F |
| CH3C.35.20.2 | . | Y | T | E | W | A | S | T | E | E | F |
| CH3C.35.20.3 | . | Y | T | E | W | V | S | T | E | E | F |
| CH3C.35.20.4 | . | Y | T | E | W | S | S | S | E | E | F |
| CH3C.35.20.5 | . | F | T | E | W | A | S | T | E | E | F |
| CH3C.35.20.6 | . | F | T | E | W | V | S | T | E | E | F |
| CH3C.35.21.a.1 | W | F | T | E | W | S | S | T | E | E | F |
| CH3C.35.21.a.2 | W | Y | T | E | W | A | S | T | E | E | F |
| CH3C.35.21.a.3 | W | Y | T | E | W | V | S | T | E | E | F |
| CH3C.35.21.a.4 | W | Y | T | E | W | S | S | S | E | E | F |
| CH3C.35.21.a.5 | W | F | T | E | W | A | S | T | E | E | F |
| CH3C.35.21.a.6 | W | F | T | E | W | V | S | T | E | E | F |
| CHI3C.35.23.1 | | F | T | E | W | S | | T | E | E | F |
| CH3C.35.23.2 | | Y | T | E | W | A | | T | E | E | F |
| CH3C.35.23.3 | | Y | T | E | W | V | | T | E | E | F |
| CH3C.35.23.4 | | Y | T | E | W | S | | S | E | E | F |
| CH3C.35.23.5 | | F | T | E | W | A | | T | E | E | F |
| CH3C.35.23.6 | | F | T | E | W | V | | T | E | E | F |
| CH3C.35.24.1 | W | F | T | E | W | S | | T | E | E | F |
| CH3C.35.24.2 | W | Y | T | E | W | A | | T | E | E | F |
| CH3C.35.24.3 | W | Y | T | E | W | V | | T | E | E | F |
| CH3C.35.24.4 | W | Y | T | E | W | S | | S | E | E | F |
| CH3C.35.24.5 | W | E | T | E | W | A | | T | E | E | F |
| CH3C.35.24.6 | W | F | T | E | W | V | | T | E | E | F |
| CH3C.35.21.17.1 | L | F | T | E | W | S | S | T | E | E | F |
| CH3C.35.21.17.2 | L | Y | T | E | W | A | S | T | E | E | F |
| CH3C.35.21.17.3 | L | Y | T | E | W | V | S | T | E | E | F |
| CH3C.35.21.17.4 | L | Y | T | E | W | S | S | S | E | E | F |
| CH3C.35.21.17.5 | L | F | T | E | W | A | S | T | E | E | F |
| CH3C.35.21.17.6 | L | F | T | E | W | V | S | T | E | E | F |
| CH3C.35.20 | | Y | T | E | W | S | S | T | E | E | F |
| CH3C.35.21 | W | Y | T | E | W | S | S | T | E | E | F |
| CH3C.35.22 | W | Y | T | E | W | S | | T | | E | F |
| CH3C.35.23 | | Y | T | E | W | S | | T | E | E | F |
| CH3C.35.24 | W | Y | T | E | W | S | | T | E | E | F |
| CH3C.35.21.17 | L | Y | T | E | W | S | S | T | E | E | F |
| CH3C.35.N390 | | Y | T | E | W | S | | T | | E | F |
| CH3C.35.20.1.1 | | F | T | E | W | S | S | S | E | E | F |
| CH3C.35.23.2.1 | | Y | T | E | W | A | | S | | E | F |
| CH3C.35.23.1.1 | | F | T | E | W | S | | S | E | E | F |
| CH3C.35.S413 | | Y | T | E | W | S | S | S | | E | F |
| CH3C.35.23.3.1 | | Y | T | E | W | V | | S | E | E | F |
| CH3C.35.N390.1 | | Y | T | E | W | S | | S | | E | F |
| CH3C.35.23.6.1 | | F | T | E | W | V | | S | E | E | F |

### e) Group Ie

In some embodiments, a TfR-binding region comprising a modified antibody heavy chain constant domain or Fc polypeptide (e.g., modified CH3 peptide) comprises six, seven, eight, nine, ten, eleven, twelve, or thirteen substitutions in a set of amino acid positions consisting of: 380, 384, 386, 387, 388, 389, 390, 391, 413, 414, 415, 416, 421, 424, and 426; wherein the positions are determined with reference to EU numbering. In some embodiments, the substitutions are selected from: a W, L, S, V, or Y at position 380; a Y, F, M, P, V, or W at position 384; a T, N, or V at position 386; an E, I, or V at position 387; a W at position 388; a S, A, I, T, or V at position 389; a S, R, or T at position 390; a S, T, or H at position 413; an E, D, G, T, P, Q, or R at position 415; an E at position 416; a F, H, K, or Y at position 421; a T or W at position 424; and a C, P, M, or W at position 426.

In some embodiments, a TfR-binding region comprising a modified antibody heavy chain constant domain or Fc polypeptide (e.g., modified CH3 peptide) comprises: a Y, F, M, P, V, or W at position 384; a T, N, or V at position 386; a W at position 388; a S, A, I, T, or V at position 389; a S, T, or H at position 413; and a F, H, K, or Y at position 421.

In some embodiments, a TfR-binding region comprising a modified antibody heavy chain constant domain or Fc polypeptide (e.g., modified CH3 peptide) comprises: a Y, F, M, P, V, or W at position 384; a T, N, or V at position 386; a W at position 388; a S, A, I, T, or V at position 389; a S, T, or H at position 413; and a F, H, K, or Y at position 421; and one, two, three, four, five, six, or seven substitutions in a set of amino acid positions consisting of 380, 387, 390, 415, 416, 424, and 426; wherein the positions are determined with reference to EU numbering. In some embodiments, the substitutions are selected from: a W, L, S, V, or Y at position 380; an E, I, or V at position 387; a S, R, or T at position 390; an E, D, G, T, P, Q, or R at position 415; an E at position 416; a T or W at position 424; and a C, P, M, or W at position 426.

In some embodiments, a TfR-binding region comprising a modified antibody heavy chain constant domain or Fc polypeptide (e.g., modified CH3 peptide) comprises: a Y, F, M, P, V, or W at position 384; a T, N, or V at position 386; a W at position 388; a S, A, I, T, or V at position 389; a S, T, or H at position 413; and a F, H, K, or Y at position 421; and one, two, three, four, five, six, or seven substitutions selected from: a W, L, S, V, or Y at position 380; an E, I, or V at position 387; a S, R, or T at position 390; an E, D, G, T, P, Q, or R at position 415; an E at position 416; a T or W at position 424; and a C, P, M, or W at position 426.

### f) Group If

In some embodiments, a TfR-binding region comprising a modified antibody heavy chain constant domain or Fc polypeptide (e.g., modified CH3 peptide) comprises six, seven, eight, or nine substitutions in a set of amino acid positions consisting of: 384, 386, 387, 389, 390, 391, 413, 416, and 421; wherein the positions are determined with reference to EU numbering. In some embodiments, the substitutions are selected from: a V, Y, L, or W at position 384; a T, L, H, P, or F at position 386; a V or E at position 387; an A, S, V, or G at position 389; an A, D, E, G, H, L, Q, T, or V at position 390; a T, F, Q, or V at position 391; a L, S, E, A, or P at position 413; an E, D, T, or N at position 416; and a W, Y, H, or F at position 421.

In some embodiments, a TfR-binding region comprising a modified antibody heavy chain constant domain or Fc polypeptide (e.g., modified CH3 peptide) comprises: a V, Y, L, or W at position 384; a T, L, H, P, or F at position 386; an A, S, V, or G at position 389; a L, S, E, A, or P at position 413; an E, D, T, or N at position 416; and a W, Y, H, or F at position 421.

In some embodiments, a TfR-binding region comprising a modified antibody heavy chain constant domain or Fc polypeptide (e.g., modified CH3 peptide) comprises: a V, Y, L, or W at position 384; a T, L, H, P, or F at position 386; an A, S, V, or G at position 389; a L, S, E, A, or P at position 413; an E, D, T, or N at position 416; and a W, Y, H, or F at position 421; and one, two, or three substitutions in a set of amino acid positions consisting of: 387, 390, and 391; wherein the positions are determined with reference to EU numbering. In some embodiments, the substitutions are selected from: a V or E at position 387; an A, D, E, G, H, L, Q, T, or V at position 390; and a T, F, Q, or V at position 391.

In some embodiments, a TfR-binding region comprising a modified antibody heavy chain constant domain or Fc polypeptide (e.g., modified CH3 peptide) comprises: a V, Y, L, or W at position 384; a T, L, H, P, or F at position 386; an A, S, V, or G at position 389; a L, S, E, A, or P at position 413; an E, D, T, or N at position 416; and a W, Y, H, or F at position 421; and one, two, or three substitutions selected from: a V or E at position 387; an A, D, E, G, H, L, Q, T, or V at position 390; and a T, F, Q, or V at position 391.

In some embodiments, a TfR-binding region comprises a modified CH3 peptide having the amino acid substitutions of any of the clones provided in Table 5. Affinities (Kd) for human TfR are shown in Table 5 for the indicated TfR-binding domains.

TABLE 5
Modified CH3 peptides: possible amino acids and the indicated positions.

| | | | | | | | | | | | K_{D} for | |
|---|---|---|---|---|---|---|---|---|---|---|---|---|
| | Clone | | | | | | | | | | huTfR | No. of |
| | Position (according to EU numbering) | | | | | | | | | | Apical | mutations |
| | 384 | 386 | 387 | 388 | 389 | 390 | 413 | 415 | 416 | 421 | Domain | from |
| Wild-type | N | Q | P | E | N | N | D | S | R | N | (μM) | CH3C.1.35 |
| CH3C.1.35 | Y | T | E | W | S | S | T | | E | F | 0.610 | 0 |
| 35.d1.1 | Y | N | E | W | S | S | T | | E | F | 6.2 | 1 |
| 35.d1.2 | Y | T | E | W | T | S | T | | E | F | 4.3 | 1 |
| 35.d1.3 | Y | T | E | W | S | S | T | | E | Y | 1.3 | 1 |
| 35.d1.4 | F | T | E | W | S | S | S | | E | F | 1.4 | 2 |
| 35.d1.5 | Y | N | E | W | S | S | S | | E | F | 13.6 | 2 |
| 35.d1.6 | Y | T | E | W | T | S | S | | E | F | 11.2 | 2 |
| 35.d1.7 | Y | T | E | W | S | S | S | | E | Y | 3.0 | 2 |
| 35.d1.8 | Y | N | E | W | S | | T | | E | F | 13.2 | 2 |
| 35.d1.9 | Y | T | E | W | T | | T | | E | F | 8.4 | 2 |
| 35.d1.10 | Y | T | E | W | V | | T | | E | F | 2.5 | 2 |
| 35.d1.11 | Y | T | E | W | S | | T | | E | Y | 2.5 | 2 |
| 35.d1.12 | F | T | E | W | S | | S | | E | F | 1.6 | 3 |
| 35.d1.13 | Y | N | E | W | S | | S | | E | F | 18.5 | 3 |
| 35.d1.14 | Y | T | E | W | T | | S | | E | F | 11.7 | 3 |
| 35.d1.15 | Y | T | E | W | S | | S | | E | Y | 3.6 | 3 |
| 35.d1.16 | F | N | E | W | S | | S | | E | F | 35.6 | 4 |
| 35.d1.17 | F | T | E | W | T | | S | | E | F | 18.8 | 4 |
| 35.d1.18 | F | T | E | W | S | | S | | E | Y | 4.4 | 4 |
| 35.d1.19 | F | T | E | W | V | | S | | E | Y | 16.0 | 5 |
| 35.d1.20 | Y | T | E | W | V | | S | | E | F | 3.6 | 3 |
| 35.d1.21 | Y | T | E | W | V | | S | | E | Y | 14.1 | 4 |
| 35.d1.22 | Y | N | E | W | S | | S | E | E | F | 5.2 | 4 |
| 35.d1.23 | Y | T | E | W | T | | S | E | E | F | 3.5 | 4 |
| 35.d1.24 | Y | T | E | W | S | | S | E | E | Y | 1.1 | 4 |
| All | Y/F | N/T | E | W | T/S/V | N/S | S/T | S/E | E | F/Y | | |
| positions | | | | | | | | | | | | |

### 2. Group II Modified CH3 Peptide TfR Binding Regions

### a) Group IIa

In some embodiments, a TfR-binding region comprising a modified antibody heavy chain constant domain or Fc polypeptide (e.g., modified CH3 peptide) comprises six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, or thirty-one amino acid substitutions in a set of amino acid positions consisting of 378, 380, 382, 383, 384, 385, 386, 387, 388, 389, 390, 391, 414.417, 419, 420, 421, 422.424, 426.427, 428, 429, 433, 434.437, 438.439, 440, 442, and 443, and 426; wherein the positions are determined with reference to EU numbering. In some embodiments, the substitutions are selected from: an E, L, or I at position 378; a N, R, Y, I, S, or F at position 380; a F at position 382; a Y, A, G, T, or a deletion at position 383; an A, D, E, F, G, or T at position 384; an A, D, or N at position 385; an A, G, K, N, S, or Y at position 386; a G, 1, K, N, R, S, or T at position 387; a L, Q, or D at position 388; an 1. P, Q, R, S, or T at position 389; a G, T, Y, or L at position 390; a S, T, I, L, or P at position 391; a P at position 414; a K at position 417; a P at position 419; a R or Q at position 420; an A, F, G, S, or Y at position 421; a L at position 422; an A at position 424; an E at position 426; an E at position 427; an E at position 428; a G at position 429; an E at position 433; a G at position 434; a D at position 437; a Y at position 438; a D, E, or S at position 439; a L at position 440; a G or W at position 442; and an E or Y at position 443.

In some embodiments, a TfR-binding region comprising a modified antibody heavy chain constant domain or Fc polypeptide (e.g., modified CH3 peptide) comprises: a F at position 382, a L at position 422, an A at position 424, an E at position 426, a Y at position 438, and a L at position 440.

In some embodiments, a TfR-binding region comprising a modified antibody heavy chain constant domain or Fc polypeptide (e.g., modified CH3 peptide) comprises: a F at position 382, a L at position 422, an A at position 424, an E at position 426, a Y at position 438, and a L at position 440; and one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, or twenty-five substitutions in a set of amino acid positions consisting of 378, 380, 383, 384, 385, 386, 387, 388, 389, 390, 391, 414, 417, 419, 420, 421, 427, 428, 429, 433, 434, 437, 439, 442, and 443; wherein the positions are determined with reference to EU numbering. In some embodiments, the substitutions are selected from: an E, L, or I at position 378; a N, R, Y, I, S, or F at position 380; a Y, A, G, T, or a deletion at position 383; an A, D, E, F, G, or T at position 384; an A, D, or N at position 385; an A, G, K, N, S, or Y at position 386; a G, I, K, N, R, S, or T at position 387; a L, Q, or D at position 388; an I, P, Q, R, S, or T at position 389; a G, T, Y, or L at position 390; a S, T, I, L, or P at position 391; a P at position 414; a K at position 417; a P at position 419; a R or Q at position 420; an A, F, G, S, or Y at position 421; an E at position 427; an E at position 428; a G at position 429; an E at position 433; a G at position 434; a D at position 437; a D, E, or S at position 439; a G or W at position 442; and an E or Y at position 443.

In some embodiments, a TfR-binding region comprising a modified antibody heavy chain constant domain or Fc polypeptide (e.g., modified CH3 peptide) comprises: a F at position 382, a L at position 422, an A at position 424, an E at position 426, a Y at position 438, and a L at position 440; and one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, or twenty-five substitutions selected from: an E, L, or I at position 378; a N, R, Y, I, S, or F at position 380; a Y, A, G, T, or a deletion at position 383; an A, D, E, F, G, or T at position 384; an A, D, or N at position 385; an A, G, K, N, S, or Y at position 386; a G, I, K, N, R, S, or T at position 387; a L, Q, or D at position 388; an I, P, Q, R, S, or T at position 389; a G, T, Y, or L at position 390; a S, T, I, L, or P at position 391; a P at position 414; a K at position 417; a P at position 419; a R or Q at position 420; an A, F, G, S, or Y at position 421; an E at position 427; an E at position 428; a G at position 429; an E at position 433; a G at position 434; a D at position 437; a D, E, or S at position 439; a G or W at position 442; and an E or Y at position 443.

In some embodiments, a TfR-binding region comprising a modified CH3 peptide comprises six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, or thirty-one amino acid substitutions in a set of amino acid positions consisting of: 378, 380, 382, 383, 384, 385, 386, 387.388, 389, 390, 391, 414, 417, 419.420, 421, 422, 424, 426, 427, 428.429, 433, 434, 437, 438, 439, 440, 442, and 443; wherein the amino acid at each of positions 378, 380, 382, 383, 384, 385, 386, 387, 388, 389, 390, 391, 414, 417, 419, 420, 421, 422, 424, 426, 427, 428, 429, 433, 434, 437, 438, 439, 440, 442, and 443 can be any of the amino acids provided in Table 6.

In some embodiments, a TfR-binding region comprises a modified CH3 peptide having the amino acid substitutions of any of the clones provided in Table 7.

In some embodiments, a TfR-binding region comprises a modified CH3 peptide having an amino acid at least 85%, at least 90%, at least 95%, or 100% identical to any of the clones provided in Table 8.

Affinities (Kd) for human TfR are shown in Table 9 for the indicated TfR-binding domains.

TABLE 6
Modified CH3 peptides: possible amino acids and the indicated positions.

| | name | | | | | | | | | | | | | | | |
|---|---|---|---|---|---|---|---|---|---|---|---|---|---|---|---|---|
| | Position (according to EU numbering) | | | | | | | | | | | | | | | |
| | 378 | 380 | 382 | 383 | 384 | 385 | 386 | 387 | 388 | 389 | 390 | 391 | 414 | 417 | 419 | 420 |
| WT Fc | A | E | E | S | N | G | Q | P | E | N | N | Y | K | W | Q | G |
| Possible | A | E | F | S | N | G | Q | P | E | N | N | Y | K | W | Q | G |
| amino | E | N | | Y | A | A | A | G | L | I | G | S | P | K | P | R |
| acids at | L | R | | A | D | D | G | I | Q | P | T | T | | | | Q |
| each | I | Y | | Δ | E | N | K | K | D | Q | Y | I | | | | |
| position | | I | | G | F | | N | N | | R | L | L | | | | |
| | | S | | T | G | | S | R | | S | | P | | | | |
| | | F | | | T | | Y | S | | T | | | | | | |

| | name | | | | | | | | | | | | | | |
|---|---|---|---|---|---|---|---|---|---|---|---|---|---|---|---|
| | Position (according to EU numbering) | | | | | | | | | | | | | | |
| | 421 | 422 | 424 | 426 | 427 | 428 | 429 | 433 | 434 | 437 | 438 | 439 | 440 | 442 | 443 |
| WT Fc | N | V | S | S | M | H | N | H | N | T | Q | K | S | S | L |
| Possible | N | L | A | E | M | H | N | H | N | T | Y | K | L | S | L |
| amino | A | | | | E | E | G | E | G | D | | D | | G | E |
| acids at | F | | | | | | | | | | | E | | W | Y |
| each | G | | | | | | | | | | | S | | | |
| position | S | | | | | | | | | | | | | | |
| | Y | | | | | | | | | | | | | | |

Δ = deletion

TABLE 7
Modified CH3 peptides: possible amino acids and the indicated positions.

| | name | | | | | | | | | | | | | | | | | |
|---|---|---|---|---|---|---|---|---|---|---|---|---|---|---|---|---|---|---|
| | Position (according to EU numbering | | | | | | | | | | | | | | | | | |
| | 382 | 383 | 384 | 385 | 386 | 387 | 388 | 389 | 419 | 420 | 421 | 422 | 424 | 426 | 438 | 440 | 442 | |
| Wild-type | E | S | N | G | Q | P | E | N | Q | G | N | V | S | S | Q | S | S | 443 |
| 6.5.11.5.42.1 | F | | G | | A | K | | T | | | | L | A | E | Y | L | | |
| 6.5.11.5.42.2 | F | | D | | S | K | | T | | | | L | A | E | Y | L | | |
| 42.2.19 | F | Y | D | D | S | K | L | T | P | R | G | L | A | E | Y | L | G | E |
| 42.8.15 | F | | G | A | | K | | S | | | | L | A | E | Y | L | | |
| 42.8.17 | F | | G | A | | K | L | T | | | | L | A | E | Y | L | | |
| 42.8.80 | F | | E | | S | K | | T | | | | L | A | E | Y | L | | |
| 42.8.143 | F | Y | G | D | S | K | L | T | | | | L | A | E | Y | L | | |
| 42.8.196 | F | Y | E | A | | K | L | | | | | L | A | E | Y | L | | |
| 42.2.3-1H | F | Y | G | N | A | K | | T | | | | L | A | E | Y | L | | |

TABLE 8
Modified CH3 peptides: possible amino acids and the indicated positions.

| Clone | Sequence |
|---|---|
| 6.5.11.5.42.1 | APELLGGPSVFLFPPKPKDTLMISRTPEVTCVVVDVSHEDPEVKENWYVDGVEVHNAKT |
| | KPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWFSGGAKETNYKTTPPVLDSDGSFFLY |
| | SKLTVDKSRWQQGNLFACEVMHEALHNHYTYKLLSLSPGK (SEQ ID NO: 57) |
| 6.5.11.5.42.2 | APELLGGPSVFLFPPKPKDTLMISRTPEVTCVVVDVSHEDPEVKFNWYVDGVEVHNAKT |
| | KPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESDGSKETNYKTTPPVLDSDGSFFLY |
| | SKLTVDKSRWQQGNLFACEVMHEALHNHYTYKLLSLSPGK (SEQ ID NO: 58) |
| 42.2.19 | APELLGGPSVFLFPPKPKDTLMISRTPEVTCVVVDVSHEDPEVKENWYVDGVEVHNAKT |
| | KPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWFYDDSKLTNYKTTPPVLDSDGSFFLY |
| | SKLTVDKSRWQPRGLFACEVMHEALHNHYTYKLLGESPGK (SEQ ID NO: 59) |
| 42.2.3-1H | APELLGGPSVFLFPPKPKDTLMISRTPEVTCVVVDVSHEDPEVKENWYVDGVEVHNAKT |
| | KPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWFYGNAKETNYKTTPPVLDSDGSFFLY |
| | SKLTVDKSRWQQGNLFACEVMHEALHNHYTYKLLSLSPGK (SEQ ID NO: 60) |
| 42.8.143 | APELLGGPSVFLFPPKPKDTLMISRTPEVTCVVVDVSHEDPEVKENWYVDGVEVHNAKT |
| | KPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWFYGDSKLTNYKTTPPVLDSDGSFFLY |
| | SKLTVDKSRWQQGNLFACEVMHEALHNHYTYKLLSLSPGK (SEQ ID NO: 126) |
| 42.8.196 | APELLGGPSVFLFPPKPKDTLMISRTPEVTCVVVDVSHEDPEVKENWYVDGVEVHNAKT |
| | KPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWFYEAQKLNNYKTTPPVLDSDGSEFLY |
| | SKLTVDKSRWQQGNLFACEVMHEALHNHYTYKLLSLSPGK (SEQ ID NO: 61) |
| 42.8.80 | APELLGGPSVFLFPPKPKDTLMISRTPEVTCVVVDVSHEDPEVKENWYVDGVEVHNAKT |
| | KPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWFSEGSKETNYKTTPPVLDSDGSFFLY |
| | SKLTVDKSRWQQGNLFACEVMHEALHNHYTYKLLSLSPGK (SEQ ID NO: 62) |
| 42.8.15 | APELLGGPSVFLFPPKPKDTLMISRTPEVTCVVVDVSHEDPEVKENWYVDGVEVHNAKT |
| | KPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESGAQKESNYKTTPPVLDSDGSFFLY |
| | SKLTVDKSRWQQGNLFACEVMHEALHNHYTYKLLSLSPGK (SEQ ID NO: 63) |
| 42.8.17 | APELLGGPSVFLFPPKPKDTLMISRTPEVTCVVVDVSHEDPEVKENWYVDGVEVHNAKT |
| | KPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWFSGAQKLTNYKTTPPVLDSDGSFFLY |
| | SKLTVDKSRWQQGNLFACEVMHEALHNHYTYKLLSLSPGK (SEQ ID NO: 64) |

TABLE 9
Affinity of modified CD3 peptides to human TfR.

| | K_{D} for huTfR Apical | Clearance |
|---|---|---|
| Clone | Domain (nM) | (mL/day/kg) |
| 6.5.11.5.42.1 | 840 | 10 |
| 6.5.11.5.42.2 | 650 | 12 |
| 42.2.19 | 80 | |
| 42.2.3-1H | 220 | |
| 42.8.196 | 924 | |
| 42.8.80 | 2380 | |
| 42.8.15 | 5960 | |
| 42.8.17 | 10400 | |

### b) Group IIb

In some embodiments, a TfR-binding region comprising a modified antibody heavy chain constant domain or Fc polypeptide (e.g., modified CH3 peptide) comprises eight, nine, ten, eleven, twelve, thirteen, fourteen, fifteen, sixteen, seventeen, eighteen, nineteen, twenty, twenty-one, substitutions in a set of amino acid positions consisting of: 378, 380, 382, 384, 386, 387, 388, 389, 390, 391, 414, 417, 421, 422, 424, 426, 437, 438, 439, 440, and 442; wherein the positions are determined with reference to EU numbering. In some embodiments, the substitutions are selected from: an E, L, or I at position 378; a N, R, Y, I, S, or F at position 380; a F at position 382; a G, F, D, or E at position 384; an A, S, or N at position 386; an I, R, T, K, N, or S at position 387; a L at position 388; a Q, T, I, S, or P at position 389; a G, T, Y, or L at position 390; a S, T, I, L, or P at position 391; a P at position 414; a K at position 417; an A, F, G, S, or Y at position 421; a L at position 422; an A at position 424; an E at position 426; a D at position 437; a Y at position 438; a D, E, or S at position 439; a L at position 440; and a W at position 442.

In some embodiments, a TfR-binding region comprising a modified antibody heavy chain constant domain or Fc polypeptide (e.g., modified CH3 peptide) comprises: a F at position 382; a G, F, D, or E at position 384; an I, R, T, K, N, or S at position 387; a L at position 422; an A at position 424; an E at position 426; a Y at position 438; and a L at position 440.

In some embodiments, a TfR-binding region comprising a modified antibody heavy chain constant domain or Fc polypeptide (e.g., modified CH3 peptide) comprises: a F at position 382; a G, F, D, or E at position 384; an I, R, T, K, N, or S at position 387; a L at position 422; an A at position 424; an E at position 426; a Y at position 438; and a L at position 440; and one, two, three, four, five, six, seven, eight, nine, ten, eleven, twelve, or thirteen substitutions in a set of amino acid positions consisting of 378, 380, 386, 388, 389, 390, 391, 414, 417, 421, 437, 439, and 442; wherein the positions are determined with reference to EU numbering. In some embodiments, the substitutions are selected from: an E, L, or I at position 378; a N, R, Y, I, S, or F at position 380; an A, S, or N at position 386; a L at position 388: a Q, T, I, S, or P at position 389; a G, T, Y, or L at position 390; a S, T, I, L, or P at position 391; a P at position 414; a K at position 417; an A, F, G, S, or Y at position 421; a D at position 437; a D, E, or S at position 439; and a W at position 442.

In some embodiments, a TfR-binding region comprising a modified antibody heavy chain constant domain or Fc polypeptide (e.g., modified CH3 peptide) comprises: a F at position 382; a G, F, D, or E at position 384; an I, R, T, K, N, or S at position 387; a L at position 422; an A at position 424; an E at position 426; a Y at position 438; and a L at position 440; and one, two, three, four, five, six, seven, eight, nine, ten, eleven, twelve, or thirteen substitutions selected from: an E, L, or I at position 378; a N, R, Y, I, S, or F at position 380; an A, S, or N at position 386; a L at position 388; a Q, T, I, S, or P at position 389; a G, T, Y, or L at position 390; a S, T, I, L, or P at position 391; a P at position 414; a K at position 417; an A, F, G, S, or Y at position 421; a D at position 437; a D, E, or S at position 439; and a W at position 442.

### c) Group IIc

In some embodiments, a TfR-binding region comprising a modified antibody heavy chain constant domain or Fc polypeptide (e.g., modified CH3 peptide) comprises six, seven, eight, nine, ten, or eleven substitutions in a set of amino acid positions consisting of: 382, 384, 385, 386, 387, 389, 422, 424, 426, 438, and 440; wherein the positions are determined with reference to EU numbering. In some embodiments, the substitutions are selected from: a F at position 382; a N, F, or D at position 384; an A at position 385; an S, G, or A at position 386; a K or T at position 387; a S or T at position 389; a L at position 422; an A at position 424: an E at position 426; a Y at position 438; and a L at position 440.

In some embodiments, a TfR-binding region comprising a modified antibody heavy chain constant domain or Fc polypeptide (e.g., modified CH3 peptide) comprises: a F at position 382; a L at position 422; an A at position 424; an E at position 426; a Y at position 438; and a L at position 440.

In some embodiments, a TfR-binding region comprising a modified antibody heavy chain constant domain or Fc polypeptide (e.g., modified CH3 peptide) comprises: a F at position 382; a L at position 422; an A at position 424; an E at position 426; a Y at position 438; and a L at position 440; and one, two, three, four, or five substitutions in a set of amino acid positions consisting of 384, 385, 386, 387, and 389; wherein the positions are determined with reference to EU numbering. In some embodiments, the substitutions are selected from: a N, F, or D at position 384; an A at position 385; an S, G, or A at position 386; a K or T at position 387; and a S or T at position 389.

In some embodiments, a TfR-binding region comprising a modified antibody heavy chain constant domain or Fc polypeptide (e.g., modified CH3 peptide) comprises: a F at position 382; a L at position 422; an A at position 424; an E at position 426; a Y at position 438; and a L at position 440; and one, two, three, four, or five substitutions selected from: a N, F, or D at position 384; an A at position 385; an S, G, or A at position 386; a K or T at position 387; and a S or T at position 389.

### d) Group IId

In some embodiments, a TfR-binding region comprising a modified antibody heavy chain constant domain or Fc polypeptide (e.g., modified CH3 peptide) comprises ten, eleven, twelve, thirteen, fourteen, fifteen, sixteen, seventeen, eighteen, nineteen, twenty, twenty-one, substitutions in a set of amino acid positions consisting of: 382, 383, 384, 385, 386, 387, 388, 389, 419, 420, 421, 422, 424, 426, 427, 428, 429, 438, 440, 442, and 443; wherein the positions are determined with reference to EU numbering. In some embodiments, the substitutions are selected from: a F at position 382; a Y, A, G, T or a deletion at position 383; a D, E, G, or T at position 384; a D, A, or N at position 385; a S, N, A, K, Y, or G at position 386; a K or G at position 387; a L, Q, or D at position 388; a T or R at position 389; a P at position 419; a R or Q at position 420; a G at position 421; a L at position 422; an A at position 424; an E at position 426; an E at position 427; an E at position 428; a G at position 429; a Y at position 438; a L at position 440; a G at position 442; and an E or Y at position 443.

In some embodiments, a TfR-binding region comprising a modified antibody heavy chain constant domain or Fc polypeptide (e.g., modified CH3 peptide) comprises: a F at position 382; a D, E, G, or T at position 384; a S, N, A, K, Y, or G at position 386; a K or G at position 387; a T or R at position 389; a L at position 422; an A at position 424; an E at position 426; a Y at position 438; and a L at position 440.

In some embodiments, a TfR-binding region comprising a modified antibody heavy chain constant domain or Fc polypeptide (e.g., modified CH3 peptide) comprises: a F at position 382; a D, E, G, or T at position 384; a S, N, A, K, Y, or G at position 386; a K or G at position 387; a T or R at position 389; a L at position 422; an A at position 424; an E at position 426; a Y at position 438; and a L at position 440; and one, two, three, four, five, six, seven, eight, nine, ten, or eleven substitutions in a set of amino acid positions consisting of: 383, 385, 388, 419, 420, 421, 427, 428, 429, 442, and 443; wherein the positions are determined with reference to EU numbering. In some embodiments, the substitutions are selected from: a Y, A, G, T or a deletion at position 383; a D, A, or N at position 385; a L, Q, or D at position 388; a P at position 419; a R or Q at position 420; a G at position 421; an E at position 427; an E at position 428; a G at position 429; a G at position 442; and an E or Y at position 443.

In some embodiments, a TfR-binding region comprising a modified antibody heavy chain constant domain or Fc polypeptide (e.g., modified CH3 peptide) comprises: a F at position 382; a D, E, G, or T at position 384; a S, N, A, K, Y, or G at position 386; a K or G at position 387; a T or R at position 389; a L at position 422; an A at position 424; an E at position 426; a Y at position 438; and a L at position 440; and one, two, three, four, five, six, seven, eight, nine, ten, or eleven selected from: a Y, A, G, T or a deletion at position 383; a D, A, or N at position 385; a L, Q, or D at position 388; a P at position 419; a R or Q at position 420; a G at position 421; an E at position 427; an E at position 428; a G at position 429; a G at position 442; and an E or Y at position 443.

### e) Group IIe

In some embodiments, a TfR-binding region comprising a modified antibody heavy chain constant domain or Fc polypeptide (e.g., modified CH3 peptide) comprises seven, eight, nine, ten, eleven, twelve, thirteen, fourteen, fifteen, or sixteen substitutions in a set of amino acid positions consisting of: 380, 382, 383, 384, 385, 386, 387, 388, 389, 422, 424, 426, 433, 434, 438, and 440; wherein the positions are determined with reference to EU numbering. In some embodiments, the substitutions are selected from: a F, N, or Y at position 380; a F at position 382; an A, Y, or a deletion at position 383; a D, E, or G at position 384; a D, A, or N at position 385; a N, A, G, or S at position 386; a G, I, K, or R at position 387; a L, Q, or D at position 388; a R, S, or T at position 389; a L at position 422; an A at position 424; an E at position 426; an E at position 433; a G at position 434; a Y at position 438; and a L at position 440.

In some embodiments, a TfR-binding region comprising a modified antibody heavy chain constant domain or Fc polypeptide (e.g., modified CH3 peptide) comprises: a F at position 382; a G, I, K, or R at position 387; a L at position 422; an A at position 424; an E at position 426; a Y at position 438; and a L at position 440.

In some embodiments, a TfR-binding region comprising a modified antibody heavy chain constant domain or Fc polypeptide (e.g., modified CH3 peptide) comprises: a F at position 382; a G, I, K, or R at position 387; a L at position 422; an A at position 424; an E at position 426; a Y at position 438; and a L at position 440; and one, two, three, four, five, six, seven, eight, or nine, substitutions in a set of amino acid positions consisting of: 380, 383, 384, 385, 386, 388, 389, 433, and 434; wherein the positions are determined with reference to EU numbering. In some embodiments, the substitutions are selected from: a F, N, or Y at position 380; an A, Y, or a deletion at position 383; a D, E, or G at position 384; a D, A, or N at position 385; a N, A, G, or S at position 386; a L, Q, or D at position 388; a R, S, or T at position 389; an E at position 433; and a G at position 434.

In some embodiments, a TfR-binding region comprising a modified antibody heavy chain constant domain or Fc polypeptide (e.g., modified CH3 peptide) comprises: a F at position 382; a G, I, K, or R at position 387; a L at position 422; an A at position 424; an E at position 426; a Y at position 438; and a L at position 440; and one, two, three, four, five, six, seven, eight, or nine, substitutions selected from: a F, N, or Y at position 380; an A, Y, or a deletion at position 383; a D, E, or G at position 384; a D, A, or N at position 385; a N, A, G, or S at position 386; a L, Q, or D at position 388; a R, S, or T at position 389; an E at position 433; and a G at position 434.

3. Group III Modified CH3 peptide TfR Binding Regions

In some embodiments, a TfR-binding region comprising a modified antibody heavy chain constant domain or Fc polypeptide (e.g., modified CH3 peptide) comprises four, five, six, seven, eight, nine, ten, eleven, twelve, thirteen, fourteen, fifteen, or sixteen substitutions in a set of amino acid positions consisting of: 378, 380, 382, 383, 384, 385, 386, 421, 422, 424, 426, 428, 434, 438, 440, and 442; wherein the positions are determined with reference to EU numbering. In some embodiments, the substitutions are selected from: a D, E, F, H, N, Q, S, V, or Y at position 378; an A, D, F, H, I, K, L, M, Q, S, T, or Y at position 380; a G at position 382; a T at position 383; an A, E, F, H, I, K, L, Q, S, V, or Y at position 384; an I, T, or V at position 385; an A, H, N, S, T, or V at position 386; an A, F, H, K, L, M, Q, S, T, V, or Y at position 421; an A, F, H, I, K, L, R, T, or Y at position 422; an A, G, or P at position 424; an A, I, L, T, or V at position 426; an A or L at position 428; a S at position 434; an I, F, L, V, or Y at position 438; an A, G, I, M, N, P, T, or V at position 440; and an A, K, M, R, T, or V at position 442.

In some embodiments, a TfR-binding region comprising a modified antibody heavy chain constant domain or Fc polypeptide (e.g, modified CH3 peptide) comprises: a G at position 382; an A, E, F, H, I, K, L, Q, S, V, or Y at position 384; an I, T, or V at position 385; and an I, F, L, V, or Y at position 438.

In some embodiments, a TfR-binding region comprising a modified antibody heavy chain constant domain or Fc polypeptide (e.g., modified CH3 peptide) comprises a G at position 382; an A, E, F, H, I, K, L, Q, S, V, or Y at position 384; an I, T, or V at position 385; and an I, F, L, V, or Y at position 438 and one, two, three, four, five, six, seven, eight, nine, ten, eleven, or twelve substitutions in a set of amino acid positions consisting of 378, 380, 383, 386, 421, 422, 424, 426, 428, 434, 440, and 442; wherein the positions are determined with reference to EU numbering. In some embodiments, the substitutions are selected from: a D, E, F, H, N, Q, S, V, or Y at position 378; an A, D, F, H, I, K, L, M, Q, S, T, or Y at position 380; a T at position 383; an A, H, N, S, T, or V at position 386; an A, F, H, K, L, M, Q, S, T, V, or Y at position 421; an A, F, H, I, K, L, R, T, or Y at position 422; an A, G, or P at position 424; an A, I, L, T, or V at position 426; an A or L at position 428; a S at position 434; an A, G, I, M, N, P, T, or V at position 440; and an A, K, M, R, T, or V at position 442.

In some embodiments, a TfR-binding region comprising a modified antibody heavy chain constant domain or Fc polypeptide (e.g., modified CH3 peptide) comprises a G at position 382; an A, E, F, H, I, K, L, Q, S, V, or Y at position 384; an I, T, or V at position 385; and an I, F, L, V, or Y at position 438 and one, two, three, four, five, six, seven, eight, nine, ten, eleven, twelve, thirteen, fourteen, fifteen, or sixteen substitutions selected from: a D, E, F, H, N, Q, S, V, or Y at position 378; an A, D, F, H, I, K, L, M, Q, S, T, or Y at position 380; a T at position 383; an A, H, N, S, T, or V at position 386; an A, F, H, K, L, M, Q, S, T, V, or Y at position 421; an A, F, H, I, K, L, R, T, or Y at position 422; an A, G, or P at position 424; an A, I, L, T, or V at position 426; an A or L at position 428; a S at position 434; an A, G, I, M, N, P, T, or V at position 440; and an A, K, M, R, T, or V at position 442.

In some embodiments, a TfR-binding region comprising a modified CH3 peptide has four, five, six, seven, eight, nine, ten, eleven, twelve, thirteen, fourteen, fifteen, or sixteen amino acid substitutions in a set of amino acid positions consisting of 378, 380, 382, 383, 384, 385, 386, 421, 422, 424, 426, 428, 434, 438, 440, and 442; wherein the amino acid at each of positions 378, 380P 382, 383, 384, 385, 386, 421, 422, 424m 426, 428, 434, 438, 440, and 442 can be any of the amino acids provided in Table 10.

In some embodiments, a TfR-binding region comprises a modified CH3 peptide having the amino acid substitutions of any of the clones provided in Table 11-12. Affinities (Kd) for human and cyno TfR are shown in Table 11 for the indicated TfR-binding domains.

TABLE 10
Modified CH3 peptides: possible amino acids and the indicated positions.

| | Position (according to EU numbering) | | | | | | | | | | | | | | | | |
|---|---|---|---|---|---|---|---|---|---|---|---|---|---|---|---|---|---|
| | 378 | 380 | 382 | 383 | 384 | 385 | 386 | 387 | 421 | 422 | 424 | 426 | 428 | 434 | 438 | 440 | 442 |
| WT Fc | A | E | E | S | N | G | Q | P | N | V | S | S | M | N | Q | S | S |
| Possible | A | A | G | S | A | I | A | P | A | A | A | A | A | N | I | A | A |
| Amino | D | D | | T | E | T | H | | F | F | G | I | M | S | F | G | K |
| Acids at | E | E | | | F | V | N | | H | H | P | L | L | | L | I | M |
| Each | F | F | | | H | | Q | | K | I | S | S | | | V | M | R |
| Position | H | H | | | I | | S | | L | K | | T | | | Y | N | S |
| | N | I | | | K | | T | | M | L | | V | | | | P | T |
| | Q | K | | | L | | V | | N | R | | | | | | S | V |
| | S | L | | | Q | | | | Q | T | | | | | | T | |
| | V | M | | | S | | | | S | V | | | | | | V | |
| | Y | Q | | | V | | | | T | Y | | | | | | | |
| | | S | | | Y | | | | V | | | | | | | | |
| | | T | | | | | | | Y | | | | | | | | |
| | | Y | | | | | | | | | | | | | | | |

TABLE 11
TfR modified CH3 peptides derived from Clone 1-112

| | | | | | | | | | | | | | | K_{D} for |
|---|---|---|---|---|---|---|---|---|---|---|---|---|---|---|
| | Clone | | | | | | | | | | | | | huTfR |
| | Position (according to EU numbering | | | | | | | | | | | | | Apical |
| | 378 | 380 | 382 | 384 | 385 | 386 | 421 | 422 | 424 | 426 | 438 | 440 | 442 | Domain |
| Wild type | A | E | E | N | G | QI | N | V | S | S | Q | S | S | (nM) |
| Clone 1-112 | A | E | G | L | V | Q | N | I | A | T | I | G | S | 380 |
| Clone 1-112-2 | | A | | | | | | | | | | | | 240 |
| Clone 1-112-3 | | D | | | | | | | | | | | | 220 |
| Clone 1-112-4 | | F | | | | | | | | | | | | 120 |
| Clone 1-112-5 | | H | | | | | | | | | | | | 400 |
| Clone 1-112-6 | | I | | | | | | | | | | | | 230 |
| Clone 1-112-7 | | K | | | | | | | | | | | | 240 |
| Clone 1-112-8 | | L | | | | | | | | | | | | 130 |
| Clone 1-112-9 | | M | | | | | | | | | | | | 80 |
| Clone 1-112-10 | | Q | | | | | | | | | | | | 200 |
| Clone 1-112-11 | | | A | | | | | | | | | | | >4000 |
| Clone 1-112-12 | | | E | | | | | | | | | | | >4000 |
| Clone 1-112-13 | | | | A | | | | | | | | | | 750 |
| Clone 1-112-14 | | | | D | | | | | | | | | | >4000 |
| Clone 1-112-15 | | | | E | | | | | | | | | | 1400 |
| Clone 1-112-16 | | | | F | | | | | | | | | | 1400 |
| Clone 1-112-17 | | | | G | | | | | | | | | | >4000 |
| Clone 1-112-18 | | | | H | | | | | | | | | | 4000 |
| Clone 1-112-19 | | | | I | | | | | | | | | | 1400 |
| Clone 1-112-20 | | | | K | | | | | | | | | | 2200 |
| Clone 1-112-21 | | | | N | | | | | | | | | | >4000 |
| Clone 1-112-22 | | | | P | | | | | | | | | | >4000 |
| Clone 1-112-23 | | | | Q | | | | | | | | | | 1100 |
| Clone 1-112-24 | | | | S | | | | | | | | | | 3300 |
| Clone 1-112-25 | | | | T | | | | | | | | | | >4000 |
| Clone 1-112-26 | | | | V | | | | | | | | | | 1300 |
| Clone 1-112-27 | | | | Y | | | | | | | | | | 2100 |
| Clone 1-112-28 | | | | | A | | | | | | | | | >4000 |
| Clone 1-112-29 | | | | | G | | | | | | | | | >4000 |
| Clone 1-112-30 | | | | | I | | | | | | | | | 3400 |
| Clone 1-112-31 | | | | | L | | | | | | | | | >4000 |
| Clone 1-112-32 | | | | | T | | | | | | | | | 2000 |
| Clone 1-112-33 | | | | | | A | | | | | | | | 280 |
| Clone 1-112-34 | | | | | | G | | | | | | | | >4000 |
| Clone 1-112-35 | | | | | | H | | | | | | | | 470 |
| Clone 1-112-36 | | | | | | N | | | | | | | | 380 |
| Clone 1-112-37 | | | | | | S | | | | | | | | 400 |
| Clone 1-112-38 | | | | | | T | | | | | | | | 490 |
| Clone 1-112-39 | | | | | | | | A | | | | | | 3900 |
| Clone 1-112-40 | | | | | | | | H | | | | | | 3600 |
| Clone 1-112-41 | | | | | | | | K | | | | | | 2300 |
| Clone 1-112-42 | | | | | | | | L | | | | | | >4000 |
| Clone 1-112-43 | | | | | | | | R | | | | | | 1700 |
| Clone 1-112-44 | | | | | | | | T | | | | | | 630 |
| Clone 1-112-45 | | | | | | | | V | | | | | | 420 |
| Clone 1-112-46 | | | | | | | | Y | | | | | | 3000 |
| Clone 1-112-47 | | | | | | | | | G | | | | | 2200 |
| Clone 1-112-48 | | | | | | | | | H | | | | | >4000 |
| Clone 1-112-49 | | | | | | | | | P | | | | | 1100 |
| Clone 1-112-50 | | | | | | | | | S | | | | | 700 |
| Clone 1-112-51 | | | | | | | | | | A | | | | 2800 |
| Clone 1-112-52 | | | | | | | | | | G | | | | >4000 |
| Clone 1-112-53 | | | | | | | | | | H | | | | >4000 |
| Clone 1-112-54 | | | | | | | | | | I | | | | 560 |
| Clone 1-112-55 | | | | | | | | | | L | | | | 930 |
| Clone 1-112-56 | | | | | | | | | | S | | | | 1500 |
| Clone 1-112-57 | | | | | | | | | | V | | | | 450 |
| Clone 1-112-61 | | | | | | | | | | | A | | | >4000 |
| Clone 1-112-62 | | | | | | | | | | | D | | | >4000 |
| Clone 1-112-63 | | | | | | | | | | | E | | | >4000 |
| Clone 1-112-64 | | | | | | | | | | | F | | | 2800 |
| Clone 1-112-65 | | | | | | | | | | | G | | | >4000 |
| Clone 1-112-66 | | | | | | | | | | | H | | | >4000 |
| Clone 1-112-67 | | | | | | | | | | | K | | | >4000 |
| Clone 1-112-68 | | | | | | | | | | | L | | | 1200 |
| Clone 1-112-69 | | | | | | | | | | | N | | | >4000 |
| Clone 1-112-70 | | | | | | | | | | | P | | | >4000 |
| Clone 1-112-71 | | | | | | | | | | | Q | | | >4000 |
| Clone 1-112-72 | | | | | | | | | | | S | | | >4000 |
| Clone 1-112-73 | | | | | | | | | | | T | | | >4000 |
| Clone 1-112-74 | | | | | | | | | | | V | | | 1200 |
| Clone 1-112-75 | | | | | | | | | | | Y | | | 340 |
| Clone 1-112-76 | | | | | | | | | | | | A | | 360 |
| Clone 1-112-77 | | | | | | | | | | | | D | | >4000 |
| Clone 1-112-78 | | | | | | | | | | | | E | | >4000 |
| Clone 1-112-79 | | | | | | | | | | | | F | | >4000 |
| Clone 1-112-80 | | | | | | | | | | | | H | | >4000 |
| Clone 1-112-81 | | | | | | | | | | | | I | | 2400 |
| Clone 1-112-82 | | | | | | | | | | | | K | | >4000 |
| Clone 1-112-83 | | | | | | | | | | | | L | | >4000 |
| Clone 1-112-84 | | | | | | | | | | | | M | | 340 |
| Clone 1-112-85 | | | | | | | | | | | | N | | 2100 |
| Clone 1-112-86 | | | | | | | | | | | | P | | 3900 |
| Clone 1-112-87 | | | | | | | | | | | | Q | | >4000 |
| Clone 1-112-88 | | | | | | | | | | | | S | | 610 |
| Clone 1-112-89 | | | | | | | | | | | | T | | 450 |
| Clone 1-112-90 | | | | | | | | | | | | V | | 550 |
| Clone 1-112-91 | | | | | | | | | | | | Y | | >4000 |
| Clone 1-112-92 | | | | | | | | | P | I | | | | 310 |
| Clone 1-112-93 | | | | E | | | | | P | | | | | >4000 |
| Clone 1-112-94 | | | | E | | | | | P | I | | | | 770 |
| Clone 1-112-95 | | | | | | | | V | | | | S | | 2100 |
| Clone 1-112-96 | | | | N | | | | V | | | | S | | >4000 |
| Clone 1-112-97 | Y | | | | | | | | | | | | | 180 |
| Clone 1-112-98 | | | | | | | L | | | | | | | 80 |
| Clone 1-112-99 | | | | | | | | | | | | | R | 50 |
| Clone 1-112-100 | Y | L | G | L | V | A | L | I | P | I | Y | A | R | 410 |
| Clone 1-112-101 | A | E | G | L | V | A | L | I | P | I | Y | T | R | 180 |
| Clone 1-112-102 | A | E | G | L | V | Q | L | I | P | I | I | T | R | 41 |
| Clone 1-112-103 | A | E | G | L | V | Q | L | I | P | I | Y | T | R | 57 |
| Clone 1-112-104 | A | E | G | L | V | Q | L | I | A | T | Y | T | R | 390 |
| Clone 1-112-106 | A | E | G | L | V | Q | N | V | S | S | Y | S | R | >10000 |
| Clone 1-112-107 | A | E | G | L | V | Q | L | V | S | S | Y | S | R | >10000 |
| Clone 1-112-108 | A | E | G | L | V | Q | N | V | S | T | Y | S | R | 6100 |
| Clone 1-112-109 | A | E | G | L | V | Q | L | V | S | T | Y | S | R | 1900 |
| Clone 1-112-110 | A | E | G | L | V | Q | N | V | A | T | Y | S | R | 4300 |
| Clone 1-112-111 | A | E | G | L | V | Q | L | V | A | T | Y | S | S | >10000 |
| Clone 1-112-112 | A | E | G | L | V | Q | L | V | A | T | Y | S | R | 1200 |
| Clone 1-112-113 | A | E | G | L | V | Q | N | I | A | T | Y | A | S | 2700 |
| Clone 1-112-114 | A | E | G | L | V | Q | N | I | P | I | Y | A | S | 1200 |

TABLE 12
Modified CH3 peptides: possible amino acids and the indicated positions.

| | Clone | | | | | | | | | | | | |
|---|---|---|---|---|---|---|---|---|---|---|---|---|---|
| | Position (according to EU numbering) | | | | | | | | | | | | |
| | 378 | 380 | 382 | 384 | 385 | 386 | | 422 | 424 | 426 | 438 | 440 | |
| WT Fc | A | E | E | N | G | Q | 421 | V | S | S | Q | S | 442 |

| | | | | | | | | | |
|---|---|---|---|---|---|---|---|---|---|
| 1-112-115 | A | G | L | V | I | A | T | I | G |
| 1-112-116 | D | G | L | V | I | A | T | I | G |
| 1-112-117 | F | G | L | V | I | A | T | I | G |
| 1-112-119 | L | G | L | V | I | A | T | I | G |
| 1-112-120 | Q | G | L | V | I | A | T | I | G |
| 1-112-121 | | G | E | V | I | A | T | I | G |
| 1-112-122 | K | G | E | V | I | A | T | I | G |
| 1-112-123 | | G | L | V | | A | T | I | G |
| 1-112-124 | | G | L | V | I | | T | I | G |
| 1-112-125 | | G | L | V | I | A | T | Y | G |
| 1-112-126 | | G | L | V | I | A | T | I | |
| 1-112-127 | | G | L | V | I | A | T | I | T |
| 1-112-128 | | G | L | V | I | P | I | I | G |

In some embodiments, a TfR-binding region comprising a modified antibody heavy chain constant domain or Fc polypeptide (e.g., modified CH3 peptide) comprises four, five, six, seven, eight, nine, ten, or eleven substitutions in a set of amino acid positions consisting of: 380, 382, 384, 385, 386, 422, 424, 426, 436, 438, and 440; wherein the positions are determined with reference to EU numbering. In some embodiments, the substitutions are selected from: a K, L, I, Q, V, F, or M at position 380; a G at position 382; an A, E, or L at position 384; a V at position 385; an A, M, S or V at position 386; an I, H, R, or L at position 422; a P or A at position 424; a T, I, or V at position 426; a F at position 436; a L, V, I, or Y at position 438; and a M, G, A, T, or V at position 440.

In some embodiments, a TfR-binding region comprising a modified antibody heavy chain constant domain or Fc polypeptide (e.g., modified CH3 peptide) comprises: a G at position 382; a V at position 385; and a L, V, I, or Y at position 438.

In some embodiments, a TfR-binding region comprising a modified antibody heavy chain constant domain or Fc polypeptide (e.g., modified CH3 peptide) comprises a G at position 382; a V at position 385; and a L, V, I, or Y at position 438 and one, two, three, four, five, six, seven, eight, or nine substitutions in a set of amino acid positions consisting of: 380, 384, 386, 422, 424, 426, 436, and 440; wherein the positions are determined with reference to EU numbering. In some embodiments, the substitutions are selected from: a K, L, I, Q, V, F, or M at position 380; an A, E, or L at position 384; an A, M, S or V at position 386; an I, H, R, or L at position 422; a P or A at position 424; a T, I, or V at position 426; a F at position 436; and a M, G, A, T, or V at position 440.

In some embodiments, a TfR-binding region comprising a modified antibody heavy chain constant domain or Fc polypeptide (e.g., modified CH3 peptide) comprises a G at position 382; a V at position 385; and a L, V, I, or Y at position 438 and one, two, three, four, five, six, seven, eight, or nine substitutions selected from: a K, L, I, Q, V, F, or M at position 380; an A, E, or L at position 384; an A, M, S or V at position 386; an I, H, R, or L at position 422; a P or A at position 424; a T, I, or V at position 426; a F at position 436; and a M, G, A, T, or V at position 440.

In some embodiments, a TfR-binding region comprising a modified antibody heavy chain constant domain or Fe polypeptide (e.g., modified CH3 peptide) comprises: a G at position 382; an A, E, or L at position 384; a V at position 385; a P or A at position 424; a T, I, or V at position 426; a L, V, I, or Y at position 438; and a M, G, A, T, or V at position 440.

In some embodiments, a TfR-binding region comprising a modified antibody heavy chain constant domain or Fc polypeptide (e.g., modified CH3 peptide) a G at position 382: an A, E, or L at position 384; a V at position 385; a P or A at position 424; a T, I, or V at position 426; a L, V, I, or Y at position 438; and a M, G, A, T, or V at position 440 and one, two, three, or four substitutions in a set of amino acid positions consisting of: 380, 386, 422, and 436; wherein the positions are determined with reference to EU numbering. In some embodiments, the substitutions are selected from: a K, L, I, Q, V, F, or M at position 380; an A, M, S or V at position 386; an I, H, R, or L at position 422; and a F at position 436.

In some embodiments, a TfR-binding region comprising a modified antibody heavy chain constant domain or Fc polypeptide (e.g., modified CH3 peptide) comprises a G at position 382; an A, E, or L at position 384; a V at position 385; a P or A at position 424; a T, I, or V at position 426; a L, V, I, or Y at position 438; and a M, G, A, T, or V at position 440 and one, two, three, or four substitutions selected from: a K, L, I, Q, V, F, or M at position 380; an A, M, S or V at position 386; an I, H, R, or L at position 422; and a F at position 436.

In some embodiments, a TfR-binding region comprises a modified CH3 peptide having the amino acid substitutions of any of the clones provided in Table 13.

TABLE 13
Modified CH3 peptides: possible amino acids and the indicated positions.

| | Clone | | | | | | | | | | |
|---|---|---|---|---|---|---|---|---|---|---|---|
| | Position (according to EU numbering) | | | | | | | | | | |
| | 380 | 382 | 384 | 385 | 386 | 422 | 424 | 426 | 436 | 438 | 440 |
| WT Fc | E | E | N | G | Q | V | S | S | Y | Q | S |
| Clone 1 | K | G | E | V | A | I | A | T | Y | L | M |
| Clone 3 | L | G | A | V | A | V | A | T | Y | V | G |
| Clone 1-15 | E | G | E | V | Q | I | A | T | Y | I | G |
| Clone 1-34 | I | G | E | V | A | I | A | T | E | V | A |
| Clone 1-40 | E | G | L | V | Q | H | A | T | Y | I | G |
| Clone 1-41 | Q | G | E | V | V | I | A | T | Y | I | T |
| Clone 1-44 | V | G | E | V | S | I | A | T | Y | V | G |
| Clone 1-50 | F | G | E | V | A | I | A | T | Y | Y | G |
| Clone 1-55 | I | G | E | V | A | I | A | T | Y | L | M |
| Clone 1-57 | I | G | E | V | Q | I | A | T | Y | I | G |
| Clone 1-69 | M | G | A | V | S | V | A | T | Y | I | G |
| Clone 1-70 | M | G | E | V | A | I | A | T | Y | I | G |
| Clone 1-79 | M | G | E | V | M | R | P | I | E | I | V |
| Clone 1-84 | M | G | E | V | Q | L | A | T | F | Y | V |
| Clone 1-92 | Q | G | E | V | A | I | A | T | Y | L | M |
| Clone 1-105 | E | G | A | V | A | I | A | T | Y | I | G |
| Clone 1-110 | E | G | L | V | A | I | A | T | Y | V | G |
| Clone 1-112 | E | G | L | V | Q | I | A | T | Y | I | G |
| Clone 1-117 | L | G | E | V | Q | I | P | V | F | Y | A |
| Clone 1-120 | M | G | E | V | A | I | A | T | Y | I | A |
| Clone 1-123 | M | G | E | V | A | I | P | I | Y | I | G |
| Clone 1-131 | M | G | E | V | Q | I | P | I | Y | L | M |
| Clone 1-133 | M | G | E | V | S | R | P | I | Y | L | V |
| Clone 1-135 | M | G | L | V | Q | I | A | T | Y | I | T |
| Clone 1-142 | M | G | E | V | Q | R | P | I | Y | L | M |
| Clone 1-143 | M | G | E | V | Q | I | P | I | Y | Y | A |
| Clone 1-144 | M | G | E | V | Q | I | P | I | Y | I | G |

In some embodiments, a TfR-binding region comprising a modified antibody heavy chain constant domain or Fc polypeptide (e.g., modified CH3 peptide) comprises four, five, six, seven, eight, nine, ten, or eleven substitutions in a set of amino acid positions consisting of: 378, 380, 382, 384, 385, 386, 422, 424, 426, 438, 440; wherein the positions are determined with reference to EU numbering. In some embodiments, the substitutions are selected from: a D, Y, H, S, V, F, or Y at position 378; a F, Y, or D at position 380; a G at position 382; an A, E or L at position 384; a V at position 385; an A or S at position 386; a F, L, or I at position 422; a P or A at position 424; an I at position 426; an I or Y at position 438; and a V, G, or T at position 440.

In some embodiments, a TfR-binding region comprising a modified antibody heavy chain constant domain or Fc polypeptide (e.g., modified CH3 peptide) comprises: a G at position 382; a V at position 385; and an I or Y at position 438.

In some embodiments, a TfR-binding region comprising a modified antibody heavy chain constant domain or Fc polypeptide (e.g., modified CH3 peptide) comprises a G at position 382; a V at position 385; and an I or Y at position 438; and one, two, three, four, five, six, seven, or eight substitutions in a set of amino acid positions consisting of: 378, 380, 384, 386, 422, 424, 426, 440; wherein the positions are determined with reference to EU numbering. In some embodiments, the substitutions are selected from: a D, Y, H, S, V, F, or Y at position 378; a F, Y, or D at position 380; an A, E or L at position 384; an A or S at position 386; a F, L, or I at position 422; a P or A at position 424; an I at position 426; and a V, G, or T at position 440.

In some embodiments, a TfR-binding region comprising a modified antibody heavy chain constant domain or Fc polypeptide (e.g., modified CH3 peptide) comprises a G at position 382; a V at position 385; and an I or Y at position 438; and one, two, three, four, five, six, seven, or eight substitutions selected from: a D, Y, H, S, V, F, or Y at position 378; a F, Y, or D at position 380; an A, E or L at position 384; an A or S at position 386; a F, L, or I at position 422; a P or A at position 424; an I at position 426; and a V, G, or T at position 440.

In some embodiments, a TfR-binding region comprising a modified antibody heavy chain constant domain or Fc polypeptide (e.g., modified CH3 peptide) comprises: a G at position 382; an A. E or L at position 384; a V at position 385; a P or A at position 424; an I at position 426; an I or Y at position 438.

In some embodiments, a TfR-binding region comprising a modified antibody heavy chain constant domain or Fc polypeptide (e.g., modified CH3 peptide) comprises: a G at position 382; an A. E or L at position 384; a V at position 385; a P or A at position 424; an I at position 426; an I or Y at position 438; and one, two, three, or four substitutions in a set of amino acid positions consisting of: 378, 382, 383, 389, 421, 440, and 442, wherein the positions are determined with reference to EU numbering. In some embodiments, the substitutions are selected from: a D, Y, H, S, V, F, or Y at position 378; a F, Y, or D at position 380; an A or S at position 386; and a F, L, or I at position 422.

In some embodiments, a TfR-binding region comprising a modified antibody heavy chain constant domain or Fc polypeptide (e.g, modified CH3 peptide) comprises: a G at position 382; an A. E or L at position 384; a V at position 385; a P or A at position 424; an I at position 426; an I or Y at position 438; and one, two, three, four, five, six, or seven substitutions selected from: a D, Y, H, S, V, F, or Y at position 378; a F, Y, or D at position 380; an A or S at position 386; and a F, L, or I at position 422.

In some embodiments, a TfR-binding region comprising a modified CH3 peptide having the amino acid substitutions of any of the clones provided in Table 14.

TABLE 14
Exemplary TfR-binding regions comprising a modified CH3.

| | Clone | | | | | | | | | | | | | | | | |
|---|---|---|---|---|---|---|---|---|---|---|---|---|---|---|---|---|---|
| | Positions (according to EU numbering) | | | | | | | | | | | | | | | | |
| | 378 | 379 | 380 | 381 | 382 | 383 | 384 | 385 | 386 | 422 | 423 | 424 | 425 | 426 | 438 | 439 | 440 |
| WT Fc | A | V | E | W | E | S | N | G | Q | V | F | S | C | S | Q | K | S |
| Clone 1-244 | D | | F | | G | | A | V | | F | | P | | I | I | | V |
| Clone 1-252 | Y | | Y | | G | | L | V | | L | | P | | I | Y | | V |
| Clone 1-265 | H | | F | | G | | L | V | | F | | P | | I | Y | | V |
| Clone 1-285 | S | | | | G | | L | V | | F | | P | | I | Y | | V |
| Clone 1-292 | | | D | | G | | L | V | A | | | A | | I | I | | G |
| Clone 1-300 | V | | F | | G | | E | V | | F | | P | | I | Y | | V |
| Clone 1-303 | | | D | | G | | L | V | A | I | | A | | I | I | | T |
| Clone 1-317 | D | | | | G | | L | V | A | F | | P | | I | I | | V |
| Clone 1-320 | F | | F | | G | | E | V | | L | | P | | I | Y | | V |
| Clone 1-321 | H | | | | G | | L | V | | I | | P | | I | I | | T |
| Clone 1-331 | S | | | | G | | L | V | S | F | | P | | I | I | | V |
| Clone 1-334 | Y | | | | G | | L | V | | I | | P | | I | I | | T |

### 4. Group IV TfR Binding Regions

In some embodiments, a TfR-binding region comprises an anti-TfR antibody antigen binding domain. An anti-TfR antibody antigen binding domain can comprise an antibody, a Fab (including a F(ab′)2), a scFab, a Fv fragment, an scFv, or a nanobody. Anti-TfR antibody antigen binding domains include, but are not limited to: a 17H10 anti-TfR Fab or scFv; a 17H10.1 anti-TfR Fab or scFv; a JC-141 anti-TfR antibody; a JC-141 anti-TfR Fab; a JC-141 anti-TfR scFv: an anti-TfR antibody, Fab, scFab, Fv fragment, or scFv having the heavy chain and light chain CDR1, CDR2, and CDR3 sequences of the JR-141 antibody (WO2016208695); a JC-171 anti-TfR antibody; a JC-171 anti-TfR Fab; a JC-171 anti-TfR scFv: an anti-TfR antibody, Fab, scFab, Fv fragment, or scFv having the heavy chain and light chain CDR1, CDR2, and CDR3 sequences of the JR-171 antibody (WO2018124121): a “Brain shuttle” (BS) anti-TfR Fab; an anti-TfR antibody, Fab, scFab, Fv fragment, or scFv having the heavy chain and light chain CDR1, CDR2, and CDR3 sequences of the BS anti-TfR Fab (WO2018210898, WO2015101588, and WO2014033074); a 13E4v2ii anti-TfR antibody; a 13E4v2ii anti-TfR Fab: a 13E4v2ii anti-TfR scFv: an anti-TfR antibody. Fab, scFab. Fv fragment, or scFv having the heavy chain and light chain CDR1, CDR2, and CDR3 sequences of the 13E4v2ii antibody (WO2020132584); a TfR12 anti-TfR scFv; an anti-TfR antibody, Fab, scFab, Fv fragment, or scFv having the heavy chain and light chain CDR1, CDR2, and CDR3 sequences of the TfR12 anti-TfR scFv (WO2021/205358); a TfR13 anti-TfR scFv; or an anti-TfR antibody. Fab, scFab, Fv fragment, or scFv having the heavy chain and light chain CDR1, CDR2, and CDR3 sequences of the Tfrt3 anti-TfR scFv (WO2021/205358) (Sequences shown in Table 15).

Additional anti-TfR antibodies are described in WO2021/205358, and the dual binding molecules of the invention can include any antibody antigen binding domain with the CDRs or variable regions of any one of TfR1, TfR2, TfR3, TfR4, TfR5, TfR6, TfR7, TfR8, TfR9, TfR10, TfR11, TfR12, TfR13, TfR14, TfR15. TfR16, TfR17, TfR18, TfR19, TfR20, TfR21, TfR22, TfR23. TfR24, TfR25, TfR26, TfR27, TfR28, TfR29, TfR30, TfR31, TfR32, TfR33, TfR34, TfR35, TfR36, TfR37, and TfR38 described therein.

TABLE 15
Exemplary TfR-binding regions comprising antibody antigen binding domains.

| | |
|---|---|
| 17H10 | Heavy chain: |
| anti-TfR Fab | EVQLVESGGGLVQPGGSLRLSCAVSGFSLTTYGVDWVRQAPGKGLEWVGIIWGDGTTHYHSAL |
| | ISRFTISKDNSKNTVYLQMNSLRAEDTAVYYCAKPGGASWGQGTLVTVSSASTKGPSVFPLAP |
| | SSKSTSGGTAALGCLVKDYFPEPVTVSWNSGALTSGVHTFPAVLQSSGLYSLSSVVTVPSSSL |
| | GTQTYICNVNHKPSNTKVDKKVEPKSCDKTH (SEQ ID NO: 127) |
| | Light chain: |
| | DIQMTQSPSSLSASVGDRVTITCKASQLVGTNVAWYQQKPGKSPKALFYSASYRNQGVPSRES |
| | GSGSGTDFTLTISSLQPEDFATYFCQHYNSYPFTFGQGTKVEIKRTVAAPSVFIFPPSDEQLK |
| | SGTASVVCLLNNFYPREAKVQWKVDNALQSGNSQESVTEQDSKDSTYSLSSTLTLSKADYEKH |
| | KVYACEVTHQGLSSPVTKSENRGEC (SEQ ID NO: 128) |
| 17H10.1 | Heavy chain: |
| anti-TfR Fab | EVQLVESGGGLVQPGGSLRLSCAVSGFSLTTYGVDWVRQAPGKGLEWVGIIWGDGTTHYHSAL |
| | ISRFTISKDNSKNTVYLQMNSLRAEDTAVYYCAKPGGASWGQGTLVTVSSASTKGPSVFPLAP |
| | SSKSTSGGTAALGCLVKDYFPEPVTVSWNSGALTSGVHTFPAVLQSSGLYSLSSVVTVPSSSL |
| | GTQTYICNVNHKPSNTKVDKKVEPKSCDKTH (SEQ ID NO: 129) |
| | Light chain: |
| | DIQMTQSPSSLSASVGDRVTITCKASQFVGTAVAWYQQKPGKSPKALFYSASYRNEGVPSRES |
| | GSGSGTDFTLTISSLQPEDFATYFCQHYNSYPFTFGQGTKVEIKRTVAAPSVFIFPPSDEQLK |
| | SGTASVVCLLNNFYPREAKVQWKVDNALQSGNSQESVTEQDSKDSTYSLSSTLTLSKADYEKH |
| | KVYACEVTHQGLSSPVTKSENRGEC (SEQ ID NO: 130) |
| 17H10 | EVQLVESGGGLVQPGGSLRLSCAVSGFSLTTYGVDWVRQAPGKGLEWVGIIWGDGTTHYHSAL |
| anti-TfR scFv | ISRFTISKDNSKNTVYLQMNSLRAEDTAVYYCAKPGGASWGQGTLVTVSSGGGGSGGGGSGGG |
| | GSDIQMTQSPSSLSASVGDRVTITCKASQLVGTNVAWYQQKPGKSPKALFYSASYRNQGVPSR |
| | FSGSGSGTDETLTISSLQPEDFATYFCQHYNSYPFTFGQGTKVEIKEPKSS |
| | (SEQ ID NO: 131) |
| 17H10.1 | EVQLVESGGGLVQPGGSLRLSCAVSGFSLTTYGVDWVRQAPGKGLEWVGIIWGDGTTHYHSAL |
| anti-TfR scFv | ISRFTISKDNSKNTVYLQMNSLRAEDTAVYYCAKPGGASWGQGTLVTVSSGGGGSGGGGSGGG |
| | GSDIQMTQSPSSLSASVGDRVTITCKASQFVGTAVAWYQQKPGKSPKALFYSASYRNEGVPSR |
| | FSGSGSGTDFTLTISSLQPEDFATYFCQHYNSYPFTFGQGTKVEIKEPKSS |
| | (SEQ ID NO: 132) |
| JC-141 | Heavy chain: |
| anti-TfR | EVQLVQSGAEVKKPGESLKISCKGSGYSFTNYWLGWVRQMPGKGLEWMGDIYPGGDYPTYSEK |
| antibody | FKVQVTISADKSISTAYLQWSSLKASDTAMYYCARSGNYDEVAYWGQGTLVTVSSASTKGPSV |
| | FPLAPSSKSTSGGTAALGCLVKDYFPEPVTVSWNSGALTSGVHTEPAVLQSSGLYSLSSVVTV |
| | PSSSLGTQTYICNVNHKPSNTKVDKKVEPKSCDKTHTCPPCPAPELLGGPSVFLFPPKPKDTL |
| | MISRTPEVTCVVVDVSHEDPEVKENWYVDGVEVHNAKTKPREEQYNSTYRVVSVLTVLHQDWL |
| | NGKEYKCKVSNKALPAPIEKTISKAKGQPREPQVYTLPPSRDELTKNQVSLTCLVKGFYPSDI |
| | AVEWESNGQPENNYKTTPPVLDSDGSFFLYSKLTVDKSRWQQGNVFSCSVMHEALHNHYTQKS |
| | LSLSPGK (SEQ ID NO: 65) |
| | Light chain: |
| | DIVMTQTPLSLSVTPGQPASISCRSSQSLVHSNGNTYLHWYLQKPGQSPQLLIYKVSNRFSGV |
| | PDRFSGSGSGTDFTLKISRVEAEDVGVYYCSQSTHVPWTFGQGTKVEIKRTVAAPSVFIFPPS |
| | DEQLKSGTASVVCLLNNFYPREAKVQWKVDNALQSGNSQESVTEQDSKDSTYSLSSTLTLSKA |
| | DYEKHKVYACEVTHQGLSSPVTKSENRGEC (SEQ ID NO: 66) |
| JC-141 | Heavy chain: |
| anti-TfR Fab | EVQLVQSGAEVKKPGESLKISCKGSGYSFTNYWLGWVRQMPGKGLEWMGDIYPGGDYPTYSEK |
| | FKVQVTISADKSISTAYLQWSSLKASDTAMYYCARSGNYDEVAYWGQGTLVTVSSASTKGPSV |
| | FPLAPSSKSTSGGTAALGCLVKDYFPEPVTVSWNSGALTSGVHTFPAVLQSSGLYSLSSVVTV |
| | PSSSLGTQTYICNVNHKPSNTKVDKKVEPKSC (SEQ ID NO: 67) |
| | Light chain: |
| | DIVMTQTPLSLSVTPGQPASISCRSSQSLVHSNGNTYLHWYLQKPGQSPQLLIYKVSNRESGV |
| | PDRFSGSGSGTDFTLKISRVEAEDVGVYYCSQSTHVPWTFGQGTKVEIKRTVAAPSVEIFPPS |
| | DEQLKSGTASVVCLLNNEYPREAKVQWKVDNALQSGNSQESVTEQDSKDSTYSLSSTLTLSKA |
| | DYEKHKVYACEVTHQGLSSPVTKSENRGEC (SEQ ID NO: 68) |
| JC-141 | Heavy chain: |
| anti-TfR | EVQLVQSGAEVKKPGESLKISCKGSGYSFTNYWLGWVRQMPGKGLEWMGDIYPGGDYPTYSEK |
| scFv | FKVQVTISADKSISTAYLQWSSLKASDTAMYYCARSGNYDEVAYWGQGTLVTVSS (SEQ ID |
| sequences | NO: 69) |
| | Light chain: |
| | DIVMTQTPLSLSVTPGQPASISCRSSQSLVHSNGNTYLHWYLQKPGQSPQLLIYKVSNRFSGV |
| | PDRFSGSGSGTDFTLKISRVEAEDVGVYYCSQSTHVPWTFGQGTKVEIK (SEQ ID |
| | NO: 70) |
| JC-141 | Heavy chain: |
| anti-TfR | CDR1: GYSFTNYWLG (SEQ ID NO: 71) |
| CDR | CDR2: DIYPGGDYPTYSEKFKV (SEQ ID NO: 72) |
| sequences | CDR3: SGNYDEVAY (SEQ ID NO: 73) |
| | Light chain: |
| | CDR1: RSSQSLVHSNGNTYLH (SEQ ID NO: 74) |
| | CDR2: KVSNRFS (SEQ ID NO: 75) |
| | CDR3: SQSTHVPWT (SEQ ID NO: 76) |
| JC-171 | Heavy Chain: |
| anti-TfR | EVQLVQSGAEVKKPGESLKISCKGSGYSFMNYWLGWVRQMPGKGLEWMGDIYPGGDYPTYSEK |
| antibody | FKVQVTISADKSISTAYLQLSSLKASDTAMYYCARSGNYDEVAYWGQGTLVTVSSASTKGPSV |
| | FPLAPSSKSTSGGTAALGCLVKDYFPEPVTVSWNSGALTSGVHTEPAVLQSSGLYSLSSVVTV |
| | PSSSLGTQTYICNVNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYNSTYRVVSVLTVLHQDWL |
| | NGKEYKCKVSNKALPAPIEKTISKAKGQPREPQVYTLPPSRDELTKNQVSLTCLVKGFYPSDI |
| | AVEWESNGQPENNYKTTPPVLDSDGSFFLYSKLTVDKSRWQQGNVFSCSVMHEALHNHYTQKS |
| | LSLSPGK (SEQ ID NO: 77) |
| | Light chain: |
| | DIVMTQTPLSLSVTPGQPASISCRSSQSLVHSNGNTYLHWYLQKPGQSPQLLIYKVSNRFSGV |
| | PDRFSGSGSGTDFTLKISRVEAEDVGVYYCSQSTHVPWTFGQGTKVEIKRTVAAPSVFIFPPS |
| | DEQLKSGTASVVCLLNNEYPREAKVQWKVDNALQSGNSQESVTEQDSKDSTYSLSSTLTLSKA |
| | DYEKHKVYACEVTHQGLSSPVTKSENRGEC (SEQ ID NO: 78) |
| JC-171 | Heavy Chain: |
| anti-TfR | EVQLVQSGAEVKKPGESLKISCKGSGYSFMNYWLGWVRQMPGKGLEWMGDIYPGGDYPTYSEK |
| Fab | FKVQVTISADKSISTAYLQLSSLKASDTAMYYCARSGNYDEVAYWGQGTLVTVSSASTKGPSV |
| | FPLAPSSKSTSGGTAALGCLVKDYFPEPVTVSWNSGALTSGVHTFPAVLQSSGLYSLSSVVTV |
| | PSSSLGTQTYICNVNHKPSNTKVDKKVEPKSC (SEQ ID NO: 79) |
| | Light chain: |
| | DIVMTQTPLSLSVTPGQPASISCRSSQSLVHSNGNTYLHWYLQKPGQSPQLLIYKVSNRFSGV |
| | PDRFSGSGSGTDFTLKISRVEAEDVGVYYCSQSTHVPWTFGQGTKVEIKRTVAAPSVFIFPPS |
| | DEQLKSGTASVVCLLNNFYPREAKVQWKVDNALQSGNSQESVTEQDSKDSTYSLSSTLTLSKA |
| | DYEKHKVYACEVTHQGLSSPVTKSENRGEC (SEQ ID NO: 80) |
| JC-171 | Heavy Chain: |
| anti-TfR | EVQLVQSGAEVKKPGESLKISCKGSGYSFMNYWLGWVRQMPGKGLEWMGDIYPGGDYPTYSEK |
| scFv | FKVQVTISADKSISTAYLQLSSLKASDTAMYYCARSGNYDEVAYWGQGTLVTVSS (SEQ ID |
| sequences | NO: 81) |
| | Light chain: |
| | DIVMTQTPLSLSVTPGQPASISCRSSQSLVHSNGNTYLHWYLQKPGQSPQLLIYKVSNRFSGV |
| | PDRFSGSGSGTDFTLKISRVEAEDVGVYYCSQSTHVPWTFGQGTKVEIK (SEQ ID |
| | NO: 82) |
| JC-171 | Heavy chain: |
| anti-TfR | CDR1: GYSEMNYWLG (SEQ ID NO: 83) |
| CDR | CDR2: DIYPGGDYPTYSEKFKV (SEQ ID NO: 84) |
| sequences | CDR3: SGNYDEVAY (SEQ ID NO: 85) |
| | Light chain: |
| | CDR1: RSSQSLVHSNGNTYLH (SEQ ID NO: 86) |
| | CDR2: KVSNRFS (SEQ ID NO: 87) |
| | CDR3: SQSTHVPWT (SEQ ID NO: 88) |
| BS anti-TIR | Heavy Chain: |
| Fab | QSMQESGPGLVKPSQTLSLTCTVSGFSLSSYAMSWIRQHPGKGLEWIGYIWSGGSTDYASWAK |
| | SRVTISKTSTTVSLKLSSVTAADTAVYYCARRYGTSYPDYGDASGEDPWGQGTLVTVSSASVA |
| | APSVFIFPPSDEQLKSGTASVVCLLNNFYPREAKVQWKVDNALQSGNSQESVTEQDSKDSTYS |
| | LSSTLTLSKADYEKHKVYACEVTHQGLSSPVTKSENRGEC (SEQ ID NO: 89) |
| | Light chain: |
| | AIQLTQSPSSLSASVGDRVTITCRASQSISSYLAWYQQKPGKAPKLLIYRASTLASGVPSRES |
| | GSGSGTDFTLTISSLQPEDFATYYCQQNYASSNVDNTFGGGTKVEIKSSASTKGPSVFPLAPS |
| | SKSTSGGTAALGCLVKDYFPEPVTVSWNSGALTSGVHTFPAVLQSSGLYSLSSVVTVPSSSLG |
| | TQTYICNVNHKPSNTKVDKKVEPKSC (SEQ ID NO: 90) |
| BS anti-TfR | Heavy chain: |
| Fab CDR | CDR1: GFSLSSYAMS (SEQ ID NO: 91) |
| sequences | CDR2: YIWSGGSTDYASWAKS (SEQ ID NO: 92) |
| | CDR3: RYGTSYPDYGDASGEDP (SEQ ID NO: 93) |
| | Light chain: |
| | CDR1: RASQSISSYLA (SEQ ID NO: 94) |
| | CDR2: RASTLAS (SEQ ID NO: 95) |
| | CDR3: QQNYASSNVDNT (SEQ ID NO: 96) |
| 13E4v2ii | Heavy Chain (with knob mutation): |
| anti-TfR | QVQLVQSGAEVKKPGASVKVSCKASGYTFTNYWMHWVRQAPGQGLEWIGEINPINGRSNYAEK |
| antibody | FQGRVTLTVDTSSSTAYMELSRLRSDDTAVYYCARGTRAMHYWGQGTLVTVSSASTKGPSVEP |
| | LAPSSKSTSGGTAALGCLVKDYFPEPVTVSWNSGALTSGVHTFPAVLQSSGLYSLSSVVTVPS |
| | SSLGTQTYICNVNHKPSNTKVDKKVEPKSCDKTHTCPPCPAPEAAGGPSVFLFPPKPKDTLMI |
| | SRTPEVTCVVVDVSHEDPEVKENWYVDGVEVHNAKTKPREEQYNSTYRVVSVLTVLHQDWLNG |
| | KEYKCKVSNKARPAPIEKTISKAKGQPREPQVYTLPPSRDELTKNQVSLWCLVKGFYPSDIAV |
| | EWESNGQPENNYKTTPPVLDSDGSFFLYSKLTVDKSRWQQGNVESCSVMHEALHNHYTQKSLS |
| | LSPGK (SEQ ID NO: 97) |
| | Heavy Chain (with hole mutation): |
| | QVQLVQSGAEVKKPGASVKVSCKASGYTFTNYWMHWVRQAPGQGLEWIGEINPINGRSNYAEK |
| | FQGRVTLTVDTSSSTAYMELSRLRSDDTAVYYCARGTRAMHYWGQGTLVTVSSASTKGPSVEP |
| | LAPSSKSTSGGTAALGCLVKDYFPEPVTVSWNSGALTSGVHTFPAVLQSSGLYSLSSVVTVPS |
| | SSLGTQTYICNVNHKPSNTKVDKKVEPKSCDKTHTCPPCPAPEAAGGPCVFLFPPKPKDTLMI |
| | SRTPEVTCVVVDVSHEDPEVKFNWYVDGVEVHNAKTKPREEQYNSTYRVVSVLTVLHQDWING |
| | KEYKCKVSNKARPAPIEKTISKAKGQPREPQVYTLPPSRDELTKNQVSLSCAVKGFYPSDIAV |
| | EWESNGQPENNYKTTPPVLDSDGSFFLVSKLTVDKSRWQQGNVFSCSVMHEALHNHYTQKSLS |
| | LSPGK (SEQ ID NO: 98) |
| | Light chain: |
| | DIQMTQSPSSLSASVGDRVTITCRTSENIYNNLAWYQQKPGKSPKLLIYAATNLADGVPSRFS |
| | GSGSGTDYTLTISSLQPEDFATYYCQHFWGTPLTFGGGTKVEIKRTVAAPSVFIFPPSDEQLK |
| | SGTASVVCLLNNFYPREAKVQWKVDNALQSGNSQESVTEQDSKDSTYSLSSTLTLSKADYEKH |
| | KVYACEVTHQGLSSPVTKSENRGEC (SEQ ID NO: 99) |
| 13E4v2ii | Heavy Chain: |
| anti-TfR | QVQLVQSGAEVKKPGASVKVSCKASGYTFTNYWMHWVRQAPGQGLEWIGEINPINGRSNYAEK |
| Fab | FQGRVTLTVDTSSSTAYMELSRLRSDDTAVYYCARGTRAMHYWGQGTLVTVSSASTKGPSVEP |
| | LAPSSKSTSGGTAALGCLVKDYFPEPVTVSWNSGALTSGVHTFPAVLQSSGLYSLSSVVTVPS |
| | SSLGTQTYICNVNHKPSNTKVDKKVEPKSC (SEQ ID NO: 100) |
| | Light chain: |
| | DIQMTQSPSSLSASVGDRVTITCRTSENIYNNLAWYQQKPGKSPKLLIYAATNLADGVPSRES |
| | GSGSGTDYTLTISSLQPEDFATYYCQHFWGTPLTFGGGTKVEIKRTVAAPSVFIFPPSDEQLK |
| | SGTASVVCLLNNFYPREAKVQWKVDNALQSGNSQESVTEQDSKDSTYSLSSTLTLSKADYEKH |
| | KVYACEVTHQGLSSPVTKSENRGEC (SEQ ID NO: 101) |
| 13E4v2ii | Heavy Chain: |
| anti-TfR | QVQLVQSGAEVKKPGASVKVSCKASGYTFTNYWMHWVRQAPGQGLEWIGEINPINGRSNYAEK |
| scFv | FQGRVTLTVDTSSSTAYMELSRLRSDDTAVYYCARGTRAMHYWGQGTLVTVSS (SEQ ID |
| sequences | NO: 102) |
| | Light chain: |
| | DIQMTQSPSSLSASVGDRVTITCRTSENIYNNLAWYQQKPGKSPKLLIYAATNLADGVPSRES |
| | GSGSGTDYTLTISSLQPEDFATYYCQHFWGTPLTFGGGTKVEIK (SEQ ID NO: 103) |
| 13E4v2ii | Heavy chain: |
| anti-TfR | CDR1: GYTFTNYWMH (SEQ ID NO: 104) |
| CDR | CDR2: EINPINGRSNYAEKFQG (SEQ ID NO: 105) |
| sequences | CDR3: ARGTRAMHY (SEQ ID NO: 106) |
| | Light chain: |
| | CDR1: RTSENIYNNLA (SEQ ID NO: 107) |
| | CDR2: AATNLAD (SEQ ID NO: 108) |
| | CDR3: QHFWGTPLT (SEQ ID NO: 109) |
| Tfr12 anti- | QVQLQQWGAGLLKPSETLSLTCAVYGGSFSGYYWTWIRQPPGKGLEWIGEFIHSGSTNYNPSL |
| TfR scFv | KSRVTISVDTSKNQFSLKLSSVTAADTAVYFCARGSMDSSGFYSFDFWGQGTLVTVSSGTEGK |
| | SSGSGSESKSTSYEVTQPPSVSVSPGQTASITCSGHKLGDKFASWYQQKPGQSPVLVIYQDRK |
| | RPSGIPERFSGSNSGNTATLTISGTQAMDEADYYCQTWYSSTVIFGGGTKLTVL (SEQ ID |
| | NO: 110) |
| Tfr12 anti- | Heavy Chain: |
| TfR scFv | CDR1: GGSFSGYY (SEQ ID NO: 111) |
| CDR | CDR2: FIHSGST (SEQ ID NO: 112) |
| sequences | CDR3: ARGSMDSSGFYSFDF (SEQ ID NO: 113) |
| | Light Chain: |
| | CDR1: KLGDKF (SEQ ID NO: 114) |
| | CDR2: QDRKRP (SEQ ID NO: 115) |
| | CDR3: QTWYSSTVI (SEQ ID NO: 116) |
| Tfr13 anti- | EVQLLESGGGLVQPGGSLRLSCADSGFTESSYAMNWVRQAPGKGLEWVSGISGSGGHTYYADS |
| TfR scFv | VKGRFTVSRDNSKNTLYLQMNSLRAEDTAVYYCAREGYDSSGYNPFDYWGQGTQVTVSSGTEG |
| | KSSGSGSESKSTSYELTQPPSVSVSPGQTASITCSGDKLGDKYASWYQQKPGQSPVLVIYQDS |
| | KRPSGIPERFSGSNSGNTATLTISGTQAMDEADYYCQAWDSSTVVFGGGTKLTVL (SEQ ID |
| | NO: 117) |
| Tfr13 anti- | Heavy Chain: |
| TfR scFv | CDR1: GFTFSSYAMNW (SEQ ID NO: 118) |
| CDR | CDR2: ISGSGGHT (SEQ ID NO: 119) |
| sequences | CDR3: AREGYDSSGYNPEDY (SEQ ID NO: 120) |
| | Light Chain: |
| | CDR1: KLGDKYA (SEQ ID NO: 121) |
| | CDR2: QDSKRPS (SEQ ID NO: 122) |
| | CDR3: QAWDSSTVV (SEQ ID NO: 123) |

Additional anti-TfR antibodies are known in the art and are available from various commercial sources. In some embodiments, the anti-TfR antibody or the TfR-binding fragment of an anti-TfR antibody binds to an apical domain of the TfR. In some embodiments, binding of the anti-TfR antibody or the TfR-binding fragment of an anti-TfR antibody to the TfR does not inhibit binding of transferrin to the TfR. Exemplary anti-TfR antibodies include, but are not limited to, B3/25, RBC4, 7579, E2.3. A27.15, D65.30, D2C, ch128.1Av, ch128.1/IgG3, ch128.1/IgG1, hul28.1, (Candelaria et al. Front. Immunol. 12 (17 Mar. 2021), 2021), Ri7, 8D3 (Weber et al. Cell Reports 22:149-162, 2018). Exemplary anti-TfR antibodies are also described in U.S. patent publications: US2018282408A1, US2020071413A1, US20210138083A1, US20190092870A1, and US20130028891 (each of which is incorporated herein by reference).

Exemplary anti-TfR vNARs are described in WO 2022/103769.

Brain shuttles containing anti-TfR or other anti-BBB protein binding domains are described in WO 2014/033074 and WO 2015/101588 (each of which is incorporated herein by reference).

Antibody antigen binding domains that specifically bind BBB proteins can be identified using methods available in the art for generating and/or identifying antibodies that specifically bind to a target protein. Such methods include, but are not limited to, immunization, phage display, and ribosome display.

In some embodiments, a TfR-binding region comprises a fibronectin type III domain peptide modified to specifically bind TfR, for example, any of the domains described in WO2021/076546 and WO2022/221505 (each of which is incorporated herein by reference).

In some embodiments, a TfR-binding region comprises a bicyclic peptide that specifically binds TfR, for example, any of the bicyclic peptides described in WO2022/101633 (incorporated herein by reference).

A TfR-binding region can be derived from a protein known to bind the TfR, such as, P. vivax reticulocyte-binding protein 2b (PvRBP2b) or a viral protein such as an arenavirus protein (e.g., Machupo, Sabiá, Junin, Guanarito or Chapare virus) known to bind TfR.

In some embodiments, the TfR-binding region comprises an engineered polypeptide. An engineered polypeptide can be a polypeptide (e.g., an antibody Fc polypeptide) or antigen-binding region of an anti-TfR antibody modified to alter affinity of the polypeptide or the antigen-binding region of the anti-TfR antibody to TfR. Engineered peptides can be identified or generated using methods available in the art for identifying or generating a peptide having affinity to a known target (e.g., TfR). Such methods include, but are not limited to, phage display, yeast display (e.g., yest surface display), and directed evolution, and combinations thereof.

In some embodiments, a TfR-binding region binds human TfR with an affinity of about 15 nM to about 6000 nM (e.g., about 15 nM, about 50 nM, about 100 nM, about 200 nM, about 300 nM, about 400 nM, about 500 nM, about 600 nM, about 700 nM, about 800 nM, about 900 nM, about 1000 nM, about 1500 nM, about 2000 nM, about 2500 nM, about 3000 nM, about 3500 nM, about 4000 nM, about 4500 nM, about 5000 nM, about 5500 nM, or about 6000 nM). In some embodiments, a TfR-binding region binds human TfR with an affinity of about 100 nM to about 1000 nM or about 100 nM to about 600 nM. In some embodiments, a TfR-binding region binds human TfR with an affinity of about 600 nM to about 6000 nM or about 600 to about 1000 nM. In some embodiments, a TfR-binding region binds human TfR with an affinity of about 200 nM to about 1000 nM, about 300 nM to about 1000 nM, about 400 nM to about 1000 nM, or about 500 to about 1000 nM. In some embodiments, a TfR-binding region binds human TfR with an affinity of about 100 nM to about 1000 nM (e.g., about 100 nM, about 150 nM, about 200 nM, about 250 nM, about 300 nM, about 350 nM, about 400 nM, about 450 nM, about 500 nM, about 550 nM, about 600 nM, about 650 nM, about 700 nM, about 750 nM, about 800 nM, about 850 nM, about 900 nM, about 950 nM, or about 1000 nM). In some embodiments, a TfR-binding region binds human TfR with an affinity of about 600 nM to about 1000 nM (e.g., about 600 nM, about 650 nM, about 700 nM, about 750 nM, about 800 nM, about 850 nM, about 900 nM, about 950 nM, or about 1000 nM). In some embodiments, the TfR-binding region has cynomolgus monkey (cyno) cross-reactivity. In some embodiments, the TfR-binding region binds to cyno TfR with an affinity of 80 nM to 5 μM (e.g., 80 nM, 100 nM. 200 nM, 300 nM, 400 nM, 500 nM, 600 nM, 700 nM, 800 nM, 900 nM, 1 μM. 1.5 μM, 2 μM. 2.5 μM, 3 μM. 3.5 μM, 4 μM. 4.5 μM, or 5 μM).

### B. CD98hc Binding Regions:

A CD98hc-binding region (means for binding CD98hc) is a molecule such as a polypeptide or a region or domain of a larger polypeptide or protein that specifically binds to CD98hc, such as a human TfR.

In some embodiments, a CD98hc-binding region comprises a modified antibody heavy chain constant domain or Fc polypeptide (e.g., modified CH3 peptide). In some embodiments, the CD98hc-binding Fc-Fab fusion comprises a modified CH3 peptide.

In some embodiments, a CD98hc-binding region comprising a modified antibody heavy chain constant domain or Fc polypeptide (e.g., modified CH3 peptide) comprises five, six, seven, eight, or nine substitutions in a set of amino acid positions consisting of 382, 384, 385, 387, 422, 424, 426, 438, and 440; and wherein the positions are determined with reference to EU numbering. In some embodiments, the substitutions are selected from the group consisting of: a R, Y, F, S, W, Y, K, or N at position 382; a L, Y, A, S, or F at position 384; a F, K, D, M, I, N, Y, L, or H at position 385; a N, L, Y, R, F, G, S, D, or T at position 387; an I, K, L, R, T, F, or H at position 422; a V, W, G, L, I, P, or Y at position 424; a D, A, Q, W, L, or P at position 426; an I, V, F, N, P, or S at position 438; and a K, T, P, I, or F at position 440.

In some embodiments, a CD98hc-binding region comprising a modified antibody heavy chain constant domain or Fc polypeptide (e.g., modified CH3 peptide) comprises five, six, seven, eight, or nine substitutions in a set of amino acid positions consisting of 382, 383, 384, 385, 386, 387, 389, 421, 422, 424, 426, 428, 434, 436, 438, 440, and 442; and wherein the positions are determined with reference to EU numbering. In some embodiments, the substitutions are selected from: a S or V at position 378; a D, M, N, P, F, or H at position 380; a R, Y, F, S, W, Y, K, or N at position 382; a T at position 383; a L, Y, A, S, or F at position 384; a F, K, D, M, I, N, Y, L, or H at position 385; a T, P, E, K, A, V, D, T, or F at position 386; a N, L, Y, R, F, G, S, D, or T at position 387; a T, Y, or F at position 389; a D, E, or Q at position 421; an I, K, L, R, T, F, or H at position 422; a V, W, G, L, I, P, or Y at position 424; a D, A, Q, W, L, or P at position 426; a L or Y at position 428; a S at position 434; a F at position 436; an I, V, F, N, P, or S at position 438; a K, T, P, I, or F at position 440; and Q or M at position 442.

### 1. Group I Modified CH3 Peptide CD98hc Binding Regions

In some embodiments, a CD98hc-binding region comprising a modified antibody heavy chain constant domain (e.g. modified CH3 peptide) or a Fc polypeptide comprises at least eleven, twelve, thirteen, fourteen, or fifteen substitutions in a set of amino acid positions consisting of 378, 380, 382, 383, 384, 385, 386, 387, 389, 391, 421, 422, 424, 426, 428, 434, 436, 438, 440, 441, and 442, wherein the positions are determined according to EU numbering. In some embodiments, the substitutions are selected from: a S, V, D, E, or Y at position 378: a L, I, M, A, Q, V, or K at position 380; a N, S, L, M, P, Y, K, A, or T at position 382; a T, F, N, P, D, L, H, or Q at position 383; a K, R, H, I, L, F, Y, V, or Q at position 384; a F or Y at position 385; a V, L, A, I, F, Y, S, T, H, R, or E at position 386; a L or I at position 387; a D, Q, A, T, H, or V at position 389; a T, V, or A at position 391; an E, Q, or A at position 421; a L, M, I, T, or P at position 422; an A at position 424; a N at position 426; a L, T, P, Y F, I, A, K, H, or W at position 428; a S at position 434; a L, V, H, F, P, R or W at position 436; a F or W at position 438; a L, P, E, N, V, A, I, or D at position 440; a P at position 441; and an A, V, M, Q, F, P, L, Y, K, R, H, or M at position 442.

In some embodiments, a CD98hc-binding region comprising a modified antibody heavy chain constant domain (e.g., modified CH3 peptide) or a Fc polypeptide comprises at least thirteen substitutions in a set of amino acid positions consisting of 378, 380, 382, 383, 384, 385, 386, 387, 389, 391, 421, 422, 424, 426, 428, 434, 436, 438, 440, 441, and 442, wherein the positions are determined according to EU numbering. In some embodiments, the substitutions are selected from: a S, V, D, E, or Y at position 378; a L, I, M, A, Q, V, or K at position 380; a N, S, L, M, P, Y, K, A, or T at position 382; a T, F, N, P, D, L, H, or Q at position 383; a K, R, H, I, L, F, Y, V, or Q at position 384; a F or Y at position 385; a V, L, A, I, F, Y, S, T, H, R, or E at position 386; a L or I at position 387; a D, Q, A, T, H, or V at position 389; a T, V, or A at position 391; an E, Q, or A at position 421; a L, M, I, T, or P at position 422; an A at position 424; a N at position 426; a L, T, P, Y F, I, A, K, H, or W at position 428; a S at position 434; a L, V, H, F, P, R or W at position 436; a F or W at position 438; a L, P, E, N, V, A, I, or D at position 440; a P at position 441; and an A, V, M, Q, F, P, L, Y, K, R, H, or M at position 442.

In some embodiments, a CD98hc-binding region comprising a modified antibody heavy chain constant domain (e.g., modified CH3 peptide) or a Fc polypeptide comprises a sequence having at least 85%, at least 90%, or at least 95% sequence identity to the sequence of any one of SEQ ID NOs:15-30, wherein the modified constant domain comprises at least eleven, twelve, thirteen, fourteen, or fifteen substitutions in a set of amino acid positions consisting of: a S, V, D, E, or Y at position 378; a L, I, M, A, Q, V, or K at position 380; a N, S, L, M, P, Y, K, A, or T at position 382; a T, F, N, P, D, L, H, or Q at position 383; a K, R, H, I, L, F, Y, V, or Q at position 384; a F or Y at position 385; a V, L, A, I, F, Y, S, T, H, R, or E at position 386; a L or I at position 387; a D, Q, A, T, H, or V at position 389; a T, V, or A at position 391; an E, Q, or A at position 421; a L, M, I, T, or P at position 422; an A at position 424; a N at position 426; a L, T, P, Y F, I, A, K, H, or W at position 428; a S at position 434; a L, V, H, F, P, R or W at position 436; a F or W at position 438; a L, P, E, N, V, A, I, or D at position 440; a P at position 441; and an A, V, M, Q, F, P, L, Y, K, R, H, or M at position 442, wherein the positions are determined according to EU numbering.

In some embodiments, a CD98hc-binding region comprising a modified antibody heavy chain constant domain (e.g., modified CH3 peptide) or a Fc polypeptide comprises a sequence having at least 85%, at least 90%, or at least 95% sequence identity to the sequence of any one of SEQ ID NOs:15-30, wherein the modified constant domain comprises at least thirteen substitutions in a set of amino acid positions consisting of: a S, V, D, E, or Y at position 378; a L, I, M, A, Q, V, or K at position 380; a N, S, L, M, P, Y, K, A, or T at position 382; a T, F, N, P, D, L, H, or Q at position 383; a K, R, H, I, L, F, Y, V, or Q at position 384; a F or Y at position 385; a V, L, A, I, F, Y, S, T, H, R, or E at position 386; a L or I at position 387; a D, Q, A, T, H, or V at position 389; a T, V, or A at position 391; an E, Q, or A at position 421; a L, M, I, T, or P at position 422; an A at position 424; a N at position 426; a L, T, P, Y F, I, A, K, H, or W at position 428; a S at position 434; a L, V, H, F, P, R or W at position 436; a F or W at position 438; a L, P, E, N, V, A, I, or D at position 440; a P at position 441; and an A, V, M, Q, F, P, L, Y, K, R, H, or M at position 442, wherein the positions are determined according to EU numbering.

In some embodiments, a CD98hc-binding region comprising a modified antibody heavy chain constant domain (e.g., modified CH3 peptide) or a Fc polypeptide comprises a sequence having at least 85%, at least 90%, or at least 95% sequence identity to amino acids 111-217 of the sequence of any one of SEQ ID NOs:15-30, wherein the modified constant domain comprises at least eleven, twelve, thirteen, fourteen, or fifteen substitutions in a set of amino acid positions consisting of: a S, V, D, E, or Y at position 378; a L, I, M, A, Q, V, or K at position 380; a N, S, L, M, P, Y, K, A, or T at position 382; a T, F, N, P, D, L, H, or Q at position 383; a K, R, H, I, L, F, Y, V, or Q at position 384; a F or Y at position 385; a V, L, A, I, F, Y, S, T, H, R, or E at position 386; a L or I at position 387; a D, Q, A, T, H, or V at position 389; a T, V, or A at position 391; an E, Q, or A at position 421; a L, M, I, T, or P at position 422; an A at position 424; a N at position 426; a L, T, P, Y F, I, A, K, H, or W at position 428; a S at position 434; a L, V, H, F, P, R or W at position 436; a F or W at position 438; a L, P, E, N, V, A, I, or D at position 440; a P at position 441; and an A, V, M, Q, F, P, L, Y, K, R, H, or M at position 442, wherein the positions are determined according to EU numbering.

In some embodiments, a CD98hc-binding region comprising a modified antibody heavy chain constant domain (e.g., modified CH3 peptide) or a Fc polypeptide comprises a sequence having at least 85%, at least 90%, or at least 95% sequence identity to amino acids 111-217 of the sequence of any one of SEQ ID NOs:15-30, wherein the modified constant domain comprises at least thirteen substitutions in a set of amino acid positions consisting of: a S, V, D, E, or Y at position 378; a L, I, M, A, Q, V, or K at position 380; a N, S, L, M, P, Y, K, A, or T at position 382; a T, F, N, P, D, L, H, or Q at position 383; a K, R, H, I, L, F, Y, V, or Q at position 384; a F or Y at position 385; a V, L, A, I, F, Y, S, T, H, R, or E at position 386; a L or I at position 387; a D, Q, A, T, H, or V at position 389; a T, V, or A at position 391; an E, Q, or A at position 421; a L, M, I, T, or P at position 422; an A at position 424; a N at position 426; a L, T, P, Y F, I, A, K, H, or W at position 428; a S at position 434; a L, V, H, F, P, R or W at position 436; a F or W at position 438; a L, P, E, N, V, A, I, or D at position 440; a P at position 441; and an A, V, M, Q, F, P, L, Y, K, R, H, or M at position 442, wherein the positions are determined according to EU numbering.

In some embodiments, a CD98hc-binding region comprising a modified antibody heavy chain constant domain (e.g., modified CH3 peptide) or a Fc polypeptide comprises amino acids 111-217 of any one of SEQ ID NOs:15-30.

In some embodiments, a CD98hc-binding region comprising a modified antibody heavy chain constant domain (e.g., modified CH3 peptide) or a Fc polypeptide comprises of any one of SEQ ID NOs:15-30.

TABLE 16
Fc peptides having modified CH3 domains that specifically bind CD98hc.

| SEQ | | |
|---|---|---|
| ID | | |
| NO | Sequence | Clone |
| 15 | APELLGGPSVFLFPPKPKDTLMISRTPEVTCVVVDVSHEDPEVKFNWYVDGVEVH | Clone ILB2- |
| | NAKTKPREEQYNSTYRVVSVLTVLHQDWINGKEYKCKVSNKALPAPIEKTISKA | 10-6 |
| | KGQPREPQVYTLPPSRDELTKNQVSLTCLVKGFYPSDIAVLWNSRFVLENNYKTT | |
| | PPVLDSDGSFFLYSKLTVDKSRWQQGNIFACNVYHEALHNHYTFKNLALSPGK | |
| 16 | APELLGGPSVFLFPPKPKDTLMISRTPEVTCVVVDVSHEDPEVKFNWYVDGVEVH | Clone LIB2- |
| | NAKTKPREEQYNSTYRVVSVLTVLHQDWINGKEYKCKVSNKALPAPIEKTISKA | 10-8 |
| | KGQPREPQVYTLPPSRDELTKNQVSLTCLVKGFYPSDIAVLWNSRFVLENNYKTT | |
| | PPVLDSDGSFFLYSKLTVDKSRWQQGEIFACNVYHEALHNHYTFKNLALSPGK | |
| 17 | APELLGGPSVFLFPPKPKDTLMISRTPEVTCVVVDVSHEDPEVKFNWYVDGVEVH | 10-8-d18 |
| | NAKTKPREEQYNSTYRVVSVLTVLHQDWLNGKEYKCKVSNKALPAPIEKTISKA | Clone LLB2- |
| | KGQPREPQVYTLPPSRDELTKNQVSLTCLVKGFYPSDIAVLWNSQYELENNYKTT | |
| | PPVLDSDGSFFLYSKLTVDKSRWQQGNVFACNVYHEALHNHYTFKNLALSPGK | |
| 18 | APELLGGPSVFLFPPKPKDTLMISRTPEVTCVVVDVSHEDPEVKFNWYVDGVEVH | Clone LLB2- |
| | NAKTKPREEQYNSTYRVVSVLTVLHQDWLNGKEYKCKVSNKALPAPIEKTISKA | 10-8-d12 |
| | KGQPREPQVYTLPPSRDELTKNQVSLTCLVKGFYPSDIAVLWNSHYELENNYKTT | |
| | PPVIDSDGSFFLYSKLTVDKSRWQQGNVFACNVYHEALHNHYTFKNLALSPGK | |
| 19 | APELLGGPSVFLFPPKPKDTLMISRTPEVTCVVVDVSHEDPEVKFNWYVDGVEVH | Clone LLB2- |
| | NAKTKPREEQYNSTYRVVSVLTVLHQDWLNGKEYKCKVSNKALPAPIEKTISKA | 10-8-d6 |
| | KGQPREPQVYTLPPSRDELTKNQVSLTCLVKGFYPSDIAVLWNSRFVLENNYKTT | |
| | PPVLDSDGSFFLYSKLTVDKSRWQQGNVFACNVYHEALHNHYTFKNLALSPGK | |
| 20 | APELLGGPSVFLFPPKPKDTLMISRTPEVTCVVVDVSHEDPEVKFNWYVDGVEVH | 10-8-d3 |
| | NAKTKPREEQYNSTYRVVSVLTVLHQDWLNGKEYKCKVSNKALPAPIEKTISKA | Clone LLB2- |
| | KGQPREPQVYTLPPSRDELTKNQVSLTCLVKGFYPSDIAVLWNSRFVLENNYKTT | |
| | PPVLDSDGSFFLYSKLTVDKSRWQQGEVFACNVYHEALHNHYTFKNLALSPGK | |
| 21 | APELLGGPSVFLFPPKPKDTLMISRTPEVTCVVVDVSHEDPEVKFNWYVDGVEVH | Clone LLB2- |
| | NAKTKPREEQYNSTYRVVSVLTVLHQDWLNGKEYKCKVSNKALPAPIEKTISKA | 10-8-d1 |
| | KGQPREPQVYTLPPSRDELTKNQVSLTCLVKGFYPSDIAVLWNSRFVLENNYKTT | |
| | PPVLDSDGSFFLYSKLTVDKSRWQQGEIFACNVYHEALHNHYTFKNLSLSPGK | |
| 22 | APELLGGPSVFLFPPKPKDTLMISRTPEVTCVVVDVSHEDPEVKFNWYVDGVEVH | Clone |
| | NAKTKPREEQYNSTYRVVSVLTVLHQDWLNGKEYKCKVSNKALPAPIEKTISKA | LLB2.10.8.10.3 |
| | KGQPREPQVYTLPPSRDELTKNQVSLTCLVKGFYPSDIAVLWNSRFVLENNYKTT | |
| | PPVLDSDGSFFLYSKLTVDKSRWQQGEIFACNVYHEALHNHYTFKNLRLSPGK | |
| 23 | APELLGGPSVFLFPPKPKDTLMISRTPEVTCVVVDVSHEDPEVKFNWYVDGVEVH | Clone |
| | NAKTKPREEQYNSTYRVVSVLTVLHQDWLNGKEYKCKVSNKALPAPIEKTISKA | LLB2.10.8.10.3 |
| | KGQPREPQVYTLPPSRDELTKNQVSLTCLVKGFYPSDIAVLWNSRFVLENNYKTT | |
| | PPVLDSDGSFFLYSKLTVDKSRWQQGEIFACNVYHEALHNHYTFKNLHLSPGK | |
| 24 | APELLGGPSVFLFPPKPKDTLMISRTPEVTCVVVDVSHEDPEVKFNWYVDGVEVH | Clone |
| | NAKTKPREEQYNSTYRVVSVLTVLHQDWLNGKEYKCKVSNKALPAPIEKTISKA | LLB2.10.8.14.3 |
| | KGQPREPQVYTLPPSRDELTKNQVSLTCLVKGFYPSDIAVLWNSRFVLENNYKTT | |
| | PPVLDSDGSFFLYSKLTVDKSRWQQGEIFACNVYHEALHNHRTFKNLRLSPGK | |
| 25 | APELLGGPSVFLFPPKPKDTLMISRTPEVTCVVVDVSHEDPEVKFNWYVDGVEVH | Clone |
| | NAKTKPREEQYNSTYRVVSVLTVLHQDWLNGKEYKCKVSNKALPAPIEKTISKA | LLB2.10.8.12.5 |
| | KGQPREPQVYTLPPSRDELTKNQVSLTCLVKGFYPSDIAVLWNSHYELENNYKTT | |
| | PPVLDSDGSFFLYSKLTVDKSRWQQGEIFACNVYHEALHNHYTFKNLALSPGK | |
| 26 | APELLGGPSVFLFPPKPKDTLMISRTPEVTCVVVDVSHEDPEVKFNWYVDGVEVH | Clone LLB2-37 |
| | NAKTKPREEQYNSTYRVVSVLTVLHQDWLNGKEYKCKVSNKALPAPIEKTISKA | |
| | KGQPREPQVYTLPPSRDELTKNQVSLTCLVKGFYPSDIAVLWNSQFHLENNYKTT | |
| | PPVLDSDGSFFLYSKLTVDKSRWQQGNIFACNVYHEALHNHYTFKNLLLSPGK | |
| 27 | APELLGGPSVFLFPPKPKDTLMISRTPEVTCVVVDVSHEDPEVKFNWYVDGVEVH | Clone |
| | NAKTKPREEQYNSTYRVVSVLTVLHQDWLNGKEYKCKVSNKALPAPIEKTISKA | LLB2.10.8.9.11.N |
| | KGQPREPQVYTLPPSRDELTKNQVSLTCLVKGFYPSDIAVLWNSRFVLENNYKTT | |
| | PPVLDSDGSFFLYSKLTVDKSRWQQGNTFACNVYHEALHNHYTFKNLALSPGK | |
| 28 | APELLGGPSVFLFPPKPKDTLMISRTPEVTCVVVDVSHEDPEVKFNWYVDGVEVH | Clone |
| | NAKTKPREEQYNSTYRVVSVLTVLHQDWLNGKEYKCKVSNKALPAPIEKTISKA | LLB2.10.8.10.1 |
| | KGQPREPQVYTLPPSRDELTKNQVSLTCLVKGFYPSDIAVLWNSRFVLENNYKTT | |
| | PPVLDSDGSFFLYSKLTVDKSRWQQGEIFACNVYHEALHNHYTFKNLKLSPGK | |
| 29 | APELLGGPSVFLFPPKPKDTLMISRTPEVTCVVVDVSHEDPEVKFNWYVDGVEVH | Clone |
| | NAKTKPREEQYNSTYRVVSVLTVLHQDWLNGKEYKCKVSNKALPAPIEKTISKA | LLB2.10.8.4.12 |
| | KGQPREPQVYTLPPSRDELTKNQVSLTCLVKGFYPSDIAVLWNSRFVLENNYKTT | |
| | PPVLDSDGSFFLYSKLTVDKSRWQQGEIFACNVYHEALHNHWTFKNLRLSPGK | |
| 30 | APELLGGPSVFLFPPKPKDTLMISRTPEVTCVVVDVSHEDPEVKFNWYVDGVEVH | LLB2.10.8.2.1 |
| | NAKTKPREEQYNSTYRVVSVLTVLHQDWLNGKEYKCKVSNKALPAPIEKTISKA | Clone |
| | KGQPREPQVYTLPPSRDELTKNQVSLTCLVKGFYPSDIAVLWNSQYLLENNYKTT | |
| | PPVLDSDGSFFLYSKLTVDKSRWQQGEIFACNVYHEALHNHYTFKNLALSPGK | |
| 135 | APELLGGPSVFLFPPKPKDTLMISRTPEVTCVVVDVSHEDPEVKFNWYVDGVEVH | Clone |
| | NAKTKPREEQYNSTYRVVSVLTVLHQDWLNGKEYKCKVSNKALPAPIEKTISKA | LLB2.10.9d28 |
| | KGQPREPQVYTLPPSRDELTKNQVSLTCLVKGFYPSDIAVLWSSRYVLENNYKTT | |
| | PPVLDSDGSFFLYSKLTVDKSRWQQGNVFACNVYHEALHNHYTFKNLALSPGK | |

In some embodiments, a CD98hc-binding region comprising a modified antibody heavy chain constant domain or Fc polypeptide (e.g., modified CH3 peptide) comprises eleven, twelve, thirteen, fourteen, or fifteen substitutions in a set of amino acid positions consisting of 380, 382, 384, 385, 386, 387, 421, 422, 424, 426, 428, 436, 438, 440, and 442, wherein the positions are determined according to EU numbering. In some embodiments, the substitutions are selected from: a L at position 380; a N at position 382; a R, H, or Q at position 384; a F or Y at position 385; a V, L, I, F, Y, or E at position 386; a L at position 387; an E, Q or A at position 421; an I, T, or P at position 422; an A at position 424; a N at position 426; a Y or W at position 428; a R or W at position 436; a F or W at position 438; a N at position 440; and an A, Q, K, R, H, or M at position 442.

In some embodiments, a CD98hc-binding region comprising a modified antibody heavy chain constant domain or Fc polypeptide (e.g., modified CH3 peptide) comprises at least thirteen or at least fourteen substitutions in a set of amino acid positions consisting of 380, 382, 384, 385, 386, 387, 421, 422, 424, 426, 428, 436, 438, 440, and 442, wherein the positions are determined according to EU numbering. In some embodiments, the substitutions are selected from: a L at position 380; a N at position 382; a R, H, or Q at position 384; a F or Y at position 385; a V, L, I, F, Y, or E at position 386; a L at position 387; an E, Q or A at position 421: an I, T, or P at position 422; an A at position 424; a N at position 426; a Y or W at position 428; a R or W at position 436; a F or W at position 438; a N at position 440; and an A, Q, K, R, H, or M at position 442.

In some embodiments, a CD98hc-binding region comprising a modified antibody heavy chain constant domain (e.g., modified CH3 peptide) or a Fc polypeptide comprises a sequence having at least 85%, at least 90%, or at least 95% sequence identity to the sequence of any one of SEQ ID NOs:15-30, wherein the modified constant domain comprises eleven, twelve, thirteen, fourteen, or fifteen substitutions in a set of amino acid positions consisting of a L at position 380; a N at position 382; a R, H, or Q at position 384; a F or Y at position 385; a V, L, I, F, Y, or E at position 386; a L at position 387; an E, Q or A at position 421; an I, T, or P at position 422; an A at position 424; a N at position 426; a Y or W at position 428; a R or W at position 436; a F or W at position 438; a N at position 440; and an A, Q, K, R, H, or M at position 442.

In some embodiments, a CD98hc-binding region comprising a modified antibody heavy chain constant domain (e.g., modified CH3 peptide) or a Fc polypeptide comprises a sequence having at least 85%, at least 90%, or at least 95% sequence identity to the sequence of any one of SEQ ID NOs:15-30, wherein the modified constant domain comprises at least thirteen or at least fourteen substitutions in a set of amino acid positions consisting of: a L at position 380; a N at position 382; a R, H, or Q at position 384; a F or Y at position 385; a V, L, I, F, Y, or E at position 386; a L at position 387; an E, Q or A at position 421; an I, T, or P at position 422; an A at position 424; a N at position 426; a Y or W at position 428; a R or W at position 436; a F or W at position 438; a N at position 440; and an A, Q, K, R, H, or M at position 442.

In some embodiments, the polypeptide comprises a modified constant domain (e.g., a modified CH3 peptide) that comprises a sequence having at least 85%, at least 90%, or at least 95% sequence identity to amino acids 111-217 of the sequence of SEQ ID NOs:15-30, wherein the modified constant domain comprises eleven, twelve, thirteen, fourteen, or fifteen substitutions in a set of amino acid positions consisting of a L at position 380; a N at position 382; a R, H, or Q at position 384; a F or Y at position 385; a V, L, I, F, Y, or E at position 386; a L at position 387; an E, Q or A at position 421; an I, T, or P at position 422; an A at position 424; a N at position 426; a Y or W at position 428; a R or W at position 436; a F or W at position 438; a N at position 440; and an A, Q, K, R, H, or M at position 442.

In some embodiments, the polypeptide comprises a modified constant domain (e.g., a modified CH3 peptide) that comprises a sequence having at least 85%, at least 90%, or at least 95% sequence identity to amino acids 111-217 of the sequence of SEQ ID NOs:15-30, wherein the modified constant domain comprises at least thirteen or at least fourteen substitutions in a set of amino acid positions consisting of: a L at position 380; a N at position 382; a R, H, or Q at position 384; a F or Y at position 385; a V, L, I, F, Y, or E at position 386; a L at position 387; an E, Q or A at position 421; an I, T, or P at position 422; an A at position 424; a N at position 426; a Y or W at position 428; a R or W at position 436; a F or W at position 438; a N at position 440; and an A, Q, K, R, H, or M at position 442.

In some embodiments, a CD98hc-binding region comprising a modified CH3 peptide comprises:
(a) a L at position 380; a N at position 382; a R at position 384; a F at position 385; a V at position 386; a L at position 387; an I at position 422; an A at position 424; a N at position 426; a Y at position 428; a F at position 438; a N at position 440; and an A at position 442.
(b) a L at position 380; a N at position 382; a R at position 384; a F at position 385; a V at position 386; a L at position 387; an E at position 421; an I at position 422; an A at position 424; a N at position 426; a Y at position 428; a F at position 438; a N at position 440; and an A at position 442;
(c) a L at position 380; a N at position 382; a Q at position 384; a Y at position 385; an E at position 386; a L at position 387; an A at position 424; a N at position 426; a Y at position 428; a F at position 438; a N at position 440; and an A at position 442;
(d) a L at position 380; a N at position 382; a H at position 384; a Y at position 385; an E at position 386; a L at position 387; an A at position 424; a N at position 426; a Y at position 428; a F at position 438; a N at position 440; and an A at position 442;
(e) a L at position 380; a N at position 382; a R at position 384; a F at position 385; a V at position 386; a L at position 387; an A at position 424; a N at position 426; a Y at position 428; a F at position 438; a N at position 440; and an A at position 442;
(f) a L at position 380; a N at position 382; a R at position 384; a F at position 385; a V at position 386; a L at position 387; an E at position 421; an A at position 424; a N at position 426; a Y at position 428; a F at position 438; a N at position 440; and an A at position 442;
(g) a L at position 380; a N at position 382; a R at position 384; a F at position 385; a V at position 386; a L at position 387; an E at position 421; an I at position 422; an A at position 424; a N at position 426; a Y at position 428; a F at position 438; and a N at position 440;
(h) a L at position 380; a N at position 382; a R at position 384; a F at position 385; a V at position 386; a L at position 387; an I at position 422; an A at position 424; a N at position 426; a Y at position 428; a F at position 438; a N at position 440; and a R at position 442;
(i) a L at position 380; a N at position 382; a R at position 384; a F at position 385; a V at position 386; a L at position 387; an I at position 422; an A at position 424; a N at position 426; a Y at position 428; a F at position 438; a N at position 440; and a H at position 442;
(j) a L at position 380; a N at position 382; a R at position 384; a F at position 385; a V at position 386; a L at position 387; an I at position 422; an A at position 424; a N at position 426; a Y at position 428; a R at position 436; a F at position 438; a N at position 440; and a R at position 442;
(k) a L at position 380; a N at position 382; a H at position 384; a Y at position 385; an E at position 386; a L at position 387; an I at position 422; an A at position 424; a N at position 426; a Y at position 428; a F at position 438; a N at position 440; and an A at position 442;
(l) a L at position 380; a N at position 382; a Q at position 384; a F at position 385; a H at position 386; a L at position 387; an I at position 422; an A at position 424; a N at position 426; a Y at position 428; a F at position 438; a N at position 440; and a L at position 442;
(m) a L at position 380; a N at position 382; a R at position 384; a F at position 385; a V at position 386; a L at position 387; a T at position 422; an A at position 424; a N at position 426; a Y at position 428; a F at position 438; a N at position 440; a and an A at position 442;
(n) a L at position 380; a N at position 382; a R at position 384; a F at position 385; a V at position 386; a L at position 387; an I at position 422; an A at position 424; a N at position 426; a Y at position 428; a F at position 438; a N at position 440; a and a K at position 442;
(o) a L at position 380; a N at position 382; a R at position 384; a F at position 385; a V at position 386; a L at position 387: I at position 422; an A at position 424; a N at position 426; a Y at position 428; a W at position 436; a F at position 438; a N at position 440; a and a R at position 442; or
(p) a L at position 380; a N at position 382; a Q at position 384; a Y at position 385; a L at position 386; a L at position 387; an E at position 421; an I at position 422; an A at position 424; a N at position 426; a Y at position 428; a F at position 438; a N at position 440; and an A at position 442;
wherein the positions are determined according to EU numbering.

In some embodiments, a CD98hc-binding region comprising a modified CH3 peptide comprises the amino acid sequence of: SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27. SEQ ID NO:28, SEQ ID NO:29, SEQ ID NO:30, or SEQ ID NO: 135.

In some embodiments, a CD98hc-binding region comprising a modified CH3 peptide comprises:
(i) a first amino acid sequence of LX¹NX₂X₃X₄X₅L (SEQ ID NO:31), wherein X₁ is any amino acid, X₂ is R, H, or Q, X₃ is F or Y, X₄ is V, L, I, F, Y, or E, X₅ is any amino acid;
(ii) a second amino acid sequence of X₁X₂X₃AX₄X₅X₆X₇ (SEQ ID NO:32), wherein X₁ is E, N, Q, or A, X₂ is I, V, T, or P, X₃ and X₄ are any amino acid, X₅ is N or S. X₆ is any amino acid, X₇ is Y or W; and
(iii) a third amino acid sequence of X₁X₂X₃X₄NX₅X₆ (SEQ ID NO:33), wherein X₁ is Y, R, or W, X₂ is any amino acid, X₃ is F or W. X₄ and X₅ are any amino acid; and X₆ is A, Q, K, R, H, M, or S.

### 2. Group II Modified CH3 Peptide CD98hc Binding Regions

In some embodiments, a CD98hc-binding region comprising a modified antibody heavy chain constant domain (e.g., modified CH3 peptide) or a Fc polypeptide comprises eight, nine, ten, eleven, twelve, thirteen, fourteen, fifteen, sixteen, seventeen, eighteen, or nineteen substitutions in a set of amino acid positions consisting of: 378, 380, 382, 383, 384, 385, 386, 387, 389, 421, 422, 424, 426, 428, 434, 436, 438, 440, and 442, wherein the positions are determined according to EU numbering. In some embodiments, the substitutions are selected from: a S or V at position 378; a D, M, N, P, F, or H at position 380; a R, Y, F, S, W, Y, K, or N at position 382; a T at position 383; a L, Y, A, S, or F at position 384; a F, K, D, M, I, N, Y, L, or H at position 385; a T, P, E, K, A, V, D, T, or F at position 386; a N, L, Y, R, F, G, S, D, or T at position 387; a T, Y, or F at position 389; a D, E, or Q at position 421; a I, K, L, R, T, F, or H at position 422; a V, W, G, L, I, P, or Y at position 424; a D, A, Q, W, L, or P at position 426; a L or Y at position 428; a S at position 434; a F at position 436; a I, V, F, N, P, or S at position 438; a K, T, P, I, or F at position 440; and a Q or M at position 442.

In some embodiments, a CD98hc-binding region comprising a modified antibody heavy chain constant domain (e.g., modified CH3 peptide) or a Fc polypeptide comprises at least eleven, at least twelve, or at least thirteen substitutions in a set of amino acid positions consisting of: 378, 380, 382, 383, 384, 385, 386, 387, 389, 421, 422, 424, 426, 428, 434, 436, 438, 440, and 442, wherein the positions are determined according to EU numbering. In some embodiments, the substitutions are selected from: a S or V at position 378; a D, M, N, P, F, or H at position 380; a R, Y, F, S, W, Y, K, or N at position 382; a T at position 383; a L, Y, A, S, or F at position 384; a F, K, D, M, I, N, Y, L, or H at position 385; a T, P, E, K, A, V, D, T, or F at position 386; a N, L, Y, R, F, G, S, D, or T at position 387; a T, Y, or F at position 389; a D, E, or Q at position 421; a I, K, L, R, T, F, or H at position 422; a V, W, G, L, 1, P, or Y at position 424; a D, A, Q, W, L, or P at position 426; a L or Y at position 428; a S at position 434; a F at position 436; a I, V, F, N, P, or S at position 438; a K, T, P, I, or F at position 440; and a Q or M at position 442.

In some embodiments, a CD98hc-binding region comprising a modified antibody heavy chain constant domain (e.g., modified CH3 peptide) or a Fc polypeptide comprises a sequence having at least 85%, 90%, or 95% sequence identity to the sequence of SEQ ID NO:34 or SEQ ID NO:35, wherein the modified constant domain comprises at least eight, nine, ten, eleven, twelve, thirteen, fourteen, fifteen, sixteen, seventeen, eighteen, or nineteen substitutions in a set of amino acid positions consisting of: a S or V at position 378; a D, M, N, P, F, or H at position 380; a R, Y, F, S, W, Y, K, or N at position 382; a T at position 383; a L, Y, A, S, or F at position 384; a F, K, D, M, I, N, Y, L, or H at position 385; a T, P, E, K, A, V, D, T, or F at position 386; a N, L, Y, R, F, G, S, D, or T at position 387; a T, Y, or F at position 389; a D, E, or Q at position 421; a I, K, L, R, T, F, or H at position 422; a V, W, G, L, I, P, or Y at position 424; a D, A, Q, W, L, or P at position 426; a L or Y at position 428; a S at position 434; a F at position 436; a I, V, F, N, P, or S at position 438; a K, T, P, I, or F at position 440; and a Q or M at position 442.

In some embodiments, a CD98hc-binding region comprising a modified antibody heavy chain constant domain (e.g., modified CH3 peptide) or a Fc polypeptide comprises a sequence having at least 85%, 90%, or 95% sequence identity to the sequence of SEQ ID NO:34 or SEQ ID NO:35, wherein the modified constant domain comprises at least eleven, at least twelve, or at least thirteen substitutions in a set of amino acid positions consisting of: a S or V at position 378; a D, M, N, P, F, or H at position 380; a R, Y, F, S, W, Y, K, or N at position 382; a T at position 383; a L, Y, A, S, or F at position 384; a F, K, D, M, I, N, Y, L, or H at position 385; a T, P, E, K, A, V, D, T, or F at position 386; a N, L, Y, R, F, G, S, D, or T at position 387; a T, Y, or F at position 389; a D, E, or Q at position 421; a I, K, L, R, T, F, or H at position 422; a V, W, G, L, I, P, or Y at position 424; a D, A, Q, W, L, or P at position 426; a L or Y at position 428; a S at position 434; a F at position 436; a I, V, F, N, P, or S at position 438; a K, T, P, I, or F at position 440; and a Q or M at position 442.

In some embodiments, a CD98hc-binding region comprising a modified antibody heavy chain constant domain (e.g., modified CH3 peptide) or a Fc polypeptide comprises a sequence having at least 85%, at least 90%, or at least 95% sequence identity to amino acids 111-217 of the sequence of SEQ ID NO:34 or SEQ ID NO:35, wherein the modified constant domain comprises at least eight, nine, ten, eleven, twelve, thirteen, fourteen, fifteen, sixteen, seventeen, eighteen, or nineteen substitutions in a set of amino acid positions consisting of: a S or V at position 378; a D, M, N, P, F, or H at position 380; a R, Y, F, S, W, Y, K, or N at position 382; a T at position 383; a L, Y, A, S, or F at position 384; a F, K, D, M, I, N, Y, L, or H at position 385; a T, P, E, K, A, V, D, T, or F at position 386; a N, L, Y, R, F, G, S, D, or T at position 387; a T, Y, or F at position 389; a D, E, or Q at position 421; a I, K, L, R, T, F, or H at position 422; a V, W, G, L, I, P, or Y at position 424; a D, A, Q, W, L, or P at position 426; a L or Y at position 428; a S at position 434; a F at position 436; a I, V, F, N, P, or S at position 438; a K, T, P, I, or F at position 440; and a Q or M at position 442.

In some embodiments, a CD98hc-binding region comprising a modified antibody heavy chain constant domain (e.g., modified CH3 peptide) or a Fc polypeptide comprises a sequence having at least 85%, at least 90%, or at least 95% sequence identity to amino acids 111-217 of the sequence of SEQ ID NO:34 or SEQ ID NO:35, wherein the modified constant domain comprises at least eleven, at least twelve, or at least thirteen substitutions in a set of amino acid positions consisting of: a S or V at position 378; a D, M, N, P, F, or H at position 380; a R, Y, F, S, W, Y, K, or N at position 382; a T at position 383; a L, Y, A, S, or F at position 384; a F, K, D, M, I, N, Y, L, or H at position 385; a T, P, E, K, A, V, D, T, or F at position 386; a N, L, Y, R, F, G, S, D, or T at position 387; a T, Y, or F at position 389; a D, E, or Q at position 421; a I, K, L, R, T, F, or H at position 422; a V, W, G, L, I, P, or Y at position 424; a D, A, Q, W, L, or P at position 426; a L or Y at position 428; a S at position 434; a F at position 436; a I, V, F, N, P, or S at position 438; a K, T, P, I, or F at position 440; and a Q or M at position 442.

In some embodiments, a CD98hc-binding region comprising a modified antibody heavy chain constant domain (e.g., modified CH3 peptide) or a Fc polypeptide comprises amino acids 111-217 of any one of SEQ ID NO:34 or SEQ ID NO:35.

In some embodiments, a CD98hc-binding region comprising a modified antibody heavy chain constant domain (e.g., modified CH3 peptide) or a Fc polypeptide comprises of any one of SEQ ID NO:34 or SEQ ID NO:35.

In some embodiments, a CD98hc-binding region comprising a modified antibody heavy chain constant domain (e.g., modified CH3 peptide) or a Fc polypeptide comprises eight, nine, ten, eleven, twelve, or thirteen substitutions in a set of amino acid positions consisting of 380, 382, 384, 385, 386, 387, 422, 424, 426, 428, 434, 438, and 440, wherein the positions are determined according to EU numbering. In some embodiments, the substitutions are selected from: a D, M, N, P, F, or H at position 380; a R, Y, F, S, W, Y, K, or N at position 382; a L, Y, A, S, or F at position 384; a F, K, D, M, I, N, Y, L, or H at position 385; a T, P, E, K, A, V, D, T, or F at position 386; a N, L, Y, R, G, S, D, or T at position 387; an I, K, R, T, F, or H at position 422; a V, W, G, L, I, P, or Y at position 424; a D, A, Q, W, L, or P at position 426; a L at position 428; a S at position 434; an I, F, N, P, or S at position 438; and a K, T, I, or F at position 440.

In some embodiments, a CD98hc-binding region comprising a modified antibody heavy chain constant domain (e.g., modified CH3 peptide) or a Fc polypeptide comprises eleven, twelve, or thirteen substitutions in a set of amino acid positions consisting of 380, 382, 384, 385, 386, 387, 422, 424, 426, 428, 434, 438, and 440, wherein the positions are determined according to EU numbering. In some embodiments, the substitutions are selected from: a D, M, N, P, F, or H at position 380; a R, Y, F, S, W, Y, K, or N at position 382; a L, Y, A, S, or F at position 384; a F, K, D, M, I, N, Y, L, or H at position 385; a T, P, E, K, A, V, D, T, or F at position 386; a N, L, Y, R, G, S, D, or T at position 387; an I, K, R, T, F, or H at position 422; a V, W, G, L, I, P, or Y at position 424; a D, A, Q, W, L, or P at position 426; a L at position 428; a S at position 434; an I, F, N, P, or S at position 438; and a K, T, I, or F at position 440.

In some embodiments, a CD98hc-binding region comprising a modified antibody heavy chain constant domain (e.g., modified CH3 peptide) or a Fc polypeptide comprises a sequence having at least 85%, at least 90%, or at least 95% sequence identity to the sequence of SEQ ID NO:34 or SEQ ID NO:35, wherein the modified constant domain comprises eight, nine, ten, eleven, twelve, or thirteen substitutions in a set of amino acid positions consisting of a D, M, N, P, F, or H at position 380; a R, Y, F, S, W, Y, K, or N at position 382; a L, Y, A, S, or F at position 384; a F, K, D, M, I, N, Y, L, or H at position 385; a T, P, E, K, A, V, D, T, or F at position 386; a N, L, Y, R, G, S, D, or T at position 387; an I, K, R, T, F, or H at position 422; a V, W, G, L, I, P, or Y at position 424; a D, A, Q, W, L, or P at position 426; a L at position 428; a S at position 434; an I, F, N, P, or S at position 438; and a K, T, I, or F at position 440.

In some embodiments, a CD98hc-binding region comprising a modified antibody heavy chain constant domain (e.g., modified CH3 peptide) or a Fc polypeptide comprises a sequence having at least 85%, at least 90%, or at least 95% sequence identity to the sequence of SEQ ID NO:34 or SEQ ID NO:35, wherein the modified constant domain comprises eleven, twelve, or thirteen substitutions in a set of amino acid positions consisting of: a D, M, N, P, F, or H at position 380; a R, Y, F, S, W, Y, K, or N at position 382; a L, Y, A, S, or F at position 384; a F, K, D, M, I, N, Y, L, or H at position 385; a T, P, E, K, A, V, D, T, or F at position 386; a N, L, Y, R, G, S, D, or T at position 387; an I, K, R, T, F, or H at position 422; a V, W, G, L, I, P, or Y at position 424; a D, A, Q, W, L, or P at position 426; a L at position 428; a S at position 434; an I, F, N, P, or S at position 438; and a K, T, I, or F at position 440.

In some embodiments, a CD98hc-binding region comprising a modified antibody heavy chain constant domain (e.g., modified CH3 peptide) or a Fc polypeptide comprises a sequence having at least 85%, at least 90%, or at least 95% sequence identity to amino acids 111-217 of the sequence of any one of SEQ ID NO:34 or SEQ ID NO:35, wherein the modified constant domain comprises eight, nine, ten, eleven, twelve, or thirteen substitutions in a set of amino acid positions consisting of: a D, M, N, P, F, or H at position 380; a R, Y, F, S, W, Y, K, or N at position 382; a L, Y, A, S, or F at position 384; a F, K, D, M, I, N, Y, L, or H at position 385; a T, P, E, K, A, V, D, T, or F at position 386; a N, L, Y, R, G, S, D, or T at position 387; an I, K, R, T, F, or H at position 422; a V, W, G, L, I, P, or Y at position 424; a D, A, Q, W, L, or P at position 426; a L at position 428; a S at position 434; an I, F, N, P, or S at position 438; and a K, T, I, or F at position 440.

In some embodiments, a CD98hc-binding region comprising a modified antibody heavy chain constant domain (e.g., modified CH3 peptide) or a Fc polypeptide comprises a sequence having at least 85%, at least 90%, or at least 95% sequence identity to amino acids 111-217 of the sequence of any one of SEQ ID NO:34 or SEQ ID NO:35, wherein the modified constant domain comprises eleven, twelve, or thirteen substitutions in a set of amino acid positions consisting of: a D, M, N, P, F, or H at position 380; a R, Y, F, S, W, Y, K, or N at position 382; a L, Y, A, S, or F at position 384; a F, K, D, M, I, N, Y, L, or H at position 385; a T, P, E, K, A, V, D, T, or F at position 386; a N, L, Y, R, G, S, D, or T at position 387; an I, K, R, T, F, or H at position 422; a V, W, G, L, I, P, or Y at position 424; a D, A, Q, W, L, or P at position 426; a L at position 428; a S at position 434; an I, F, N, P, or S at position 438; and a K, T, I, or F at position 440.

In some embodiments, a CD98hc-binding region comprising a modified antibody heavy chain constant domain (e.g., modified CH3 peptide) or a Fc polypeptide comprises eight, nine, ten, eleven, twelve, thirteen, fourteen, fifteen, sixteen, seventeen, eighteen, or nineteen substitutions in a set of amino acid positions consisting of 378, 380, 382, 383, 384, 385, 386, 387, 389, 421, 422, 424, 426, 428, 434, 436, 438, 440, and 442, wherein the positions are determined according to EU numbering. In some embodiments, the substitutions are selected from: a S or V at position 378; a D at position 380; a R at position 382; a T at position 383; a Y at position 384; a K at position 385; a P at position 386; a Y at position 387; a T, Y, or F at position 389; a D, E, or Q at position 421; an I at position 422; a V at position 424; a D at position 426; a L or Y at position 428; a S at position 434; a F at position 436; an I or V at position 438; a K at position 440; and a Q or M at position 442.

In some embodiments, a CD98hc-binding region comprising a modified antibody heavy chain constant domain (e.g., modified CH3 peptide) or a Fc polypeptide comprises at least eleven, at least twelve, or at least thirteen substitutions in a set of amino acid positions consisting of 378, 380, 382, 383, 384, 385, 386, 387, 389, 421, 422, 424, 426, 428, 434, 436, 438, 440, and 442, wherein the positions are determined according to EU numbering. In some embodiments, the substitutions are selected from: a S or V at position 378; a D at position 380; a R at position 382; a T at position 383; a Y at position 384; a K at position 385; a P at position 386; a Y at position 387; a T, Y, or F at position 389; a D, E, or Q at position 421; an I at position 422; a V at position 424; a D at position 426; a L or Y at position 428; a S at position 434; a F at position 436; an I or V at position 438; a K at position 440; and a Q or M at position 442.

In some embodiments, a CD98hc-binding region comprising a modified antibody heavy chain constant domain (e.g., modified CH3 peptide) or a Fc polypeptide comprises a sequence having at least 85%, at least 90%, or at least 95% sequence identity to the sequence of SEQ ID NO:34 or SEQ ID NO:35, wherein the modified constant domain comprises eight, nine, ten, eleven, twelve, thirteen, fourteen, fifteen, sixteen, seventeen, eighteen, or nineteen substitutions in a set of amino acid positions consisting of: a S or V at position 378; a D at position 380; a R at position 382; a T at position 383; a Y at position 384; a K at position 385; a P at position 386; a Y at position 387; a T, Y, or F at position 389; a D, E, or Q at position 421; an I at position 422; a V at position 424; a D at position 426; a L or Y at position 428; a S at position 434; a F at position 436; an I or V at position 438; a K at position 440; and a Q or M at position 442.

In some embodiments, a CD98hc-binding region comprising a modified antibody heavy chain constant domain (e.g., modified CH3 peptide) or a Fc polypeptide comprises a sequence having at least 85%, at least 90%, or at least 95% sequence identity to the sequence of SEQ ID NO:34 or SEQ ID NO:35, wherein the modified constant domain comprises at least eleven, at least twelve, or at least thirteen substitutions in a set of amino acid positions consisting of: a S or V at position 378; a D at position 380; a R at position 382; a T at position 383; a Y at position 384; a K at position 385; a P at position 386; a Y at position 387; a T, Y, or F at position 389; a D, E, or Q at position 421; an I at position 422; a V at position 424; a D at position 426; a L or Y at position 428; a S at position 434; a F at position 436; an I or V at position 438; a K at position 440; and a Q or M at position 442.

In some embodiments, a CD98hc-binding region comprising a modified antibody heavy chain constant domain (e.g., modified CH3 peptide) or a Fc polypeptide comprises a sequence having at least 85%, 90%, or 95% sequence identity to amino acids 111-217 of the sequence SEQ ID NO:34 or SEQ ID NO:35, wherein the modified constant domain comprises eight, nine, ten, eleven, twelve, thirteen, fourteen, fifteen, sixteen, seventeen, eighteen, or nineteen substitutions in a set of amino acid positions consisting of: a S or V at position 378; a D at position 380; a R at position 382; a T at position 383; a Y at position 384; a K at position 385; a P at position 386; a Y at position 387; a T, Y, or F at position 389; a D, E, or Q at position 421; an I at position 422; a V at position 424; a D at position 426; a L or Y at position 428; a S at position 434; a F at position 436; an I or V at position 438; a K at position 440; and a Q or M at position 442.

In some embodiments, a CD98hc-binding region comprising a modified antibody heavy chain constant domain (e.g., modified CH3 peptide) or a Fc polypeptide comprises a sequence having at least 85%, 90%, or 95% sequence identity to amino acids 111-217 of the sequence SEQ ID NO:34 or SEQ ID NO:35, wherein the modified constant domain comprises at least eleven, at least twelve, or at least thirteen, substitutions in a set of amino acid positions consisting of a S or V at position 378; a D at position 380; a R at position 382; a T at position 383; a Y at position 384; a K at position 385; a P at position 386; a Y at position 387; a T, Y, or F at position 389; a D, E, or Q at position 421; an I at position 422; a V at position 424; a D at position 426; a L or Y at position 428; a S at position 434; a F at position 436; an I or V at position 438; a K at position 440; and a Q or M at position 442.

In some embodiments, a CD98hc-binding region comprising a modified antibody heavy chain constant domain (e.g., modified CH3 peptide) or a Fc polypeptide comprises eight, nine, ten, eleven, twelve, thirteen, fourteen, or fifteen substitutions in a set of amino acid positions consisting of 382, 383, 384, 385, 386, 387, 389, 421, 422, 424, 426, 428, 436, 438, and 440, wherein the positions are determined according to EU numbering. In some embodiments, the substitutions are selected from: a R at position 382; a T at position 383; a Y at position 384; a K at position 385; a P at position 386; a Y at position 387; a T at position 389; a D at position 421; an I at position 422; a V at position 424; a D at position 426; a L at position 428; a F at position 436; an I at position 438; and a K at position 440.

In some embodiments, a CD98hc-binding region comprising a modified antibody heavy chain constant domain (e.g., modified CH3 peptide) or a Fc polypeptide comprises at least eleven, at least twelve, or at least thirteen substitutions in a set of amino acid positions consisting of 382, 383, 384, 385, 386, 387, 389, 421, 422, 424, 426, 428, 436, 438, and 440, wherein the positions are determined according to EU numbering. In some embodiments, the substitutions are selected from: a R at position 382; a T at position 383; a Y at position 384; a K at position 385; a P at position 386; a Y at position 387; a T at position 389; a D at position 421; an I at position 422; a V at position 424; a D at position 426; a L at position 428; a F at position 436; an I at position 438; and a K at position 440.

In some embodiments, a CD98hc-binding region comprising a modified antibody heavy chain constant domain (e.g., modified CH3 peptide) or a Fc polypeptide comprises a sequence having at least 85%, at least 90%, or at least 95% sequence identity to the sequence of SEQ ID NO:34 or SEQ ID NO:35, wherein the modified constant domain comprises eight, nine, ten, eleven, twelve, thirteen, fourteen, or fifteen substitutions in a set of amino acid positions consisting of a R at position 382; a T at position 383; a Y at position 384; a K at position 385; a P at position 386; a Y at position 387; a T at position 389; a D at position 421; an I at position 422; a V at position 424; a D at position 426; a L at position 428; a F at position 436; an I at position 438; and a K at position 440.

In some embodiments, a CD98hc-binding region comprising a modified antibody heavy chain constant domain (e.g., modified CH3 peptide) or a Fc polypeptide comprises a sequence having at least 85%, at least 90%, or at least 95% sequence identity to the sequence of SEQ ID NO:34 or SEQ ID NO:35, wherein the modified constant domain comprises at least eleven, at least twelve, or at least thirteen substitutions in a set of amino acid positions consisting of a R at position 382; a T at position 383; a Y at position 384; a K at position 385; a P at position 386; a Y at position 387; a T at position 389; a D at position 421; an I at position 422; a V at position 424; a D at position 426; a L at position 428; a F at position 436; an I at position 438; and a K at position 440.

In some embodiments, a CD98hc-binding region comprising a modified antibody heavy chain constant domain (e.g., modified CH3 peptide) or a Fc polypeptide comprises a sequence having at least 85%, at least 90%, or at least 95% sequence identity to amino acids 111-217 of the sequence of SEQ ID NO:34 or SEQ ID NO:35, wherein the modified constant domain comprises eight, nine, ten, eleven, twelve, thirteen, fourteen, or fifteen substitutions in a set of amino acid positions consisting of a R at position 382; a T at position 383; a Y at position 384; a K at position 385; a P at position 386; a Y at position 387; a T at position 389; a D at position 421; an I at position 422; a V at position 424; a D at position 426; a L at position 428; a F at position 436; an I at position 438; and a K at position 440.

In some embodiments, a CD98hc-binding region comprising a modified antibody heavy chain constant domain (e.g., modified CH3 peptide) or a Fc polypeptide comprises a sequence having at least 85%, at least 90%, or at least 95% sequence identity to amino acids 111-217 of the sequence of SEQ ID NO:34 or SEQ ID NO:35, wherein the modified constant domain comprises at least eleven, at least twelve, or at least thirteen substitutions in a set of amino acid positions consisting of a R at position 382; a T at position 383; a Y at position 384; a K at position 385; a P at position 386; a Y at position 387; a T at position 389; a D at position 421; an I at position 422; a V at position 424; a D at position 426; a L at position 428; a F at position 436; an I at position 438; and a K at position 440.

In some embodiments, a CD98hc-binding region comprising a modified antibody heavy chain constant domain (e.g., modified CH3 peptide) or a Fc polypeptide comprises amino acids 111-217 of SEQ ID NO:34 or SEQ ID NO:35.

In some embodiments, a CD98hc-binding region comprising a modified antibody heavy chain constant domain (e.g., modified CH3 peptide) or a Fc polypeptide comprises the amino acid sequence of SEQ ID NO:34 or SEQ ID NO:35.

TABLE 17
Fc peptides having modified CH3 domains that specifically bind CD98hc.

| SEQ | | |
|---|---|---|
| ID | | |
| NO. | Sequence | Clone |
| 34 | APELLGGPSVFLFPPKPKDTLMISRTPEVTCVVVDVSHEDPEVKFNWYVDGVEV | Clone LLB1-3-16-2 |
| | HNAKTKPREEQYNSTYRVVSVLTVLHQDWLNGKEYKCKVSNKALPAPIEKTIS | |
| | KAKGQPREPQVYTLPPSRDELTKNQVSLTCLVKGFYPSDIAVEWRTYKPYETNY | |
| | KTTPPVLDSDGSFFLYSKLTVDKSRWQQGDIFVCDVMHEALHNHFTIKKLSLSP | |
| | GK | |
| 35 | APELLGGPSVFLFPPKPKDTLMISRTPEVTCVVVDVSHEDPEVKFNWYVDGVEV | Clone LLB1-3-16 |
| | HNAKTKPREEQYNSTYRVVSVLTVLHQDWLNGKEYKCKVSNKALPAPIEKTIS | |
| | KAKGQPREPQVYTLPPSRDELTKNQVSLTCLVKGFYPSDIAVEWRTYKPYETNY | |
| | KTTPPVLDSDGSFFLYSKLTVDKSRWQQGDIFVCDVLHEALHNHFTIKKLSLSPG | |
| | K | |

In some embodiments, a CD98hc-binding region comprising a modified CH3 peptide comprises: (a) a R at position 382; a T at position 383; a Y at position 384; a K at position 385; a P at position 386; a Y at position 387; a T at position 389; a D at position 421; an I at position 422; a V at position 424; a D at position 426; a F at position 436; an I at position 438; and a K at position 440; or (b) a R at position 382; a T at position 383; a Y at position 384; a K at position 385; a P at position 386; a Y at position 387; a T at position 389; a D at position 421; an I at position 422; a V at position 424; a D at position 426; a L at position 428; a F at position 436; an I at position 438; and a K at position 440; wherein the positions are determined according to EU numbering.

In some embodiments, a CD98hc-binding region comprising a modified CH3 peptide comprises:
(i) a first amino acid sequence of X₁X₂YKPYX₃T (SEQ ID NO:54), wherein X₁ is E or R, X₂ is S or T, X₃ is any amino acid;
(ii) a second amino acid sequence of X₁X₂X₃VX₄DX₅X₆ (SEQ ID NO:55), wherein X₁ is N or D, X₂ is V or I, X₃, X₄, and X₅ are any amino acid, X₆ is M or L; and
(iii) a third amino acid sequence of X₁X₂IX₃X₄ (SEQ ID NO:56), wherein X₁ is Y or F, X₂ and X₃ are any amino acid; and X₄ is S or K.

Additional CH3 domains modified to specifically bind CD98hc are described in PCT/US2022/053220.

### 3. Group III CD98hc Binding Regions

In some embodiments, a CD98hc-binding region comprises an anti-CD98hc antibody antigen binding domain. An anti-CD98hc antibody antigen binding domain can comprise an antibody, a Fab (including an F(ab′)2), a scFab, a Fv fragment, an scFv, or a nanobody. Anti-CD98hc antibody antigen binding domains include, but are not limited to: a 1C03 anti-CD98hc VHH (e.g., 1C03-4 or 1C03-5 anti-CD98hc VHH): a CD98hc2 anti-CD98hc nanobody: an anti-CD98hc heavy chain antibody or nanobody having the CDR1, CDR2, and CDR3 sequences of the CD98hc2 anti-CD98hc nanobody; a CD98hc4 anti-CD98hc scFv; an anti-CD98hc antibody, Fab, scFab, Fv fragment, or scFv having the heavy chain and light chain CDR1, CDR2, and CDR3 sequences of the CD98hc4 anti-CD98hc scFv; a CD98hc5 anti-CD98hc scFv; or an anti-CD98hc antibody, Fab, scFab, Fv fragment, or scFv having the heavy chain and light chain CDR1, CDR2, and CDR3 sequences of the CD98hc5 anti-CD98hc scFv (as disclosed in WO 2021/205361), Sequence are shown in Table 18). The dual transporters of the present invention can include antigen binding domains that have the CDRs or variable chains of any of antibodies CD98hc1, CD98hc2. CD98hc3. CD98hc4, CD98hc5, CD98hc6, CD98hc7, CD98hc8, CD98hc9, CD98hc10, CD98hc11, CD98hc12, CD98hc13, CD98hc14, and CD98hc15 as described in WO 2021/205361 (incorporated herein by reference).

TABLE 18
Exemplary CD98hc-binding regions comprising antibody antigen binding domains.

| | |
|---|---|
| 1C03-4 | EVQLVESGGGLVQPGGSLRLSCVASGIIFSNHAVSWYRRAPGKQREVVAFLGSGTSTYYADSV |
| anti-CD98hc | KGRFT ISRDNAKNTGYLQMNSLKPEDTGVYYCNVYRYEGPGTQVTVSSASEPKSSDKTH |
| VHH | (SEQ ID NO: 133) |
| 1C03-5 | EVQLVESGGGLVQPGGSLRLSCVASGIIFSNHAVSWYRRAPGKQREVVAFLGSGSSTYYADSV |
| anti-CD98hc | KGRFTISRDNAKNTGYLQMNSLKPEDTGVYYCNVYRYEGPGTQVTVSSASEPKSSDKTH |
| VHH | (SEQ ID NO: 134) |
| 1C03-8 | EVQLVESGGGLVQPGGSLRLSCVASGIIFSNHAVSWYRRAPGKQREVVAFLGSGNSYYYADSV |
| | KGRFTISRDNAKNTGYLQMNSLKPEDTGVYYCNVYRYEGPGTQVTVSS (SEQ ID |
| | NO: 136) |
| 1C03-8.ala31 | EVQLVESGGGLVQPGGSLRLSCVASGIIFSNHAVSWYRRAPGKQREVVAFLGSGNSYYYADSV |
| | KGRFTISRDNAKNTGYLQMNSLKPEDTGVYYCNVARYEGPGTQVTVSS (SEQ ID |
| | NO: 137) |
| CD98hc2 | Heavy chain: |
| anti-CD98hc | QVQLVESGGGLVQAGGSLRLSCAASGRAFSSNGMGWFRQAPGKEREFVATISCSGTSTYYADS |
| nanobody | VKGRFTISRDNAKNTVYLQMNSLKPEDTAVYFCAADTRHCMGYTKDEGYAYWGQGTQVTVSS |
| | (SEQ ID NO: 36) |
| CD98hc2 | Heavy chain: |
| anti-CD98hc | CDR1: GRAFSSNG (SEQ ID NO: 37) |
| nanobody | CDR2: ISCSGTST (SEQ ID NO: 38) |
| CDR | CDR3: AADTRHCMGYTKDEGY (SEQ ID NO: 39) |
| sequences | |
| CD98hc4 | QVQLVESGGGVVQPGRSLRLSCAASGFTFSSYGMHWVRQAPGKGLEWVAIISYDGSNKHYADS |
| anti-CD98hc | VKGRFTISRDNSKNTLYLQMNSLRAEDTAVYYCARAPSSFYFDYWGQGTLVTVSSGTEGKSSG |
| scFv | SGSESKSTDIVMTQSPDSLAVSLGERATINCKSSQSVLESSNNKNYLAWYQQKPGQPPKLLIY |
| | WASTRESGVPDRESGSGSGTDETLTISSLQAEDVAVYYCQQYYSTPPTFGQGTKVVIK (SEQ |
| | ID NO: 40) |
| CD98hc4 | Heavy chain: |
| anti-CD98hc | CDR1: GFTFSSYG (SEQ ID NO: 41) |
| scFv | CDR2: ISYDGSNK (SEQ ID NO: 42) |
| CDR | CDR3: ARAPSSFYFDY (SEQ ID NO: 43) |
| sequences | Light chain: |
| | CDR1: QSVLESSNNKNY (SEQ ID NO: 44) |
| | CDR2: WAS (SEQ ID NO: 45) |
| | CDR3: QQYYSTPPT (SEQ ID NO: 46) |
| CD98hc5 | QVQLQESGPGLVKPSETLSLTCTVSGGSISSSYWSWIRQPPGKGLEWIGYIYNSGSTNYNPSL |
| anti-CD98hc | KSRVTISVDTSKNQFSLKLSSVTAADTAVYYCARETYYFDYWGQGTLVTVSSGTEGKSSGSGS |
| scFv | ESKSTDIVMTQSQKFMSTSVGDRVSVTCKASQNVGINVAWYQKKPGQPPKALIYSAAYRYSGV |
| | PDRFTGSGSGTDFTLTISNVQSEDLAEYFCQQYNSYPLTFGSGTKLEIK (SEQ ID |
| | NO: 47) |
| CD98hc5 | Heavy chain: |
| anti-CD98hc | CDR1: GGSISSSY (SEQ ID NO: 48) |
| scFv | CDR2: IYNSGST (SEQ ID NO: 49) |
| CDR | CDR3: ARETYYEDY (SEQ ID NO: 50) |
| sequences | Light chain: |
| | CDR1: QNVGIN (SEQ ID NO: 51) |
| | CDR2: SAA (SEQ ID NO: 52) |
| | CDR3: QQYNSYPLT (SEQ ID NO: 53) |

Additional anti-CD98hc antibodies are known in the art and are available from various commercial sources. Exemplary anti-CD98hc antibodies include, but are not limited to, IGN523 (Hayes G M et al. Int J Cancer 2015 137(3):710-720). Exemplary anti-CD98hc antibodies are also described in patent publications: WO 2008/017828, WO 2013/078377, WO 2021/205361, WO2022/252167, WO2015/146132, US20130052197, and U.S. Pat. No. 7,943,745 (each of which is incorporated herein by reference).

Brain shuttles containing anti-CD98hc antibodies are described in Pomnoppadol G et al. “Bispecific antibody shuttles targeting CD98hc mediate efficient and long-lived brain delivery of IgGs” bioRxiv Apr. 29, 2023 (doi: 10.1101/2023.04.29.538811. Preprint) (which is incorporated herein by reference).

Anti-CD98hc vNARs are described in WO 2021/205361, WO 2019/246288, and WO 2023/023166.

Antibody antigen binding domains that specifically bind BBB proteins can be identified using methods available in the art for generating and/or identifying antibodies that specifically bind to a target protein. Such methods include, but are not limited to, immunization, phage display, and ribosome display.

In some embodiments, a CD98hc-binding region comprises a heavy chain complementary determining region 3 (CDRH3) engineered into an antibody constant domain as described in WO 2023/087017.

In some embodiments, a CD98hc-binding region comprises a fibronectin type III domain peptide modified to specifically bind CD98hc.

In some embodiments, a CD98hc-binding region comprises a bicyclic peptide that specifically binds CD98hc.

In some embodiments, a CD98hc-binding region binds human CD98hc with an affinity of less than about 15 nM to about 6000 nM, or less than about 15 nM to about 5000 nM (e.g., about 15 nM, about 50 nM, about 100 nM, about 200 nM, about 300 nM, about 400 nM, about 500 nM, about 600 nM, about 700 nM, about 800 nM, about 900 nM, about 1000 nM, about 1500 nM, about 2000 nM, about 2500 nM, about 3000 nM, about 3500 nM, about 4000 nM, about 4500 nM, about 5000 nM, about 5500 nM, or about 6000 nM). In some embodiments, a CD98hc-binding region binds human CD98hc with an affinity of about 100 nM to about 500 nM (e.g., about 100 nM, about 150 nM, about 200 nM, about 250 nM, about 300 nM, about 350 nM, about 400 nM, about 450 nM, or about 500 nM). In some embodiments, the CD98hc-binding region has cynomolgus monkey (cyno) cross-reactivity. In some embodiments, the CD98hc-binding region binds to cyno CD98hc with an affinity of about 80 nM to about 5 μM (e.g., 80 nM, 100 nM. 200 nM, 300 nM, 400 nM, 500 nM, 600 nM, 700 nM, 800 nM, 900 nM, 1 μM, 1.5 μM, 2 μM, 2.5 μM, 3 μM, 3.5 μM, 4 μM, 4.5 μM, or 5 μM).

### C. MOG Binding Polypeptides:

A MOG binding region (means for binding MOG) is a molecule such as a polypeptide or a region or domain of a larger polypeptide or protein that specifically binds to a MOG, such as a human MOG.

In some embodiments, a MOG-binding region comprises an anti-MOG antibody antigen binding domain. An anti-MOG antibody antigen binding domain can comprise an antibody, a Fab (including a F(ab′)2), a scFab, a Fv fragment, an scFv, or a nanobody. Anti-MOG antibody antigen binding domains include, an anti-MOG antibody as described in WO2018123979 (e.g., SEQ ID NO: 9), WO2023035002 (see e.g., paragraph [0032]), CN117683131B (see e.g., paragraphs [0006]-[0008], or Nakano R et al (“A new technology for increasing therapeutic protein levels in the brain over extended periods.” PLoS One. 2019 Apr. 12:14(4):e0214404.), each of which is incorporated herein by reference, or an anti-MOG, Fab, scFab, Fv fragment, or scFv having the heavy chain and light chain CDR1, CDR2, and CDR3 sequences of the any of the anti-MOG antibodies described in WO2018123979, WO2023035002, CN117683131B, or Nakano R et al. Additional anti-MOG antibodies are known in the art and are available from various commercial sources.

In some embodiments, a MOG-binding region binds human MOG with an affinity of less than about 15 nM to about 6000 nM, or less than about 15 nM to about 5000 nM (e.g., about 15 nM, about 50 nM, about 100 nM, about 200 nM, about 300 nM, about 400 nM, about 500 nM, about 600 nM, about 700 nM, about 800 nM, about 900 nM, about 1000 nM, about 1500 nM, about 2000 nM, about 2500 nM, about 3000 nM, about 3500 nM, about 4000 nM, about 4500 nM, about 5000 nM, about 5500 nM, or about 6000 nM). In some embodiments, a MOG-binding region binds human MOG with an affinity of about 100 nM to about 500 nM (e.g., about 100 nM, about 150 nM, about 200 nM, about 250 nM, about 300 nM, about 350 nM, about 400 nM, about 450 nM, or about 500 nM). In some embodiments, the MOG-binding region has cynomolgus monkey (cyno) cross-reactivity. In some embodiments, the MOG-binding region binds to cyno MOG with an affinity of about 80 nM to about 5 μM (e.g., 80 nM, 100 nM. 200 nM, 300 nM, 400 nM, 500 nM, 600 nM, 700 nM, 800 nM, 900 nM, 1 μM, 1.5 μM, 2 μM. 2.5 μM, 3 μM. 3.5 μM, 4 μM. 4.5 μM, or 5 μM).

### D. Additional Polypeptide Modifications

Any of the antibodies or Fc polypeptides described herein, including those comprising a modified CH3 peptide, may further comprise additional mutations, e.g., to promote heterodimer formation, to modulate effector function, to extend serum half-life and/or stability, to influence glycosylation, and/or to reduce immunogenicity in humans.

### 1. Polypeptide Modifications for Heterodimerization

Any of the antibodies or Fc polypeptides described herein, including those comprising a modified CH3 peptide, may further comprise one or more mutations that promote heterodimer formation and hinder homodimer formation. These modifications are useful, for example, where it is desired to from a bi-specific antibody or to form a heteromeric heavy chain dimer or heteromeric Fc dimer. Formation of a heterodimeric Fc dimer can be used to from, e.g., an Fc dimer having one Fc polypeptide that binds a BBB transport protein (e.g., TfR or CD98hc) and a second Fc polypeptide that binds a second, different protein that is a BBB surface protein or a brain retention protein, or to form an Fc dimer that is monovalent for binding to the BBB transport protein or the second protein. In some embodiments the Fc dimer binds both TfR and CD98hc.

Exemplary mutations that promote Fc heterodimer formation include knob and hole mutations. The knobs-into-holes approach generally involves introducing a protuberance (“knob”) at the interface of a polypeptide (e.g., an Fc polypeptide) and a corresponding cavity (“hole”) in the interface of a second polypeptide (e.g., an Fc polypeptide), such that the protuberance can be positioned in the cavity so as to promote heterodimer formation and hinder homodimer formation. Protuberances are constructed by replacing amino acid having smaller side chains from the interface of the first polypeptide (e.g., an Fc polypeptide) with amino acids having larger side chains (e.g., Tyr or Trp). Compensatory cavities of identical or similar size to the protuberances are created in the interface of the second polypeptide (e.g., an Fc polypeptide) by replacing amino acids having larger side chains with amino acids having smaller side chains (e.g., Ala or Thr). In some embodiments, such additional mutations are at a position in the polypeptide (e.g., an Fc polypeptide) that does not have a negative effect on binding of the polypeptide to the BBB transport protein or to the second protein (e.g., CD98hc or TfR).

By way of example, a knob and hole approach for dimerization can comprise replacing a native Thr at position 366 of one of the polypeptides of a dimer (e.g., an Fc polypeptide) with a Trp to form a knob (i.e., a T366W knob mutation), and replacing a native Tyr at position 407 of the other polypeptide of the dimer (e.g., an Fc polypeptide) with a Val to form a hole (i.e., Y407V hole mutation). The other polypeptide (e.g., Fc polypeptide) may further comprise substitution of a native Thr at position 366 with Ser and substitution of a native Leu at position 368 with an Ala (i.e., T366S and L368A hole mutations). In some embodiments, one of the polypeptides of a dimer (e.g., an Fc polypeptide) has the T366W knob mutation and the other polypeptide (e.g., an Fc polypeptide) has the Y407V hole mutation, which is typically accompanied by the T366S and L368A hole mutations. All positions are numbered per EU numbering.

The knobs-into-holes approach, e.g., a T366W knob substitution on one polypeptide (e.g., an Fc polypeptide) with T366S, L368A, and Y407V hole substitutions on the other polypeptide (e.g., an Fc polypeptide) can be used with any of the dual transporters comprising antibodies, heavy chain dimers, or Fc dimers, including any of the described CD98hc-binding Fc polypeptides and/or TfR-binding Fc polypeptides.

In some embodiments, a dual transporter comprises a first Fc polypeptide having a CH3 peptide modified to bind a BBB transport protein (e.g., TfR or CD98hc) and a second Fc polypeptide having a CH3 peptide modified to bind a second, different protein that is a BBB surface protein or a brain retention protein. In some embodiments, a dual transporter comprises a first Fc polypeptide having a CH3 peptide modified to bind the BBB transport protein or the second protein and a second Fc polypeptide that does not have having a CH3 peptide modified to bind the BBB protein transport protein or the second protein. In some embodiments, the first Fc polypeptide comprises a knob mutation (e.g., T366W) and the second Fc polypeptide contains a hole mutation (e.g. T366S, L368A, and Y407V). In some embodiments, the first Fc polypeptide comprises a hole mutation (e.g., T366S, L368A, and Y407V) and the second Fc polypeptide contains a knob mutation (e.g., T366W).

In some embodiments, one or both polypeptides (e.g., Fc polypeptides) present in a polypeptide dimer (e.g., an Fc polypeptide dimer) can also be engineered to contain other modifications the promote heterodimerization, e.g., electrostatic engineering of contact residues within a CH3-CH3 interface that are naturally charged or hydrophobic patch modifications.

### 2. Polypeptide Modifications for Modulating Effector Function

Any of the antibodies or Fc polypeptides described herein, including those comprising a modified CH3 peptide, may further comprise one or more mutations that reduce or eliminate effector function. Reducing or eliminating effector function includes reducing or eliminating the ability of the Fc polypeptide or Fc dimer to induce certain biological functions upon binding of the Fc polypeptide or dimer to an Fc receptor expressed on an effector cell that mediates the effector function. Effector cells include, but are not limited to, monocytes, macrophages, neutrophils, dendritic cells, eosinophils, mast cells, platelets, B cells, large granular lymphocytes, Langerhans' cells, natural killer (NK) cells, and cytotoxic T cells. Examples of antibody effector functions include, but are not limited to, C1q binding and complement dependent cytotoxicity (CDC), Fc receptor binding, antibody-dependent cell-mediated cytotoxicity (ADCC), antibody-dependent cell-mediated phagocytosis (ADCP), down-regulation of cell surface receptors (e.g., B cell receptor), and B-cell activation.

In some embodiments, any of the antibodies or Fc polypeptides described herein may comprise a CH2 peptide having one or more mutations that reduce effector function. Mutations that reduce effector function can be, but are not limited to, mutations at positions 234, 235, and 390, according to EU numbering. The mutations may be present in a single heavy chain of an antibody, a single Fc polypeptide of an Fc dimer, in both heavy chains of an antibody, or in both Fc polypeptides of an Fc dimer.

In some embodiments, any of the antibodies or Fc polypeptides described herein may comprise a CH2 peptide having an A at position 234, an A at position 235; a G at position 329 (“P329G” or “PG” mutation); a S at position 329 (“P329S” or “PS” mutation); an A at position 234 and an A at position 235 (“LALA” mutation), A at position 234, an A at position 235, and a G at position 329 (“LALA PG” mutation), or an A at position 234, an A at position 235, and a S at position 329 (“LALA PS” herein).

In some embodiments, any of the antibodies or Fe dimers described can comprise an A at position 234, an A at position 235; a G at position 329 (“P329G” or “PG” mutation); a S at position 329 (“P329S” or “PS” mutation); an A at position 234 and an A at position 235 (“LALA” mutation), A at position 234, an A at position 235, and a G at position 329 (“LALA PG” mutation), or an A at position 234, an A at position 235, and a S at position 329 (“LALA PS” herein) in both CH2 peptides.

In some embodiments, any of the antibodies or Fc dimers described herein can comprise an A at position 234, an A at position 235; a G at position 329 (“P329G” or “PG” mutation); a S at position 329 (“P329S” or “PS” mutation); an A at position 234 and an A at position 235 (“LALA” mutation), A at position 234, an A at position 235, and a G at position 329 (“LALA PG” mutation), or an A at position 234, an A at position 235, and a S at position 329 (“LALA PS” herein) in a single CH2 peptide.

Additional mutations that modulate an effector function include, but are not limited to: (a) substitution of the native P at position 329 for a G, A, S, R, or an amino acid residue large enough to inhibit formation of the Fc/Fcγ receptor interface that is formed between P329 of the Fc and Trp87 and Trp110 of FcγRIII; and/or (b) one or more of S228P, E233P, L235E, N297A, N297D, or P331S substitutions, according to the EU numbering scheme.

Combination of substitutions that modulate an effector function include, but are not limited to: L234A, L235A, and P329G of human IgG1; S228P and L235E of human IgG4; L234A and G237A of human IgG1; L234A, L235A, and G237A of human IgG1; V234A and G237A of human IgG2; L235A. G237A, and E318A of human IgG4; and S228P and L236E of human IgG4, according to the EU numbering scheme.

### 3. Polypeptide Modifications for Extending Serum Half-Life

Any of the antibodies or Fc polypeptides described herein, including those comprising a modified CH3 peptide, may further comprise one or more mutations that alter serum half-life. In some embodiments, any of the antibodies or Fc polypeptides described herein, including those comprising a modified CH3 peptide, may further comprise one or more mutations that enhance or prolong serum half-life.

Exemplary mutations that enhance serum half-life include, but are not limited to: a M428L substitution, a N434S substitution, a M428L and a N434S substitutions (“LS” substitutions), a N434S substitution, a N434A substitution, a M428L substitution, a M252Y substitution, a S254T substitution, a T256E substitution, or combination of M252Y, S254T, and T256E substitutions, as numbered according to the EU numbering scheme.

The one or more mutations that enhance serum half-life may be present in a single heavy chain of an antibody, a single Fc polypeptide of an Fc dimer, in both heavy chains of an antibody, or in both Fc polypeptides of an Fc dimer.

4. Polypeptide with C-terminal Lysine Residue Removed

A C-terminal Lys (e.g., the Lys residue at position 447, according to EU numbering) can be removed from any of the antibodies or Fc polypeptides described herein, including those comprising a modified CH3 peptide. The C-terminal lysine residue is highly conserved in immunoglobulins across many species and may be fully or partially removed by cellular machinery during protein production. Removal of the C-terminal lysines in the Fc polypeptides may improve the stability of the proteins. In some embodiments, any of the antibodies or Fc polypeptides described herein, including those comprising a modified CH3 peptide, may comprise a deletion of a C-terminal Lys. In some embodiments, a heavy chain of any of the described antibodies or any of the described Fc polypeptides can be produced without a C-terminal Lys. In some embodiments, a C-terminal Lys may be removed from any of the antibodies or Fc polypeptides described herein. A C-terminal Lys can be removed from a single heavy chain of an antibody, from a single Fc polypeptide of an Fc dimer, from both heavy chains of an antibody, or from both Fc polypeptides of an Fc dimer.

### III. Methods of Manufacture

Described are methods of identifying a dual transporter comprising: (a) providing (i) a first polypeptide comprising a first binding region that specifically binds to a BBB transport protein and (ii) a second polypeptide comprising a second binding region that specifically binds a second protein, wherein the second protein is expressed on the luminal surface of the BBB or is a brain retention protein, and wherein the second protein is different from the BBB transport protein; (b) forming a single molecule that incorporates the first polypeptide and the second polypeptide; and (c) measuring the brain concentration of the single molecule at a predetermined time and identifying the single molecule as a dual transporter if the single molecule is present in the brain at a higher concentration than a corresponding control molecule that binds only to the BBB transport protein or the second protein. The single molecule can be administered peripherally into an animal prior to step (c). The animal can be, but is not limited to, a mouse, rat, or non-human primate. Presence in the brain at a higher concentration can be the result of an increased rate of transport to the brain and/or increased brain retention (i.e., prolonged brain exposure or brain exposure time) relative to the corresponding control molecule that binds only to the BBB transport protein or only to the second protein. The BBB transport protein can be, but is not limited to, TfR of CD98hc. In some embodiments, the BBB transport protein is TfR. The second protein can be, but is not limited to, TfR, CD98hc, Large neutral amino acids transporter small subunit 1 (CD98 light chain), GLUT1, MFSD2A, CA-IV, Low density lipoprotein receptor, IGF1R, Insulin-like growth factor 2 receptor. IgG receptor FcRn large subunit p51, Low density lipoprotein receptor-related protein 1, Low density lipoprotein receptor-related protein 2, Insulin receptor, Cell cycle control protein 50A, Transmembrane protein 50A, Basigin, Leptin Receptor, Claudin-5, P-selectin, Lactoferrin receptor, Folate receptor, Sodium-dependent lysophosphatidylcholine symporter 1, Solute carrier organic anion transporter family member 1C1, Sodium-coupled neutral amino acid transporter 5, LDL receptor-related protein 8, High affinity cationic amino acid transporter 1, Sodium- and chloride-dependent taurine transporter, Insulin-like growth factor-binding protein 7, Solute carrier family 40 member 1, Zinc transporter 6, heparin-binding epidermal growth factor-like growth factor, and MOG. In some embodiments, the second protein is CD98hc. In some embodiments, the second protein is MOG. In some embodiments, the second protein is selected from the group consisting of GLUT1, MFSD2A, CA-IV, and IGF1R.

The first binding region can be any polypeptide known to bind to the BBB transport protein, including any of the TfR or CD98hc binding regions disclosed or described herein. The second binding region can be any polypeptide known to bind to the second protein, including any of the TfR or CD98hc binding regions disclosed or described herein. In some embodiments, the first binding region binds to TfR.

In some embodiments, the method further comprises determining the binding affinity of the first binding region to the BBB transport protein and determining the binding affinity of the second binding region to the second protein. In some embodiments, the method further comprises determining the binding affinity of single molecule to the BBB transport protein, the second protein, or both the BBB transport protein and the second protein. Binding affinity or avidity can be determined using methods available in the art for determining binding affinity of a molecule to a cell, protein, or cell surface protein. Such methods include, but are not limited to, surface plasmon resonance, biolayer interferometry, ELISA, and flow cytometry. Binding of the first binding region, second binding region, or the single molecule (e.g., dual transporter) can be measured on the surface of a cell that expresses on its surface the BBB transport protein, the second protein, or both the BBB transport protein and the second protein. In some embodiments, affinity is measure using a Surface Plasmon Resonance (SPR) methods (e.g., using a Biacore™ system), kinetic exclusion assays (e.g., using a KinExA® system), and/or BioLayer interferometry (e.g., using the ForteBio® Octet® platform).

Described are methods of determining whether a dual transporter has increased and/or prolonged exposure in the brain of an animal comprising: (a) providing a single molecule that comprises (i) a first binding region that specifically binds to a BBB transport protein; and (ii) a second binding region that specifically binds to a second protein, wherein the second protein is expressed on the luminal surface of the BBB or is a brain retention protein, and wherein the second protein is different from the BBB transport protein; (b) administering the molecule peripherally to the animal; and (c) measuring concentration of the molecule in the brain to determine whether the molecule is present in the brain at a higher concentration than a corresponding control molecule that binds to only the BBB transport protein or only the second protein. Measuring concentration of the molecule in the brain can include measuring concentration of the molecule in the brain at one or more predetermined times. Presence in the brain at a higher concentration can be the result of an increased rate of transport to the brain and/or increased brain retention (i.e., prolonged brain exposure or brain exposure time) relative to the corresponding control molecule that binds only to the BBB transport protein or only to the second protein. The BBB transport protein can be, but is not limited to, TfR of CD98hc. In some embodiments, the BBB transport protein is TfR. The second protein can be, but is not limited to, TfR, CD98hc, Large neutral amino acids transporter small subunit 1 (CD98 light chain), GLUT1, MFSD2A, CA-IV, Low density lipoprotein receptor, IGF1R, Insulin-like growth factor 2 receptor, IgG receptor FcRn large subunit p51, Low density lipoprotein receptor-related protein 1, Low density lipoprotein receptor-related protein 2, Insulin receptor, Cell cycle control protein 50A, Transmembrane protein 50A, Basigin. Leptin Receptor, Claudin-5, P-selectin, Lactoferrin receptor, Folate receptor, Sodium-dependent lysophosphatidylcholine symporter 1, Solute carrier organic anion transporter family member 1C1, Sodium-coupled neutral amino acid transporter 5, LDL receptor-related protein 8, High affinity cationic amino acid transporter 1, Sodium- and chloride-dependent taurine transporter, Insulin-like growth factor-binding protein 7, Solute carrier family 40 member 1, Zinc transporter 6, heparin-binding epidermal growth factor-like growth factor, and MOG. In some embodiments, the second protein is CD98hc. In some embodiments, the second protein is MOG. In some embodiments, the second protein is selected from the group consisting of: GLUT1, MFSD2A, CA-IV, and IGF1R.

The first binding region can be any polypeptide known to bind to the BBB transport protein, including any of the TfR or CD98hc binding regions disclosed or described herein. The second binding region can be any polypeptide known to bind to the second protein, including any of the TfR or CD98hc binding regions disclosed or described herein.

In some embodiments, the method further comprises determining the binding affinity of the first binding region to the BBB transport protein and determining the binding affinity of the second binding region to the second protein. In some embodiments, the method further comprises determining the binding affinity of single molecule to the BBB transport protein, the second protein, or both the BBB transport protein and the second protein. Binding affinity or avidity can be determined using methods available in the art for determining binding affinity or avidity of a molecule to a cell, protein, or cell surface protein. Such methods include, but are not limited to, surface plasmon resonance, biolayer interferometry, ELISA, and flow cytometry. Binding of the first binding region, second binding region, or the single molecule (e.g., dual transporter) can be measured on the surface of a cell that expresses on its surface the BBB transport protein, the second protein, or both the BBB transport protein and the second protein. In some embodiments, affinity is measure using a Surface Plasmon Resonance (SPR) methods (e.g., using a Biacore™ system), kinetic exclusion assays (e.g., using a KinExA® system), and/or BioLayer interferometry (e.g., using the ForteBio® Octet® platform).

Described are methods of manufacturing a dual transporter comprising: (a) providing (i) a first polypeptide comprising a first binding region that specifically binds to a BBB transport protein and (ii) a second polypeptide comprising a second binding region that specifically binds a second protein, wherein the second protein is expressed on the luminal surface of the BBB or is a brain retention protein, and wherein the second protein is different from the BBB transport protein; and (b) forming a single molecule that incorporates the first polypeptide and the second polypeptide, thereby forming the dual transporter; wherein the dual transporter has increased and/or prolonged brain exposure in an animal as compared to a corresponding control molecule that binds only to the BBB transport protein or only to the second protein. In some embodiments, the first binding region specifically binds TfR, some embodiments, the first binding region specifically binds CD98hc. In some embodiments, the second binding region specifically binds CD98hc. In some embodiments, the second binding region specifically binds MOG. In some embodiments, the second binding region specifically binds a protein selected from the group consisting of: GLUT1, MFSD2A, CA-IV, and IGF1R.

The first binding region can be any polypeptide known to bind to the BBB transport protein, including any of the TfR or CD98hc binding regions (means for binding TfR or Cd98hs, respectively) disclosed or described herein. The second binding region can be any polypeptide known to bind to the second protein, including any of the TfR. CD98hc, or MOG binding regions (means for binding TfR, CD98hs, or MOG, respectively) disclosed or described herein.

The first and second polypeptides can be synthesized or encoded by one or more nucleic acids. The one or more nucleic acids can be expressed in a host cell (or host cells) and the encoding polypeptide(s) purified from the host cells using methods available in the art for producing proteins from host cells. The host cell can be a bacterial cell, a eukaryotic cell, an insect cell, or a mammalian cell.

### IV. Nucleic Acids, Vectors, And Host Cells

The dual transporters as described herein can be prepared using recombinant methods. Accordingly, isolated nucleic acids comprising sequences encoding any of the dual transporters described herein or portions thereof are readily generated using methods available in the art. Host cells into which the nucleic acids are introduced and that can be used to replicate the polypeptide-encoding nucleic acids and/or to express the polypeptides are also available in the art. A host cell can be, but is not limited to, a prokaryotic cell or a eukaryotic, the eukaryotic call be, but is not limited to, a yeast cell, an insect cell, or a mammalian cell (e.g., a human cell).

A nucleic acid encoding a dual transporter or a portion thereof can be DNA, RNA. cDNA, mRNA, single-stranded, double-stranded, linear or circular.

A dual transporter may comprise two or more (e.g., three) polypeptides, each of which may be encoded by a separate nucleic sequence. The separate nucleic acid sequence may be present on the same plasmid or vector or different plasmids or vectors. If present on the same plasmid or vector, the separate nucleic acid sequences may be expressed from a single promoter or from different promoters. Method of expressing nucleic acids encoding separate polypeptides from a single promoter are known in the art and include, but are not limited to, the use of 2A elements and internal ribosome entry sites.

A nucleic acid encoding a dual transporter or a portion thereof can be provided in a plasmid or vector. The plasmid or vector can be used to replicate the nucleic acid or facilitate expression of the nuclei acid. A plasmid or vector can be, but is not limited to, a viral vector, a phagemid, a yeast chromosomal vector, or a non-episomal mammalian vector.

In some embodiments, the nucleic acid encoding a dual transporter or a portion thereof operably linked to one or more regulatory sequences in an expression construct. The expression constructs can be adapted for expression of the polypeptide in a system that production of the dual transporter. Such a system can be, but is not limited to, mammalian cell expression system, an insect cell expression system, a yeast cell expression system, or a bacterial cell expression system.

Expression vehicles for production of a recombinant polypeptide include plasmids and other vectors. For instance, suitable vectors include plasmids of the following types: pBR322-derived plasmids, pEMBL-derived plasmids, pEX-derived plasmids, pBTac-derived plasmids, and pETC-derived plasmids for expression in prokaryotic cells, such as E. coli. The pcDNAI/amp, pcDNAEneo, pRc/CMV, pSV2gpt, pSV2neo, pSV2-dhfr, pTk2, pRSVneo, pMSG, pSVT7, pko-neo, and pHyg-derived vectors are examples of mammalian expression vectors suitable for transfection of eukaryotic cells. Alternatively, derivatives of viruses such as the bovine papilloma virus (BPV-l), or Epstein-Barr virus (pHEBo, pREP-derived, and p205) can be used for transient expression of polypeptides in eukaryotic cells. In some embodiments, it may be desirable to express the recombinant polypeptide by the use of a baculovirus expression system. Examples of such baculovirus expression systems include pVL-derived vectors (such as pVLl392, pVLl393, and pVL941), pAcUW-derived vectors (such as pAcUWl), and pBlueBac-derived vectors. Additional expression systems include adenoviral, adeno-associated virus, and other viral expression systems.

An expression vector for expressing a dual transporter or a portion thereof, of a plasmid or vector containing the nucleic acid can be transformed, transfected, or transduced into a host cell. The host cell can be, but is not limited to, a mammalian cell, a yeast cell, an insect cell, prokaryotic cell, Chinese hamster ovary (CHO) cell, a baby hamster kidney (BHK) cell, a NSO cell, a YO cell, a HEK293 cell, a COS cell, a Vero cell, or a HeLa cell. The host cell containing the expression vector can be cultured under appropriate conditions to allow expression of the dual transporter protein or a portion thereof.

A dual transporter can be manufactured by culturing a host cell comprising one or more nucleic acids encoding the dual transporter, expressing the dual transporter, and isolating the expressed dual transporter from the culture.

### V. Formulations

Any of the described dual transporters can be prepared, provided, or formulated as a salt, mixed salt, or a free acid. Suitable pharmaceutically acceptable salts include, but are not limited to, sodium, potassium, calcium, and magnesium salts.

Any of the described dual transporters can be provided in or formulated in a pharmaceutical composition. A pharmaceutical composition or medicament includes a pharmaceutically effective amount of at least one of the described dual transporters and optionally one or more pharmaceutically acceptable excipients. Pharmaceutically acceptable excipients (excipients) are substances other than the Active Pharmaceutical ingredient (API, therapeutic product (e.g., dual transporter)) that are intentionally included in the pharmaceutical composition. Excipients do not exert or are not intended to exert a therapeutic effect at the intended dosage. Excipients may act to (a) aid in processing of the API during manufacture, (b) protect, support or enhance stability, bioavailability, or patient acceptability of the API, (c) assist in product identification, and/or (d) enhance any other attribute of the overall safety, effectiveness, or delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

The carrier can be, but is not limited to, a solvent or dispersion medium containing, for example, water, saline, phosphate buffered saline, Ringer's solution, ethanol, polyol (for example, glycerol, propylene glycol, and liquid polyethylene glycol), and suitable mixtures thereof. A carrier may also contain adjuvants or additives such as preservatives, wetting agents, emulsifying agents and dispersing agents. A carrier may also contain isotonic agents, such as sugars, polyalcohols, sodium chloride, and the like.

The pharmaceutical compositions can contain other additional components commonly found in pharmaceutical compositions. Such additional components can include, but are not limited to: anti-pruritics, astringents, local anesthetics, or anti-inflammatory agents (e.g., antihistamine, diphenhydramine, etc.).

Pharmaceutically acceptable refers to those properties and/or substances which are acceptable to the subject from a pharmacological/toxicological point of view. The phrase pharmaceutically acceptable refers to molecular entities, compositions, and properties that are physiologically tolerable and do not typically produce an allergic or other untoward or toxic reaction when administered to a subject. In some embodiments, a pharmaceutically acceptable compound is approved by a regulatory agency of the Federal or a state government or listed in the U.S. Pharmacopeia or other generally recognized pharmacopeia for use in animals and more particularly in humans.

In some embodiments, the pharmaceutical compositions further comprise one or more additional active ingredients. The additional active pharmaceutical ingredient can be, but is not limited to, a small molecule drug.

A dual transporter or pharmaceutical composition containing a dual transporter can be formulated as a liquid formulation or as a solid formulation (including a powder or lyophilized formulation; e.g., a lyophilized cake or powder).

In some embodiments, the pharmaceutical compositions described herein can be formulated for administration to a subject.

As disclosed above, a dual transporter or pharmaceutical composition containing a dual transporter can be formulated for parenteral administration by injection, e.g., by bolus injection or continuous infusion. For injection, the dual transporter can be formulated into preparations by dissolving, suspending, or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers, and preservatives. In some embodiments, polypeptides can be formulated in aqueous solutions, preferably in physiologically compatible buffers such as Hanks' solution, Ringer's solution, or physiological saline buffer. Formulations for injection can be presented in unit dosage form, e.g., in ampules or in multi-dose containers, with an added preservative. The compositions can take such forms as suspensions, solutions, or emulsions in oily or aqueous vehicles, and can contain formulatory agents such as suspending, stabilizing, and/or dispersing agents.

Typically, a pharmaceutical composition for use in in vivo administration is sterile. Sterilization can be accomplished according to methods known in the art, e.g., heat sterilization, steam sterilization, sterile filtration, or irradiation.

Dosages and desired drug concentration of pharmaceutical compositions of the invention may vary depending on the particular use envisioned. The determination of the appropriate dosage or route of administration is well within the skill of one in the art.

### Kits

In some embodiments, kits comprising a dual transporter as described herein are provided. In some embodiments, the kits are for use in preventing or treating a neurological disorder condition of the central nervous system (CNS).

The described dual transporters and pharmaceutical compositions comprising dual transporters disclosed herein may be packaged or included in a kit, container, pack, or dispenser. The dual transporters and pharmaceutical compositions comprising the dual transporters may be packaged in pre-filled syringes or vials. Any of the dual transporters or pharmaceutical compositions containing dual transporters described herein can be formulated or packaged in single-dose or multi-dose format. Any of the dual transporters or pharmaceutical compositions containing the dual transporters described identified herein can be formulated for repeat dosing.

In some embodiments, the kit further comprises one or more additional therapeutic agents. For example, in some embodiments, the kit comprises a dual transporter as described herein and further comprises one or more additional therapeutic agents for use in the treatment of a neurological disorder or condition of the CNS.

The kit or package may further contain instructions for use. Instructions include documents describing relevant materials or methodologies pertaining to the kit. The instructions may include one or more of: background information, list of components and their availability information (purchase information, etc.), brief or detailed protocols for using the kit, trouble-shooting guidance, references, technical support, indications, usage, dosage, administration, contraindications, and/or warnings concerning the use of the drug, and any other related documents. Instructions can be supplied with the kit or as a separate member component, either as a paper form or an electronic form. The instructions may include a notice in a form prescribed by a governmental agency regulating the manufacture, use or sale of pharmaceuticals or biological products, which notice reflects approval by the agency of manufacture, use or sale for human administration.

### VI. Therapeutic Methods

The described dual transporters may be used deliver a therapeutic agent into a brain of a subject, i.e., across the blood-brain barrier. In some embodiments, the described dual transporters may be used to deliver a therapeutic agent across the BBB to an extracellular target on or near an astrocyte, microglia, oligodendrocyte, or a cancer cell. The described dual transporters may be used to provide brain uptake and delivery to specific extracellular or neuro-oncology targets in the brain. The described dual transporters may be used to facilitate or increase transcytosis of a therapeutic agent across the blood brain barrier.

The therapeutic agent can be an agent that targets, for example, ABCA1, ABCA7, ADAM17, ALK, alpha-synuclein or a derivative or fragment thereof, an amyloid-beta peptide or a derivative or fragment thereof, AXL, B7H3, BCMA, CD125, CD166, CD19, CD20, CD205, CD22. CD25, CD25, CD30, CD33, CD37. CD39. CD44v6. CD46, CD56 (NCAM), CD73, CD79b, CDH6 (cadherin 6), CEACAM 5 (CD66E), CR1, EGFR viii, EGFR, ETBR, FGFR (1-4), Folate Receptor alpha, GAL-3BP (galectin binding protein), GD2, GD3, GloboH (globohexasylceramide), gp100, gpNMB, HER2, HER3, HER4, HLA-DRT, HLA-DR5, huntingtin, IGFR1, IL1RAP, IL-34, KIT, LIV1A, LRRC15 (leucine rich repeat containing 15), MET, MS4A4A. MS4A4E, MS4A6A, NaPi2B. TAR DNA-binding protein 43 (TDP-43) or a derivative or fragment thereof, PDL1, PILRA, PMELI7, PRAME, PSMA, pTau, PTK7 (CCK4; colon carcinoma kinase), RON, ROR1, Siglec11, SORL1, Tau or a derivative or fragment thereof, TF (tissue factor), transthyretin, TREM2, TREML2, TROP2, or a tumor cell.

The described dual transporters can be administered to a subject to treat a disease or condition of the brain or CNS. After administration to a subject, the described dual transporters are translocated across the BBB to the brain.

Described are methods of treating a subject suffering from a neurological disorder or condition of the CNS, comprising administering to the subject a dual transporter linked to a therapeutic agent or a composition containing a dual transporter linked to a therapeutic agent. The neurological disorder or condition of the CNS can be, but is not limited to, neurodegenerative disease, Alzheimer's disease, or an infection.

Described are methods of treating neurological disorder or condition of the CNS comprising, administering to the subject a dual transporter or a composition containing a dual transporter. In some embodiments, the subject has or is diagnosed with a neurological disorder or condition of the CNS or is at increased risk of developing a neurological disorder or condition of the CNS. The neurological disorder or condition of the CNS can be, but is not limited to, a neurodegenerative disease. Alzheimer's disease, cognitive impairment, memory loss, dementia.

Described are methods of delaying or preventing one or more symptoms or pathological conditions associated with a neurological disorder or condition of the CNS in a subject comprising administering to the subject a dual transporter or a composition containing a dual transporter.

A dual transporter can be administered parenterally, intravenously, or intrathecally, by epidural administration, or intracerebroventricularly. Administration can be by bolus injection, infusion, or a combination thereof. Infusion can occur over a period of from about 10 to about 30 minutes, or over a period of at least 1 hour to about 4 hours or more.

A dual transporter can be administered in combination with one or more additional therapies.

In some embodiments, the method further comprises administering to the subject one or more additional therapeutic agents. For example, in some embodiments for treating a disease of the brain or central nervous system, the method may comprise administering to the subject a neuroprotective agent, e.g., an anticholinergic agent, a dopaminergic agent, a glutamatergic agent, a histone deacetylase (HDAC) inhibitor, a cannabinoid, a caspase inhibitor, melatonin, an anti-inflammatory agent, a hormone (e.g., estrogen or progesterone), or a vitamin. In some embodiments, the method comprises administering to the subject an agent for use in treating a cognitive or behavioral symptom of a neurological disorder (e.g., an antidepressant, a dopamine agonist, or an anti-psychotic).

### EXAMPLES

### Example 1. Discovery and Characterization of TV^{CD98hc} Clones for Use in Dual Transporters

The engineering and characterization of TV^{CD98hc} variants from the LLB1 and LLB2 families is described in PCT/US2022/053220 and herein.

LLB1 and LLB2 clones were selected for incorporation into dual transporters based on their affinities to CD98hc. The affinity of TV^{CD98hc} variants to human CD98hc were assessed by surface plasmon resonance (SPR) using a Biacore™ 8K instrument. For these measurements, the indicated CD98hc binding domain (TV^{CD98hc}) was incorporated into a single Fc peptide of an anti-DNP antibody in the absence of a BBB second (e.g., TfR) binding region. A set of clones encompassing a broad affinity range was chosen to enable the assessment of how CD98hc affinity impacts dual transporter brain uptake and biodistribution. Exemplary clones and their affinity to human CD98hc are given below.

TABLE 19
Binding affinity of CD98hc clones to human CD98hc.

| | | Expected |
|---|---|---|
| | Kd average | Measurement |
| CD98Hc clone | (nM) | Range* (nM) |
| LLB2-37 | 610 | 305-1220 |
| LLB2-10-8 | 170 | 85-340 |
| LLB2.10.8.14.3 | 20 | 10-40 |
| LLB2-10-8-d6 MV | 550 | 275-1100 |
| LLB2-10-8-d12 BV | 2100 | 1050-4200 |
| LLB2-10-8.d28 | 4200 | 2100-8400 |
| LLB2.10.8.10.3 | 43 | 21.5-84 |
| LLB1-3-16 | 32 | 16-64 |
| LLB1-3-16-2 | 175 | 87.5-350 |

*It will be appreciated by those skilled the in the art that affinity measurements are subject to a degree of variability. The expected measurement range indicates a reasonable range of measured affinities (~95% confidence interval) that may not be substantially different from the Kd average.

### Example 2. Discovery and Characterization of TV^{TfR} Clones for Use in Dual Transporters

The engineering and characterization of TV^{TfR} variants (modified CH3 regions that specifically bind TfR) from the TV42 family are described in PCT/US2022/053220 (incorporated herein by reference) and herein (e.g., group II Modified CH3 peptide TfR Binding Regions). The engineering and characterization of TV^{TfR} variants from the TV35 family are described in WO 2018/152326 (incorporated herein by reference), Kariolis et al., Sci Transl Med 12(545).eaay1359, 2020 (incorporated herein by reference), and herein (e.g., group I Modified CH3 peptide TfR Binding Regions). The engineering and characterization of TV^{TfR} variants from additional families are described in PCT/US2022/053220 and PCT/US2022/053234 (each of which is incorporated herein by reference) and herein (e.g., group III Modified CH3 peptide TfR Binding Regions).

TV35 and TV42 clones were selected for incorporation into dual transporters based on their affinities to TfR. The affinity of TV^{TfR} variants to human TfR were assessed by surface plasmon resonance (SPR) using a Biacore™ 8K instrument. For these measurements, the indicated TfR binding domain (TV^{TfR}) was incorporated into a single Fc peptide of an anti-DNP antibody in the absence of a BBB second (e.g., CD98hc) binding region. A set of clones encompassing a broad affinity range was chosen to enable the assessment of how TfR affinity impacts dual transporter brain uptake and biodistribution. Exemplary clones and their affinity to human TfR are given below.

TABLE 20
Binding affinity of modified CH3 domains
to human TfR apical domain.

| | | Expected |
|---|---|---|
| | K_{D} average | Measurement |
| TfR clone | (nM) | Range* (nM) |
| CH3C.35.23.2 (TV35.23.2) | 100 | 50-200 |
| CH3C.35.23.4 (TV35.23.4) | 620 | 310-1240 |
| TV35.d1.10 (35.d1.10) | 2500 | 1250-5000 |
| LALAPG knob TV35.d1.9 (35.d1.9) | 8000 | 4000-16000 |
| 6.5.11.5.42.2 mono (TV42.2)* | 650 | 325-1300 |
| 42.8.143 | 450 | 225-900 |

*It will be appreciated by those skilled the in the art that affinity measurements are subject to a degree of variability. The expected measurement range indicates a reasonable range of measured affinities (~95% confidence interval) that may not be substantially different from the Kd average.

Any of the above modified CH3 TfR binding regions can further contain additional mutations to promote heterodimer formation (e.g., know or hole mutations), modulate effector function (e.g., LALA or LALAPG mutations), extend serum half-life and/or stability, influence glycosylation, and/or reduce immunogenicity in humans.

### Example 3. Design, Expression, and Characterization of Dual Transporter Clones

Dual transporters were constructed by creating a heterodimeric Fc domain, wherein one Fc polypeptide of a Fc dimer was engineered to bind to TfR (TV^{TfR}) while the second Fc polypeptide of the Fc dimer was engineered to bind to CD98hc (TV^{CD98hc}). Knob-into-hole mutations were incorporated to facilitate proper pairing of the heterodimer, while reducing or preventing the formation of homodimeric species. A diversity of TV^{TfR} and TV^{CD98hc} sequences derived from multiple TV families were explored in combination to assess the impact of binding affinity, epitope, and orientation on brain uptake and CNS biodistribution.

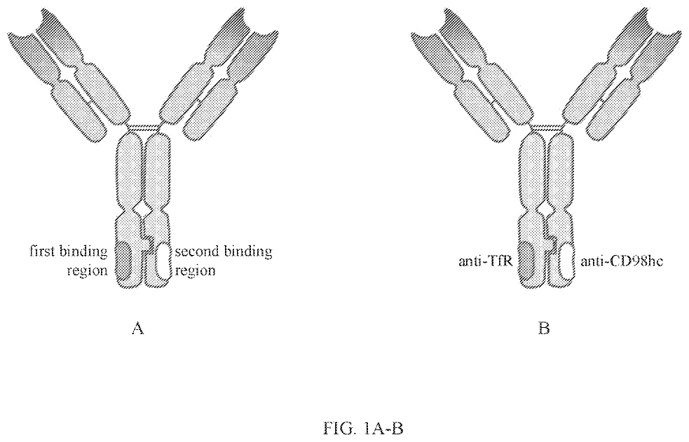

TV^{TfR} and TV^{CD98hc} variants were used to generate the heavy chains of an antibody transport vehicle (ATV) by fusing the engineered Fc domains to a Fab that binds the hapten, dinitrophenyl (DNP; FIG. 1). The knob mutation was added to TV^{TfR} ATV heavy chains while hole mutations were incorporated into the TV^{CD98hc} ATV heavy chains. Both included mutations (LALAPG) to reduce binding to FcγRs and attenuate effector function in vivo.

Heavy chain sequences and the corresponding light chain of the anti-DNP Fab were cloned into a mammalian expression vector with a CMV promoter and a signal peptide for secretion. Knob TV^{TfR} heavy chain, hole TV^{CD98hc} heavy chain, and the anti-DNP light chain were co-transfected in Expi293 cells. After five days in culture, supernatants were harvested and filtered, and dual transporters were purified by protein A affinity chromatography followed by size exclusion chromatography, if necessary. Permutations of dual transporters were explored by altering the combination of the knob TV^{TfR} and hole TV^{CD98hc} that were co-expressed.

The affinities of the dual transporters for human TfR and human CD98hc were assessed by surface plasmon resonance (SPR) using a Biacore™ 8K instrument. To interrogate the importance of the relative affinities for TfR and CD98hc, combinations of different TV^{TfR} variants, from both the TV35 and TV42 families, were paired with various TV^{CD98hc} clones from the LLB1 and LLB2 families. Representative dual transporter variants and their affinities to TfR and CD98hc are shown below.

TABLE 21A
Dual transporters: Experiment 1. Measured binding affinities of
TfR binding regions to human TfR apical domain and measured binding
affinities of CD98hc binding regions to human CD98hc.

| | | |
|---|---|---|
| | TfR affinity | CD98hc affinity |
| Name | (nM) | (nM) |

Experiment 1 (affinities measured in this single experiment)

| | | |
|---|---|---|
| LALAPG Dual TV35.23.4.knob/LLB2-10-8-d6.hole | 220 | 579 |
| LALAPG Dual TV35.23.4.knob/LLB2-10-8.d28.hole | 276 | 6527 |
| LALAPG Dual TV35.23.2.knob/LLB2.10.8.14.3.hole | 72 | 20 |
| LALAPG Dual TV35.23.2.knob/LLB2-10-8.hole | 76 | 96 |
| LALAPG Dual TV35.23.2.knob/LLB2-10-8-d6.hole | 76 | 606 |
| LALAPG Dual TV35.23.2.knob/LLB2-10-8.d28. | 67 | 6300 |
| LALAPG Dual TV35.d1.10.knob/LLB2-10-8.hole | 2210 | 170 |
| LALAPG Dual TV35.d1.10.knob/LLB2-10-8-d6.hole | 2409 | 550 |
| LALAPG Dual TV35.d1.10.knob/LLB2-10-8.d28.hole | 2359 | 6300 |

Expected affinities based on standard molecule format:

| | | |
|---|---|---|
| LALAPG Dual TV35.23.4/LLB2-10-8 | 620 | 170 |
| LALAPG Dual TV35.23.4.knob/LLB2-10-8-d6.hole | 620 | 550 |
| LALAPG Dual TV35.23.4.knob/LLB2-10-8.d28.hole | 620 | 6300 |
| LALAPG Dual TV35.23.2.knob/LLB2-10-8.hole | 100 | 170 |
| LALAPG Dual TV35.23.2.knob/LLB2-10-8-d6.hole | 100 | 550 |
| LALAPG Dual TV35.23.2.knob/LLB2-10-8.d28. | 100 | 6300 |
| LALAPG Dual TV35.d1.10.knob/LLB2-10-8.hole | 2500 | 170 |
| LALAPG Dual TV35.d1.10.knob/LLB2-10-8-d6.hole | 2500 | 550 |
| LALAPG Dual TV35.d1.10.knob/LLB2-10-8.d28.hole | 2500 | 6300 |

LALAPG indicates the presence of LALA and PG mutations
knob indicates the presence of knob mutations
hole indicates the presence of a hole mutation.

For any of the above dual transporters, the modified CH3 peptide TfR binding region can have a hole mutation and the modified CH3 peptide CD98 biding region can have knob mutations. Also, for any of the above dual transporters, the modified CH3 peptides may or may not contain LALA and/or PG mutations.

### Example 4. Cell Binding of Dual Transporters

Dual transporters were tested for their ability to bind to both TfR and CD98hc on the cell surface of model BBB cells. HEK293T cells expressing both human CD98hc and human TfR were plated at 40,000 cells/well of 96 well plates in standard growth media (DMEM (GIBCO™ 11995073)+10% FBS (VWR 89510-188)+1× Pen/Strep (Gibco 15140122). Approximately 24 hours later, dual transporters were diluted into standard growth media warmed to 37° C. Old media was removed from the cells, and the diluted molecules were added to the cells. Cells were incubated at 37° C. for 45 minutes. Cells were washed with PBS and then fixed for 10 minutes in 4% PFA (Electron Microscopy Sciences 15714-S). Cells were washed with PBS and then blocked with 5% BSA, 0.3% TritonX100 in PBS for 30 minutes. Cells were stained with anti-human IgG-AlexaFluor488 (1:1000; Jackson Immuno Research 109-545-003), cell mask (1:10,000; Thermo H32721), and DAPI (1:2000; Thermo D1306) diluted in 1% BSA, 0.3% TritonX100 in PBS for at least 30 minutes. Cells were washed with PBS, imaged on an Opera Phenix, and images were analyzed with Harmony software.

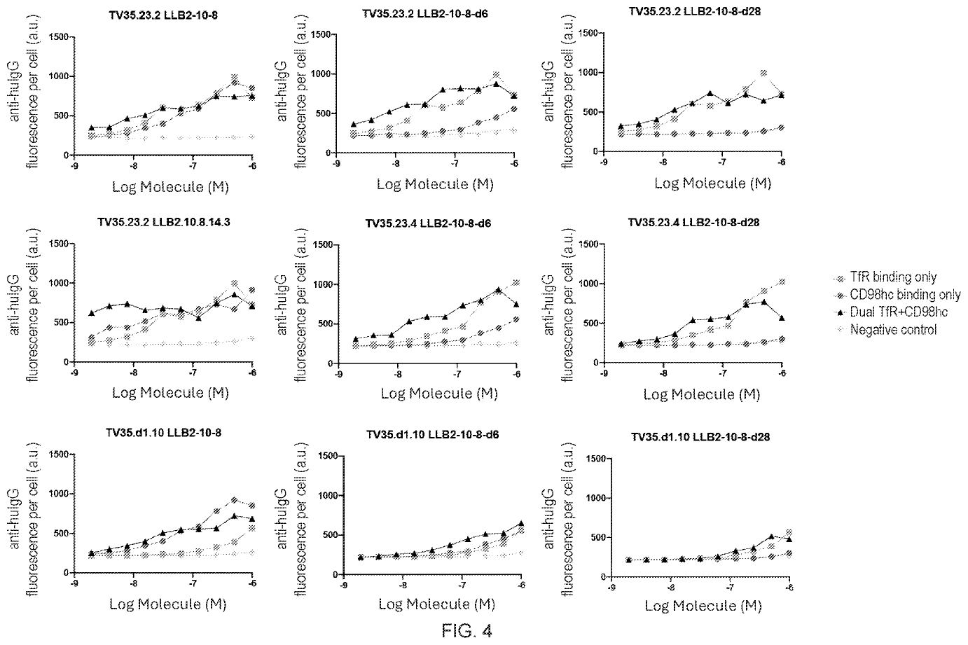

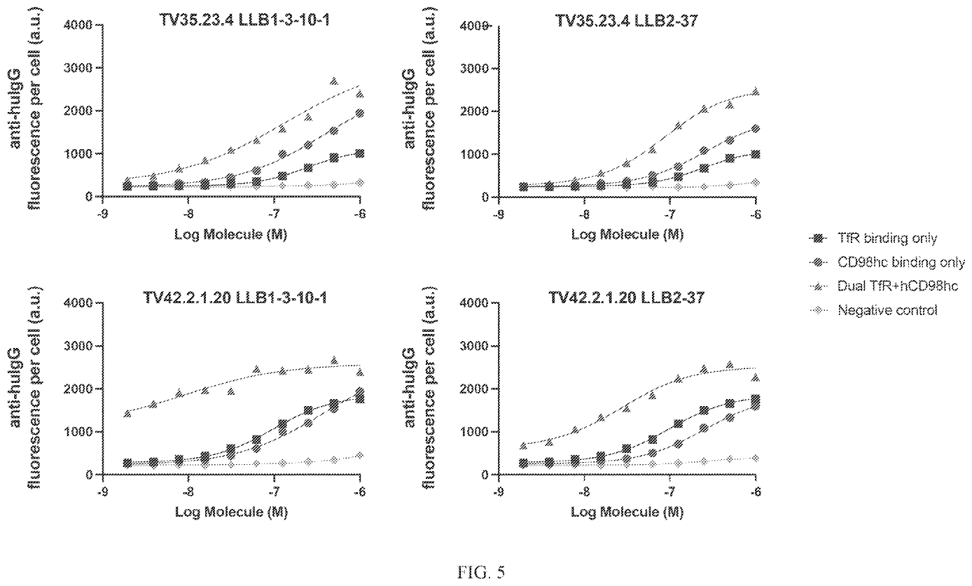

The dual transporters were found to bind to cells more at a given concentration relative to ATV^{TfR} and ATV^{CD98hc}, indicating the ability of the dual transporters to engage with both targets on the cell surface (FIGS. 4 and 5).

### Example 5. Generation of Monoclonal Antibodies Against Human Transferrin Receptor

Balb/c and SJL mice were immunized with recombinant human TfR extracellular domain via intraperitoneal route using complete/incomplete Freund adjuvant. After multiple boosting cycles, mice were sacrificed and spleens and lymph nodes were recovered. Single cells were fused to mouse myeloma cells using PEG method and fused hybridomas were plated into 96-well tissue culture plates and grown under HAT selection media. Following around two weeks of selection and growth, supernatants were screened for binding to recombinant human and cyno TfR proteins by ELISA followed by screening for binding to CHO cells that overexpressed either full-length human or cyno TfR while not binding to parental CHO cells. Hybridomas that selectively bound recombinant and cell-expressed TfR proteins were sub-cloned and sequenced using 5′-RACE method.

The variable heavy and variable light domains of TfR binders identified from the hybridoma screen were sub-cloned onto the constant heavy domains of human IgG1 containing LALA mutations or the constant kappa light domain, respectively. The expression plasmids contain a CMV promoter to drive expression and signal peptide for secretion. The heavy chain and light chain plasmids were co-transfected in Expi293 cells. Supernatants were collected after five days and monoclonal antibodies were purified by protein A chromatography. Alanine scan mutants, rationally designed variants, and humanization variants of these antibodies were generated, and were expressed and purified in the same manor.

The affinities for human and cyno TfR apical domain were measured by Biacore.

### Example 6 Development of an Anti-Human CD98hc VHH Domain

A camelid VHH domain that binds to human CD98hc was generated by immunization of two llamas with the extracellular domains of human and cyno CD98hc (the antigen). Llamas were immunized with 0.5 milligrams of antigen plus complete Freund adjuvant at week 0 followed by boosts with 0.5 milligrams of antigen plus incomplete Freund adjuvant on weeks 2, 4, 8 and 12. Production bleeds of 500 mL of whole blood were collected for PBMC isolation on weeks 10 and 12.

PBMC cells were isolated from fresh blood within 4 hours of blood-draw. Total RNA was isolated using RNeasy Maxi Kit (Qiagen), cDNA was reverse transcribed from which a VHH library generated. Library DNA was cloned into a phagemid vector and ligated vectors were transformed into TG1 cells. The resulting library was estimated to be 1.8×10⁹ in size.

VHH clones having cross-reactive binding to human and cyno CD98hc were isolated from the phage library over three successive rounds of panning. To prevent enrichment of clones that bind nonspecifically, in each round the phage library was first pre-absorbed against BSA coated plates. The library was subsequently panned against an equal mixture of human and cyno CD98hc, with the total concentration of antigen being 10 μg/mL.

After the third round of panning, screening was done on 94 clones to assess binding to human and cyno CD98hc. Periplasmic fractions containing VHH domains were prepared and clones were assessed for binding by ELISA yielding ten unique VHH sequences selected for further characterization.

Selected VHH domains were cloned into a mammalian expression vector with a CMV promoter and a signal peptide to enable secretion. Plasmid DNA encoding the VHH Fc fusions were transfected in Expi293 cells. After five days in culture, supernatants were harvested and filtered, and VHH Fc fusions were purified by protein A affinity chromatography followed by size exclusion chromatography, if necessary. Binding of the VHH domains to human and cyno CD98hc was assessed by surface plasmon resonance (SPR) using a Biacore™ 8K instrument. Clone 1C03-4 showed cross-reactive binding.

### Example 7. Design of Dual Transporters

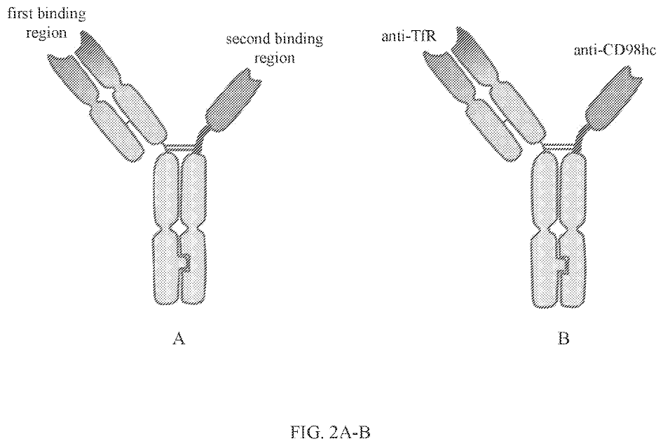

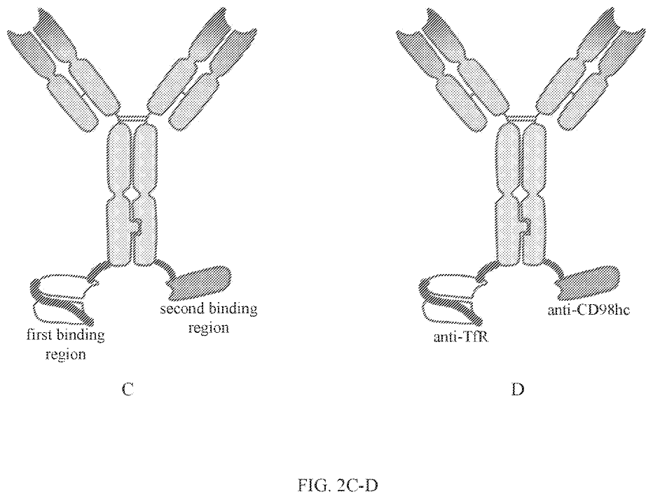

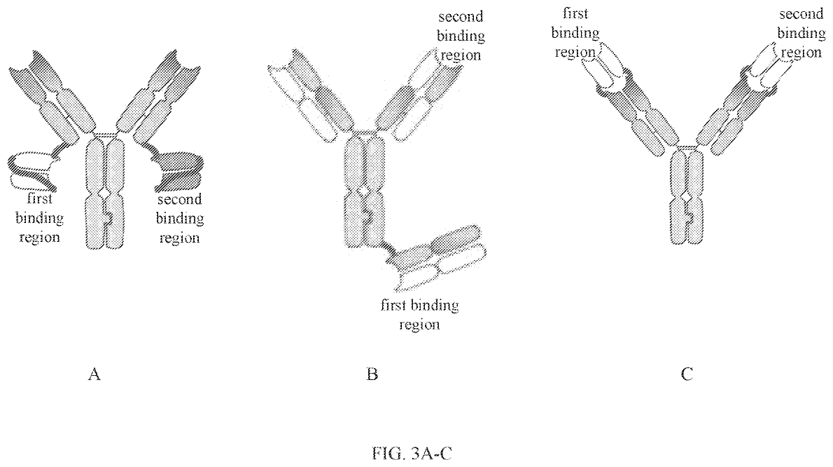

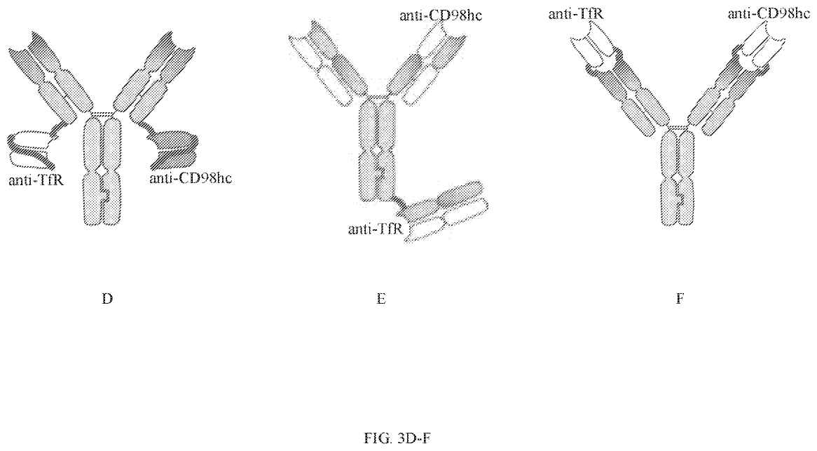

Molecules that bind to both TfR and CD98hc are designed using two different formats (FIG. 2A-D). In the first, the antibody framework is used to generate a bispecific antibody wherein one Fab arm binds to TfR (e.g., 17H10 or 17H10.1) while the other Fab arm is replaced by an anti-CD98hc VHH domain (e.g., 1C03-4 or 1C03-5), providing a Fab-Fc/VHH-Fc format. The second format utilizes an antibody with anti-DNP Fabs as a scaffold and incorporates binding to TfR and CD98hc by appending antibody fragments to the C-terminus of each heavy chain. One heavy chain has an anti-TfR scFv fused to its C-terminus while the other heavy chain has an anti-CD98hc VHH domain appended to its C-terminus (antibody fused to C-terminal VHH and scFv format). A flexible G₄S peptide linker can be used to join the antibody heavy chain and the fused antibody fragments. In both architectures, knob-into-hole mutations can be incorporated to facilitate proper pairing of the heterodimer, while reducing or preventing the formation of homodimeric species. The antibody framework dual transporters can include mutations (e.g., LALAPG mutations) in their Fc polypeptides to reduce binding to FcγRs and attenuate effector function in vivo.

Four affinity variants of each architecture are generated as outlined in Table 22 below.

TABLE 22
Exemplary TfR/CD98hc dual transporters ((a) VHH-Fc/
Fab-Fc and (b) scFv domain and VHH domain linked to
a full-length antibody formats (FIG. 2)).
Dual transporter

(a) Fab-Fc/VHH-Fc format
anti-TfR⁵⁰⁰ Fab-Fc/anti-CD98hc¹⁰⁰ VHH-Fc
anti-TfR⁷⁹ Fab-Fc/anti-CD98hc¹⁰⁰ VHH-Fc
anti-TfR⁵⁰⁰ Fab-Fc/anti-CD98hc⁶⁴ VHH-Fc
anti-TfR⁷⁹ Fab-Fc/anti-CD98hc⁶⁴ VHH-Fc
(b) antibody fused to C-terminal VHH and scFv format
anti-DNP02 C-term anti-TfR⁵⁰⁰ scFv/anti-CD98hc¹⁰⁰ VHH
anti-DNP02 C-term anti-TfR⁷⁹ scFv/anti-CD98hc¹⁰⁰ VHH
anti-DNP02 C-term anti-TfR⁵⁰⁰ scFv/anti-CD98hc⁶⁴ VHH
anti-DNP02 C-term anti-TfR⁷⁹ scFv/anti-CD98hc⁶⁴ VHH
Superscripts (^{500, 79, 100, 64}) indicate approximate measured affinities of the indicated anti-TfR Fab-Fc and anti-CD98hc VHH for TfR and CD98hc, respectively.

Measured affinity to the TfR and CD98 binding domains for their respective targets was weaker in the context of the VHH-Fc/Fab-Fc format and the scFv domain and the VHH domain linked to a full-length antibody format than was previously measured for the isolated TfR and CD98 binding domains.

### Example 8. PK Characterization of Dual Transporters in TfR^{mu/hu} KI CD98Mu Hu KI Mice at 50 mg Kg Dose

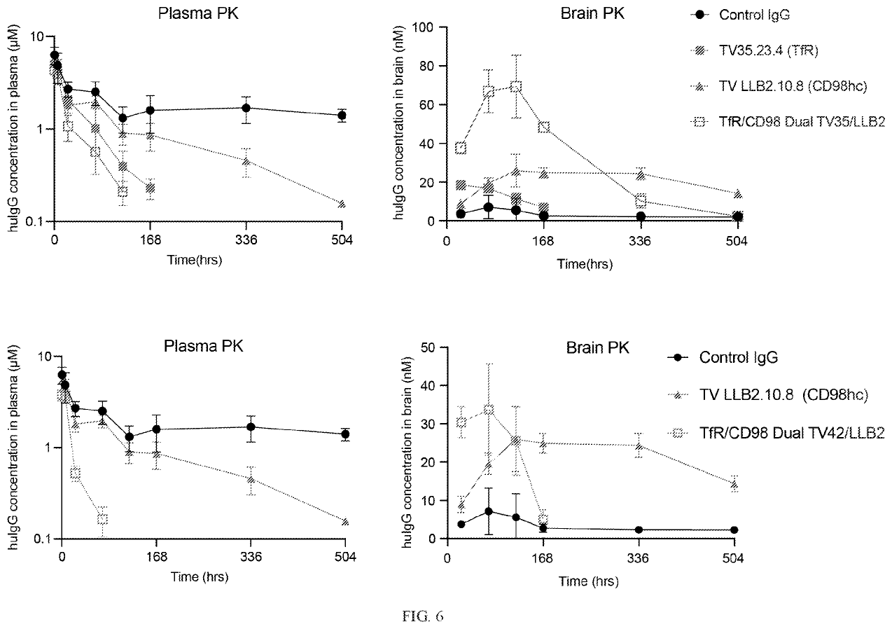

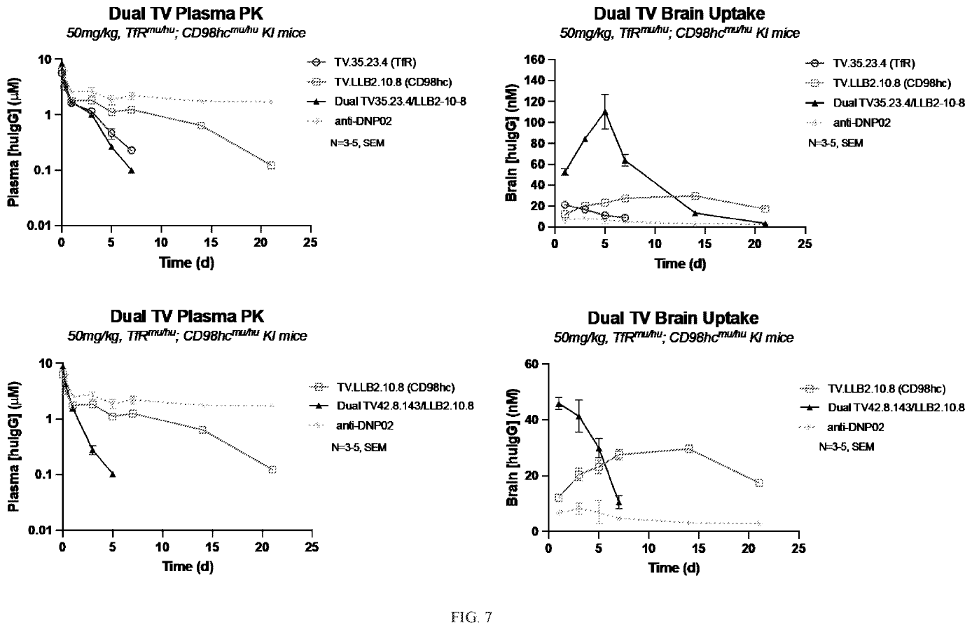

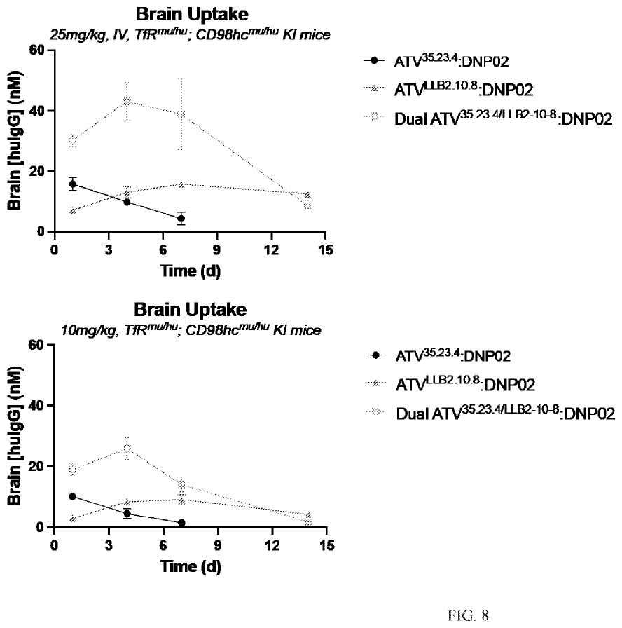

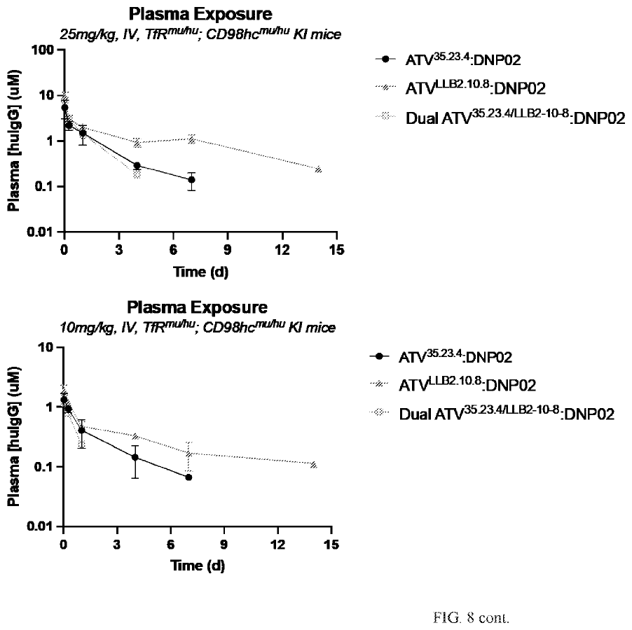

The peripheral and brain pharmacokinetic (PK) profiles of dual transporter variants were assessed in transgenic mice harboring humanized transferrin receptor and humanized CD98hc (TfR^{mu/hu} KI; CD98hc^{mu/hu} KI mice). These mice expressed (a) a TfR protein in which the native apical domain was replaced with human apical domain (knocked-in) at the endogenous locus, and (b) CD98hc in which the extracellular domain of CD98hc was humanized at the endogenous locus. Mice were intravenously dosed with 50 (FIGS. 6 and 7), 25, and 10 mg/kg of dual TVs, ATV^{TfR}, ATV^{CD98hc}, or control IgG. The single binding ATVs served as a control to benchmark PK profiles when TfR and CD98hc are engaged independently (FIG. 6, 7, 8). At timepoints indicated in the figures, blood was collected via cardiac puncture (in life bleeds were collected via submandibular bleeds), and at terminal timepoints the mice were perfused with PBS. Brain tissue was homogenized using a Qiagen TissueLyser in 10× tissue weight of lysis buffer containing 1% NP-40 in PBS with protease inhibitors. Blood was collected in EDTA tubes to prevent clotting and spun at 14000 rpm for 7 minutes to isolate plasma. The total huIgG concentrations in plasma and brain were quantified using a generic anti-human IgG sandwich-format ELISA. Briefly, plates were coated overnight at 4° C. with donkey anti-human IgG (JIR #709-006-098) at 1 μg/mL in sodium bicarbonate solution (Sigma #C3041-50CAP) with gentle agitation. Plates were then washed 3× with wash buffer (PBS+0.05% Tween 20). Assay standards and samples were diluted in PBS+0.05% Tween 20+1% BSA (10 mg/mL). Standard curve preparation ranged from 0.003 to 10 nM (BLQ<0.03 nM). Standards and diluted samples were incubated with agitation for 2 hr at room temperature. After incubation, plates were washed 3× with wash buffer. Detection antibody, goat anti-human IgG (JIR #109-036-098), was diluted in blocking buffer (PBS+0.05% Tween 20+5% BSA (50 mg/mL)) to a final concentration of 0.02 μg/mL and plates were incubated with agitation for 1 hr at room temperature. After a final 3× wash, plates were developed by adding TMB substrate and incubated for 5-10 minutes. Reaction was quenched by adding 4 N H₂SO₄ and read using 450 nM absorbance. After our original analysis, it was determined that in this assay, dual TVs are more accurately quantified using a dual TV standard. The graphs in FIG. 7 present updated measurements of the samples also presented in FIG. 6.

Dual transporters that engage both CD98hc and TfR exhibit both higher maximal brain concentrations, and/or prolonged brain exposure as compared to the corresponding single binding ATVs. For example, at a 50 mg/kg dose level the single ATV TfR binder (TV35.23.4) had a maximum brain concentration of about 20 nM, whereas the single ATV CD98hc binder (LLB2) had a maximum brain concentration of about 25 nM. In contrast, the corresponding dual construct had a maximum brain concentration of about >100 nM (updated from previously determined maximum value of 70 nM), demonstrating an effect that is far more than additive. This greater than additive effect persisted until at least one week. This effect is even more dramatic when considering that the peripheral concentrations of the dual transporters are far less than either of the single TVs at all timepoints.

TABLE 23A
Brain huIgG concentration (nM) for 50 mg/kg dose (see FIG. 6.)

| | | | | Dual transporter | Dual transporter |
|---|---|---|---|---|---|
| | Control | TfR alone | CD98hc alone | TfR TV35/ | TfR TV42/ |
| Timepoint | IgG | (TV35.23.4) | (LLB2.10.8) | CD98hc LLB2 | CD98hc LLB2 |
| 24 | 3.73 | 18.54 | 8.91 | 37.65 | 30.37 |
| 72 | 7.09 | 16.83 | 19.57 | 66.8 | 33.68 |
| 120 | 5.57 | 11.48 | 25.95 | 69.27 | 25.53 |
| 168 | 2.67 | 7.03 | 24.88 | 48.29 | 5.02 |

TABLE 23B
Revised determination of Brain huIgG concentration
(nM) for 50 mg/kg dose (see FIG. 7).

| | | | | Dual transporter | Dual transporter |
|---|---|---|---|---|---|
| | Control | TfR alone | CD98hc alone | TfR TV35/ | TfR TV42/ |
| Timepoint | IgG | (TV35.23.4) | (LLB2.10.8) | CD98hc LLB2 | CD98hc LLB2 |
| 24 | 6.69 | 21.21 | 12.06 | 51.10 | 45.71 |
| 72 | 8.25 | 16.62 | 20.40 | 84.43 | 41.37 |
| 120 | 6.95 | 11.07 | 23.19 | 107.04 | 29.97 |
| 168 | 4.72 | 9.00 | 27.55 | 63.75 | 10.47 |

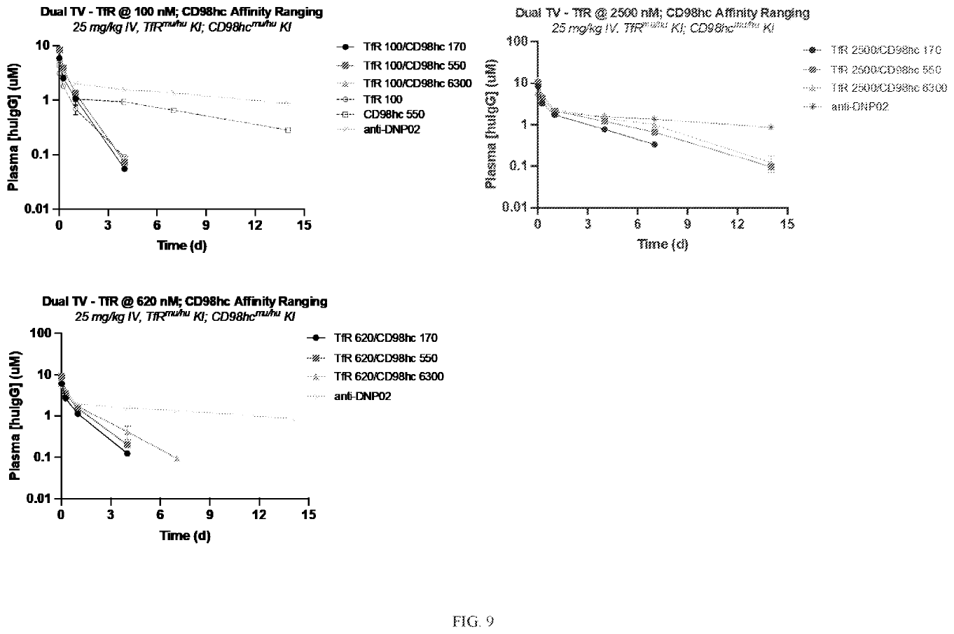

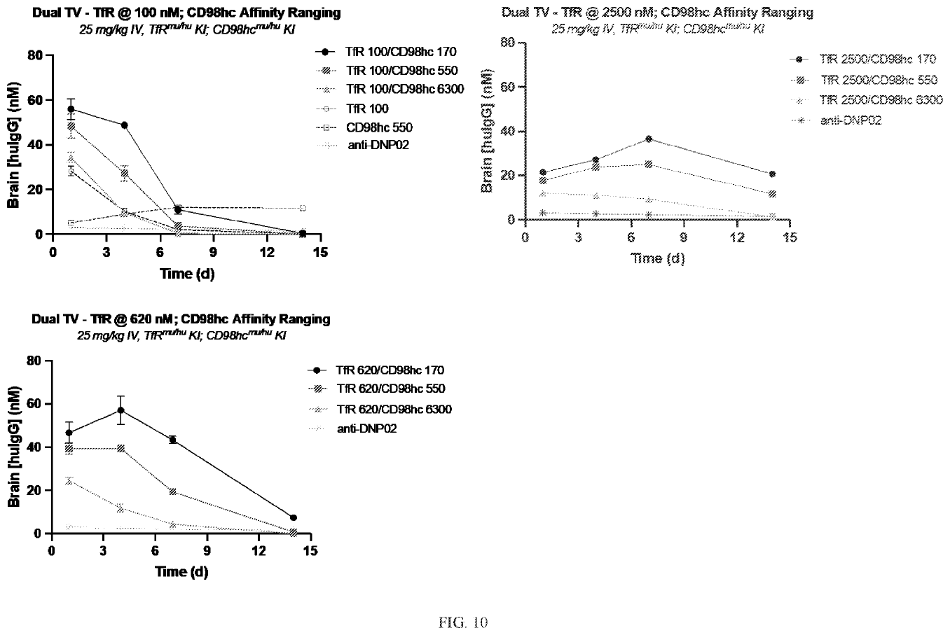

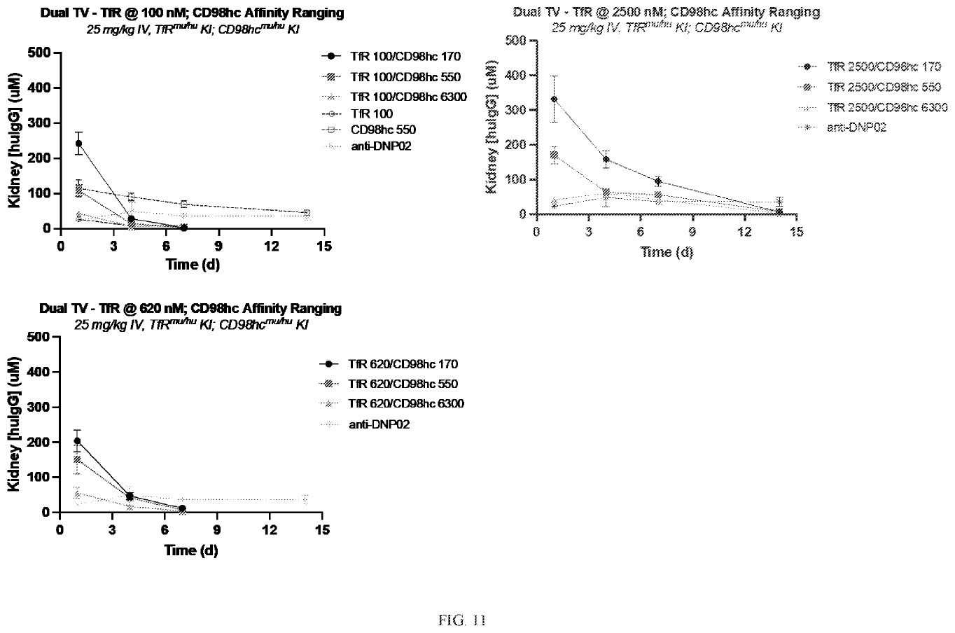

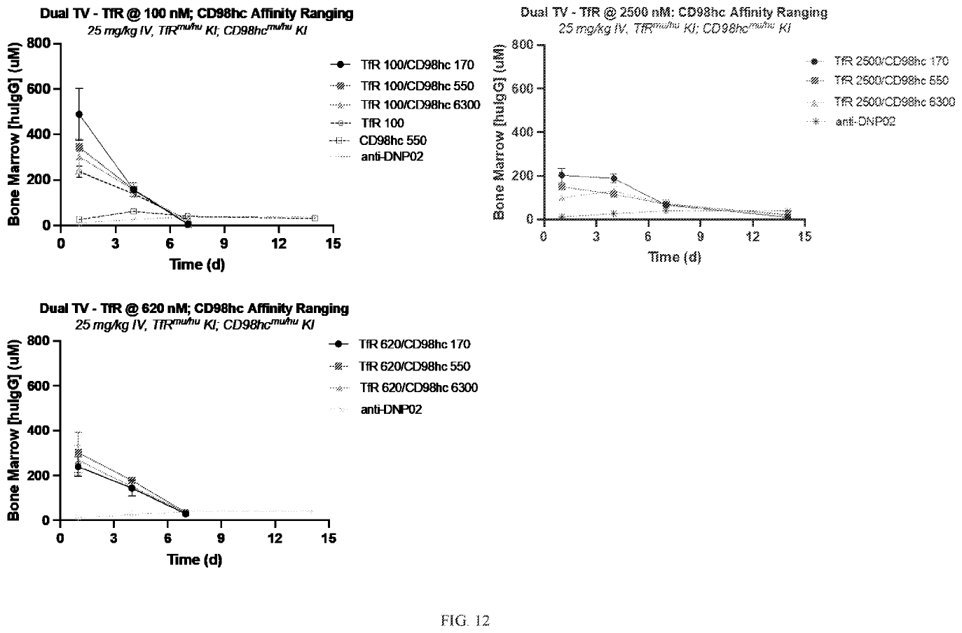

The plasma, brain, kidney, and bone marrow pharmacokinetic (PK) profiles of dual transporter variants with varying TfR and CD98hc affinities were assessed in TfR^{mu/hu} KI CD98hc^{mu/hu} KI mice (FIG. 9-12). Mice were intravenously dosed with 25 mg/kg of dual TVs, ATV^{TfR}, ATV^{CD98hc}, or control IgG. huIgG concentrations in plasma, kidney, and bone marrow were measured as described above. Brain concentrations were quantified using a generic anti-human IgG sandwich-format electrochemiluminescence immunoassay (ECLIA) on a Meso Scale Discovery (MSD) platform. Briefly. 1% casein-based PBS blocking buffer (Thermo Scientific, 37528) was added to an MSD GOLD 96-well smallspot streptavidin-coated microtiter plate (Meso Scale Discovery, L45SA) and incubated for approximately 1 h. Following the plate blocking and wash steps, biotinylated goat anti-human IgG (SouthernBiotech 2049-08) at a working concentration of 0.5 μg/mL was added to coat the assay plate and allowed to incubate for 1-2 hr. Subsequently, test samples were diluted (MRD of 1:100 in 0.5% casein-based PBS assay buffer) and added to the assay plate. Following the 1-2 hr incubation in the capture step, a pre-adsorbed secondary ruthenylated (SULFO-TAG) goat anti-human IgG antibody (Meso Scale Discovery. R32AJ) at a working solution of 0.5 μg/mL was added to the assay plate and incubated for approximately 1 hr. An assay read buffer (1×MSD Read Buffer T, R92TC) was then added to generate the electrochemiluminescence (ECL) assay signal, expressed in ECL units (ECLU). All assay reaction steps were performed at ambient temperature with shaking on a plate shaker (where appropriate), brain lysate sample concentrations were back calculated off the standard curve, which was fitted with a weighed four-parameter non-linear logistic regression.

Brain concentrations of dual transporters at early times was correlated with stronger TfR affinity; whereas higher dual transporter concentrations at later timepoints was correlated with stronger CD98hc affinity. Specific brain exposure kinetics can be optimized by modulating transport (e.g., TfR) and retention (e.g., CD98c) affinities to drive varying amounts of brain uptake and retention. Increasing TfR affinity can increase BBB transport. However, increasing TfR affinity can also reduce brain retention.

Peripheral distribution to bone marrow was correlated with TfR affinity and not substantially impacted by CD98hc affinity. Similarly, kidney localization was correlated with CD98hc affinity and was only slightly reduced with strong TfR affinity. These data indicated that the peripheral distribution of dual transporters reflect the distribution of each target combined.

### Example 10

Following 25 mg/kg IV dose of dual transporters with varying TfR and CD98hc affinities, mice were perfused with PBS, and hemi-brains were drop fixed in 4% PFA overnight. Sagittal brain sections (40 μm) were cut using a microtome (MultiBrain® Technology by NeuroScience Associates), blocked for 2 hr at room temperature in 5% BSA+0.3% Triton X-100. Sections were probed with goat anti-huIgG-Alexa-647 (Jackson ImmunoResearch 709-606-149, 1:500), rabbit anti-Aquaporin4 (Millipore, AB2218 1:500), and mouse anti-NeuN (Millipore MAB377, 1:500) overnight at 4° C. Then sections were incubated with by donkey anti-rabbit-488 (Thermofisher A-21206, 1:500) and goat anti-mouse IgG1 (ThermoFisher A-21124, 1:500) overnight at 4° C. Sections were washed and mounted with ProLong Glass (ThermoFisher P36982). Images of whole sagittal sections were captured using a Zeiss Axioscan ZI with a 20× objective, and representative confocal images of the cortex were taken on a Leica SP8 Lightning confocal microscope with a 25× objective.

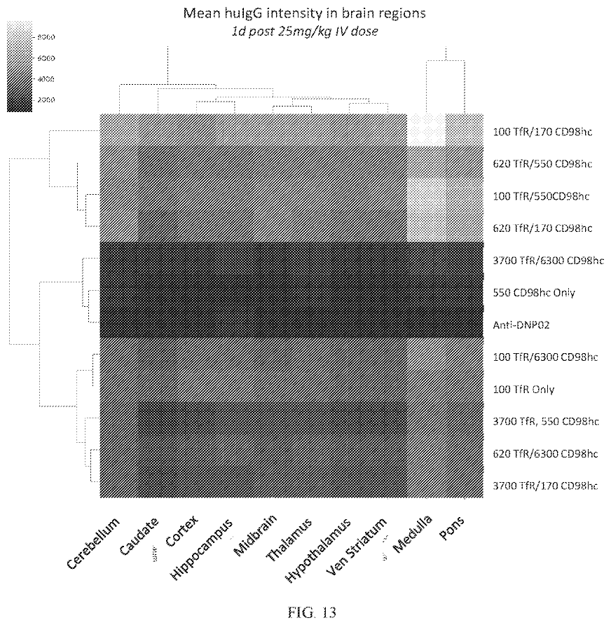

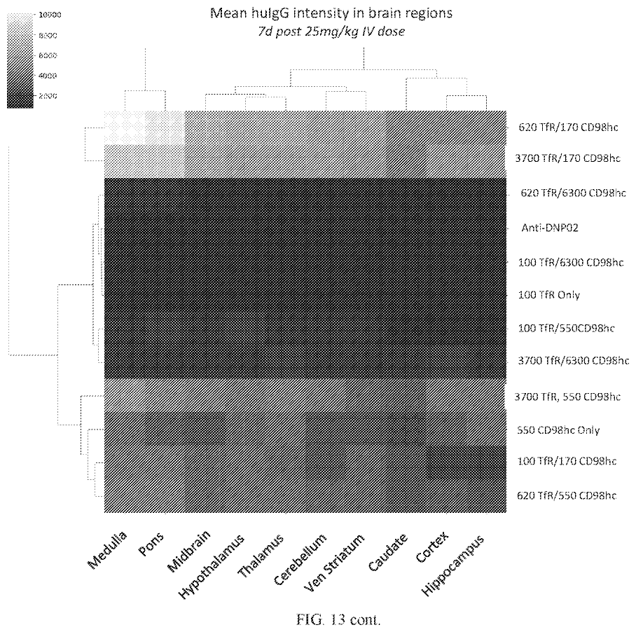

Images of the sagittal sections were registered to a brain atlas using the rigid, affine, and bspline warps in itk-elastix v0.19.1 (K. Ntatsis et al. “itk-elastix: Medical image registration in Python”, Proceedings of the 22nd Python in Science Conference, pp. 101-105, 2023) then quantified using the regionprops tools in scikit-image v0.22.0 (Stefan van der Walt S et al. “scikit-image: Image processing in Python. PeerJ 2:e453 (2014)) and mean huIgG intensities in various brain regions are presented in FIG. 13. These data demonstrate that dual TVs broadly distribute across brain regions and have relatively enhanced distribution to brain stem regions (medulla and pons) not observed with TfR and CD98hc only binding TVs.

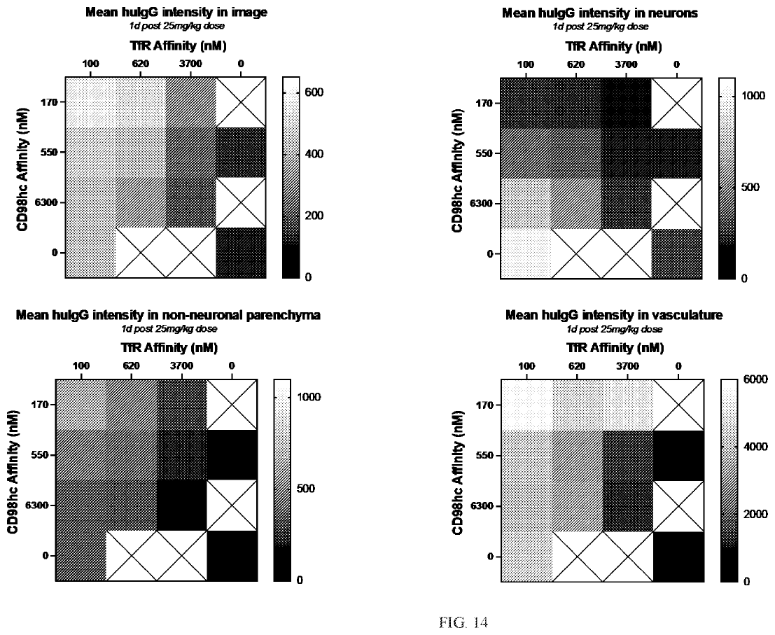

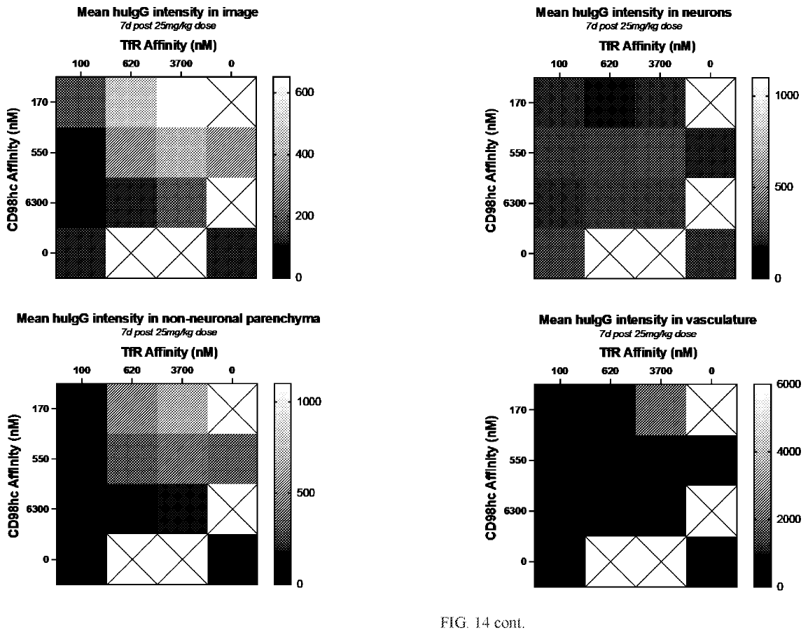

For confocal images of the cortex, bright, large aquaporin 4 positive areas were used to segment the vasculature, and NeuN positive areas were segmented as neurons using custom code built using the ndimage filtering and thresholding functions in scipy v1.11.4 (Pauli Virtanen P et al. “SciPy 1.0: Fundamental Algorithms for Scientific Computing in Python.” Nature Methods, 17(3), 261-272 (2020)). Tissue outside the vascular area was designated parenchymal, and non-neuronal parenchyma area was defined as non-vascular and non-neuronal. Mean huIgG intensity for each of these segments are presented in FIG. 14. Consistent with PK measurements, strong TfR affinity is correlated with brighter anti-huIgG staining at 1 day post dose and stronger CD98hc affinity is correlated with brighter anti-huIgG staining at 7 days post dose. The intensity of anti-huIgG staining was bright in the vascular at 1 day post-dose suggestive of high brain uptake at that timepoint. 7 days post-dose most of the anti-huIgG staining was observed in the non-neuronal parenchymal area, consistent with CD98hc driven localization and retention. Neuronal localization was correlated with TfR affinity; however, increasing CD98hc affinity decreased TfR mediated neuronal localization. These data indicate modulating TfR and CD98hc affinity results in dual TVs with CNS cell type biodistribution and kinetics distinct from the profiles achieved by targeting TfR or CD98hc alone. For example, the dual TV with 100 nM affinity to TfR and 170 nM for CD98hc has high brain exposure 1 day post dose, but does not appear to localize to neurons.

### Example 11

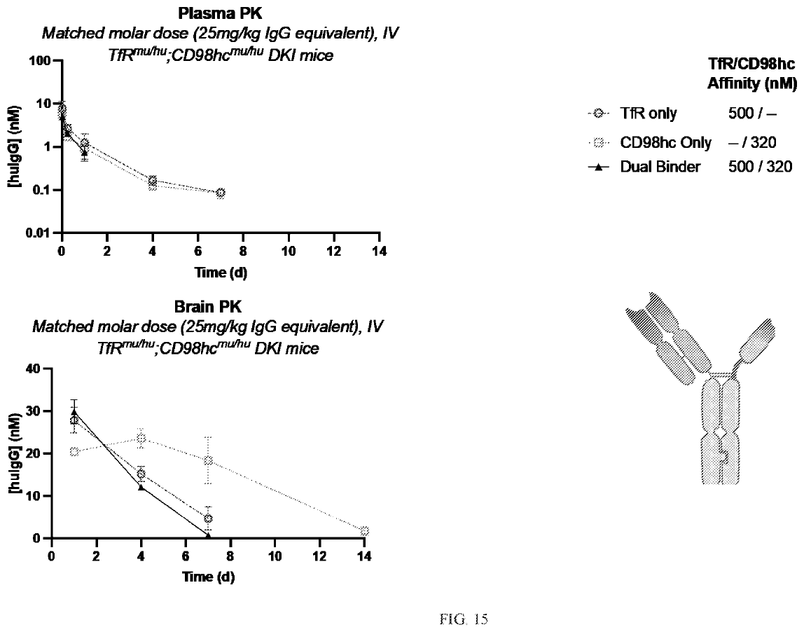

The plasma and brain pharmacokinetic (PK) profiles of Fab-Fc/VHH-Fc dual transporter variants were assessed in TfR^{mu/hu} KI; CD98hc^{mu/hu} KI mice. Mice were intravenously dosed with 25 mg/kg of dual binding Fab-Fc/VHH-Fc, TfR only binding Fab-Fc, or CD98hc only binding VHH-Fc. Concentrations of huIgG were measured by ELISA as described above. In this format, additive brain exposure of the dual binding molecule was not observed compared to the TfR and CD98hc only binding control molecules (FIG. 15).

### Example 12

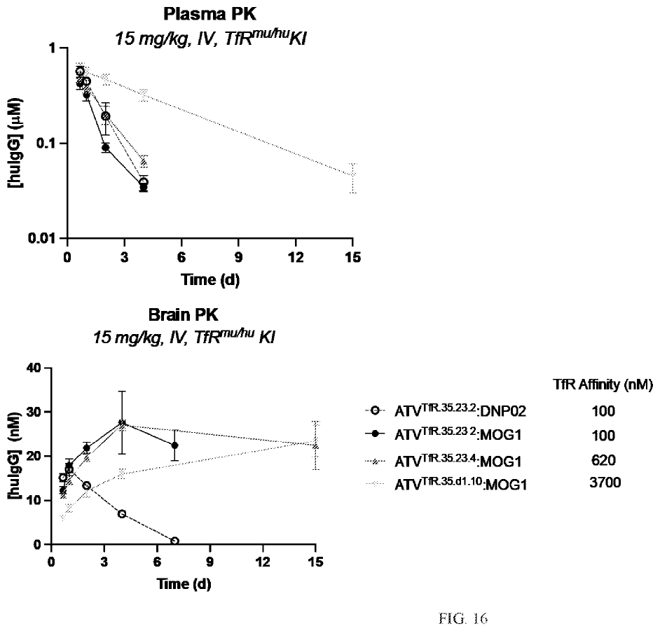

The plasma and brain pharmacokinetic (PK) profiles of ATV^{TfR}:MOG1 variants were assessed in TfR^{mu/hu} KI mice. The MOG1 Fab (“MOG01”, SEQ ID NO. 9) was used as described in US 2019/0352397. Mice were intravenously dosed with 15 mg/kg of ATV^{TfR}:MOG1 variants. Concentrations of huIgG were measured by ELISA as described above. These data demonstrate that a TfR and MOG1 dual binding molecule undergo brain uptake via TfR while MOG1 reduces TfR mediated clearance from brain resulting in prolonged brain exposure (FIG. 16).

1. A dual transporter comprising:
(a) a first binding region that specifically binds to a blood brain barrier (BBB) transport protein, wherein the BBB transport protein is the transferrin receptor (TfR) or CD98hc; and
(b) a second binding region that specifically binds to a second protein wherein the second protein is expressed on the luminal surface of the BBB or is a brain retention protein, and wherein the second protein is different from the BBB transport protein.

2. The dual transporter of claim 1, wherein the second protein is selected from the group consisting of: TfR, CD98hc, Large neutral amino acids transporter small subunit 1 (CD98 light chain), glucose transporter 1 (GLUT1), major facilitator superfamily domain-containing protein 2A (MFSD2A), carbonic anhydrase IV (CA-IV), Low density lipoprotein receptor, Insulin-like growth factor 1 receptor (IGF1R), Insulin-like growth factor 2 receptor, IgG receptor FcRn large subunit p51, Low density lipoprotein receptor-related protein 1, Low density lipoprotein receptor-related protein 2, Insulin receptor, Cell cycle control protein 50A, Transmembrane protein 50A, Basigin, Leptin Receptor, Claudin-5, P-selectin, Lactoferrin receptor, Folate receptor, Sodium-dependent lysophosphatidylcholine symporter 1, Solute carrier organic anion transporter family member 1C1, Sodium-coupled neutral amino acid transporter 5, LDL receptor-related protein 8, High affinity cationic amino acid transporter 1, Sodium- and chloride-dependent taurine transporter, Insulin-like growth factor-binding protein 7, Solute carrier family 40 member 1, Zinc transporter 6, heparin-binding epidermal growth factor-like growth factor, and Myelin-oligodendrocyte glycoprotein (MOG).

3. The dual transporter of claim 1, wherein the second protein is a brain retention protein.

4. The dual transporter of claim 3, wherein the brain retention protein is CD98hc, MOG, GLUT1, IGF1R, or CA-IV, or MFSD2A.

5. The dual transporter of any one of claims 1-4, wherein the transferrin receptor is human TfR1.

6. The dual transporter of any one of claims 1-4, wherein the CD98hc is human CD98hc.

7. The dual transporter of claim 2, wherein
(a) the BBB transport protein is human TfR1 and the second protein is human CD98hc;
(b) the BBB transport protein is human TfR1 and the second protein is human MOG;
(c) the BBB transport protein is human TfR1 and the second protein is human GLUT1;
(d) the BBB transport protein is human TfR1 and the second protein is human IGF1R;
(d) the BBB transport protein is human TfR1 and the second protein is human CA-IV; or
(d) the BBB transport protein is human TfR1 and the second protein is human MFSD2A.

8. The dual transporter of any one of claims 1-7, wherein the second protein undergoes receptor-mediated transcytosis.

9. The dual transporter of any one of claims 1-8, wherein the first binding region comprises:
(a) a first peptide that specifically binds the BBB transport protein;
(b) a first antibody antigen binding domain that specifically binds the BBB transport protein;
(c) a first Fc polypeptide modified to specifically bind the BBB transport protein;
(d) a first CH3 peptide modified to specifically bind the BBB transport protein;
(e) a first fibronectin type III domain peptide modified to specifically bind the BBB transport protein; or
(f) a first bicyclic peptide that specifically binds the BBB transport protein.

10. The dual transporter of claim 9, wherein the first antibody antigen binding domain comprises: an antibody, a single chain antibody, a F(ab′)2 fragment, a Fab fragment, a single chain Fab (scFab), a Fv fragment, a single chain variable fragment (scFv), or a heavy chain only antibody variable domain (nanobody).

11. The dual transporter of any one of claims 1-10, wherein the second binding region comprises:
(a) a second peptide that specifically binds the second protein,
(b) a second antibody antigen binding domain that specifically binds the second protein,
(c) a second Fc polypeptide modified to specifically bind the second protein,
(d) a second CH3 peptide modified to specifically bind the second protein,
(e) a second fibronectin type III domain peptide modified to specifically bind the second protein, or
(f) a second bicyclic peptide that specifically binds the second protein.

12. The dual transporter of claim 11, wherein the second antibody antigen binding domain comprises: an antibody, a single chain antibody, a F(ab′)2 fragment, a Fab fragment, a single chain Fab (scFab), a Fv fragment, a single chain variable fragment (scFv), or a heavy chain only antibody variable domain (nanobody).

13. The dual transporter of any one of claims 1-12, wherein the first binding region comprises a first antibody antigen binding domain that specifically binds the BBB transport protein and the second binding region comprises a second antibody antigen binding domain that specifically binds the second protein.

14. The dual transporter of claim 13, comprising a bispecific antibody selected from the group consisting of a full-length IgG bispecific antibody, a bispecific heavy chain antibody, and a bispecific F(ab′)2 comprising the first antibody antigen binding domain and the second antibody antigen binding.

15. The dual transporter of claim 13, wherein the first binding region comprises a Fab fragment, a scFab, a Fv fragment, a scFv, or a nanobody and the second binding region comprises a Fab fragment, scFab, a Fv fragment, a scFv, or a nanobody.

16. The dual transporter of claim 15, wherein
(a) the first binding region comprises a Fab fragment and the second binding region comprises a Fab fragment;
(b) the first binding region comprises a scFab and the second binding region comprises a scFab;
(c) the first binding region comprises a Fv fragment and the second binding region comprises a Fv fragment;
(d) the first binding region comprises a scFv and the second binding region comprises a scFv; or
(e) the first binding region comprises a nanobody and the second binding region comprises a nanobody.

17. The dual transporter of claim 11 or 12, wherein
(a) the first binding region comprises the first antibody antigen binding domain and the second binding region comprises the second peptide, the second Fc polypeptide, the second CH3 peptide, the second fibronectin type III domain peptide, or the second bicyclic peptide; or
(b) the first binding region comprises the first peptide, the first Fc polypeptide, the first CH3 peptide, the first fibronectin type III domain peptide, or the first bicyclic peptide and the second binding region comprises the second antibody antigen binding domain.

18. The dual transporter of claim 11 or 12, wherein
(a) the first binding region comprises the first Fc polypeptide and the second binding region comprises the second peptide, the second antibody antigen binding domain, the second Fc polypeptide, the second CH3 peptide, the second fibronectin type III domain peptide, or the second bicyclic peptide;
(b) the first binding region comprises the first CH3 peptide and the second binding region comprises the second peptide, the second antibody antigen binding domain, the second Fc polypeptide, the second CH3 peptide, the second fibronectin type III domain peptide, or the second bicyclic peptide;
(c) the first binding region comprises the first peptide, the first antibody antigen binding domain, the first Fc polypeptide, the first CH3 peptide, the first fibronectin type III domain peptide, or the first bicyclic peptide and the second binding region comprises the second Fc polypeptide; or
(d) the first binding region comprises the first peptide, the first antibody antigen binding domain, the first Fc polypeptide, the first CH3 peptide, the first fibronectin type III domain peptide, or the first bicyclic peptide and the second binding region comprises the second CH3 peptide.

19. The dual transporter of claim 11 or 12, wherein
(a) the first binding region comprises the first Fc polypeptide and the second binding region comprises the second Fc polypeptide, optionally wherein the first Fc polypeptide and the second Fc polypeptide form a Fc dimer;
(b) the first binding region comprises the first CH3 peptide and the second binding region comprises the second CH3 polypeptide;
(c) the first binding region comprises the first fibronectin type III domain peptide and the second binding region comprises the second fibronectin type III domain peptide; or
(d) the first binding region comprises the first bicyclic peptide and the second binding region comprises the second bicyclic peptide.

20. The dual transporter of any one of claims 1-19, wherein the first binding region is linked to the second binding region.

21. The dual transporter of any one of claims 1-19, wherein
(a) the first binding region forms a dimer with the second binding region;
(b) the first binding region is linked to a polypeptide that forms a dimer with the second binding region;
(c) the second binding region is linked to a polypeptide that forms a dimer with the first binding region; or
(d) the first binding region is linked to a first polypeptide and the second binding region is linked to a second polypeptide, wherein the first polypeptide and the second polypeptide form a dimer.

22. The dual transporter of any one of claims 1-19, wherein the first binding region, the second binding region, or both the first binding region and the second binding region are linked to a scaffold.

23. The dual transporter of claim 22, wherein the scaffold comprises an antibody or a fragment thereof.

24. The dual transporter of claim 23, wherein the scaffold comprises an antibody and wherein
(a) the first binding region is linked to a heavy chain of the antibody, the second binding region is linked to a heavy chain of the antibody, or the first and the second binding regions are both linked to a heavy chain of the antibody, optionally wherein the first binding region is linked to the C terminal end of a heavy chain of the antibody and/or the second binding region is linked to the C terminal end a heavy chain of the antibody;
(b) the first binding region is linked to a Fc region of the antibody, the second binding region is linked to a Fc region of the antibody, or the first and the second binding regions are both linked to a Fc region of the antibody, optionally wherein the first binding region is linked to the C terminal end of a Fc region of the antibody and/or the second binding region is linked to the C terminal end a Fc region of the antibody;
(c) the first binding region is linked to a light chain of the antibody, the second binding region is linked to a light chain of the antibody, or the first and the second binding regions are both linked to a light chain of the antibody, optionally wherein the first binding region is linked to the C terminal end of a light chain of the antibody and/or the second binding region is linked to the C terminal end of a light chain of the antibody; or
(d) the first binding region is linked to a light chain of the antibody and the second binding region is linked to a heavy chain of the antibody or the first binding region is linked to a heavy chain of the antibody and the second binding region is linked to a light chain of the antibody.

25. The dual transporter of claim 24, wherein the first binding region comprises a Fab fragment, a scFab, a Fv fragment, a scFv, a nanobody, and the second binding region comprises a Fab fragment, a scFab, a Fv′ fragment, a scFv, or a nanobody.

26. The dual transporter of claim 24 or 25, wherein the scaffold is a full-length IgG antibody that binds to a therapeutic brain target, and the first and the second binding regions are each attached to a respective C-terminal end of a heavy chain of the full-length IgG antibody.

27. The dual transporter of claim 26, wherein the first and second binding regions are each an antibody antigen binding domain.

28. The dual transporter of claim 27, wherein each antibody antigen binding domain independently comprises a Fab fragment or an scFab.

29. The dual transporter of claim 27, wherein the first and second binding regions are each an scFv.

30. The dual transporter of any one of claims 1-29, wherein the dual transport binds to the BBB transport protein with greater affinity than the dual transporter binds to the second protein, optionally wherein the dual transporter binds to the BBB transport protein with an affinity of about 50 nM to about 1000 nM and binds to the second protein with an affinity of about 5- to about 10-fold less than the affinity to the BBB transport protein.

31. The dual transporter of any one of claims 1-29, wherein the dual transport binds to the BBB transport protein with lesser affinity than the dual transporter binds to the second protein, optionally wherein the dual transporter binds to the BBB transport protein with an affinity of about 50 nM to about 6000 nM and binds to second protein with an affinity of about 5- to about 10-fold greater than the affinity to BBB transport protein.

32. The dual transporter of any one of claims 1-29, wherein the first binding region has moderate or weak affinity to the BBB transport protein and the second binding region has strong affinity to the second protein.

33. The dual transporter of any one of claims 24-29, wherein the first and second binding regions bind hTfR1 and human CD98hc, respectively.

34. The dual transporter of claim 33, wherein the first binding region comprises the CDRs or the variable chain regions of a Brain shuttle anti-TfR antibody, a TfR12 anti-TfR antibody, a TfR13 anti-TfR scFv, a 13E4v2ii anti-TfR antibody, a JC-141 anti-TfR antibody, a JC-171 anti-TfR antibody, a 17H10 anti-TfR Fab or scFv, or a 17H10.1 anti-TfR Fab or scFv.

35. The dual transporter of any one of claim 33 or 34, wherein the second binding region comprises the CDRs or the variable chain regions of 1C03-4 anti-CD98hc VHH, a 1C03-5 anti-CD98hc VHH, a CD98hc2 anti-CD98hc nanobody, a CD98hc4 anti-CD98hc scFv, or a CD98hc5 anti-CD98hc scFv.

36. The dual transporter of any one of claims 23-25, wherein the scaffold comprises a full-length antibody directed to a therapeutic brain target and the first and second binding regions are each respectively linked to a C-terminal end of a light chain of the full-length antibody.

37. The dual transporter of any one of claims 23-25, wherein the antibody comprises a bispecific antibody.

38. The dual transporter of any one of claims 23-37, wherein the antibody specifically binds to a therapeutic target.

39. The dual transporter of any one of claims 23-25 and 37-38, wherein the dual transport molecule comprises a bispecific antibody, wherein
(a) the bispecific antibody specifically binds to the BBB transport protein and specifically binds to a therapeutic target and wherein the second binding region is linked to the bispecific antibody; or
(b) the bispecific antibody specifically binds to the second protein and specifically binds to a therapeutic target and wherein the first binding region is linked to the bispecific antibody.

40. The dual transporter of any one of claims 1-13, wherein the dual transport molecule comprises a tri-specific antibody, a tri-specific antibody-like molecule, a tetra-specific antibody, or tetra-specific antibody-like molecule.

41. The dual transporter of any one of claims 1-13, wherein the dual transport molecule comprises a DVD-Lg, wherein a first Fab arm of the DVD-Ig comprises a first Fv fragment that specifically binds to a first target protein and a second Fv fragment that specifically binds to the BBB transport protein, and a second Fab arm of the DVD-Ig comprises a third Fv fragment that specifically binds to a second target protein and a fourth Fv fragment that specifically binds to the second protein.

42. The dual transporter of claim 19, wherein the first binding region comprises the first Fc polypeptide and the second binding region comprises the second Fc polypeptide, wherein the first Fc polypeptide and the second Fc polypeptide from a Fc dimer, and wherein the Fc dimer is linked to one or more Fabs, one or more F(ab′)2s, one or more scFabs, one or more scFvs, or one or more nanobodies, optionally wherein the Fab, F(ab′)2, scFab, scFv, or nanobody comprises a therapeutic Fab, F(ab′)2, scFab, scFv, or nanobody.

43. The dual transporter of any one of claims 1-42, wherein the dual transport molecule is linked to a therapeutic agent.

44. The dual transporter of claim 18 or 19, wherein the dual transport molecule comprises a therapeutic antibody comprising the first Fc polypeptide or CH3 peptide modified to specifically bind the BBB transport protein and the second Fc polypeptide or CH3 peptide modified to specifically bind a second protein.

45. The dual transporter of any one of claims 1-4, 6-19, and 44, wherein the BBB transport protein comprises TfR.

46. The dual transporter of claim 45, wherein the first binding region binds to the apical domain of TfR.

47. The dual transporter of claim 46, wherein first binding region binds TfR without inhibiting binding of transferrin to the TfR.

48. The dual transporter of any one of claims 45-47, wherein the first binding region comprises a first antibody antigen binding domain that specifically binds TfR.

49. The dual transporter of claim 48, wherein the first antibody antigen binding domain comprises the CDR sequences of a JC-141 anti-TfR antibody, a JC-171 anti-TfR antibody, a Brain shuttle anti-TfR antibody, a 13E4v2ii anti-TfR antibody, a TfR12 anti-TfR antibody, a TfR13 anti-TfR antibody, a 17H10 anti-TfR Fab or scFv, or a 17H10.1 anti-TfR Fab or scFv.

50. The dual transporter of claim 49, wherein the first antibody antigen binding domain comprises an antibody antigen binding domain from the JC-141 anti-TfR antibody, the JC-171 anti-TfR antibody, the Brain shuttle anti-TfR antibody, the 13E4v2ii anti-TfR antibody, the TfR12 anti-TfR antibody, the TfR13 anti-TfR antibody, the 17H10 anti-TfR Fab or scFv, or the 17H10.1 anti-TfR Fab or scFv.

51. The dual transporter of any one of claims 45-47, wherein the first binding region comprises a first Fc polypeptide or a first CH3 peptide modified to specifically bind TfR.

52. The dual transporter of any one of claims 45-51, wherein the first binding region specifically binds TfR with an affinity of less than about 100 nM.

53. The dual transporter of any one of claims 45-51, wherein the first binding region specifically binds TfR with an affinity of about 100 nM to about 1000 nM or about 100 nM to about 600 nM.

54. The dual transporter of any claim 45-51, wherein the first binding region specifically binds TfR with an affinity of about 600 nM to about 6000 nM or about 600 nM to about 1000 nM.

55. The dual transporter of claim 51, wherein the first CH3 peptide modified to specifically bind TfR comprises five, six, seven, eight, nine, ten, eleven, twelve, thirteen, fourteen, fifteen, or sixteen substitutions in a set of amino acid positions consisting of 380, 384, 386, 387, 388, 389, 390, 391, 392, 413, 414, 415, 416, 421, 424, and 426 wherein the positions are determined with reference to EU numbering.

56. The dual transporter of claim 55, wherein the substitutions are selected from: a L, Q, S, V, W, or Y at position 380; a F, L, M, P, V, W, Y, or I at position 384; a F, H, I, L, N, P, T, or V at position 386; a D, E, I, or V at position 387; a W, F, or Y at position 388; an A, G, I, S, T, or V at position 389; an A, D, E, F, G, H, K, L, Q, R, S, T, V, or Y at position 390; a F, Q, S, T, or V at position 391; a Q, F, or H at position 392; an A, E, H, I, L, P, S, or T at position 413; a R, G, or P at position 414; a D, E, G, T, P, Q, or R at position 415; a D, E, N, or T at position 416; a F, H, K, W, or Y at position 421; a T, W, E, or K at position 424; and a C, P, M, W, or G at position 426.

57. The dual transporter of claim 46, wherein the first CH3 peptide modified to specifically bind TfR comprises a F, L, M, P, V, W, Y, or I at position 384; a F, H, I, L, N, P, T, or V at position 386; a W, F, or Y at position 388; an A, G, I, S, T, or V at position 389; and; a F, H, K, W, or Y at position 421.

58. The dual transporter of claim 57, wherein the first CH3 peptide modified to specifically bind TfR further comprises one, two, three, four, five, six, seven, eight, nine, ten, or eleven substitutions selected from: a L, Q, S, V, W, or Y at position 380; a D, E, I, or V at position 387; an A, D, E, F, G, H, K, L, Q, R, S, T, V, or Y at position 390; a F, Q, S, T, or V at position 391; a Q, F, or H at position 392; an A, E, H, I, L, P, S, or T at position 413; a R, G, or P at position 414; a D, E, G, T, P, Q, or R at position 415; a D, E, N, or T at position 416; a T, W, E, or K at position 424; and a C, P, M, W, or G at position 426.

59. The dual transporter of claim 51, wherein the first CH3 peptide modified to specifically bind TfR comprises: four, five, six, seven, eight, or nine substitutions in a set of amino acid positions consisting of 384, 386, 387, 388, 389, 413, 415, 416, and 421; wherein the positions are determined with reference to EU numbering.

60. The dual transporter of claim 59, wherein the substitutions are selected from: a Y at position 384; a T at position 386; an E at position 387; a W at position 388; a V, S, or A at position 389; a T or S at position 413; an E at position 415; an E at position 416; and a F at position 421.

61. The dual transporter of claim 60, wherein the first CH3 peptide modified to specifically bind TfR comprises: a Y at position 384; a T at position 386; an E at position 387; a W at position 388; a V, S, or A at position 389; a T or S at position 413; an E at position 415; an E at position 416; and a F at position 421.

62. The dual transporter of any one of claims 55-61, wherein the first CH3 peptide modified to specifically bind TfR comprises Y at position 391, a K at position 392, a S at position 424; and S at position 426.

63. The dual transporter of any one of claims 55-61, wherein the first CH3 peptide modified to specifically bind TfR comprises (a) a polypeptide having at least 85%, at least 90%, at least 95%, or 100% identify to the amino acid sequence of any of SEQ ID NOs:6-14 and 124-125; or (b) a polypeptide having at least 85%, at least 90%, at least 95%, or 100% identify to the amino acids 111-217 of any of SEQ ID NOs:6-14 and 124-125.

64. The dual transporter of claim 51, wherein the first CH3 peptide modified to specifically bind TfR comprises six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, thirty, or thirty-one amino acid substitutions in a set of amino acid positions consisting of 378, 380, 382, 383, 384, 385, 386, 387, 388, 389, 390, 391, 414, 417, 419, 420, 421, 422, 424, 426, 427, 428, 429, 433, 434, 437, 438, 439, 440, 442, and 443, and 426; wherein the positions are determined with reference to EU numbering.

65. The dual transporter of claim 64, wherein the substitutions are selected from:
an E, L, or I at position 378; a N, R, Y, I, S, or F at position 380; a F at position 382; a Y, A, G, T, or a deletion at position 383; an A, D, E, F, G, or T at position 384; an A, D, or N at position 385; an A, G, K, N, S, or Y at position 386; a G, I, K, N, R, S, or T at position 387; a L, Q, or D at position 388; an I, P, Q, R, S, or T at position 389; a G, T, Y, or L at position 390; a S, T, I, L, or P at position 391; a P at position 414; a K at position 417; a P at position 419; a R or Q at position 420; an A, F, G, S, or Y at position 421; a L at position 422; an A at position 424; an E at position 426; an E at position 427; an E at position 428; a G at position 429; an E at position 433; a G at position 434; a D at position 437; a Y at position 438; a D, E, or S at position 439; a L at position 440; a G or W at position 442; and an E or Y at position 443.

66. The dual transporter of claim 65, wherein the first CH3 peptide modified to specifically bind TfR comprises a F at position 382, a L at position 422, an A at position 424, an E at position 426, a Y at position 438, and a L at position 440.

67. The dual transporter of claim 66, wherein the first CH3 peptide modified to specifically bind TfR further comprises one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, or twenty-five substitutions selected from: an E, L, or I at position 378; a N, R, Y, I, S, or F at position 380; a Y, A, G, T, or a deletion at position 383; an A, D, E, F, G, or T at position 384; an A, D, or N at position 385; an A, G, K, N, S, or Y at position 386; a G, I, K, N, R, S, or T at position 387; a L, Q, or D at position 388; an I, P, Q, R, S, or T at position 389; a G, T, Y, or L at position 390; a S, T, I, L, or P at position 391; a P at position 414; a K at position 417; a P at position 419; a R or Q at position 420; an A, F, G, S, or Y at position 421; an E at position 427; an E at position 428; a G at position 429; an E at position 433; a G at position 434; a D at position 437; a D, E, or S at position 439; a G or W at position 442; and an E or Y at position 443.

68. The dual transporter of any one of claims 64-67, wherein the first CH3 peptide modified to specifically bind TfR comprises (a) a polypeptide having at least 85%, at least 90%, at least 95%, or 100% identify to the amino acid sequence of any of SEQ ID NOs:57-64 and 126; or (b) a polypeptide having at least 85%, at least 90%, at least 95%, or 100% identify to the amino acids 111-217 of any of SEQ ID NOs:57-64 and 126.

69. The dual transporter of claim 51, wherein the first CH3 peptide modified to specifically bind TfR comprises four, five, six, seven, eight, nine, ten, eleven, twelve, thirteen, fourteen, fifteen, or sixteen substitutions in a set of amino acid positions consisting of: 378, 380, 382, 383, 384, 385, 386, 421, 422, 424, 426, 428, 434, 438, 440, and 442; wherein the positions are determined with reference to EU numbering.

70. The dual transporter of claim 69, wherein the substitutions are selected from: a D, E, F, H, N, Q, S, V, or Y at position 378; an A, D, F, H, I, K, L, M, Q, S, T, or Y at position 380; a G at position 382; a T at position 383; an A, E, F, H, I, K, L, Q, S, V, or Y at position 384; an I, T, or V at position 385; an A, H, N, S, T, or V at position 386; an A, F, H, K, L, M, Q, S, T, V, or Y at position 421; an A, F, H, I, K, L, R, T, or Y at position 422; an A, G, or P at position 424; an A, I, L, T, or V at position 426; an A or L at position 428; a S at position 434; an I, F, L, V, or Y at position 438; an A, G, I, M, N, P, T, or V at position 440:
and an A, K, M, R, T, or V at position 442.

71. The dual transporter of claim 70, wherein the first CH3 peptide modified to specifically bind TfR comprises: a G at position 382; an A, E, F, H, I, K, L, Q, S, V, or Y at position 384; an I, T, or V at position 385; and an I, F, L, V, or Y at position 438.

72. The dual transporter of claim 71, wherein the first CH3 peptide modified to specifically bind TfR further comprises one, two, three, four, five, six, seven, eight, nine, ten, eleven, twelve, thirteen, fourteen, fifteen, or sixteen substitutions selected from: a D, E, F, H, N, Q, S, V, or Y at position 378; an A, D, F, H, I, K, L, M, Q, S, T, or Y at position 380; a T at position 383; an A, H, N, S, T, or V at position 386; an A, F, H, K, L, M, Q, S, T, V, or Y at position 421; an A, F, H, I, K, L, R, T, or Y at position 422; an A, G, or P at position 424; an A, I, L, T, or V at position 426; an A or L at position 428; a S at position 434; an A, G, I, M, N, P, T, or V at position 440; and an A, K, M, R, T, or V at position 442.

73. The dual transporter of any one of claims 1-72, wherein the second protein comprises CD98hc.

74. The dual transporter of claim 73, wherein the second binding region comprises a second antibody antigen binding domain that specifically binds CD98hc.

75. The dual transporter of claim 74, wherein the second antibody antigen binding domain comprises the CDR sequences of: a 1C03-4 anti-CD98hc VHH, a 1C03-5 anti-CD98hc VHH, a CD98hc2 anti-CD98hc nanobody, a CD98hc4 anti-CD98hc scFv, or a CD98hc5 anti-CD98hc scFv.

76. The dual transporter of claim 75, wherein the second antibody antigen binding domain comprises: the 1C03-4 anti-CD98hc VHH, the 1C03-5 anti-CD98hc VHH, the CD98hc2 anti-CD98hc nanobody, the CD98hc4 anti-CD98hc scFv, or the CD98hc5 anti-CD98hc scFv.

77. The dual transporter of claim 73, wherein the second binding region comprises a second Fc polypeptide or a second CH3 peptide modified to specifically bind CD98hc.

78. The dual transporter of any one of claims 73-77, wherein the second binding region specifically binds CD98hc with an affinity of less than about 15 nM to about 5000 nM, or about 100 nM to about 500 nM.

79. The dual transporter of claim 77 or 78, wherein the second CH3 peptide modified to specifically bind CD98hc comprises five, six, seven, eight, or nine substitutions in a set of amino acid positions consisting of 382, 384, 385, 387, 422, 424, 426, 438, and 440;
wherein the positions are determined according to EU numbering.

80. The dual transporter of claim 79, wherein the substitutions are selected from: a R, Y, F, S, W, Y, K, or N at position 382; a L, Y, A, S, or F at position 384; a F, K, D, M, I, N, Y, L, or H at position 385; a N, L, Y, R, F, G, S, D, or T at position 387; an I, K, L, R, T, F, or H at position 422; a V, W, G, L, I, P, or Y at position 424; a D, A, Q, W, L, or P at position 426; an I, V, F, N, P, or S at position 438; and a K, T, P, I, or F at position 440.

81. The dual transporter of claim 77 or 78, wherein the second CH3 peptide modified to specifically bind CD98hc comprises at least five, six, seven, eight, or nine substitutions in a set of amino acid positions consisting of 382, 383, 384, 385, 386, 387, 389, 421, 422, 424, 426, 428, 434, 436, 438, 440, and 442; wherein the positions are determined according to EU numbering.

82. The dual transporter of claim 81, wherein the substitutions are selected from: a S or V at position 378; a D, M, N, P, F, or H at position 380; a R, Y, F, S, W, Y, K, or N at position 382; a T at position 383; a L, Y, A, S, or F at position 384; a F, K, D, M, I, N, Y, L, or H at position 385; a T, P, E, K, A, V, D, T, or F at position 386; a N, L, Y, R, F, G, S, D, or T at position 387; a T, Y, or F at position 389; a D, E, or Q at position 421; an I, K, L, R, T, F, or H at position 422; a V, W, G, L, I, P, or Y at position 424; a D, A, Q, W, L, or P at position 426; a L or Y at position 428; a S at position 434; a F at position 436; an I, V, F, N, P, or S at position 438; a K, T, P, I, or F at position 440; and Q or M at position 442.

83. The dual transporter of claim 77 or 78, wherein the second CH3 peptide modified to specifically bind CD98hc comprises at least eleven, twelve, thirteen, fourteen, or fifteen substitutions in a set of amino acid positions consisting of 378, 380, 382, 383, 384, 385, 386, 387, 389, 391, 421, 422, 424, 426, 428, 434, 436, 438, 440, 441, and 442; wherein the positions are determined according to EU numbering.

84. The dual transporter of claim 83, wherein the substitutions are selected from: a S, V, D, E, or Y at position 378; a L, I, M, A, Q, V, or K at position 380; a N, S, L, M, P, Y, K, A, or T at position 382; a T, F, N, P, D, L, H, or Q at position 383; a K, R, H, I, L, F, Y, V, or Q at position 384; a F or Y at position 385; a V, L, A, I, F, Y, S, T, H, R, or E at position 386; a L or I at position 387; a D, Q, A, T, H, or V at position 389; a T, V, or A at position 391; an E, Q, or A at position 421; a L, M, I, T, or P at position 422; an A at position 424; a N at position 426; a L, T, P, Y F, I, A, K, H, or W at position 428; a S at position 434; a L, V, H, F, P, R or W at position 436; a F or W at position 438; a L, P, E, N, V, A, I, or D at position 440; a P at position 441; and an A, V, M, Q, F, P, L, Y, K, R, H, or M at position 442.

85. The dual transporter of claim 77 or 78, wherein the second CH3 peptide modified to specifically bind CD98hc comprises eleven, twelve, thirteen, fourteen, or fifteen substitutions in a set of amino acid positions consisting of: 380, 382, 384, 385, 386, 387, 421, 422, 424, 426, 428, 436, 438, 440, and 442; wherein the positions are determined according to EU numbering.

86. The dual transporter of claim 85, wherein the substitutions are selected from: a L at position 380; a N at position 382; a R, H, or Q at position 384; a F or Y at position 385: a V, L, I, F, Y, or E at position 386; a L at position 387; an E, Q or A at position 421; an I, T, or P at position 422; an A at position 424; a N at position 426; a Y or W at position 428; a R or W at position 436; a F or W at position 438; a N at position 440; and an A, Q, K, R, H, or M at position 442.

87. The dual transporter of any one of claims 69-72, wherein the second CH3 peptide modified to specifically bind CD98hc comprises (a) a polypeptide having at least 85%, at least 90%, at least 95%, or 100% identify to the amino acid sequence of any of SEQ ID NOs:15-30; or (b) a polypeptide having at least 85%, at least 90%, at least 95%, or 100% identify to the amino acids 111-217 of any of SEQ ID NOs:15-30.

88. The dual transporter of claim 77 or 78, wherein the second CH3 peptide modified to specifically bind CD98hc comprises eight, nine, ten, eleven, twelve, thirteen, fourteen, fifteen, sixteen, seventeen, eighteen, or nineteen substitutions in a set of amino acid positions consisting of: 378, 380, 382, 383, 384, 385, 386, 387, 389, 421, 422, 424, 426, 428, 434, 436, 438, 440, and 442; wherein the positions are determined according to EU numbering.

89. The dual transporter of claim 88, wherein the substitutions are selected from: a S or V at position 378; a D, M, N, P, F, or H at position 380; a R, Y, F, S, W, Y, K, or N at position 382; a T at position 383; a L, Y, A, S, or F at position 384; a F, K, D, M, I, N, Y, L, or H at position 385; a T, P, E, K, A, V, D, T, or F at position 386; a N, L, Y, R, F, G, S, D, or T at position 387; a T, Y, or F at position 389; a D, E, or Q at position 421; a I, K, L, R, T, F, or H at position 422; a V, W, G, L, I, P, or Y at position 424; a D, A, Q, W, L, or P at position 426; a L or Y at position 428; a S at position 434; a F at position 436; a I, V, F, N, P, or S at position 438; a K, T, P, I, or F at position 440; and a Q or M at position 442.

90. The dual transporter of claim 77 or 78, wherein the second CH3 peptide modified to specifically bind CD98hc comprises eight, nine, ten, eleven, twelve, or thirteen substitutions in a set of amino acid positions consisting of 380, 382, 384, 385, 386, 387, 422, 424, 426, 428, 434, 438, and 440; wherein the positions are determined according to EU numbering.

91. The dual transporter of claim 90, wherein the substitutions are selected from: a D, M, N, P, F, or H at position 380; a R, Y, F, S, W, Y, K, or N at position 382; a L, Y, A, S, or F at position 384; a F, K, D, M, L N, Y, L, or H at position 385; a T, P, E, K, A, V, D, T, or F at position 386; a N, L, Y, R, G, S, D, or T at position 387; an I, K, R, T, F, or H at position 422; a V, W, G, L, I, P, or Y at position 424; a D, A, Q, W, L, or P at position 426; a L at position 428; a S at position 434; an I, F, N, P, or S at position 438; and a K, T, I, or F at position 440.

92. The dual transporter of claim 77 or 78, wherein the second CH3 peptide modified to specifically bind CD98hc comprises eight, nine, ten, eleven, twelve, thirteen, fourteen, fifteen, sixteen, seventeen, eighteen, or nineteen substitutions in a set of amino acid positions consisting of 378, 380, 382, 383, 384, 385, 386, 387, 389, 421, 422, 424, 426, 428, 434, 436, 438, 440, and 442; wherein the positions are determined according to EU numbering.

93. The dual transporter of claim 92, wherein the substitutions are selected from: a S or V at position 378; a D at position 380; a R at position 382; a T at position 383; a Y at position 384; a K at position 385; a P at position 386; a Y at position 387; a T, Y, or F at position 389; a D, E, or Q at position 421; an I at position 422; a V at position 424; a D at position 426; a L or Y at position 428; a S at position 434; a F at position 436; an I or V at position 438; a K at position 440; and a Q or M at position 442.

94. The dual transporter of any one of claims 88-93, wherein the first CH3 peptide modified to specifically bind TfR comprises (a) a polypeptide having at least 85%, at least 90%, at least 95%, or 100% identify to the amino acid sequence of SEQ ID NOs:34 or 35; or (b) a polypeptide having at least 85%, at least 90%, at least 95%, or 100% identify to the amino acids 111-217 of SEQ ID NOs: 34 or 35.

95. The dual transporter of any one of claims 1-94, wherein the dual transporter comprises an Fc polypeptide having one or more mutations that (a) promote heterodimer formation; (b) modulate effector function; or (c) extend serum half-life.

96. The dual transporter of any one of claims 1-32 and 36-72, wherein the first binding region specifically binds TfR and the second binding region specifically binds CD98hc.

97. The dual transporter of any one of claims 1-9, 11, 18-24, 30-32, 36-38, 42-47, 51, and 55-73, wherein the first binding region comprises a first Fc polypeptide or CH3 peptide modified to specifically bind TfR and the second binding region comprises a second Fc polypeptide or CH3 peptide modified to specifically bind CD98hc.

98. The dual transporter of any one of claims 77-97, wherein the first Fc polypeptide and the second Fc polypeptide form a Fc dimer.

99. The dual transporter of claim 98, wherein the dual transporter comprises an antibody comprising the first Fc polypeptide and the second Fc polypeptide.

100. The dual transporter of any one of claims 1-13, 16, 22-25, 30-38, 43, 45-50, and 73-76, wherein the first binding region comprises an anti-TfR antibody antigen binding domain linked to the C terminal end of a first light chain of an antibody and the second binding region comprises an anti-CD98hc antibody antigen binding domain linked to the C terminal end of a second light chain of the antibody.

101. The dual transporter of any one of claims 1-13, 16, 22-36, 37-38, 43, 45-50, and 73-76, wherein the first binding region comprises an anti-TfR antibody antigen binding domain linked to the C terminal end of a first heavy chain of an antibody and the second binding region comprises an anti-CD98hc antibody antigen binding domain linked to the C terminal end of a second heavy chain of the antibody.

102. The dual transporter of claim 100 or 101, wherein the antibody comprises a monospecific antibody, a bispecific antibody, a monospecific therapeutic antibody, or a bispecific therapeutic antibody.

103. The dual transporter of any one of claims 100-102, wherein the first binding region comprises a Fab fragment, a scFab, a Fv fragment, a scFv, a nanobody, and the second binding region comprises a Fab fragment, a scFab, a Fv fragment, a scFv, or a nanobody.

104. The dual transporter of any one of claims 1-16, 22-36, 43, 45-50, and 73-76, wherein the dual transporter comprises a bispecific antibody wherein one Fab arm of the antibody specifically binds TfR and the other Fab arm of the antibody specifically binds CD98hc.

105. The dual transporter of any one of claims 1-16, 22-25, 30-35, 37-40, 46-50, and 73-76, wherein the dual transporter comprises a bispecific antibody wherein one Fab arm of the antibody specifically binds TfR or CD98hc and the other Fab arm of the antibody specifically binds a target protein.

106. The dual transporter of any one of claims 1-12, 19, and 30-33, wherein
(a) the first binding region comprises a first fibronectin type III domain peptide modified to specifically bind TfR and the second binding region comprises a second fibronectin type III domain peptide modified to specifically bind, wherein the first and second binding regions are linked to an antibody; or
(b) the first binding region comprises a first bicyclic peptide that specifically binds TfR and the second binding region comprises a second bicyclic peptide that specifically binds CD98hc, wherein the first and second binding regions are linked to an antibody.

107. The dual transporter of any one of claims 1-18, 20-25, 30-35, 46-50, and 73-76, wherein the dual transporter comprises
(a) an anti-TfR monospecific or bispecific antibody linked to the second binding region; or
(b) an anti-CD98hc monospecific or bispecific antibody linked to the first binding region.

108. The dual transporter of any one of claims 1-29, 33-51, 55-77, and 79-107, wherein the first binding region has moderate or weak affinity to TfR and the second binding region has strong affinity to CD98hc.

109. The dual transporter of any one of claims 1-29, 33-51, 55-77, and 79-107, wherein
(a) the first binding region binds to TfR with an affinity of about 1000 nM to about 6000 nM and the second binding region binds to CD98hc with an affinity of about 1000 nM to about 6000 nM;
(b) the first binding region binds to TfR with an affinity of about 900 nM to about 6000 nM and the second binding region binds to CD98hc with an affinity of about 900 nM to about 6000 nM;
(c) the first binding region binds to TfR with an affinity of about 1000 nM to about 6000 nM and the second binding region binds to CD98hc with an affinity of about 300 nM to about 900 nM;
(d) the first binding region binds to TfR with an affinity of about 900 nM to about 6000 nM and the second binding region binds to CD98hc with an affinity of about 300 nM to about 900 nM;
(e) the first binding region binds to TfR with an affinity of about 1000 nM to about 6000 nM and the second binding region binds to CD98hc with an affinity of less than about 250 nM; or
(f) the first binding region binds to TfR with an affinity of about 900 nM to about 6000 nM and the second binding region binds to CD98hc with an affinity of about 50 nM to about 250 nM.

110. The dual transporter of any one of claims 1-29, 33-51, 55-77, and 79-107, wherein
(a) the first binding region binds to TfR with an affinity of about 300 nM to about 900 nM and the second binding region binds to CD98hc with an affinity of about 1000 nM to about 6000 nM;
(b) the first binding region binds to TfR with an affinity of about 300 nM to about 900 nM and the second binding region binds to CD98hc with an affinity of about 300 nM to about 900 nM;
(c) the first binding region binds to TfR with an affinity of about 300 nM to about 900 nM and the second binding region binds to CD98hc with an affinity of less than about 250 nM; or
(d) the first binding region binds to TfR with an affinity of about 300 nM to about 900 nM and the second binding region binds to CD98hc with an affinity of about 50 nM to about 250 nM.

111. The dual transporter of any one of claims 1-29, 33-51, 55-77, and 79-107, wherein
(a) the first binding region binds to TfR with an affinity of less than about 250 nM and the second binding region binds to CD98hc with an affinity of about 1000 nM to about 6000 nM;
(b) the first binding region binds to TfR with an affinity of less than about 250 nM and the second binding region binds to CD98hc with an affinity of about 300 nM to about 900 nM;
(c) the first binding region binds to TfR with an affinity of about 50 nM to about 250 nM and the second binding region binds to CD98hc with an affinity of about 300 nM to about 900 nM;
(d) the first binding region binds to TfR with an affinity of less than about 250 nM and the second binding region binds to CD98hc with an affinity of less than about 250 nM; or
(e) the first binding region binds to TfR with an affinity of about 50 nM to about 250 nM and the second binding region binds to CD98hc with an affinity of about 50 nM to about 250 nM.

112. The dual transporter of any one of claims 1-29, 33-51, 55-77, and 79-107, wherein
(a) the first binding region binds to TfR with an affinity of about 300 nM to about 6000 nM and the second binding region binds to CD98hc with an affinity of about 1000 nM to about 6000 nM;
(b) the first binding region binds to TfR with an affinity of about 300 nM to about 6000 nM and the second binding region binds to CD98hc with an affinity of about 900 nM to about 6000 nM;
(c) the first binding region binds to TfR with an affinity of about 300 nM to about 6000 nM and the second binding region binds to CD98hc with an affinity of about 300 nM to about 900 nM;
(d) the first binding region binds to TfR with an affinity of about 300 nM to about 6000 nM and the second binding region binds to CD98hc with an affinity of less than about 250 nM;
(e) the first binding region binds to TfR with an affinity of about 300 nM to about 6000 nM and the second binding region binds to CD98hc with an affinity of about 50 nM to 250 nM; or
(f) the first binding region binds to TfR with an affinity of about 300 nM to about 6000 nM and the second binding region binds to CD98hc with an affinity of about 50 nM to 900 nM.

113. The dual transporter of any one of claims 1-29, 33-51, 55-77, and 79-107, wherein
(a) the first binding region binds to TfR with an affinity of about 50 nM to about 1000 nM and the second binding region binds to CD98hc with an affinity of about 1 nM to about 500 nM;
(b) the first binding region binds to TfR with an affinity of about 100 nM to about 600 nM and the second binding region binds to CD98hc with an affinity of about 50 nM to about 300 nM; or
(c) the first binding region binds to TfR with an affinity of about 100 nM to about 400 nM and the second binding region binds to CD98hc with an affinity of about 50 nM to about 200 nM.

114. The dual transporter of any one of claims 1-29, 33-51, 55-77, and 79-107, wherein
(a) the first binding region binds to TfR with an affinity of about 250 nM to about 6000 nM and the second binding region binds to CD98hc with an affinity of about 1 nM to about 250 nM;
(b) the first binding region binds to TfR with an affinity of about 250 nM to about 1000 nM and the second binding region binds to CD98hc with an affinity of about 1 nM to about 100 nM; or
(c) the first binding region binds to TfR with an affinity of about 50 nM to about 250 nM and the second binding region binds to CD98hc with an affinity of about 1 nM to about 100 nM.

115. The dual transporter of any one of claims 1-29, 33-51, 55-77, and 79-107, wherein
(a) the first binding region binds to TfR with an affinity of about 1000 nM to about 6000 nM and the second binding region binds to CD98hc with an affinity of about 1 nM to about 500 nM;
(b) the first binding region binds to TfR with an affinity of about 250 nM to about 1000 nM and the second binding region binds to CD98hc with an affinity of about 250 nM to about 1000 nM;
(c) the first binding region binds to TfR with an affinity of about 50 nM to about 250 nM and the second binding region binds to CD98hc with an affinity of about 500 nM to about 6000 nM.

116. The dual transporter of any one of claims 1-29, 33-51, 55-77, and 79-107, wherein the dual transporter binds to TfR with an affinity of about 50 nM to about 1000 nM and binds to CD98hc with an affinity of about 5- to about 10-fold less than the affinity to TfR.

117. The dual transporter of any one of claims 1-29, 33-51, 55-77, and 79-107, wherein the dual transporter binds to TfR with an affinity of about 50 nM to about 6000 nM and binds to CD98hc with an affinity of about 5- to about 10-fold greater than the affinity to TfR.

118. The dual transporter of any one of claims 1-117, wherein the dual transporter is capable of being actively transported across the blood brain barrier.

119. The dual transporter of any one of claims 1-117, wherein the dual transporter is capable facilitating transport of a therapeutic agent across the blood brain barrier.

120. The dual transporter of claim 119, wherein the therapeutic agent targets: ABCA1, ABCA7, ADAM17, ALK, alpha-synuclein or a derivative or fragment thereof, an amyloid-beta peptide or a derivative or fragment thereof, AXL, B7H3, BCMA, CD125, CD166, CD19, CD20, CD205, CD22, CD25, CD25, CD30, CD33, CD37, CD39, CD44v6, CD46, CD56 (NCAM), CD73, CD79b, CDH6 (cadherin 6), CEACAM 5 (CD66E), CR1, EGFR viii, EGFR, ETBR, FGFR (1-4), Folate Receptor alpha, GAL-3BP (galectin binding protein), GD2, GD3, GloboH (globohexasylceramide), gp100, gpNMB, HER2, HER3, HER4, HLA-DR1, HLA-DR5, huntingtin, IGFR1, IL1RAP, IL-34, KIT, LIV1A, LRRC15 (leucine rich repeat containing 15), MET, MS4A4A, MS4A4E, MS4A6A, NaPi2B, TAR DNA-binding protein 43 (TDP-43) or a derivative or fragment thereof, PDL1, PILRA, PMEL17, PRAME, PSMA, pTau, PTK7 (CCK4; colon carcinoma kinase), RON, ROR1, Siglec11, SORL1, Tau or a derivative or fragment thereof, TF (tissue factor), transthyretin, TREM2, TREML2, TROP2, or a tumor cell.

121. A nucleic acid encoding the dual transporter of any one of claims 1-120.

122. A vector comprising the nucleic acid of claim 121.

123. A host cell expressing the dual transporter of any one of claims 1-120.

124. The host cell of claim 110, wherein the cell comprises the nucleic acid of claim 121 or the vector of claim 121.

125. A method for producing a dual transporter comprising:
(a) culturing a host cell comprising one or more nucleic acids encoding the dual transporter of any one of claims 1-120 under conditions in which the dual transporter encoded by the nucleic acid or vector is expressed; and
(b) isolating the dual transporter from the culture.

126. A composition comprising the dual transporter of any one of claims 1-120.

127. A pharmaceutical composition comprising the dual transporter of any one of claims 1-120 or the composition of claim 126, and a pharmaceutically acceptable excipient.

128. A method of delivering a molecule to the brain of a subject comprising administering the dual transporter of any one of claims 1-120 to the subject, the composition of claim 126, or the pharmaceutical composition of claim 127, wherein the dual transporter is linked to the molecule.

129. The method of claim 128, wherein the molecule comprises a therapeutic agent.

130. A method of delivering a molecule across the BBB into the brain of a subject comprising administering the dual transporter of any one of claims 1-120, the composition of claim 126, or the pharmaceutical composition of claim 127 to the subject, wherein the molecule is linked to the dual transporter.

131. The method of claim 130, wherein the molecule comprises a therapeutic agent

132. The method of claim 130 or 131, wherein the therapeutic agent targets: ABCA1, ABCA7, ADAM17, ALK, alpha-synuclein or a derivative or fragment thereof, an amyloid-beta peptide or a derivative or fragment thereof, AXL, B7H3, BCMA, CD125, CD166, CD19, CD20, CD205, CD22, CD25, CD25, CD30, CD33, CD37, CD39, CD44v6, CD46, CD56 (NCAM), CD73, CD79b, CDH6 (cadherin 6), CEACAM 5 (CD66E), CR1, EGFR viii, EGFR, ETBR, FGFR (1-4), Folate Receptor alpha, GAL-3BP (galectin binding protein), GD2, GD3, GloboH (globohexasylceramide), gp100, gpNMB, HER2, HER3, HER4, HLA-DR1, HLA-DR5, huntingtin, IGFR1, IL1RAP, IL-34, KIT, LIV1A, LRRC15 (leucine rich repeat containing 15), MET, MS4A4A, MS4A4E, MS4A6A, NaPi2B, TAR DNA-binding protein 43 (TDP-43) or a derivative or fragment thereof, PDL1, PILRA, PMEL17, PRAME, PSMA, pTau, PTK7 (CCK4; colon carcinoma kinase), RON, ROR1, Siglec11, SORL1, Tau or a derivative or fragment thereof, TF (tissue factor), transthyretin, TREM2, TREML2, TROP2, or a tumor cell.

133. A method of treating a disease or condition of the central nervous system (CNS) in a subject comprising administering the dual transporter of any one of claims 1-120, the composition of claim 126, or the pharmaceutical composition of claim 127 to the subject.

134. The method of claim 133, wherein the disease or condition of the CNS comprises: a neurological disease or condition, a neurodegenerative disease or condition, cancer, or an infection.

135. The method of claim 133, wherein the subject has or has been diagnosed with a neurological disease or condition, a neurodegenerative disease or condition, a cancer of the CNS or brain, or an infection of the CNS or brain.

136. A method of manufacturing a dual transporter comprising:
(a) providing (i) a first polypeptide comprising a first binding region known to specifically binds to a BBB transport protein and (ii) a second polypeptide comprising a second binding region known to specifically binds a second protein, wherein the second protein is expressed on the luminal surface of the BBB or is a brain retention protein, and wherein the second protein is different from the BBB transport protein; and
(b) forming a single molecule that incorporates the first polypeptide and the second polypeptide, thereby forming the dual transporter;
wherein the dual transporter has increased and/or prolonged brain exposure in an animal as compared to a corresponding control molecule that binds to only the BBB transport protein or only the second protein.

137. The method of claim 136, wherein the BBB transport protein is TfR or CD98hc.

138. The method of claim 136, wherein the BBB transport protein is TfR.

139. The method of any one of claims 136-138, wherein the second protein is selected from the group consisting of: TfR, CD98hc, Large neutral amino acids transporter small subunit 1 (CD98 light chain), GLUT1, MFSD2A, CA-IV, Low density lipoprotein receptor, IGF1R, Insulin-like growth factor 2 receptor, IgG receptor FcRn large subunit p51, Low density lipoprotein receptor-related protein 1, Low density lipoprotein receptor-related protein 2, Insulin receptor, Cell cycle control protein 50A, Transmembrane protein 50A, Basigin, Leptin Receptor, Claudin-5, P-selectin, Lactoferrin receptor, Folate receptor, Sodium-dependent lysophosphatidylcholine symporter 1, Solute carrier organic anion transporter family member 1C1, Sodium-coupled neutral amino acid transporter 5, LDL receptor-related protein 8, High affinity cationic amino acid transporter 1, Sodium- and chloride-dependent taurine transporter, Insulin-like growth factor-binding protein 7, Solute carrier family 40 member 1, Zinc transporter 6, heparin-binding epidermal growth factor-like growth factor, and MOG.

140. The method of claim 138 or 139, wherein the second protein is CD98hc.

141. The method of claim 140, wherein the first binding domain comprises the first binding domain of any one of claims 9-10, 13-29, 34, and 45-72; and the second binding domain comprises the second binding domain of any one of claims 11-29, 35, and 73-94.

142. The method of claim 136 or 137, wherein the second binding region specifically binds CD98hc.

143. A method of identifying a dual transporter comprising:
(a) providing (i) a first polypeptide comprising a first binding region known to specifically binds to a BBB transport protein and (ii) a second polypeptide comprising a second binding region known to specifically binds a second protein, wherein the second protein is expressed on the luminal surface of the BBB or is a brain retention protein, and wherein the second protein is different from the BBB transport protein;
(b) forming a single molecule that incorporates the first polypeptide and the second polypeptide; and
(c) measuring the brain concentration of the single molecule at a predetermined time and identifying the single molecule as a dual transporter if the single molecule is present in the brain at a higher concentration than a corresponding control molecule that binds only to the BBB transport protein or the second protein.

144. The method of claim 143, further comprising administering the single molecule peripherally to the animal prior to step (c).

145. The method of claim 144, wherein the animal is a non-human animal, optionally wherein the animal is a mouse, rat, or non-human primate.

146. The method of any one of claims 143-145, wherein presence in the brain at a higher concentration comprises increased rate of transport to the brain or prolonged brain exposure.

147. The method of any one of claims 143-146, wherein the method further comprises determining the affinity or avidity of the first binding region for the BBB transport protein and determining the affinity or avidity of the second binding region for the second protein.

148. The method of any one of claims 146-147, wherein the BBB transport protein is TfR or CD98hc.

149. The method of claim 148, wherein the BBB transport protein is TfR.

150. The method of any one of claims 143-149, wherein the second protein is selected from the group consisting of: TfR, CD98hc, CD981c, GLUT1, MFSD2A, CA-IV, Low density lipoprotein receptor, IGF1R, Insulin-like growth factor 2 receptor, IgG receptor FcRn large subunit p51, Low density lipoprotein receptor-related protein 1, Low density lipoprotein receptor-related protein 2, Insulin receptor, Cell cycle control protein 50A, Transmembrane protein 50A, Basigin, Leptin Receptor, Claudin-5, P-selectin, Lactoferrin receptor, Folate receptor, Sodium-dependent lysophosphatidylcholine symporter 1, Solute carrier organic anion transporter family member 1C1, Sodium-coupled neutral amino acid transporter 5, LDL receptor-related protein 8, High affinity cationic amino acid transporter 1, Sodium- and chloride-dependent taurine transporter, Insulin-like growth factor-binding protein 7, Solute carrier family 40 member 1, Zinc transporter 6, heparin-binding epidermal growth factor-like growth factor, and MOG.

151. The method of claim 149 or 150, wherein the second protein is CD98hc.

152. The method of claim 151, wherein the first binding domain comprises the first binding domain of any one of claims 9-10, 13-29, 34, and 45-72; and the second binding domain comprises the second binding domain of any one of claims 11-29, 35, and 73-94.

153. The method of any one of claims 143-152, further comprising measuring the binding of the single molecule to the BBB transport protein and/or the second protein.

154. The method of claim 153, wherein the binding is measured using surface plasmon resonance, ELISA, or flow cytometry.

155. The method of claim 153 or 154, wherein the binding is measured on the surface of a cell that expresses on its surface the BBB transport protein, the second protein, or both.

156. A method for improving delivery to the central nervous system of a therapeutic agent linked to a delivery vehicle having a first binding region that specially binds to a BBB transport protein, the method comprising linking the delivery vehicle to a second binding region that specifically binds to a second protein wherein the second protein is expressed on the luminal surface of the BBB or is a brain retention protein, and wherein the second protein is different from the BBB transport protein.

157. A method for improving delivery to the central nervous system of a delivery vehicle having a first binding region the specially binds to a BBB transport protein, the method comprising linking the delivery vehicle to a second binding region that specifically binds to a second protein wherein the second protein is expressed on the luminal surface of the BBB or is a brain retention protein, and wherein the second protein is different from the BBB transport protein.

158. The method of claim 156 or 157, wherein the BBB transport protein is TfR or CD98hc.

159. The method of claim 156 or 157, wherein the BBB transport protein is TfR and the second protein is CD98hc.

160. A method for improving delivery of a therapeutic agent to the central nervous system wherein the therapeutic agent is linked to a polypeptide that specifically binds TfR, the method comprising linking the polypeptide that specifically binds TfR to a second binding region that specifically binds to a second protein wherein the second protein is expressed on the luminal surface of the BBB or is a brain retention protein, and wherein the second protein is not TfR.

161. A method for improving delivery of a therapeutic agent to the central nervous system wherein the therapeutic agent is linked to a polypeptide that specifically binds CD98hc, the method comprising linking the polypeptide that specifically binds CD98hc to a second binding region that specifically binds to a second protein wherein the second protein is expressed on the luminal surface of the BBB or is a brain retention protein, and wherein the second protein is not CD98hc.
